SEQUENCE LISTING (126 sequences)

SEQ ID NO: 1 - protein, 5 aa, synthetic construct
SYGMH

SEQ ID NO: 2 - protein, 17 aa, synthetic construct
VIWFDGTKKYYTDSVKG

SEQ ID NO: 3 - protein, 15 aa, synthetic construct
DRGIGARRGPYYMDV

SEQ ID NO: 4 - protein, 11 aa, synthetic construct
RASQSISSYLN

SEQ ID NO: 5 - protein, 6 aa, synthetic construct
ASSLQS

SEQ ID NO: 6 - protein, 9 aa, synthetic construct
QQSYSTPLT

SEQ ID NO: 7 - protein, 124 aa, synthetic construct
QVQLVESGGGVVQPGRSLRLSCAASGFAFSSYGMHWVRQAPGKGLEWVAVIWFDGTKKYYTDSVKGRFTISRDNSKNTLYLQMNTLRAEDTAVYYCARDRGIGARRGPYYMDVWGKGTTVTVSS

SEQ ID NO: 8 - protein, 110 aa, synthetic construct
DIQMTQSPSSLSASVGDRVTITCRASQSISSYLNWYQQKPGKAPKLLIYAASSLQSGVPSRFSGSGSGTDFTLTISSLQPEDFATYYCQQSYSTPLTFGGGTKVEIKRTV

SEQ ID NO: 9 - DNA, 372 bp, synthetic construct
  source 1..372: mol_type = other DNA
caggtgcagctggtggaaagcggcggcggcgtggtgcagccgggccgcagcctgcgcctgagctgcgcggcgagcggctttgcgtttagcagctatggcatgcattgggtgcgccaggcgccgggcaaaggcctggaatgggtggcggtgatttggtttgatggcaccaaaaaatattataccgatagcgtgaaaggccgctttaccattagccgcgataacagcaaaaacaccctgtatctgcagatgaacaccctgcgcgcggaagataccgcggtgtattattgcgcgcgcgatcgcggcattggcgcgcgccgcggcccgtattatatggatgtgtggggcaaaggcaccaccgtgaccgtgagcagc

SEQ ID NO: 10 - DNA, 324 bp, synthetic construct
  source 1..324: mol_type = other DNA
gatattcagatgacccagagcccgagcagcctgagcgcgagcgtgggcgatcgcgtgaccattacctgccgcgcgagccagagcattagcagctatctgaactggtatcagcagaaaccgggcaaagcgccgaaactgctgatttatgcggcgagcagcctgcagagcggcgtgccgagccgctttagcggcagcggcagcggcaccgattttaccctgaccattagcagcctgcagccggaagattttgcgacctattattgccagcagagctatagcaccccgctgacctttggcggcggcaccaaagtggaaattaaacgc

SEQ ID NO: 11 - protein, 254 aa, synthetic construct
QVQLVESGGGVVQPGRSLRLSCAASGFAFSSYGMHWVRQAPGKGLEWVAVIWFDGTKKYYTDSVKGRFTISRDNSKNTLYLQMNTLRAEDTAVYYCARDRGIGARRGPYYMDVWGKGTTVTVSSGGGGSGGGGSGGGGSGGGGSDIQMTQSPSSLSASVGDRVTITCRASQSISSYLNWYQQKPGKAPKLLIYAASSLQSGVPSRFSGSGSGTDFTLTISSLQPEDFATYYCQQSYSTPLTFGGGTKVEIKRTV

SEQ ID NO: 12 - protein, 254 aa, synthetic construct
DIQMTQSPSSLSASVGDRVTITCRASQSISSYLNWYQQKPGKAPKLLIYAASSLQSGVPSRFSGSGSGTDFTLTISSLQPEDFATYYCQQSYSTPLTFGGGTKVEIKRTVGGGGSGGGGSGGGGSGGGGSQVQLVESGGGVVQPGRSLRLSCAASGFAFSSYGMHWVRQAPGKGLEWVAVIWFDGTKKYYTDSVKGRFTISRDNSKNTLYLQMNTLRAEDTAVYYCARDRGIGARRGPYYMDVWGKGTTVTVSS

SEQ ID NO: 13 - DNA, 756 bp, synthetic construct
  source 1..756: mol_type = other DNA
caggtgcagctggtggaaagcggcggcggcgtggtgcagccgggccgcagcctgcgcctgagctgcgcggcgagcggctttgcgtttagcagctatggcatgcattgggtgcgccaggcgccgggcaaaggcctggaatgggtggcggtgatttggtttgatggcaccaaaaaatattataccgatagcgtgaaaggccgctttaccattagccgcgataacagcaaaaacaccctgtatctgcagatgaacaccctgcgcgcggaagataccgcggtgtattattgcgcgcgcgatcgcggcattggcgcgcgccgcggcccgtattatatggatgtgtggggcaaaggcaccaccgtgaccgtgagcagcggcggaggtggaagcggaggcggaggatcaggtggcggtggatctggcggtggcggatctgatattcagatgacccagagcccgagcagcctgagcgcgagcgtgggcgatcgcgtgaccattacctgccgcgcgagccagagcattagcagctatctgaactggtatcagcagaaaccgggcaaagcgccgaaactgctgatttatgcggcgagcagcctgcagagcggcgtgccgagccgctttagcggcagcggcagcggcaccgattttaccctgaccattagcagcctgcagccggaagattttgcgacctattattgccagcagagctatagcaccccgctgacctttggcggcggcaccaaagtggaaattaaacgc

SEQ ID NO: 14 - DNA, 763 bp, synthetic construct
  source 1..763: mol_type = other DNA
gatatccagatgactcagagccccagcagcctgtctgcctctgtgggagacagagtgaccatcacctgtagagccagccagagcatcagcagctacctgaactggtatcagcagaagcccggcaaggcccctaaactgctgatctatgccgcctccagtctgcagagcggagtgccttctagattttccggcagcggctccggcaccgatttcaccctgaccatatctagcctgcagcctgaggacttcgccacctactactgccagcagagctacagcacccctctgacttttggcggaggcaccaaggtggaaatcaagcggacagtgtggcggaggtggaagcggaggcggaggatcaggtggcggtggatctggcggtggcggatctcaggtgcagctggtggaaagcggcggcggcgtggtgcagccgggccgcagcctgcgcctgagctgcgcggcgagcggctttgcgtttagcagctatggcatgcattgggtgcgccaggcgccgggcaaaggcctggaatgggtggcggtgatttggtttgatggcaccaaaaaatattataccgatagcgtgaaaggccgctttaccattagccgcgataacagcaaaaacaccctgtatctgcagatgaacaccctgcgcgcggaagataccgcggtgtattattgcgcgcgcgatcgcggcattggcgcgcgccgcggcccgtattatatggatgtgtggggcaaaggcaccaccgtgaccgtgagcagc

SEQ ID NO: 15 - protein, 618 aa, synthetic construct
MALPVTALLLPLALLLHAARPGSQVQLVESGGGVVQPGRSLRLSCAASGFAFSSYGMHWVRQAPGKGLEWVAVIWFDGTKKYYTDSVKGRFTISRDNSKNTLYLQMNTLRAEDTAVYYCARDRGIGARRGPYYMDVWGKGTTVTVSSGGGGSGGGGSGGGGSGGGGSDIQMTQSPSSLSASVGDRVTITCRASQSISSYLNWYQQKPGKAPKLLIYAASSLQSGVPSRFSGSGSGTDFTLTISSLQPEDFATYYCQQSYSTPLTFGGGTKVEIKRTVSGTTTPAPRPPTPAPTIASQPLSLRPEACRPAAGGAVHTRGLDFACDFWVLVVVGGVLACYSLLVTVAFIIFWVRSKRSTSGSGATNFSLLKQAGDVEENPGPASMVSKGEEDNMAIIKEFMRFKVHMEGSVNGHEFEIEGEGEGRPYEGTQTAKLKVTKGGPLPFAWDILSPQFMYGSKAYVKHPADIPDYLKLSFPEGFKWERVMNFEDGGVVTVTQDSSLQDGEFIYKVKLRGTNFPSDGPVMQKKTMGWEASSERMYPEDGALKGEIKQRLKLKDGGHYDAEVKTTYKAKKPVQLPGAYNVNIKLDITSHNEDYTIVEQYERAEGRHSTGGMDELYK

SEQ ID NO: 16 - protein, 618 aa, synthetic construct
MALPVTALLLPLALLLHAARPGSDIQMTQSPSSLSASVGDRVTITCRASQSISSYLNWYQQKPGKAPKLLIYAASSLQSGVPSRFSGSGSGTDFTLTISSLQPEDFATYYCQQSYSTPLTFGGGTKVEIKRTVGGGGSGGGGSGGGGSGGGGSQVQLVESGGGVVQPGRSLRLSCAASGFAFSSYGMHWVRQAPGKGLEWVAVIWFDGTKKYYTDSVKGRFTISRDNSKNTLYLQMNTLRAEDTAVYYCARDRGIGARRGPYYMDVWGKGTTVTVSSSGTTTPAPRPPTPAPTIASQPLSLRPEACRPAAGGAVHTRGLDFACDFWVLVVVGGVLACYSLLVTVAFIIFWVRSKRSTSGSGATNFSLLKQAGDVEENPGPASMVSKGEEDNMAIIKEFMRFKVHMEGSVNGHEFEIEGEGEGRPYEGTQTAKLKVTKGGPLPFAWDILSPQFMYGSKAYVKHPADIPDYLKLSFPEGFKWERVMNFEDGGVVTVTQDSSLQDGEFIYKVKLRGTNFPSDGPVMQKKTMGWEASSERMYPEDGALKGEIKQRLKLKDGGHYDAEVKTTYKAKKPVQLPGAYNVNIKLDITSHNEDYTIVEQYERAEGRHSTGGMDELYK

SEQ ID NO: 17 - DNA, 1851 bp, synthetic construct
  source 1..1851: mol_type = other DNA
atggccctgcctgtgacagccctgctgctgcctctggctctgctgctgcatgccgctagacccggatcccaggtgcagctggtggaaagcggcggcggcgtggtgcagccgggccgcagcctgcgcctgagctgcgcggcgagcggctttgcgtttagcagctatggcatgcattgggtgcgccaggcgccgggcaaaggcctggaatgggtggcggtgatttggtttgatggcaccaaaaaatattataccgatagcgtgaaaggccgctttaccattagccgcgataacagcaaaaacaccctgtatctgcagatgaacaccctgcgcgcggaagataccgcggtgtattattgcgcgcgcgatcgcggcattggcgcgcgccgcggcccgtattatatggatgtgtggggcaaaggcaccaccgtgaccgtgagcagcggcggaggtggaagcggaggcggaggatcaggtggcggtggatctggcggtggcggatctgatattcagatgacccagagcccgagcagcctgagcgcgagcgtgggcgatcgcgtgaccattacctgccgcgcgagccagagcattagcagctatctgaactggtatcagcagaaaccgggcaaagcgccgaaactgctgatttatgcggcgagcagcctgcagagcggcgtgccgagccgctttagcggcagcggcagcggcaccgattttaccctgaccattagcagcctgcagccggaagattttgcgacctattattgccagcagagctatagcaccccgctgacctttggcggcggcaccaaagtggaaattaaacgctccggaaccacgacgccagcgccgcgaccaccaacaccggcgcccaccatcgcgtcgcagcccctgtccctgcgcccagaggcgtgccggccagcggcggggggcgcagtgcacacgagggggctggacttcgcctgtgatttttgggtgctggtggtggttggtggagtcctggcttgctatagcttgctagtaacagtggcctttattattttctgggtgaggagtaagaggagcactagtggaagcggagctactaacttcagcctgctgaagcaggctggagacgtggaggagaaccctggacctgctagcatggtgagcaagggcgaggaggataacatggccatcatcaaggagttcatgcgcttcaaggtgcacatggagggctccgtgaacggccacgagttcgagatcgagggcgagggcgagggccgcccctacgagggcacccagaccgccaagctgaaggtgaccaagggtggccccctgcccttcgcctgggacatcctgtcccctcagttcatgtacggctccaaggcctacgtgaagcaccccgccgacatccccgactacttgaagctgtccttccccgagggcttcaagtgggagcgcgtgatgaacttcgaggacggcggcgtggtgaccgtgacccaggactcctccctgcaggacggcgagttcatctacaaggtgaagctgcgcggcaccaacttcccctccgacggccccgtaatgcagaagaagaccatgggctgggaggcctcctccgagcggatgtaccccgaggacggcgccctgaagggcgagatcaagcagaggctgaagctgaaggacggcggccactacgacgctgaggtcaagaccacctacaaggccaagaagcccgtgcagctgcccggcgcctacaacgtcaacatcaagttggacatcacctcccacaacgaggactacaccatcgtggaacagtacgaacgcgccgagggccgccactccaccggcggcatggacgagctgtacaagtaa

SEQ ID NO: 18 - DNA, 1857 bp, synthetic construct
  source 1..1857: mol_type = other DNA
atggccctgcctgtgacagccctgctgctgcctctggctctgctgctgcatgccgctagacccggatccgatatccagatgactcagagccccagcagcctgtctgcctctgtgggagacagagtgaccatcacctgtagagccagccagagcatcagcagctacctgaactggtatcagcagaagcccggcaaggcccctaaactgctgatctatgccgcctccagtctgcagagcggagtgccttctagattttccggcagcggctccggcaccgatttcaccctgaccatatctagcctgcagcctgaggacttcgccacctactactgccagcagagctacagcacccctctgacttttggcggaggcaccaaggtggaaatcaagcggacagtgtggcggaggtggaagcggaggcggaggatcaggtggcggtggatctggcggtggcggatctcaggtgcagctggtggaaagcggcggcggcgtggtgcagccgggccgcagcctgcgcctgagctgcgcggcgagcggctttgcgtttagcagctatggcatgcattgggtgcgccaggcgccgggcaaaggcctggaatgggtggcggtgatttggtttgatggcaccaaaaaatattataccgatagcgtgaaaggccgctttaccattagccgcgataacagcaaaaacaccctgtatctgcagatgaacaccctgcgcgcggaagataccgcggtgtattattgcgcgcgcgatcgcggcattggcgcgcgccgcggcccgtattatatggatgtgtggggcaaaggcaccaccgtgaccgtgagcagcccggaaccacgacgccagcgccgcgaccaccaacaccggcgcccaccatcgcgtcgcagcccctgtccctgcgcccagaggcgtgccggccagcggcggggggcgcagtgcacacgagggggctggacttcgcctgtgatttttgggtgctggtggtggttggtggagtcctggcttgctatagcttgctagtaacagtggcctttattattttctgggtgaggagtaagaggagcactagtggaagcggagctactaacttcagcctgctgaagcaggctggagacgtggaggagaaccctggacctgctagcatggtgagcaagggcgaggaggataacatggccatcatcaaggagttcatgcgcttcaaggtgcacatggagggctccgtgaacggccacgagttcgagatcgagggcgagggcgagggccgcccctacgagggcacccagaccgccaagctgaaggtgaccaagggtggccccctgcccttcgcctgggacatcctgtcccctcagttcatgtacggctccaaggcctacgtgaagcaccccgccgacatccccgactacttgaagctgtccttccccgagggcttcaagtgggagcgcgtgatgaacttcgaggacggcggcgtggtgaccgtgacccaggactcctccctgcaggacggcgagttcatctacaaggtgaagctgcgcggcaccaacttcccctccgacggccccgtaatgcagaagaagaccatgggctgggaggcctcctccgagcggatgtaccccgaggacggcgccctgaagggcgagatcaagcagaggctgaagctgaaggacggcggccactacgacgctgaggtcaagaccacctacaaggccaagaagcccgtgcagctgcccggcgcctacaacgtcaacatcaagttggacatcacctcccacaacgaggactacaccatcgtggaacagtacgaacgcgccgagggccgccactccaccggcggcatggacgagctgtacaagtaa

SEQ ID NO: 19 - protein, 10 aa, synthetic construct
GFTFSSYGMS

SEQ ID NO: 20 - protein, 16 aa, synthetic construct
SINSNGGSTYYPDSVK

SEQ ID NO: 21 - skipped

SEQ ID NO: 22 - protein, 16 aa, synthetic construct
RSSQSLVYSNGDTYLH

SEQ ID NO: 23 - protein, 7 aa, synthetic construct
KVSNRFS

SEQ ID NO: 24 - protein, 9 aa, synthetic construct
SQSTHVPWT

SEQ ID NO: 25 - protein, 112 aa, synthetic construct
EVQLVESGGGLVQPGGSLRLSCAASGFTFSSYGMSWVRQAPGKGLELVASINSNGGSTYYPDSVKGRFTISRDNAKNSLYLQMNSLRAEDTAVYYCASGDYWGQGTTVTVSS

SEQ ID NO: 26 - protein, 112 aa, synthetic construct
DIVMTQSPLSLPVTPGEPASISCRSSQSLVYSNGDTYLHWYLQKPGQSPQLLIYKVSNRFSGVPDRFSGSGSGTDFTLKISRVEAEDVGVYYCSQSTHVPWTFGQGTKVEIK

SEQ ID NO: 27 - DNA, 336 bp, synthetic construct
  source 1..336: mol_type = other DNA
gaggtgcagctggttgaatctggcggaggactggttcagcctggcggatctctgagactgtcttgtgccgccagcggcttcacctttagcagctacggcatgagctgggtccgacaggctcctggcaaaggactggaactggtggccagcatcaacagcaatggcggcagcacctactatcccgacagcgtgaagggcagattcaccatcagccgggacaacgccaagaacagcctgtacctgcagatgaactccctgagagccgaggacaccgccgtgtactattgtgccagcggcgattattggggccagggcacaacagtgacagtgtctagc

SEQ ID NO: 28 - DNA, 342 bp, synthetic construct
  source 1..342: mol_type = other DNA
gacattgtgatgacacagagccctctgagcctgccagtgacacctggcgaacctgccagcatctcctgtagaagcagccagagcctggtgtacagcaacggcgatacctacctgcactggtatctgcagaagcccggccagtctcctcagctgctgatctacaaggtgtccaaccggttcagcggcgtgcccgatagattttctggcagcggctctggcaccgacttcaccctgaagatctccagagtggaagccgaggatgtgggcgtgtactactgcagccagtctacccacgtgccatggacatttggacagggcaccaaggtggaaatcaagtccgga

SEQ ID NO: 29 - protein, 244 aa, synthetic construct
EVQLVESGGGLVQPGGSLRLSCAASGFTFSSYGMSWVRQAPGKGLELVASINSNGGSTYYPDSVKGRFTISRDNAKNSLYLQMNSLRAEDTAVYYCASGDYWGQGTTVTVSSGGGGSGGGGSGGGGSGGGGSDIVMTQSPLSLPVTPGEPASISCRSSQSLVYSNGDTYLHWYLQKPGQSPQLLIYKVSNRFSGVPDRFSGSGSGTDFTLKISRVEAEDVGVYYCSQSTHVPWTFGQGTKVEIK

SEQ ID NO: 30 - protein, 244 aa, synthetic construct
DIVMTQSPLSLPVTPGEPASISCRSSQSLVYSNGDTYLHWYLQKPGQSPQLLIYKVSNRFSGVPDRFSGSGSGTDFTLKISRVEAEDVGVYYCSQSTHVPWTFGQGTKVEIKGGGGSGGGGSGGGGSGGGGSEVQLVESGGGLVQPGGSLRLSCAASGFTFSSYGMSWVRQAPGKGLELVASINSNGGSTYYPDSVKGRFTISRDNAKNSLYLQMNSLRAEDTAVYYCASGDYWGQGTTVTVSS

SEQ ID NO: 31 - DNA, 738 bp, synthetic construct
  source 1..738: mol_type = other DNA
gaggtgcagctggttgaatctggcggaggactggttcagcctggcggatctctgagactgtcttgtgccgccagcggcttcacctttagcagctacggcatgagctgggtccgacaggctcctggcaaaggactggaactggtggccagcatcaacagcaatggcggcagcacctactatcccgacagcgtgaagggcagattcaccatcagccgggacaacgccaagaacagcctgtacctgcagatgaactccctgagagccgaggacaccgccgtgtactattgtgccagcggcgattattggggccagggcacaacagtgacagtgtctagcggcggaggtggaagcggaggcggaggatcaggtggcggtggatctggcggtggcggatctgacattgtgatgacacagagccctctgagcctgccagtgacacctggcgaacctgccagcatctcctgtagaagcagccagagcctggtgtacagcaacggcgatacctacctgcactggtatctgcagaagcccggccagtctcctcagctgctgatctacaaggtgtccaaccggttcagcggcgtgcccgatagattttctggcagcggctctggcaccgacttcaccctgaagatctccagagtggaagccgaggatgtgggcgtgtactactgcagccagtctacccacgtgccatggacatttggacagggcaccaaggtggaaatcaagtccgga

SEQ ID NO: 32 - DNA, 738 bp, synthetic construct
  source 1..738: mol_type = other DNA
gacattgtgatgacacagagccctctgagcctgccagtgacacctggcgaacctgccagcatctcctgtagaagcagccagagcctggtgtacagcaacggcgatacctacctgcactggtatctgcagaagcccggccagtctcctcagctgctgatctacaaggtgtccaaccggttcagcggcgtgcccgatagattttctggcagcggctctggcaccgacttcaccctgaagatctccagagtggaagccgaggatgtgggcgtgtactactgcagccagtctacccacgtgccatggacatttggacagggcaccaaggtggaaatcaagtccggaggcggaggtggaagcggaggcggaggatcaggtggcggtggatctggcggtggcggatctgaggtgcagctggttgaatctggcggaggactggttcagcctggcggatctctgagactgtcttgtgccgccagcggcttcacctttagcagctacggcatgagctgggtccgacaggctcctggcaaaggactggaactggtggccagcatcaacagcaatggcggcagcacctactatcccgacagcgtgaagggcagattcaccatcagccgggacaacgccaagaacagcctgtacctgcagatgaactccctgagagccgaggacaccgccgtgtactattgtgccagcggcgattattggggccagggcacaacagtgacagtgtctagc

SEQ ID NO: 33 - protein, 608 aa, synthetic construct
MALPVTALLLPLALLLHAARPGSEVQLVESGGGLVQPGGSLRLSCAASGFTFSSYGMSWVRQAPGKGLELVASINSNGGSTYYPDSVKGRFTISRDNAKNSLYLQMNSLRAEDTAVYYCASGDYWGQGTTVTVSSGGGGSGGGGSGGGGSGGGGSDIVMTQSPLSLPVTPGEPASISCRSSQSLVYSNGDTYLHWYLQKPGQSPQLLIYKVSNRFSGVPDRFSGSGSGTDFTLKISRVEAEDVGVYYCSQSTHVPWTFGQGTKVEIKSGTTTPAPRPPTPAPTIASQPLSLRPEACRPAAGGAVHTRGLDFACDFWVLVVVGGVLACYSLLVTVAFIIFWVRSKRSTSGSGATNFSLLKQAGDVEENPGPASMVSKGEEDNMAIIKEFMRFKVHMEGSVNGHEFEIEGEGEGRPYEGTQTAKLKVTKGGPLPFAWDILSPQFMYGSKAYVKHPADIPDYLKLSFPEGFKWERVMNFEDGGVVTVTQDSSLQDGEFIYKVKLRGTNFPSDGPVMQKKTMGWEASSERMYPEDGALKGEIKQRLKLKDGGHYDAEVKTTYKAKKPVQLPGAYNVNIKLDITSHNEDYTIVEQYERAEGRHSTGGMDELYK

SEQ ID NO: 34 - protein, 608 aa, synthetic construct
MALPVTALLLPLALLLHAARPGSDIVMTQSPLSLPVTPGEPASISCRSSQSLVYSNGDTYLHWYLQKPGQSPQLLIYKVSNRFSGVPDRFSGSGSGTDFTLKISRVEAEDVGVYYCSQSTHVPWTFGQGTKVEIKGGGGSGGGGSGGGGSGGGGSEVQLVESGGGLVQPGGSLRLSCAASGFTFSSYGMSWVRQAPGKGLELVASINSNGGSTYYPDSVKGRFTISRDNAKNSLYLQMNSLRAEDTAVYYCASGDYWGQGTTVTVSSSGTTTPAPRPPTPAPTIASQPLSLRPEACRPAAGGAVHTRGLDFACDFWVLVVVGGVLACYSLLVTVAFIIFWVRSKRSTSGSGATNFSLLKQAGDVEENPGPASMVSKGEEDNMAIIKEFMRFKVHMEGSVNGHEFEIEGEGEGRPYEGTQTAKLKVTKGGPLPFAWDILSPQFMYGSKAYVKHPADIPDYLKLSFPEGFKWERVMNFEDGGVVTVTQDSSLQDGEFIYKVKLRGTNFPSDGPVMQKKTMGWEASSERMYPEDGALKGEIKQRLKLKDGGHYDAEVKTTYKAKKPVQLPGAYNVNIKLDITSHNEDYTIVEQYERAEGRHSTGGMDELYK

SEQ ID NO: 35 - DNA, 1832 bp, synthetic construct
  source 1..1832: mol_type = other DNA
atggccctgcctgtgacagccctgctgctgcctctggctctgctgctgcatgccgctagacccggatccgaggtgcagctggttgaatctggcggaggactggttcagcctggcggatctctgagactgtcttgtgccgccagcggcttcacctttagcagctacggcatgagctgggtccgacaggctcctggcaaaggactggaactggtggccagcatcaacagcaatggcggcagcacctactatcccgacagcgtgaagggcagattcaccatcagccgggacaacgccaagaacagcctgtacctgcagatgaactccctgagagccgaggacaccgccgtgtactattgtgccagcggcgattattggggccagggcacaacagtgacagtgtctagcggcggaggtggaagcggaggcggaggatcaggtggcggtggatctggcggtggcggatctgacattgtgatgacacagagccctctgagcctgccagtgacacctggcgaacctgccagcatctcctgtagaagcagccagagcctggtgtacagcaacggcgatacctacctgcactggtatctgcagaagcccggccagtctcctcagctgctgatctacaaggtgtccaaccggttcagcggcgtgcccgatagattttctggcagcggctctggcaccgacttcaccctgaagatctccagagtggaagccgaggatgtgggcgtgtactactgcagccagtctacccacgtgccatggacatttggacagggcaccaaggtggaaatcaagtccggaccggaaccacgacgccagcgccgcgaccaccaacaccggcgcccaccatcgcgtcgcagcccctgtccctgcgcccagaggcgtgccggccagcggcggggggcgcagtgcacacgagggggctggacttcgcctgtgatttttgggtgctggtggtggttggtggagtcctggcttgctatagcttgctagtaacagtggcctttattattttctgggtgaggagtaagaggagcactagtggaagcggagctactaacttcagcctgctgaagcaggctggagacgtggaggagaaccctggacctgctagcatggtgagcaagggcgaggaggataacatggccatcatcaaggagttcatgcgcttcaaggtgcacatggagggctccgtgaacggccacgagttcgagatcgagggcgagggcgagggccgcccctacgagggcacccagaccgccaagctgaaggtgaccaagggtggccccctgcccttcgcctgggacatcctgtcccctcagttcatgtacggctccaaggcctacgtgaagcaccccgccgacatccccgactacttgaagctgtccttccccgagggcttcaagtgggagcgcgtgatgaacttcgaggacggcggcgtggtgaccgtgacccaggactcctccctgcaggacggcgagttcatctacaaggtgaagctgcgcggcaccaacttcccctccgacggccccgtaatgcagaagaagaccatgggctgggaggcctcctccgagcggatgtaccccgaggacggcgccctgaagggcgagatcaagcagaggctgaagctgaaggacggcggccactacgacgctgaggtcaagaccacctacaaggccaagaagcccgtgcagctgcccggcgcctacaacgtcaacatcaagttggacatcacctcccacaacgaggactacaccatcgtggaacagtacgaacgcgccgagggccgccactccaccggcggcatggacgagctgtacaagtaa

SEQ ID NO: 36 - DNA, 1832 bp, synthetic construct
  source 1..1832: mol_type = other DNA
atggccctgcctgtgacagccctgctgctgcctctggctctgctgctgcatgccgctagacccggatccgacattgtgatgacacagagccctctgagcctgccagtgacacctggcgaacctgccagcatctcctgtagaagcagccagagcctggtgtacagcaacggcgatacctacctgcactggtatctgcagaagcccggccagtctcctcagctgctgatctacaaggtgtccaaccggttcagcggcgtgcccgatagattttctggcagcggctctggcaccgacttcaccctgaagatctccagagtggaagccgaggatgtgggcgtgtactactgcagccagtctacccacgtgccatggacatttggacagggcaccaaggtggaaatcaagtccggaggcggaggtggaagcggaggcggaggatcaggtggcggtggatctggcggtggcggatctgaggtgcagctggttgaatctggcggaggactggttcagcctggcggatctctgagactgtcttgtgccgccagcggcttcacctttagcagctacggcatgagctgggtccgacaggctcctggcaaaggactggaactggtggccagcatcaacagcaatggcggcagcacctactatcccgacagcgtgaagggcagattcaccatcagccgggacaacgccaagaacagcctgtacctgcagatgaactccctgagagccgaggacaccgccgtgtactattgtgccagcggcgattattggggccagggcacaacagtgacagtgtctagcccggaaccacgacgccagcgccgcgaccaccaacaccggcgcccaccatcgcgtcgcagcccctgtccctgcgcccagaggcgtgccggccagcggcggggggcgcagtgcacacgagggggctggacttcgcctgtgatttttgggtgctggtggtggttggtggagtcctggcttgctatagcttgctagtaacagtggcctttattattttctgggtgaggagtaagaggagcactagtggaagcggagctactaacttcagcctgctgaagcaggctggagacgtggaggagaaccctggacctgctagcatggtgagcaagggcgaggaggataacatggccatcatcaaggagttcatgcgcttcaaggtgcacatggagggctccgtgaacggccacgagttcgagatcgagggcgagggcgagggccgcccctacgagggcacccagaccgccaagctgaaggtgaccaagggtggccccctgcccttcgcctgggacatcctgtcccctcagttcatgtacggctccaaggcctacgtgaagcaccccgccgacatccccgactacttgaagctgtccttccccgagggcttcaagtgggagcgcgtgatgaacttcgaggacggcggcgtggtgaccgtgacccaggactcctccctgcaggacggcgagttcatctacaaggtgaagctgcgcggcaccaacttcccctccgacggccccgtaatgcagaagaagaccatgggctgggaggcctcctccgagcggatgtaccccgaggacggcgccctgaagggcgagatcaagcagaggctgaagctgaaggacggcggccactacgacgctgaggtcaagaccacctacaaggccaagaagcccgtgcagctgcccggcgcctacaacgtcaacatcaagttggacatcacctcccacaacgaggactacaccatcgtggaacagtacgaacgcgccgagggccgccactccaccggcggcatggacgagctgtacaagtaa

SEQ ID NO: 37 - protein, 5 aa, synthetic construct
NYGMS

SEQ ID NO: 38 - protein, 17 aa, synthetic construct
IRSGGGRTYYSDNVKGR

SEQ ID NO: 39 - protein, 10 aa, synthetic construct
YDHYSGSSDY

SEQ ID NO: 40 - protein, 16 aa, synthetic construct
KSSQSLLDSDGKTYLN

SEQ ID NO: 41 - protein, 6 aa, synthetic construct
LVSKLD

SEQ ID NO: 42 - protein, 9 aa, synthetic construct
WQGTHFPRT

SEQ ID NO: 43 - protein, 121 aa, synthetic construct
EVKLVESGGGLVKPGASLKLSCAASGFTFSNYGMSWVRQNSDKRLEWVASIRSGGGRTYYSDNVKGRFTISRENAKNTLYLQMSSLKSEDTALYYCVRYDHYSGSSDYWGQGTTVTVSSSG

SEQ ID NO: 44 - protein, 112 aa, synthetic construct
YVVMTQTPLTLSVTIGQPASISCKSSQSLLDSDGKTYLNWLLQRPGQSPKRLIYLVSKLDSGVPDRFTGSGSGTDFTLKISRIEAEDLGLYYCWQGTHFPRTFGGGTKLEIK

SEQ ID NO: 45 - DNA, 363 bp, synthetic construct
  source 1..363: mol_type = other DNA
gaagtgaagctggttgaaagtggcggaggcctggttaagcctggcgcctctctgaaactgtcttgtgccgccagcggcttcaccttcagcaactacggcatgagctgggtccgacagaacagcgacaagcggcttgagtgggtcgcctctattagatccggcggaggcagaacctactactccgacaacgtgaagggcagattcaccatcagcagagagaacgccaagaacaccctgtacctgcagatgagcagcctgaagtccgaggacaccgctctgtactattgcgtcagatacgaccactacagcggcagcagcgattattggggccagggcacaaccgtgaccgtgtctagctccgga

SEQ ID NO: 46 - DNA, 336 bp, synthetic construct
  source 1..336: mol_type = other DNA
tacgtggtcatgacacagacccctctgacactgagcgtgaccattggacagcctgccagcatcagctgcaagagcagtcagagcctgctggactccgacggcaagacctacctgaattggctgctgcagaggcccggacagagccccaagagactgatctacctggtgtccaagctggacagcggcgtgcccgatagattcacaggctctggcagcggcaccgacttcaccctgaagatctctagaatcgaggccgaggacctgggcctgtactactgttggcagggcacacacttccccagaacctttggcggcggaacaaagctggaaatcaaa

SEQ ID NO: 47 - protein, 253 aa, synthetic construct
EVKLVESGGGLVKPGASLKLSCAASGFTFSNYGMSWVRQNSDKRLEWVASIRSGGGRTYYSDNVKGRFTISRENAKNTLYLQMSSLKSEDTALYYCVRYDHYSGSSDYWGQGTTVTVSSSGGGGGSGGGGSGGGGSGGGGSYVVMTQTPLTLSVTIGQPASISCKSSQSLLDSDGKTYLNWLLQRPGQSPKRLIYLVSKLDSGVPDRFTGSGSGTDFTLKISRIEAEDLGLYYCWQGTHFPRTFGGGTKLEIK

SEQ ID NO: 48 - protein, 253 aa, synthetic construct
YVVMTQTPLTLSVTIGQPASISCKSSQSLLDSDGKTYLNWLLQRPGQSPKRLIYLVSKLDSGVPDRFTGSGSGTDFTLKISRIEAEDLGLYYCWQGTHFPRTFGGGTKLEIKGGGGSGGGGSGGGGSGGGGSEVKLVESGGGLVKPGASLKLSCAASGFTFSNYGMSWVRQNSDKRLEWVASIRSGGGRTYYSDNVKGRFTISRENAKNTLYLQMSSLKSEDTALYYCVRYDHYSGSSDYWGQGTTVTVSSSG

SEQ ID NO: 49 - DNA, 759 bp, synthetic construct
  source 1..759: mol_type = other DNA
gaagtgaagctggttgaaagtggcggaggcctggttaagcctggcgcctctctgaaactgtcttgtgccgccagcggcttcaccttcagcaactacggcatgagctgggtccgacagaacagcgacaagcggcttgagtgggtcgcctctattagatccggcggaggcagaacctactactccgacaacgtgaagggcagattcaccatcagcagagagaacgccaagaacaccctgtacctgcagatgagcagcctgaagtccgaggacaccgctctgtactattgcgtcagatacgaccactacagcggcagcagcgattattggggccagggcacaaccgtgaccgtgtctagctccggaggcggaggtggaagcggaggcggaggatcaggtggcggtggatctggcggtggcggatcttacgtggtcatgacacagacccctctgacactgagcgtgaccattggacagcctgccagcatcagctgcaagagcagtcagagcctgctggactccgacggcaagacctacctgaattggctgctgcagaggcccggacagagccccaagagactgatctacctggtgtccaagctggacagcggcgtgcccgatagattcacaggctctggcagcggcaccgacttcaccctgaagatctctagaatcgaggccgaggacctgggcctgtactactgttggcagggcacacacttccccagaacctttggcggcggaacaaagctggaaatcaaa

SEQ ID NO: 50 - DNA, 759 bp, synthetic construct
  source 1..759: mol_type = other DNA
tacgtggtcatgacacagacccctctgacactgagcgtgaccattggacagcctgccagcatcagctgcaagagcagtcagagcctgctggactccgacggcaagacctacctgaattggctgctgcagaggcccggacagagccccaagagactgatctacctggtgtccaagctggacagcggcgtgcccgatagattcacaggctctggcagcggcaccgacttcaccctgaagatctctagaatcgaggccgaggacctgggcctgtactactgttggcagggcacacacttccccagaacctttggcggcggaacaaagctggaaatcaaaggcggaggtggaagcggaggcggaggatcaggtggcggtggatctggcggtggcggatctgaagtgaagctggttgaaagtggcggaggcctggttaagcctggcgcctctctgaaactgtcttgtgccgccagcggcttcaccttcagcaactacggcatgagctgggtccgacagaacagcgacaagcggcttgagtgggtcgcctctattagatccggcggaggcagaacctactactccgacaacgtgaagggcagattcaccatcagcagagagaacgccaagaacaccctgtacctgcagatgagcagcctgaagtccgaggacaccgctctgtactattgcgtcagatacgaccactacagcggcagcagcgattattggggccagggcacaaccgtgaccgtgtctagctccgga

SEQ ID NO: 51 - protein, 617 aa, synthetic construct
MALPVTALLLPLALLLHAARPGSEVKLVESGGGLVKPGASLKLSCAASGFTFSNYGMSWVRQNSDKRLEWVASIRSGGGRTYYSDNVKGRFTISRENAKNTLYLQMSSLKSEDTALYYCVRYDHYSGSSDYWGQGTTVTVSSSGGGGGSGGGGSGGGGSGGGGSYVVMTQTPLTLSVTIGQPASISCKSSQSLLDSDGKTYLNWLLQRPGQSPKRLIYLVSKLDSGVPDRFTGSGSGTDFTLKISRIEAEDLGLYYCWQGTHFPRTFGGGTKLEIKSGTTTPAPRPPTPAPTIASQPLSLRPEACRPAAGGAVHTRGLDFACDFWVLVVVGGVLACYSLLVTVAFIIFWVRSKRSTSGSGATNFSLLKQAGDVEENPGPASMVSKGEEDNMAIIKEFMRFKVHMEGSVNGHEFEIEGEGEGRPYEGTQTAKLKVTKGGPLPFAWDILSPQFMYGSKAYVKHPADIPDYLKLSFPEGFKWERVMNFEDGGVVTVTQDSSLQDGEFIYKVKLRGTNFPSDGPVMQKKTMGWEASSERMYPEDGALKGEIKQRLKLKDGGHYDAEVKTTYKAKKPVQLPGAYNVNIKLDITSHNEDYTIVEQYERAEGRHSTGGMDELYK

SEQ ID NO: 52 - protein, 617 aa, synthetic construct
MALPVTALLLPLALLLHAARPGSYVVMTQTPLTLSVTIGQPASISCKSSQSLLDSDGKTYLNWLLQRPGQSPKRLIYLVSKLDSGVPDRFTGSGSGTDFTLKISRIEAEDLGLYYCWQGTHFPRTFGGGTKLEIKGGGGSGGGGSGGGGSGGGGSEVKLVESGGGLVKPGASLKLSCAASGFTFSNYGMSWVRQNSDKRLEWVASIRSGGGRTYYSDNVKGRFTISRENAKNTLYLQMSSLKSEDTALYYCVRYDHYSGSSDYWGQGTTVTVSSSGSGTTTPAPRPPTPAPTIASQPLSLRPEACRPAAGGAVHTRGLDFACDFWVLVVVGGVLACYSLLVTVAFIIFWVRSKRSTSGSGATNFSLLKQAGDVEENPGPASMVSKGEEDNMAIIKEFMRFKVHMEGSVNGHEFEIEGEGEGRPYEGTQTAKLKVTKGGPLPFAWDILSPQFMYGSKAYVKHPADIPDYLKLSFPEGFKWERVMNFEDGGVVTVTQDSSLQDGEFIYKVKLRGTNFPSDGPVMQKKTMGWEASSERMYPEDGALKGEIKQRLKLKDGGHYDAEVKTTYKAKKPVQLPGAYNVNIKLDITSHNEDYTIVEQYERAEGRHSTGGMDELYK

SEQ ID NO: 53 - DNA, 1853 bp, synthetic construct
  source 1..1853: mol_type = other DNA
atggccctgcctgtgacagccctgctgctgcctctggctctgctgctgcatgccgctagacccggatccgaagtgaagctggttgaaagtggcggaggcctggttaagcctggcgcctctctgaaactgtcttgtgccgccagcggcttcaccttcagcaactacggcatgagctgggtccgacagaacagcgacaagcggcttgagtgggtcgcctctattagatccggcggaggcagaacctactactccgacaacgtgaagggcagattcaccatcagcagagagaacgccaagaacaccctgtacctgcagatgagcagcctgaagtccgaggacaccgctctgtactattgcgtcagatacgaccactacagcggcagcagcgattattggggccagggcacaaccgtgaccgtgtctagctccggaggcggaggtggaagcggaggcggaggatcaggtggcggtggatctggcggtggcggatcttacgtggtcatgacacagacccctctgacactgagcgtgaccattggacagcctgccagcatcagctgcaagagcagtcagagcctgctggactccgacggcaagacctacctgaattggctgctgcagaggcccggacagagccccaagagactgatctacctggtgtccaagctggacagcggcgtgcccgatagattcacaggctctggcagcggcaccgacttcaccctgaagatctctagaatcgaggccgaggacctgggcctgtactactgttggcagggcacacacttccccagaacctttggcggcggaacaaagctggaaatcaaaccggaaccacgacgccagcgccgcgaccaccaacaccggcgcccaccatcgcgtcgcagcccctgtccctgcgcccagaggcgtgccggccagcggcggggggcgcagtgcacacgagggggctggacttcgcctgtgatttttgggtgctggtggtggttggtggagtcctggcttgctatagcttgctagtaacagtggcctttattattttctgggtgaggagtaagaggagcactagtggaagcggagctactaacttcagcctgctgaagcaggctggagacgtggaggagaaccctggacctgctagcatggtgagcaagggcgaggaggataacatggccatcatcaaggagttcatgcgcttcaaggtgcacatggagggctccgtgaacggccacgagttcgagatcgagggcgagggcgagggccgcccctacgagggcacccagaccgccaagctgaaggtgaccaagggtggccccctgcccttcgcctgggacatcctgtcccctcagttcatgtacggctccaaggcctacgtgaagcaccccgccgacatccccgactacttgaagctgtccttccccgagggcttcaagtgggagcgcgtgatgaacttcgaggacggcggcgtggtgaccgtgacccaggactcctccctgcaggacggcgagttcatctacaaggtgaagctgcgcggcaccaacttcccctccgacggccccgtaatgcagaagaagaccatgggctgggaggcctcctccgagcggatgtaccccgaggacggcgccctgaagggcgagatcaagcagaggctgaagctgaaggacggcggccactacgacgctgaggtcaagaccacctacaaggccaagaagcccgtgcagctgcccggcgcctacaacgtcaacatcaagttggacatcacctcccacaacgaggactacaccatcgtggaacagtacgaacgcgccgagggccgccactccaccggcggcatggacgagctgtacaagtaa

SEQ ID NO: 54 - DNA, 1853 bp, synthetic construct
  source 1..1853: mol_type = other DNA
atggccctgcctgtgacagccctgctgctgcctctggctctgctgctgcatgccgctagacccggatcctacgtggtcatgacacagacccctctgacactgagcgtgaccattggacagcctgccagcatcagctgcaagagcagtcagagcctgctggactccgacggcaagacctacctgaattggctgctgcagaggcccggacagagccccaagagactgatctacctggtgtccaagctggacagcggcgtgcccgatagattcacaggctctggcagcggcaccgacttcaccctgaagatctctagaatcgaggccgaggacctgggcctgtactactgttggcagggcacacacttccccagaacctttggcggcggaacaaagctggaaatcaaaggcggaggtggaagcggaggcggaggatcaggtggcggtggatctggcggtggcggatctgaagtgaagctggttgaaagtggcggaggcctggttaagcctggcgcctctctgaaactgtcttgtgccgccagcggcttcaccttcagcaactacggcatgagctgggtccgacagaacagcgacaagcggcttgagtgggtcgcctctattagatccggcggaggcagaacctactactccgacaacgtgaagggcagattcaccatcagcagagagaacgccaagaacaccctgtacctgcagatgagcagcctgaagtccgaggacaccgctctgtactattgcgtcagatacgaccactacagcggcagcagcgattattggggccagggcacaaccgtgaccgtgtctagctccggaccggaaccacgacgccagcgccgcgaccaccaacaccggcgcccaccatcgcgtcgcagcccctgtccctgcgcccagaggcgtgccggccagcggcggggggcgcagtgcacacgagggggctggacttcgcctgtgatttttgggtgctggtggtggttggtggagtcctggcttgctatagcttgctagtaacagtggcctttattattttctgggtgaggagtaagaggagcactagtggaagcggagctactaacttcagcctgctgaagcaggctggagacgtggaggagaaccctggacctgctagcatggtgagcaagggcgaggaggataacatggccatcatcaaggagttcatgcgcttcaaggtgcacatggagggctccgtgaacggccacgagttcgagatcgagggcgagggcgagggccgcccctacgagggcacccagaccgccaagctgaaggtgaccaagggtggccccctgcccttcgcctgggacatcctgtcccctcagttcatgtacggctccaaggcctacgtgaagcaccccgccgacatccccgactacttgaagctgtccttccccgagggcttcaagtgggagcgcgtgatgaacttcgaggacggcggcgtggtgaccgtgacccaggactcctccctgcaggacggcgagttcatctacaaggtgaagctgcgcggcaccaacttcccctccgacggccccgtaatgcagaagaagaccatgggctgggaggcctcctccgagcggatgtaccccgaggacggcgccctgaagggcgagatcaagcagaggctgaagctgaaggacggcggccactacgacgctgaggtcaagaccacctacaaggccaagaagcccgtgcagctgcccggcgcctacaacgtcaacatcaagttggacatcacctcccacaacgaggactacaccatcgtggaacagtacgaacgcgccgagggccgccactccaccggcggcatggacgagctgtacaagtaa

SEQ ID NO: 55 - protein, 5 aa, synthetic construct
KYGMS

SEQ ID NO: 56 - protein, 16 aa, synthetic construct
ISSSGSRTYYPDSVKG

SEQ ID NO: 57 - protein, 7 aa, synthetic construct
WDGAMDY

SEQ ID NO: 58 - protein, 16 aa, synthetic construct
KSSQSIVHSNGNTYLE

SEQ ID NO: 59 - protein, 6 aa, synthetic construct
KVSNRF

SEQ ID NO: 60 - protein, 9 aa, synthetic construct
FQGSLVPWA

SEQ ID NO: 61 - protein, 117 aa, synthetic construct
EVHLVESGGALVKPGGSLRLSCAASGFSFSKYGMSWVRQAPGKGLEWVATISSSGSRTYYPDSVKGRFTISRDNAKNTLYLQMNSLRAEDTAMYYCSISWDGAMDYWGQGTTVTVSS

SEQ ID NO: 62 - protein, 112 aa, synthetic construct
DVVMTQSPLSLPVTLGQPASISCKSSQSIVHSNGNTYLEWYLQKPGQSPQLLVYKVSNRFSGVPDRFSGSGSGTDFTLKISRVEAEDVGTYYCFQGSLVPWAFGGGTKVEIK

SEQ ID NO: 63 - DNA, 351 bp, synthetic construct
  source 1..351: mol_type = other DNA
gaagtgcatctggtggaaagcggcggcgcgctggtgaaaccgggcggcagcctgcgcctgagctgcgcggcgagcggctttagctttagcaaatatggcatgagctgggtgcgccaggcgccgggcaaaggcctggaatgggtggcgaccattagcagcagcggcagccgcacctattatccggatagcgtgaaaggccgctttaccattagccgcgataacgcgaaaaacaccctgtatctgcagatgaacagcctgcgcgcggaagataccgcgatgtattattgcagcattagctgggatggcgcgatggattattggggccagggcaccaccgtgaccgtgagcagc

SEQ ID NO: 64 - DNA, 336 bp, synthetic construct
  source 1..336: mol_type = other DNA
gatgtggtgatgacccagagcccgctgagcctgccggtgaccctgggccagccggcgagcattagctgcaaaagcagccagagcattgtgcatagcaacggcaacacctatctggaatggtatctgcagaaaccgggccagagcccgcagctgctggtgtataaagtgagcaaccgctttagcggcgtgccggatcgctttagcggcagcggcagcggcaccgattttaccctgaaaattagccgcgtggaagcggaagatgtgggcacctattattgctttcagggcagcctggtgccgtgggcgtttggcggcggcaccaaagtggaaattaaa

SEQ ID NO: 65 - protein, 249 aa, synthetic construct
EVHLVESGGALVKPGGSLRLSCAASGFSFSKYGMSWVRQAPGKGLEWVATISSSGSRTYYPDSVKGRFTISRDNAKNTLYLQMNSLRAEDTAMYYCSISWDGAMDYWGQGTTVTVSSGGGGSGGGGSGGGGSGGGGSDVVMTQSPLSLPVTLGQPASISCKSSQSIVHSNGNTYLEWYLQKPGQSPQLLVYKVSNRFSGVPDRFSGSGSGTDFTLKISRVEAEDVGTYYCFQGSLVPWAFGGGTKVEIK

SEQ ID NO: 66 - protein, 249 aa, synthetic construct
DVVMTQSPLSLPVTLGQPASISCKSSQSIVHSNGNTYLEWYLQKPGQSPQLLVYKVSNRFSGVPDRFSGSGSGTDFTLKISRVEAEDVGTYYCFQGSLVPWAFGGGTKVEIKGGGGSGGGGSGGGGSGGGGSEVHLVESGGALVKPGGSLRLSCAASGFSFSKYGMSWVRQAPGKGLEWVATISSSGSRTYYPDSVKGRFTISRDNAKNTLYLQMNSLRAEDTAMYYCSISWDGAMDYWGQGTTVTVSS

SEQ ID NO: 67 - DNA, 747 bp, synthetic construct
  source 1..747: mol_type = other DNA
gaagtgcatctggtggaaagcggcggcgcgctggtgaaaccgggcggcagcctgcgcctgagctgcgcggcgagcggctttagctttagcaaatatggcatgagctgggtgcgccaggcgccgggcaaaggcctggaatgggtggcgaccattagcagcagcggcagccgcacctattatccggatagcgtgaaaggccgctttaccattagccgcgataacgcgaaaaacaccctgtatctgcagatgaacagcctgcgcgcggaagataccgcgatgtattattgcagcattagctgggatggcgcgatggattattggggccagggcaccaccgtgaccgtgagcagcggcggaggtggaagcggaggcggaggatcaggtggcggtggatctggcggtggcggatctgatgtggtgatgacccagagcccgctgagcctgccggtgaccctgggccagccggcgagcattagctgcaaaagcagccagagcattgtgcatagcaacggcaacacctatctggaatggtatctgcagaaaccgggccagagcccgcagctgctggtgtataaagtgagcaaccgctttagcggcgtgccggatcgctttagcggcagcggcagcggcaccgattttaccctgaaaattagccgcgtggaagcggaagatgtgggcacctattattgctttcagggcagcctggtgccgtgggcgtttggcggcggcaccaaagtggaaattaaa

SEQ ID NO: 68 - DNA, 747 bp, synthetic construct
  source 1..747: mol_type = other DNA
gatgtggtgatgacccagagcccgctgagcctgccggtgaccctgggccagccggcgagcattagctgcaaaagcagccagagcattgtgcatagcaacggcaacacctatctggaatggtatctgcagaaaccgggccagagcccgcagctgctggtgtataaagtgagcaaccgctttagcggcgtgccggatcgctttagcggcagcggcagcggcaccgattttaccctgaaaattagccgcgtggaagcggaagatgtgggcacctattattgctttcagggcagcctggtgccgtgggcgtttggcggcggcaccaaagtggaaattaaaggcggaggtggaagcggaggcggaggatcaggtggcggtggatctggcggtggcggatctgaagtgcatctggtggaaagcggcggcgcgctggtgaaaccgggcggcagcctgcgcctgagctgcgcggcgagcggctttagctttagcaaatatggcatgagctgggtgcgccaggcgccgggcaaaggcctggaatgggtggcgaccattagcagcagcggcagccgcacctattatccggatagcgtgaaaggccgctttaccattagccgcgataacgcgaaaaacaccctgtatctgcagatgaacagcctgcgcgcggaagataccgcgatgtattattgcagcattagctgggatggcgcgatggattattggggccagggcaccaccgtgaccgtgagcagc

SEQ ID NO: 69 - protein, 613 aa, synthetic construct
MALPVTALLLPLALLLHAARPGSEVHLVESGGALVKPGGSLRLSCAASGFSFSKYGMSWVRQAPGKGLEWVATISSSGSRTYYPDSVKGRFTISRDNAKNTLYLQMNSLRAEDTAMYYCSISWDGAMDYWGQGTTVTVSSGGGGSGGGGSGGGGSGGGGSDVVMTQSPLSLPVTLGQPASISCKSSQSIVHSNGNTYLEWYLQKPGQSPQLLVYKVSNRFSGVPDRFSGSGSGTDFTLKISRVEAEDVGTYYCFQGSLVPWAFGGGTKVEIKSGTTTPAPRPPTPAPTIASQPLSLRPEACRPAAGGAVHTRGLDFACDFWVLVVVGGVLACYSLLVTVAFIIFWVRSKRSTSGSGATNFSLLKQAGDVEENPGPASMVSKGEEDNMAIIKEFMRFKVHMEGSVNGHEFEIEGEGEGRPYEGTQTAKLKVTKGGPLPFAWDILSPQFMYGSKAYVKHPADIPDYLKLSFPEGFKWERVMNFEDGGVVTVTQDSSLQDGEFIYKVKLRGTNFPSDGPVMQKKTMGWEASSERMYPEDGALKGEIKQRLKLKDGGHYDAEVKTTYKAKKPVQLPGAYNVNIKLDITSHNEDYTIVEQYERAEGRHSTGGMDELYK

SEQ ID NO: 70 - protein, 613 aa, synthetic construct
MALPVTALLLPLALLLHAARPGSDVVMTQSPLSLPVTLGQPASISCKSSQSIVHSNGNTYLEWYLQKPGQSPQLLVYKVSNRFSGVPDRFSGSGSGTDFTLKISRVEAEDVGTYYCFQGSLVPWAFGGGTKVEIKGGGGSGGGGSGGGGSGGGGSEVHLVESGGALVKPGGSLRLSCAASGFSFSKYGMSWVRQAPGKGLEWVATISSSGSRTYYPDSVKGRFTISRDNAKNTLYLQMNSLRAEDTAMYYCSISWDGAMDYWGQGTTVTVSSSGTTTPAPRPPTPAPTIASQPLSLRPEACRPAAGGAVHTRGLDFACDFWVLVVVGGVLACYSLLVTVAFIIFWVRSKRSTSGSGATNFSLLKQAGDVEENPGPASMVSKGEEDNMAIIKEFMRFKVHMEGSVNGHEFEIEGEGEGRPYEGTQTAKLKVTKGGPLPFAWDILSPQFMYGSKAYVKHPADIPDYLKLSFPEGFKWERVMNFEDGGVVTVTQDSSLQDGEFIYKVKLRGTNFPSDGPVMQKKTMGWEASSERMYPEDGALKGEIKQRLKLKDGGHYDAEVKTTYKAKKPVQLPGAYNVNIKLDITSHNEDYTIVEQYERAEGRHSTGGMDELYK

SEQ ID NO: 71 - DNA, 1841 bp, synthetic construct
  source 1..1841: mol_type = other DNA
atggccctgcctgtgacagccctgctgctgcctctggctctgctgctgcatgccgctagacccggatccgaagtgcatctggtggaaagcggcggcgcgctggtgaaaccgggcggcagcctgcgcctgagctgcgcggcgagcggctttagctttagcaaatatggcatgagctgggtgcgccaggcgccgggcaaaggcctggaatgggtggcgaccattagcagcagcggcagccgcacctattatccggatagcgtgaaaggccgctttaccattagccgcgataacgcgaaaaacaccctgtatctgcagatgaacagcctgcgcgcggaagataccgcgatgtattattgcagcattagctgggatggcgcgatggattattggggccagggcaccaccgtgaccgtgagcagcggcggaggtggaagcggaggcggaggatcaggtggcggtggatctggcggtggcggatctgatgtggtgatgacccagagcccgctgagcctgccggtgaccctgggccagccggcgagcattagctgcaaaagcagccagagcattgtgcatagcaacggcaacacctatctggaatggtatctgcagaaaccgggccagagcccgcagctgctggtgtataaagtgagcaaccgctttagcggcgtgccggatcgctttagcggcagcggcagcggcaccgattttaccctgaaaattagccgcgtggaagcggaagatgtgggcacctattattgctttcagggcagcctggtgccgtgggcgtttggcggcggcaccaaagtggaaattaaaccggaaccacgacgccagcgccgcgaccaccaacaccggcgcccaccatcgcgtcgcagcccctgtccctgcgcccagaggcgtgccggccagcggcggggggcgcagtgcacacgagggggctggacttcgcctgtgatttttgggtgctggtggtggttggtggagtcctggcttgctatagcttgctagtaacagtggcctttattattttctgggtgaggagtaagaggagcactagtggaagcggagctactaacttcagcctgctgaagcaggctggagacgtggaggagaaccctggacctgctagcatggtgagcaagggcgaggaggataacatggccatcatcaaggagttcatgcgcttcaaggtgcacatggagggctccgtgaacggccacgagttcgagatcgagggcgagggcgagggccgcccctacgagggcacccagaccgccaagctgaaggtgaccaagggtggccccctgcccttcgcctgggacatcctgtcccctcagttcatgtacggctccaaggcctacgtgaagcaccccgccgacatccccgactacttgaagctgtccttccccgagggcttcaagtgggagcgcgtgatgaacttcgaggacggcggcgtggtgaccgtgacccaggactcctccctgcaggacggcgagttcatctacaaggtgaagctgcgcggcaccaacttcccctccgacggccccgtaatgcagaagaagaccatgggctgggaggcctcctccgagcggatgtaccccgaggacggcgccctgaagggcgagatcaagcagaggctgaagctgaaggacggcggccactacgacgctgaggtcaagaccacctacaaggccaagaagcccgtgcagctgcccggcgcctacaacgtcaacatcaagttggacatcacctcccacaacgaggactacaccatcgtggaacagtacgaacgcgccgagggccgccactccaccggcggcatggacgagctgtacaagtaa

SEQ ID NO: 72 - DNA, 1841 bp, synthetic construct
  source 1..1841: mol_type = other DNA
atggccctgcctgtgacagccctgctgctgcctctggctctgctgctgcatgccgctagacccggatccgatgtggtgatgacccagagcccgctgagcctgccggtgaccctgggccagccggcgagcattagctgcaaaagcagccagagcattgtgcatagcaacggcaacacctatctggaatggtatctgcagaaaccgggccagagcccgcagctgctggtgtataaagtgagcaaccgctttagcggcgtgccggatcgctttagcggcagcggcagcggcaccgattttaccctgaaaattagccgcgtggaagcggaagatgtgggcacctattattgctttcagggcagcctggtgccgtgggcgtttggcggcggcaccaaagtggaaattaaaggcggaggtggaagcggaggcggaggatcaggtggcggtggatctggcggtggcggatctgaagtgcatctggtggaaagcggcggcgcgctggtgaaaccgggcggcagcctgcgcctgagctgcgcggcgagcggctttagctttagcaaatatggcatgagctgggtgcgccaggcgccgggcaaaggcctggaatgggtggcgaccattagcagcagcggcagccgcacctattatccggatagcgtgaaaggccgctttaccattagccgcgataacgcgaaaaacaccctgtatctgcagatgaacagcctgcgcgcggaagataccgcgatgtattattgcagcattagctgggatggcgcgatggattattggggccagggcaccaccgtgaccgtgagcagcccggaaccacgacgccagcgccgcgaccaccaacaccggcgcccaccatcgcgtcgcagcccctgtccctgcgcccagaggcgtgccggccagcggcggggggcgcagtgcacacgagggggctggacttcgcctgtgatttttgggtgctggtggtggttggtggagtcctggcttgctatagcttgctagtaacagtggcctttattattttctgggtgaggagtaagaggagcactagtggaagcggagctactaacttcagcctgctgaagcaggctggagacgtggaggagaaccctggacctgctagcatggtgagcaagggcgaggaggataacatggccatcatcaaggagttcatgcgcttcaaggtgcacatggagggctccgtgaacggccacgagttcgagatcgagggcgagggcgagggccgcccctacgagggcacccagaccgccaagctgaaggtgaccaagggtggccccctgcccttcgcctgggacatcctgtcccctcagttcatgtacggctccaaggcctacgtgaagcaccccgccgacatccccgactacttgaagctgtccttccccgagggcttcaagtgggagcgcgtgatgaacttcgaggacggcggcgtggtgaccgtgacccaggactcctccctgcaggacggcgagttcatctacaaggtgaagctgcgcggcaccaacttcccctccgacggccccgtaatgcagaagaagaccatgggctgggaggcctcctccgagcggatgtaccccgaggacggcgccctgaagggcgagatcaagcagaggctgaagctgaaggacggcggccactacgacgctgaggtcaagaccacctacaaggccaagaagcccgtgcagctgcccggcgcctacaacgtcaacatcaagttggacatcacctcccacaacgaggactacaccatcgtggaacagtacgaacgcgccgagggccgccactccaccggcggcatggacgagctgtacaagtaa

SEQ ID NO: 73 - protein, 725 aa, synthetic construct
MALPVTALLLPLALLLHAARPGSQVQLVESGGGVVQPGRSLRLSCAASGFAFSSYGMHWVRQAPGKGLEWVAVIWFDGTKKYYTDSVKGRFTISRDNSKNTLYLQMNTLRAEDTAVYYCARDRGIGARRGPYYMDVWGKGTTVTVSSGGGGSGGGGSGGGGSGGGGSDIQMTQSPSSLSASVGDRVTITCRASQSISSYLNWYQQKPGKAPKLLIYAASSLQSGVPSRFSGSGSGTDFTLTISSLQPEDFATYYCQQSYSTPLTFGGGTKVEIKRTVSGTTTPAPRPPTPAPTIASQPLSLRPEACRPAAGGAVHTRGLDFACDFWVLVVVGGVLACYSLLVTVAFIIFWVRVKFSRSADAPAYKQGQNQLYNELNLGRREEYDVLDKRRGRDPEMGGKPRRKNPQEGLYNELQKDKMAEAYSEIGMKGERRRGKGHDGLYQGLSTATKDTYDALHMQALPPRTSGSGATNFSLLKQAGDVEENPGPASMVSKGEEDNMAIIKEFMRFKVHMEGSVNGHEFEIEGEGEGRPYEGTQTAKLKVTKGGPLPFAWDILSPQFMYGSKAYVKHPADIPDYLKLSFPEGFKWERVMNFEDGGVVTVTQDSSLQDGEFIYKVKLRGTNFPSDGPVMQKKTMGWEASSERMYPEDGALKGEIKQRLKLKDGGHYDAEVKTTYKAKKPVQLPGAYNVNIKLDITSHNEDYTIVEQYERAEGRHSTGGMDELYK

SEQ ID NO: 74 - protein, 725 aa, synthetic construct
MALPVTALLLPLALLLHAARPGSDIQMTQSPSSLSASVGDRVTITCRASQSISSYLNWYQQKPGKAPKLLIYAASSLQSGVPSRFSGSGSGTDFTLTISSLQPEDFATYYCQQSYSTPLTFGGGTKVEIKRTVGGGGSGGGGSGGGGSGGGGSQVQLVESGGGVVQPGRSLRLSCAASGFAFSSYGMHWVRQAPGKGLEWVAVIWFDGTKKYYTDSVKGRFTISRDNSKNTLYLQMNTLRAEDTAVYYCARDRGIGARRGPYYMDVWGKGTTVTVSSSGTTTPAPRPPTPAPTIASQPLSLRPEACRPAAGGAVHTRGLDFACDFWVLVVVGGVLACYSLLVTVAFIIFWVRVKFSRSADAPAYKQGQNQLYNELNLGRREEYDVLDKRRGRDPEMGGKPRRKNPQEGLYNELQKDKMAEAYSEIGMKGERRRGKGHDGLYQGLSTATKDTYDALHMQALPPRTSGSGATNFSLLKQAGDVEENPGPASMVSKGEEDNMAIIKEFMRFKVHMEGSVNGHEFEIEGEGEGRPYEGTQTAKLKVTKGGPLPFAWDILSPQFMYGSKAYVKHPADIPDYLKLSFPEGFKWERVMNFEDGGVVTVTQDSSLQDGEFIYKVKLRGTNFPSDGPVMQKKTMGWEASSERMYPEDGALKGEIKQRLKLKDGGHYDAEVKTTYKAKKPVQLPGAYNVNIKLDITSHNEDYTIVEQYERAEGRHSTGGMDELYK

SEQ ID NO: 75 - DNA, 2172 bp, synthetic construct
  source 1..2172: mol_type = other DNA
atggccctgcctgtgacagccctgctgctgcctctggctctgctgctgcatgccgctagacccggatcccaggtgcagctggtggaaagcggcggcggcgtggtgcagccgggccgcagcctgcgcctgagctgcgcggcgagcggctttgcgtttagcagctatggcatgcattgggtgcgccaggcgccgggcaaaggcctggaatgggtggcggtgatttggtttgatggcaccaaaaaatattataccgatagcgtgaaaggccgctttaccattagccgcgataacagcaaaaacaccctgtatctgcagatgaacaccctgcgcgcggaagataccgcggtgtattattgcgcgcgcgatcgcggcattggcgcgcgccgcggcccgtattatatggatgtgtggggcaaaggcaccaccgtgaccgtgagcagcggcggaggtggaagcggaggcggaggatcaggtggcggtggatctggcggtggcggatctgatattcagatgacccagagcccgagcagcctgagcgcgagcgtgggcgatcgcgtgaccattacctgccgcgcgagccagagcattagcagctatctgaactggtatcagcagaaaccgggcaaagcgccgaaactgctgatttatgcggcgagcagcctgcagagcggcgtgccgagccgctttagcggcagcggcagcggcaccgattttaccctgaccattagcagcctgcagccggaagattttgcgacctattattgccagcagagctatagcaccccgctgacctttggcggcggcaccaaagtggaaattaaacgctccggaaccacgacgccagcgccgcgaccaccaacaccggcgcccaccatcgcgtcgcagcccctgtccctgcgcccagaggcgtgccggccagcggcggggggcgcagtgcacacgagggggctggacttcgcctgtgatttttgggtgctggtggtggttggtggagtcctggcttgctatagcttgctagtaacagtggcctttattattttctgggtgagagtgaagttcagcaggagcgcagacgcccccgcgtacaagcagggccagaaccagctctataacgagctcaatctaggacgaagagaggagtacgatgttttggacaagagacgtggccgggaccctgagatggggggaaagccgagaaggaagaaccctcaggaaggcctgtacaatgaactgcagaaagataagatggcggaggcctacagtgagattgggatgaaaggcgagcgccggaggggcaaggggcacgatggcctttaccagggtctcagtacagccaccaaggacacctacgacgcccttcacatgcaggccctgccccctcgcactagtggaagcggagctactaacttcagcctgctgaagcaggctggagacgtggaggagaaccctggacctgctagcatggtgagcaagggcgaggaggataacatggccatcatcaaggagttcatgcgcttcaaggtgcacatggagggctccgtgaacggccacgagttcgagatcgagggcgagggcgagggccgcccctacgagggcacccagaccgccaagctgaaggtgaccaagggtggccccctgcccttcgcctgggacatcctgtcccctcagttcatgtacggctccaaggcctacgtgaagcaccccgccgacatccccgactacttgaagctgtccttccccgagggcttcaagtgggagcgcgtgatgaacttcgaggacggcggcgtggtgaccgtgacccaggactcctccctgcaggacggcgagttcatctacaaggtgaagctgcgcggcaccaacttcccctccgacggccccgtaatgcagaagaagaccatgggctgggaggcctcctccgagcggatgtaccccgaggacggcgccctgaagggcgagatcaagcagaggctgaagctgaaggacggcggccactacgacgctgaggtcaagaccacctacaaggccaagaagcccgtgcagctgcccggcgcctacaacgtcaacatcaagttggacatcacctcccacaacgaggactacaccatcgtggaacagtacgaacgcgccgagggccgccactccaccggcggcatggacgagctgtacaagtaa

SEQ ID NO: 76 - DNA, 2177 bp, synthetic construct
  source 1..2177: mol_type = other DNA
atggccctgcctgtgacagccctgctgctgcctctggctctgctgctgcatgccgctagacccggatccgatatccagatgactcagagccccagcagcctgtctgcctctgtgggagacagagtgaccatcacctgtagagccagccagagcatcagcagctacctgaactggtatcagcagaagcccggcaaggcccctaaactgctgatctatgccgcctccagtctgcagagcggagtgccttctagattttccggcagcggctccggcaccgatttcaccctgaccatatctagcctgcagcctgaggacttcgccacctactactgccagcagagctacagcacccctctgacttttggcggaggcaccaaggtggaaatcaagcggacagtgtggcggaggtggaagcggaggcggaggatcaggtggcggtggatctggcggtggcggatctcaggtgcagctggtggaaagcggcggcggcgtggtgcagccgggccgcagcctgcgcctgagctgcgcggcgagcggctttgcgtttagcagctatggcatgcattgggtgcgccaggcgccgggcaaaggcctggaatgggtggcggtgatttggtttgatggcaccaaaaaatattataccgatagcgtgaaaggccgctttaccattagccgcgataacagcaaaaacaccctgtatctgcagatgaacaccctgcgcgcggaagataccgcggtgtattattgcgcgcgcgatcgcggcattggcgcgcgccgcggcccgtattatatggatgtgtggggcaaaggcaccaccgtgaccgtgagcagcccggaaccacgacgccagcgccgcgaccaccaacaccggcgcccaccatcgcgtcgcagcccctgtccctgcgcccagaggcgtgccggccagcggcggggggcgcagtgcacacgagggggctggacttcgcctgtgatttttgggtgctggtggtggttggtggagtcctggcttgctatagcttgctagtaacagtggcctttattattttctgggtgagagtgaagttcagcaggagcgcagacgcccccgcgtacaagcagggccagaaccagctctataacgagctcaatctaggacgaagagaggagtacgatgttttggacaagagacgtggccgggaccctgagatggggggaaagccgagaaggaagaaccctcaggaaggcctgtacaatgaactgcagaaagataagatggcggaggcctacagtgagattgggatgaaaggcgagcgccggaggggcaaggggcacgatggcctttaccagggtctcagtacagccaccaaggacacctacgacgcccttcacatgcaggccctgccccctcgcctagtggaagcggagctactaacttcagcctgctgaagcaggctggagacgtggaggagaaccctggacctgctagcatggtgagcaagggcgaggaggataacatggccatcatcaaggagttcatgcgcttcaaggtgcacatggagggctccgtgaacggccacgagttcgagatcgagggcgagggcgagggccgcccctacgagggcacccagaccgccaagctgaaggtgaccaagggtggccccctgcccttcgcctgggacatcctgtcccctcagttcatgtacggctccaaggcctacgtgaagcaccccgccgacatccccgactacttgaagctgtccttccccgagggcttcaagtgggagcgcgtgatgaacttcgaggacggcggcgtggtgaccgtgacccaggactcctccctgcaggacggcgagttcatctacaaggtgaagctgcgcggcaccaacttcccctccgacggccccgtaatgcagaagaagaccatgggctgggaggcctcctccgagcggatgtaccccgaggacggcgccctgaagggcgagatcaagcagaggctgaagctgaaggacggcggccactacgacgctgaggtcaagaccacctacaaggccaagaagcccgtgcagctgcccggcgcctacaacgtcaacatcaagttggacatcacctcccacaacgaggactacaccatcgtggaacagtacgaacgcgccgagggccgccactccaccggcggcatggacgagctgtacaagtaa

SEQ ID NO: 77 - protein, 715 aa, synthetic construct
MALPVTALLLPLALLLHAARPGSEVQLVESGGGLVQPGGSLRLSCAASGFTFSSYGMSWVRQAPGKGLELVASINSNGGSTYYPDSVKGRFTISRDNAKNSLYLQMNSLRAEDTAVYYCASGDYWGQGTTVTVSSGGGGSGGGGSGGGGSGGGGSDIVMTQSPLSLPVTPGEPASISCRSSQSLVYSNGDTYLHWYLQKPGQSPQLLIYKVSNRFSGVPDRFSGSGSGTDFTLKISRVEAEDVGVYYCSQSTHVPWTFGQGTKVEIKSGTTTPAPRPPTPAPTIASQPLSLRPEACRPAAGGAVHTRGLDFACDFWVLVVVGGVLACYSLLVTVAFIIFWVRVKFSRSADAPAYKQGQNQLYNELNLGRREEYDVLDKRRGRDPEMGGKPRRKNPQEGLYNELQKDKMAEAYSEIGMKGERRRGKGHDGLYQGLSTATKDTYDALHMQALPPRTSGSGATNFSLLKQAGDVEENPGPASMVSKGEEDNMAIIKEFMRFKVHMEGSVNGHEFEIEGEGEGRPYEGTQTAKLKVTKGGPLPFAWDILSPQFMYGSKAYVKHPADIPDYLKLSFPEGFKWERVMNFEDGGVVTVTQDSSLQDGEFIYKVKLRGTNFPSDGPVMQKKTMGWEASSERMYPEDGALKGEIKQRLKLKDGGHYDAEVKTTYKAKKPVQLPGAYNVNIKLDITSHNEDYTIVEQYERAEGRHSTGGMDELYK

SEQ ID NO: 78 - protein, 715 aa, synthetic construct
MALPVTALLLPLALLLHAARPGSDIVMTQSPLSLPVTPGEPASISCRSSQSLVYSNGDTYLHWYLQKPGQSPQLLIYKVSNRFSGVPDRFSGSGSGTDFTLKISRVEAEDVGVYYCSQSTHVPWTFGQGTKVEIKGGGGSGGGGSGGGGSGGGGSEVQLVESGGGLVQPGGSLRLSCAASGFTFSSYGMSWVRQAPGKGLELVASINSNGGSTYYPDSVKGRFTISRDNAKNSLYLQMNSLRAEDTAVYYCASGDYWGQGTTVTVSSSGTTTPAPRPPTPAPTIASQPLSLRPEACRPAAGGAVHTRGLDFACDFWVLVVVGGVLACYSLLVTVAFIIFWVRVKFSRSADAPAYKQGQNQLYNELNLGRREEYDVLDKRRGRDPEMGGKPRRKNPQEGLYNELQKDKMAEAYSEIGMKGERRRGKGHDGLYQGLSTATKDTYDALHMQALPPRTSGSGATNFSLLKQAGDVEENPGPASMVSKGEEDNMAIIKEFMRFKVHMEGSVNGHEFEIEGEGEGRPYEGTQTAKLKVTKGGPLPFAWDILSPQFMYGSKAYVKHPADIPDYLKLSFPEGFKWERVMNFEDGGVVTVTQDSSLQDGEFIYKVKLRGTNFPSDGPVMQKKTMGWEASSERMYPEDGALKGEIKQRLKLKDGGHYDAEVKTTYKAKKPVQLPGAYNVNIKLDITSHNEDYTIVEQYERAEGRHSTGGMDELYK

SEQ ID NO: 79 - DNA, 2152 bp, synthetic construct
  source 1..2152: mol_type = other DNA
atggccctgcctgtgacagccctgctgctgcctctggctctgctgctgcatgccgctagacccggatccgaggtgcagctggttgaatctggcggaggactggttcagcctggcggatctctgagactgtcttgtgccgccagcggcttcacctttagcagctacggcatgagctgggtccgacaggctcctggcaaaggactggaactggtggccagcatcaacagcaatggcggcagcacctactatcccgacagcgtgaagggcagattcaccatcagccgggacaacgccaagaacagcctgtacctgcagatgaactccctgagagccgaggacaccgccgtgtactattgtgccagcggcgattattggggccagggcacaacagtgacagtgtctagcggcggaggtggaagcggaggcggaggatcaggtggcggtggatctggcggtggcggatctgacattgtgatgacacagagccctctgagcctgccagtgacacctggcgaacctgccagcatctcctgtagaagcagccagagcctggtgtacagcaacggcgatacctacctgcactggtatctgcagaagcccggccagtctcctcagctgctgatctacaaggtgtccaaccggttcagcggcgtgcccgatagattttctggcagcggctctggcaccgacttcaccctgaagatctccagagtggaagccgaggatgtgggcgtgtactactgcagccagtctacccacgtgccatggacatttggacagggcaccaaggtggaaatcaagtccggaccggaaccacgacgccagcgccgcgaccaccaacaccggcgcccaccatcgcgtcgcagcccctgtccctgcgcccagaggcgtgccggccagcggcggggggcgcagtgcacacgagggggctggacttcgcctgtgatttttgggtgctggtggtggttggtggagtcctggcttgctatagcttgctagtaacagtggcctttattattttctgggtgagagtgaagttcagcaggagcgcagacgcccccgcgtacaagcagggccagaaccagctctataacgagctcaatctaggacgaagagaggagtacgatgttttggacaagagacgtggccgggaccctgagatggggggaaagccgagaaggaagaaccctcaggaaggcctgtacaatgaactgcagaaagataagatggcggaggcctacagtgagattgggatgaaaggcgagcgccggaggggcaaggggcacgatggcctttaccagggtctcagtacagccaccaaggacacctacgacgcccttcacatgcaggccctgccccctcgcctagtggaagcggagctactaacttcagcctgctgaagcaggctggagacgtggaggagaaccctggacctgctagcatggtgagcaagggcgaggaggataacatggccatcatcaaggagttcatgcgcttcaaggtgcacatggagggctccgtgaacggccacgagttcgagatcgagggcgagggcgagggccgcccctacgagggcacccagaccgccaagctgaaggtgaccaagggtggccccctgcccttcgcctgggacatcctgtcccctcagttcatgtacggctccaaggcctacgtgaagcaccccgccgacatccccgactacttgaagctgtccttccccgagggcttcaagtgggagcgcgtgatgaacttcgaggacggcggcgtggtgaccgtgacccaggactcctccctgcaggacggcgagttcatctacaaggtgaagctgcgcggcaccaacttcccctccgacggccccgtaatgcagaagaagaccatgggctgggaggcctcctccgagcggatgtaccccgaggacggcgccctgaagggcgagatcaagcagaggctgaagctgaaggacggcggccactacgacgctgaggtcaagaccacctacaaggccaagaagcccgtgcagctgcccggcgcctacaacgtcaacatcaagttggacatcacctcccacaacgaggactacaccatcgtggaacagtacgaacgcgccgagggccgccactccaccggcggcatggacgagctgtacaagtaa

SEQ ID NO: 80 - DNA, 2152 bp, synthetic construct
  source 1..2152: mol_type = other DNA
atggccctgcctgtgacagccctgctgctgcctctggctctgctgctgcatgccgctagacccggatccgacattgtgatgacacagagccctctgagcctgccagtgacacctggcgaacctgccagcatctcctgtagaagcagccagagcctggtgtacagcaacggcgatacctacctgcactggtatctgcagaagcccggccagtctcctcagctgctgatctacaaggtgtccaaccggttcagcggcgtgcccgatagattttctggcagcggctctggcaccgacttcaccctgaagatctccagagtggaagccgaggatgtgggcgtgtactactgcagccagtctacccacgtgccatggacatttggacagggcaccaaggtggaaatcaagtccggaggcggaggtggaagcggaggcggaggatcaggtggcggtggatctggcggtggcggatctgaggtgcagctggttgaatctggcggaggactggttcagcctggcggatctctgagactgtcttgtgccgccagcggcttcacctttagcagctacggcatgagctgggtccgacaggctcctggcaaaggactggaactggtggccagcatcaacagcaatggcggcagcacctactatcccgacagcgtgaagggcagattcaccatcagccgggacaacgccaagaacagcctgtacctgcagatgaactccctgagagccgaggacaccgccgtgtactattgtgccagcggcgattattggggccagggcacaacagtgacagtgtctagcccggaaccacgacgccagcgccgcgaccaccaacaccggcgcccaccatcgcgtcgcagcccctgtccctgcgcccagaggcgtgccggccagcggcggggggcgcagtgcacacgagggggctggacttcgcctgtgatttttgggtgctggtggtggttggtggagtcctggcttgctatagcttgctagtaacagtggcctttattattttctgggtgagagtgaagttcagcaggagcgcagacgcccccgcgtacaagcagggccagaaccagctctataacgagctcaatctaggacgaagagaggagtacgatgttttggacaagagacgtggccgggaccctgagatggggggaaagccgagaaggaagaaccctcaggaaggcctgtacaatgaactgcagaaagataagatggcggaggcctacagtgagattgggatgaaaggcgagcgccggaggggcaaggggcacgatggcctttaccagggtctcagtacagccaccaaggacacctacgacgcccttcacatgcaggccctgccccctcgcctagtggaagcggagctactaacttcagcctgctgaagcaggctggagacgtggaggagaaccctggacctgctagcatggtgagcaagggcgaggaggataacatggccatcatcaaggagttcatgcgcttcaaggtgcacatggagggctccgtgaacggccacgagttcgagatcgagggcgagggcgagggccgcccctacgagggcacccagaccgccaagctgaaggtgaccaagggtggccccctgcccttcgcctgggacatcctgtcccctcagttcatgtacggctccaaggcctacgtgaagcaccccgccgacatccccgactacttgaagctgtccttccccgagggcttcaagtgggagcgcgtgatgaacttcgaggacggcggcgtggtgaccgtgacccaggactcctccctgcaggacggcgagttcatctacaaggtgaagctgcgcggcaccaacttcccctccgacggccccgtaatgcagaagaagaccatgggctgggaggcctcctccgagcggatgtaccccgaggacggcgccctgaagggcgagatcaagcagaggctgaagctgaaggacggcggccactacgacgctgaggtcaagaccacctacaaggccaagaagcccgtgcagctgcccggcgcctacaacgtcaacatcaagttggacatcacctcccacaacgaggactacaccatcgtggaacagtacgaacgcgccgagggccgccactccaccggcggcatggacgagctgtacaagtaa

SEQ ID NO: 81 - protein, 724 aa, synthetic construct
MALPVTALLLPLALLLHAARPGSEVKLVESGGGLVKPGASLKLSCAASGFTFSNYGMSWVRQNSDKRLEWVASIRSGGGRTYYSDNVKGRFTISRENAKNTLYLQMSSLKSEDTALYYCVRYDHYSGSSDYWGQGTTVTVSSSGGGGGSGGGGSGGGGSGGGGSYVVMTQTPLTLSVTIGQPASISCKSSQSLLDSDGKTYLNWLLQRPGQSPKRLIYLVSKLDSGVPDRFTGSGSGTDFTLKISRIEAEDLGLYYCWQGTHFPRTFGGGTKLEIKSGTTTPAPRPPTPAPTIASQPLSLRPEACRPAAGGAVHTRGLDFACDFWVLVVVGGVLACYSLLVTVAFIIFWVRVKFSRSADAPAYKQGQNQLYNELNLGRREEYDVLDKRRGRDPEMGGKPRRKNPQEGLYNELQKDKMAEAYSEIGMKGERRRGKGHDGLYQGLSTATKDTYDALHMQALPPRTSGSGATNFSLLKQAGDVEENPGPASMVSKGEEDNMAIIKEFMRFKVHMEGSVNGHEFEIEGEGEGRPYEGTQTAKLKVTKGGPLPFAWDILSPQFMYGSKAYVKHPADIPDYLKLSFPEGFKWERVMNFEDGGVVTVTQDSSLQDGEFIYKVKLRGTNFPSDGPVMQKKTMGWEASSERMYPEDGALKGEIKQRLKLKDGGHYDAEVKTTYKAKKPVQLPGAYNVNIKLDITSHNEDYTIVEQYERAEGRHSTGGMDELYK

SEQ ID NO: 82 - protein, 724 aa, synthetic construct
MALPVTALLLPLALLLHAARPGSYVVMTQTPLTLSVTIGQPASISCKSSQSLLDSDGKTYLNWLLQRPGQSPKRLIYLVSKLDSGVPDRFTGSGSGTDFTLKISRIEAEDLGLYYCWQGTHFPRTFGGGTKLEIKGGGGSGGGGSGGGGSGGGGSEVKLVESGGGLVKPGASLKLSCAASGFTFSNYGMSWVRQNSDKRLEWVASIRSGGGRTYYSDNVKGRFTISRENAKNTLYLQMSSLKSEDTALYYCVRYDHYSGSSDYWGQGTTVTVSSSGSGTTTPAPRPPTPAPTIASQPLSLRPEACRPAAGGAVHTRGLDFACDFWVLVVVGGVLACYSLLVTVAFIIFWVRVKFSRSADAPAYKQGQNQLYNELNLGRREEYDVLDKRRGRDPEMGGKPRRKNPQEGLYNELQKDKMAEAYSEIGMKGERRRGKGHDGLYQGLSTATKDTYDALHMQALPPRTSGSGATNFSLLKQAGDVEENPGPASMVSKGEEDNMAIIKEFMRFKVHMEGSVNGHEFEIEGEGEGRPYEGTQTAKLKVTKGGPLPFAWDILSPQFMYGSKAYVKHPADIPDYLKLSFPEGFKWERVMNFEDGGVVTVTQDSSLQDGEFIYKVKLRGTNFPSDGPVMQKKTMGWEASSERMYPEDGALKGEIKQRLKLKDGGHYDAEVKTTYKAKKPVQLPGAYNVNIKLDITSHNEDYTIVEQYERAEGRHSTGGMDELYK

SEQ ID NO: 83 - DNA, 2173 bp, synthetic construct
  source 1..2173: mol_type = other DNA
atggccctgcctgtgacagccctgctgctgcctctggctctgctgctgcatgccgctagacccggatccgaagtgaagctggttgaaagtggcggaggcctggttaagcctggcgcctctctgaaactgtcttgtgccgccagcggcttcaccttcagcaactacggcatgagctgggtccgacagaacagcgacaagcggcttgagtgggtcgcctctattagatccggcggaggcagaacctactactccgacaacgtgaagggcagattcaccatcagcagagagaacgccaagaacaccctgtacctgcagatgagcagcctgaagtccgaggacaccgctctgtactattgcgtcagatacgaccactacagcggcagcagcgattattggggccagggcacaaccgtgaccgtgtctagctccggaggcggaggtggaagcggaggcggaggatcaggtggcggtggatctggcggtggcggatcttacgtggtcatgacacagacccctctgacactgagcgtgaccattggacagcctgccagcatcagctgcaagagcagtcagagcctgctggactccgacggcaagacctacctgaattggctgctgcagaggcccggacagagccccaagagactgatctacctggtgtccaagctggacagcggcgtgcccgatagattcacaggctctggcagcggcaccgacttcaccctgaagatctctagaatcgaggccgaggacctgggcctgtactactgttggcagggcacacacttccccagaacctttggcggcggaacaaagctggaaatcaaaccggaaccacgacgccagcgccgcgaccaccaacaccggcgcccaccatcgcgtcgcagcccctgtccctgcgcccagaggcgtgccggccagcggcggggggcgcagtgcacacgagggggctggacttcgcctgtgatttttgggtgctggtggtggttggtggagtcctggcttgctatagcttgctagtaacagtggcctttattattttctgggtgagagtgaagttcagcaggagcgcagacgcccccgcgtacaagcagggccagaaccagctctataacgagctcaatctaggacgaagagaggagtacgatgttttggacaagagacgtggccgggaccctgagatggggggaaagccgagaaggaagaaccctcaggaaggcctgtacaatgaactgcagaaagataagatggcggaggcctacagtgagattgggatgaaaggcgagcgccggaggggcaaggggcacgatggcctttaccagggtctcagtacagccaccaaggacacctacgacgcccttcacatgcaggccctgccccctcgcctagtggaagcggagctactaacttcagcctgctgaagcaggctggagacgtggaggagaaccctggacctgctagcatggtgagcaagggcgaggaggataacatggccatcatcaaggagttcatgcgcttcaaggtgcacatggagggctccgtgaacggccacgagttcgagatcgagggcgagggcgagggccgcccctacgagggcacccagaccgccaagctgaaggtgaccaagggtggccccctgcccttcgcctgggacatcctgtcccctcagttcatgtacggctccaaggcctacgtgaagcaccccgccgacatccccgactacttgaagctgtccttccccgagggcttcaagtgggagcgcgtgatgaacttcgaggacggcggcgtggtgaccgtgacccaggactcctccctgcaggacggcgagttcatctacaaggtgaagctgcgcggcaccaacttcccctccgacggccccgtaatgcagaagaagaccatgggctgggaggcctcctccgagcggatgtaccccgaggacggcgccctgaagggcgagatcaagcagaggctgaagctgaaggacggcggccactacgacgctgaggtcaagaccacctacaaggccaagaagcccgtgcagctgcccggcgcctacaacgtcaacatcaagttggacatcacctcccacaacgaggactacaccatcgtggaacagtacgaacgcgccgagggccgccactccaccggcggcatggacgagctgtacaagtaa

SEQ ID NO: 84 - DNA, 2173 bp, synthetic construct
  source 1..2173: mol_type = other DNA
atggccctgcctgtgacagccctgctgctgcctctggctctgctgctgcatgccgctagacccggatcctacgtggtcatgacacagacccctctgacactgagcgtgaccattggacagcctgccagcatcagctgcaagagcagtcagagcctgctggactccgacggcaagacctacctgaattggctgctgcagaggcccggacagagccccaagagactgatctacctggtgtccaagctggacagcggcgtgcccgatagattcacaggctctggcagcggcaccgacttcaccctgaagatctctagaatcgaggccgaggacctgggcctgtactactgttggcagggcacacacttccccagaacctttggcggcggaacaaagctggaaatcaaaggcggaggtggaagcggaggcggaggatcaggtggcggtggatctggcggtggcggatctgaagtgaagctggttgaaagtggcggaggcctggttaagcctggcgcctctctgaaactgtcttgtgccgccagcggcttcaccttcagcaactacggcatgagctgggtccgacagaacagcgacaagcggcttgagtgggtcgcctctattagatccggcggaggcagaacctactactccgacaacgtgaagggcagattcaccatcagcagagagaacgccaagaacaccctgtacctgcagatgagcagcctgaagtccgaggacaccgctctgtactattgcgtcagatacgaccactacagcggcagcagcgattattggggccagggcacaaccgtgaccgtgtctagctccggaccggaaccacgacgccagcgccgcgaccaccaacaccggcgcccaccatcgcgtcgcagcccctgtccctgcgcccagaggcgtgccggccagcggcggggggcgcagtgcacacgagggggctggacttcgcctgtgatttttgggtgctggtggtggttggtggagtcctggcttgctatagcttgctagtaacagtggcctttattattttctgggtgagagtgaagttcagcaggagcgcagacgcccccgcgtacaagcagggccagaaccagctctataacgagctcaatctaggacgaagagaggagtacgatgttttggacaagagacgtggccgggaccctgagatggggggaaagccgagaaggaagaaccctcaggaaggcctgtacaatgaactgcagaaagataagatggcggaggcctacagtgagattgggatgaaaggcgagcgccggaggggcaaggggcacgatggcctttaccagggtctcagtacagccaccaaggacacctacgacgcccttcacatgcaggccctgccccctcgcctagtggaagcggagctactaacttcagcctgctgaagcaggctggagacgtggaggagaaccctggacctgctagcatggtgagcaagggcgaggaggataacatggccatcatcaaggagttcatgcgcttcaaggtgcacatggagggctccgtgaacggccacgagttcgagatcgagggcgagggcgagggccgcccctacgagggcacccagaccgccaagctgaaggtgaccaagggtggccccctgcccttcgcctgggacatcctgtcccctcagttcatgtacggctccaaggcctacgtgaagcaccccgccgacatccccgactacttgaagctgtccttccccgagggcttcaagtgggagcgcgtgatgaacttcgaggacggcggcgtggtgaccgtgacccaggactcctccctgcaggacggcgagttcatctacaaggtgaagctgcgcggcaccaacttcccctccgacggccccgtaatgcagaagaagaccatgggctgggaggcctcctccgagcggatgtaccccgaggacggcgccctgaagggcgagatcaagcagaggctgaagctgaaggacggcggccactacgacgctgaggtcaagaccacctacaaggccaagaagcccgtgcagctgcccggcgcctacaacgtcaacatcaagttggacatcacctcccacaacgaggactacaccatcgtggaacagtacgaacgcgccgagggccgccactccaccggcggcatggacgagctgtacaagtaa

SEQ ID NO: 85 - protein, 796 aa, synthetic construct
MALPVTALLLPLALLLHAARPGSQVQLVESGGGVVQPGRSLRLSCAASGFAFSSYGMHWVRQAPGKGLEWVAVIWFDGTKKYYTDSVKGRFTISRDNSKNTLYLQMNTLRAEDTAVYYCARDRGIGARRGPYYMDVWGKGTTVTVSSGGGGSGGGGSGGGGSGGGGSDIQMTQSPSSLSASVGDRVTITCRASQSISSYLNWYQQKPGKAPKLLIYAASSLQSGVPSRFSGSGSGTDFTLTISSLQPEDFATYYCQQSYSTPLTFGGGTKVEIKRTVSGTTTPAPRPPTPAPTIASQPLSLRPEACRPAAGGAVHTRGLDFACDFWVLVVVGGVLACYSLLVTVAFIIFWVRSKRSMVSKGEEDNMAIIKEFMRFKVHMEGSVNGHEFEIEGEGEGRPYEGTQTAKLKVTKGGPLPFAWDILSPQFMYGSKAYVKHPADIPDYLKLSFPEGFKWERVMNFEDGGVVTVTQDSSLQDGEFIYKVKLRGTNFPSDGPVMQKKTMGWEASSERMYPEDGALKGEIKQRLKLKDGGHYDAEVKTTYKAKKPVQLPGAYNVNIKLDITSHNEDYTIVEQYERAEGRHSTGGMDELYKASGSGATNFSLLKQAGDVEENPGPASMPGSALLCCLLLLTGMRISRGQYSREDNNCTHFPVGQSHMLLELRTAFSQVKTFFQTKDQLDNILLTDSLMQDFKGYLGCQALSEMIQFYLVEVMPQAEKHGPEIKEHLNSLGEKLKTLRMRLRRCHRFLPCENKSKAVEQVKSDFNKLQDQGVYKAMNEFDIFINCIEAYMMIKMKS

SEQ ID NO: 86 - DNA, 1932 bp, synthetic construct
  source 1..1932: mol_type = other DNA
atggccctgcctgtgacagccctgctgctgcctctggctctgctgctgcatgccgctagacccggatcccaggtgcagctggtggaaagcggcggcggcgtggtgcagccgggccgcagcctgcgcctgagctgcgcggcgagcggctttgcgtttagcagctatggcatgcattgggtgcgccaggcgccgggcaaaggcctggaatgggtggcggtgatttggtttgatggcaccaaaaaatattataccgatagcgtgaaaggccgctttaccattagccgcgataacagcaaaaacaccctgtatctgcagatgaacaccctgcgcgcggaagataccgcggtgtattattgcgcgcgcgatcgcggcattggcgcgcgccgcggcccgtattatatggatgtgtggggcaaaggcaccaccgtgaccgtgagcagcggcggaggtggaagcggaggcggaggatcaggtggcggtggatctggcggtggcggatctgatattcagatgacccagagcccgagcagcctgagcgcgagcgtgggcgatcgcgtgaccattacctgccgcgcgagccagagcattagcagctatctgaactggtatcagcagaaaccgggcaaagcgccgaaactgctgatttatgcggcgagcagcctgcagagcggcgtgccgagccgctttagcggcagcggcagcggcaccgattttaccctgaccattagcagcctgcagccggaagattttgcgacctattattgccagcagagctatagcaccccgctgacctttggcggcggcaccaaagtggaaattaaacgctccggaaccacgacgccagcgccgcgaccaccaacaccggcgcccaccatcgcgtcgcagcccctgtccctgcgcccagaggcgtgccggccagcggcggggggcgcagtgcacacgagggggctggacttcgcctgtgatttttgggtgctggtggtggttggtggagtcctggcttgctatagcttgctagtaacagtggcctttattattttctgggtgaggagtaagaggagctcaaccgaagaacagttcaaatcagtcttggagattcagaagactggaaaatacaagaaagttgaaaccgaactcctaaccctaggtggaagcggagctactaacttcagcctgctgaagcaggctggagacgtggaggagaaccctggacctgctagcatggtgagcaagggcgaggaggataacatggccatcatcaaggagttcatgcgcttcaaggtgcacatggagggctccgtgaacggccacgagttcgagatcgagggcgagggcgagggccgcccctacgagggcacccagaccgccaagctgaaggtgaccaagggtggccccctgcccttcgcctgggacatcctgtcccctcagttcatgtacggctccaaggcctacgtgaagcaccccgccgacatccccgactacttgaagctgtccttccccgagggcttcaagtgggagcgcgtgatgaacttcgaggacggcggcgtggtgaccgtgacccaggactcctccctgcaggacggcgagttcatctacaaggtgaagctgcgcggcaccaacttcccctccgacggccccgtaatgcagaagaagaccatgggctgggaggcctcctccgagcggatgtaccccgaggacggcgccctgaagggcgagatcaagcagaggctgaagctgaaggacggcggccactacgacgctgaggtcaagaccacctacaaggccaagaagcccgtgcagctgcccggcgcctacaacgtcaacatcaagttggacatcacctcccacaacgaggactacaccatcgtggaacagtacgaacgcgccgagggccgccactccaccggcggcatggacgagctgtacaagtaa

SEQ ID NO: 87 - protein, 645 aa, synthetic construct
MALPVTALLLPLALLLHAARPGSQVQLVESGGGVVQPGRSLRLSCAASGFAFSSYGMHWVRQAPGKGLEWVAVIWFDGTKKYYTDSVKGRFTISRDNSKNTLYLQMNTLRAEDTAVYYCARDRGIGARRGPYYMDVWGKGTTVTVSSGGGGSGGGGSGGGGSGGGGSDIQMTQSPSSLSASVGDRVTITCRASQSISSYLNWYQQKPGKAPKLLIYAASSLQSGVPSRFSGSGSGTDFTLTISSLQPEDFATYYCQQSYSTPLTFGGGTKVEIKRTVSGTTTPAPRPPTPAPTIASQPLSLRPEACRPAAGGAVHTRGLDFACDFWVLVVVGGVLACYSLLVTVAFIIFWVRSKRSSTEEQFKSVLEIQKTGKYKKVETELLTLGGSGATNFSLLKQAGDVEENPGPASMVSKGEEDNMAIIKEFMRFKVHMEGSVNGHEFEIEGEGEGRPYEGTQTAKLKVTKGGPLPFAWDILSPQFMYGSKAYVKHPADIPDYLKLSFPEGFKWERVMNFEDGGVVTVTQDSSLQDGEFIYKVKLRGTNFPSDGPVMQKKTMGWEASSERMYPEDGALKGEIKQRLKLKDGGHYDAEVKTTYKAKKPVQLPGAYNVNIKLDITSHNEDYTIVEQYERAEGRHSTGGMDELYK

SEQ ID NO: 88 - DNA, 1932 bp, synthetic construct; identical to SEQ ID NO: 86
  source 1..1932: mol_type = other DNA

SEQ ID NO: 89 - protein, 765 aa, synthetic construct
MALPVTALLLPLALLLHAARPGSEVHLVESGGALVKPGGSLRLSCAASGFSFSKYGMSWVRQAPGKGLEWVATISSSGSRTYYPDSVKGRFTISRDNAKNTLYLQMNSLRAEDTAMYYCSISWDGAMDYWGQGTTVTVSSGGGGSGGGGSGGGGSGGGGSDVVMTQSPLSLPVTLGQPASISCKSSQSIVHSNGNTYLEWYLQKPGQSPQLLVYKVSNRFSGVPDRFSGSGSGTDFTLKISRVEAEDVGTYYCFQGSLVPWAFGGGTKVEIKSGTTTPAPRPPTPAPTIASQPLSLRPEACRPAAGGAVHTRGLDFACDGGGGSEQKLISEEDLSGFWVLVVVGGVLACYSLLVTVAFIIFWVRSKRSASGSGATNFSLLKQAGDVEENPGPASMALPVTALLLPLALLLHAARPGSQVQLVESGGGVVQPGRSLRLSCAASGFAFSSYGMHWVRQAPGKGLEWVAVIWFDGTKKYYTDSVKGRFTISRDNSKNTLYLQMNTLRAEDTAVYYCARDRGIGARRGPYYMDVWGKGTTVTVSSGGGGSGGGGSGGGGSGGGGSDIQMTQSPSSLSASVGDRVTITCRASQSISSYLNWYQQKPGKAPKLLIYAASSLQSGVPSRFSGSGSGTDFTLTISSLQPEDFATYYCQQSYSTPLTFGGGTKVEIKRSGTTTPAPRPPTPAPTIASQPLSLRPEACRPAAGGAVHTRGLDFACDGGGGSEQKLISEEDLSGFWVLVVVGGVLACYSLLVTVAFIIFWVRSKRS

SEQ ID NO: 90 - DNA, 2280 bp, synthetic construct
  source 1..2280: mol_type = other DNA
atggccctgcctgtgacagccctgctgctgcctctggctctgctgctgcatgccgctagacccggatccgaggtgcagctggttgaatctggcggaggactggttcagcctggcggatctctgagactgtcttgtgccgccagcggcttcacctttagcagctacggcatgagctgggtccgacaggctcctggcaaaggactggaactggtggccagcatcaacagcaatggcggcagcacctactatcccgacagcgtgaagggcagattcaccatcagccgggacaacgccaagaacagcctgtacctgcagatgaactccctgagagccgaggacaccgccgtgtactattgtgccagcggcgattattggggccagggcacaacagtgacagtgtctagcggcggaggtggaagcggaggcggaggatcaggtggcggtggatctggcggtggcggatctgatgtggtgatgacccagagcccgctgagcctgccggtgaccctgggccagccggcgagcattagctgcaaaagcagccagagcattgtgcatagcaacggcaacacctatctggaatggtatctgcagaaaccgggccagagcccgcagctgctggtgtataaagtgagcaaccgctttagcggcgtgccggatcgctttagcggcagcggcagcggcaccgattttaccctgaaaattagccgcgtggaagcggaagatgtgggcacctattattgctttcagggcagcctggtgccgtgggcgtttggcggcggcaccaaagtggaaattaaatccggaaccacgacgccagcgccgcgaccaccaacaccggcgcccaccatcgcgtcgcagcccctgtccctgcgcccagaggcgtgccggccagcggcggggggcgcagtgcacacgagggggctggacttcgcctgtgaggggggggggggagcgaacaaaaactcatctcagaagaggatctgtccggatttttgggtgctggtggtggttggtggagtcctggcttgctatagcttgctagtaacagtggcctttattattttctgggtgaggagtaagaggagcgctagcggaagcggagctactaacttcagcctgctgaagcaggctggagacgtggaggagaaccctggacctgctagcatggccctgcctgtgacagccctgctgctgcctctggctctgctgctgcatgccgctagacccggatcccaggtgcagctggtggaaagcggcggcggcgtggtgcagccgggccgcagcctgcgcctgagctgcgcggcgagcggctttgcgtttagcagctatggcatgcattgggtgcgccaggcgccgggcaaaggcctggaatgggtggcggtgatttggtttgatggcaccaaaaaatattataccgatagcgtgaaaggccgctttaccattagccgcgataacagcaaaaacaccctgtatctgcagatgaacaccctgcgcgcggaagataccgcggtgtattattgcgcgcgcgatcgcggcattggcgcgcgccgcggcccgtattatatggatgtgtggggcaaaggcaccaccgtgaccgtgagcagcggcggaggtggaagcggaggcggaggatcaggtggcggtggatctggcggtggcggatctgatattcagatgacccagagcccgagcagcctgagcgcgagcgtgggcgatcgcgtgaccattacctgccgcgcgagccagagcattagcagctatctgaactggtatcagcagaaaccgggcaaagcgccgaaactgctgatttatgcggcgagcagcctgcagagcggcgtgccgagccgctttagcggcagcggcagcggcaccgattttaccctgaccattagcagcctgcagccggaagattttgcgacctattattgccagcagagctatagcaccccgctgacctttggcggcggcaccaaagtggaaattaaacgctccggaaccacgacgccagcgccgcgaccaccaacaccggcgcccaccatcgcgtcgcagcccctgtccctgcgcccagaggcgtgccggccagcggcggggggcgcagtgcacacgagggggctggacttcgcctgtgaggggggggggggagctacccatacgatgttccagattacgcttccggatttttgggtgctggtggtggttggtggagtcctggcttgctatagcttgctagtaacagtggcctttattattttctgggtgaggagtaagaggagctaa

SEQ ID NO: 91 - protein, 592 aa, synthetic construct
MALPVTALLLPLALLLHAARPGSQVQLVESGGGVVQPGRSLRLSCAASGFAFSSYGMHWVRQAPGKGLEWVAVIWFDGTKKYYTDSVKGRFTISRDNSKNTLYLQMNTLRAEDTAVYYCARDRGIGARRGPYYMDVWGKGTTVTVSSGGGGSGGGGSGGGGSGGGGSDIQMTQSPSSLSASVGDRVTITCRASQSISSYLNWYQQKPGKAPKLLIYAASSLQSGVPSRFSGSGSGTDFTLTISSLQPEDFATYYCQQSYSTPLTFGGGTKVEIKRTVSGTTTPAPRPPTPAPTIASQPLSLRPEACRPAAGGAVHTRGLDFACDFWVLVVVGGVLACYSLLVTVAFIIFWVRSKRSMVSKGEEDNMAIIKEFMRFKVHMEGSVNGHEFEIEGEGEGRPYEGTQTAKLKVTKGGPLPFAWDILSPQFMYGSKAYVKHPADIPDYLKLSFPEGFKWERVMNFEDGGVVTVTQDSSLQDGEFIYKVKLRGTNFPSDGPVMQKKTMGWEASSERMYPEDGALKGEIKQRLKLKDGGHYDAEVKTTYKAKKPVQLPGAYNVNIKLDITSHNEDYTIVEQYERAEGRHSTGGMDELYK

SEQ ID NO: 92 - DNA, 1773 bp, synthetic construct
  source 1..1773: mol_type = other DNA
atggccctgcctgtgacagccctgctgctgcctctggctctgctgctgcatgccgctagacccggatcccaggtgcagctggtggaaagcggcggcggcgtggtgcagccgggccgcagcctgcgcctgagctgcgcggcgagcggctttgcgtttagcagctatggcatgcattgggtgcgccaggcgccgggcaaaggcctggaatgggtggcggtgatttggtttgatggcaccaaaaaatattataccgatagcgtgaaaggccgctttaccattagccgcgataacagcaaaaacaccctgtatctgcagatgaacaccctgcgcgcggaagataccgcggtgtattattgcgcgcgcgatcgcggcattggcgcgcgccgcggcccgtattatatggatgtgtggggcaaaggcaccaccgtgaccgtgagcagcggcggaggtggaagcggaggcggaggatcaggtggcggtggatctggcggtggcggatctgatattcagatgacccagagcccgagcagcctgagcgcgagcgtgggcgatcgcgtgaccattacctgccgcgcgagccagagcattagcagctatctgaactggtatcagcagaaaccgggcaaagcgccgaaactgctgatttatgcggcgagcagcctgcagagcggcgtgccgagccgctttagcggcagcggcagcggcaccgattttaccctgaccattagcagcctgcagccggaagattttgcgacctattattgccagcagagctatagcaccccgctgacctttggcggcggcaccaaagtggaaattaaacgctccggaaccacgacgccagcgccgcgaccaccaacaccggcgcccaccatcgcgtcgcagcccctgtccctgcgcccagaggcgtgccggccagcggcggggggcgcagtgcacacgagggggctggacttcgcctgtgatttttgggtgctggtggtggttggtggagtcctggcttgctatagcttgctagtaacagtggcctttattattttctgggtgaggagtaagaggagcatggtgagcaagggcgaggaggataacatggccatcatcaaggagttcatgcgcttcaaggtgcacatggagggctccgtgaacggccacgagttcgagatcgagggcgagggcgagggccgcccctacgagggcacccagaccgccaagctgaaggtgaccaagggtggccccctgcccttcgcctgggacatcctgtcccctcagttcatgtacggctccaaggcctacgtgaagcaccccgccgacatccccgactacttgaagctgtccttccccgagggcttcaagtgggagcgcgtgatgaacttcgaggacggcggcgtggtgaccgtgacccaggactcctccctgcaggacggcgagttcatctacaaggtgaagctgcgcggcaccaacttcccctccgacggccccgtaatgcagaagaagaccatgggctgggaggcctcctccgagcggatgtaccccgaggacggcgccctgaagggcgagatcaagcagaggctgaagctgaaggacggcggccactacgacgctgaggtcaagaccacctacaaggccaagaagcccgtgcagctgcccggcgcctacaacgtcaacatcaagttggacatcacctcccacaacgaggactacaccatcgtggaacagtacgaacgcgccgagggccgccactccaccggcggcatggacgagctgtacaagtaa

SEQ ID NO: 93 - DNA, 63 bp, synthetic construct
  source 1..63: mol_type = other DNA
atggccctgcctgtgacagccctgctgctgcctctggctctgctgctgcatgccgctagaccc

SEQ ID NO: 94 - DNA, 60 bp, synthetic construct
  source 1..60: mol_type = other DNA
ggcggaggtggaagcggaggcggaggatcaggtggcggtggatctggcggtggcggatct

SEQ ID NO: 95 - DNA, 82 bp, synthetic construct
  source 1..82: mol_type = other DNA
tttttgggtgctggtggtggttggtggagtcctggcttgctatagcttgctagtaacagtggcctttattattttctgggtg

SEQ ID NO: 96 - DNA, 15 bp, synthetic construct
  source 1..15: mol_type = other DNA
aggagtaagaggagc

SEQ ID NO: 97 - DNA, 66 bp, synthetic construct
  source 1..66: mol_type = other DNA
ggaagcggagctactaacttcagcctgctgaagcaggctggagacgtggaggagaaccctggacct

SEQ ID NO: 98 - DNA, 708 bp, synthetic construct
  source 1..708: mol_type = other DNA
atggtgagcaagggcgaggaggataacatggccatcatcaaggagttcatgcgcttcaaggtgcacatggagggctccgtgaacggccacgagttcgagatcgagggcgagggcgagggccgcccctacgagggcacccagaccgccaagctgaaggtgaccaagggtggccccctgcccttcgcctgggacatcctgtcccctcagttcatgtacggctccaaggcctacgtgaagcaccccgccgacatccccgactacttgaagctgtccttccccgagggcttcaagtgggagcgcgtgatgaacttcgaggacggcggcgtggtgaccgtgacccaggactcctccctgcaggacggcgagttcatctacaaggtgaagctgcgcggcaccaacttcccctccgacggccccgtaatgcagaagaagaccatgggctgggaggcctcctccgagcggatgtaccccgaggacggcgccctgaagggcgagatcaagcagaggctgaagctgaaggacggcggccactacgacgctgaggtcaagaccacctacaaggccaagaagcccgtgcagctgcccggcgcctacaacgtcaacatcaagttggacatcacctcccacaacgaggactacaccatcgtggaacagtacgaacgcgccgagggccgccactccaccggcggcatggacgagctgtacaag

SEQ ID NO: 99 - DNA, 336 bp, synthetic construct
  source 1..336: mol_type = other DNA
agagtgaagttcagcaggagcgcagacgcccccgcgtacaagcagggccagaaccagctctataacgagctcaatctaggacgaagagaggagtacgatgttttggacaagagacgtggccgggaccctgagatggggggaaagccgagaaggaagaaccctcaggaaggcctgtacaatgaactgcagaaagataagatggcggaggcctacagtgagattgggatgaaaggcgagcgccggaggggcaaggggcacgatggcctttaccagggtctcagtacagccaccaaggacacctacgacgcccttcacatgcaggccctgccccctcgc

SEQ ID NO: 100 - protein, 21 aa, synthetic construct
MALPVTALLLPLALLLHAARP

SEQ ID NO: 101 - protein, 20 aa, synthetic construct
GGGGSGGGGSGGGGSGGGGS

SEQ ID NO: 102 - protein, 45 aa, synthetic construct
TTTPAPRPPTPAPTIASQPLSLRPEACRPAAGGAVHTRGLDFACD

SEQ ID NO: 103 - protein, 26 aa, synthetic construct
WVLVVVGGVLACYSLLVTVAFIIFWV

SEQ ID NO: 104 - protein, 5 aa, synthetic construct
RSKRS

SEQ ID NO: 105 - protein, 22 aa, synthetic construct
GSGATNFSLLKQAGDVEENPGP

SEQ ID NO: 106 - protein, 235 aa, synthetic construct
MVSKGEEDNMAIIKEFMRFKVHMEGSVNGHEFEIEGEGEGRPYEGTQTAKLKVTKGGPLPFAWDILSPQFMYGSKAYVKHPADIPDYLKLSFPEGFKWERVMNFEDGGVVTVTQDSSLQDGEFIYKVKLRGTNFPSDGPVMQKKTMGWEASSERMYPEDGALKGEIKQRLKLKDGGHYDAEVKTTYKAKKPVQLPGAYNVNIKLDITSHNEDYTIVEQYERAEGRHSTGGMDELY

SEQ ID NO: 107 - protein, 112 aa, synthetic construct
RVKFSRSADAPAYKQGQNQLYNELNLGRREEYDVLDKRRGRDPEMGGKPRRKNPQEGLYNELQKDKMAEAYSEIGMKGERRRGKGHDGLYQGLSTATKDTYDALHMQALPPR

SEQ ID NO: 108 - protein, 178 aa, synthetic construct
MPGSALLCCLLLLTGMRISRGQYSREDNNCTHFPVGQSHMLLELRTAFSQVKTFFQTKDQLDNILLTDSLMQDFKGYLGCQALSEMIQFYLVEVMPQAEKHGPEIKEHLNSLGEKLKTLRMRLRRCHRFLPCENKSKAVEQVKSDFNKLQDQGVYKAMNEFDIFINCIEAYMMIKMKS

SEQ ID NO: 109 - DNA, 534 bp, synthetic construct
  source 1..534: mol_type = other DNA
atgcctggctcagcactgctatgctgcctgctcttactgactggcatgaggatcagcaggggccagtacagccgggaagacaataactgcacccacttcccagtcggccagagccacatgctcctagagctgcggactgccttcagccaggtgaagactttctttcaaacaaaggaccagctggacaacatactgctaaccgactccttaatgcaggactttaagggttacttgggttgccaagccttatcggaaatgatccagttttacctggtagaagtgatgccccaggcagagaagcatggcccagaaatcaaggagcatttgaattccctgggtgagaagctgaagaccctcaggatgcggctgaggcgctgtcatcgatttctcccctgtgaaaataagagcaaggcagtggagcaggtgaagagtgattttaataagctccaagaccaaggtgtctacaaggccatgaatgaatttgacatcttcatcaactgcatagaagcatacatgatgatcaaaatgaaaagc

SEQ ID NO: 110 - protein, 27 aa, synthetic construct
STEEQFKSVLEIQKTGKYKKVETELLT

SEQ ID NO: 111 - DNA, 81 bp, synthetic construct
  source 1..81: mol_type = other DNA
tcaaccgaagaacagttcaaatcagtcttggagattcagaagactggaaaatacaagaaagttgaaaccgaactcctaacc

SEQ ID NO: 112 - DNA, 702 bp, synthetic construct
  source 1..702: mol_type = other DNA
atgagcgagctgattaaggagaacatgcacatgaagctgtacatggagggcaccgtggacaaccatcacttcaagtgcacatccgagggcgaaggcaagccctacgagggcacccagaccatgagaatcaaggtggtcgagggcggccctctccccttcgccttcgacatcctggctactagcttcctctacggcagcaagaccttcatcaaccacacccagggcatccccgacttcttcaagcagtccttccctgagggcttcacatgggagagagtcaccacatacgaagacgggggcgtgctgaccgctacccaggacaccagcctccaggacggctgcctcatctacaacgtcaagatcagaggggtgaacttcacatccaacggccctgtgatgcagaagaaaacactcggctgggaggccttcaccgagacgctgtaccccgctgacggcggcctggaaggcagaaacgacatggccctgaagctcgtgggcgggagccatctgatcgcaaacgccaagaccacatatagatccaagaaacccgctaagaacctcaagatgcctggcgtctactatgtggactacagactggaaagaatcaaggaggccaacaacgagacctacgtcgagcagcacgaggtggcagtggccagatactgcgacctccctagcaaactggggcacaagcttaattaa

SEQ ID NO: 113 - protein, 233 aa, synthetic construct
MSELIKENMHMKLYMEGTVDNHHFKCTSEGEGKPYEGTQTMRIKVVEGGPLPFAFDILATSFLYGSKTFINHTQGIPDFFKQSFPEGFTWERVTTYEDGGVLTATQDTSLQDGCLIYNVKIRGVNFTSNGPVMQKKTLGWEAFTETLYPADGGLEGRNDMALKLVGGSHLIANAKTTYRSKKPAKNLKMPGVYYVDYRLERIKEANNETYVEQHEVAVARYCDLPSKLGHKLN

SEQ ID NO: 114 - protein, 4 aa, synthetic construct
  REPEAT 1..4: note = repeat n times, where n is an integer of 1
GGGS

SEQ ID NO: 115 - protein, 5 aa, synthetic construct
  REPEAT 1..5: note = repeat n times, where n is an integer of 1
GGGGS

SEQ ID NO: 116 - protein, 4 aa, synthetic construct
GGSG

SEQ ID NO: 117 - protein, 5 aa, synthetic construct
GGSGG

SEQ ID NO: 118 - protein, 5 aa, synthetic construct
GSGSG

SEQ ID NO: 119 - protein, 5 aa, synthetic construct
GSGGG

SEQ ID NO: 120 - protein, 5 aa, synthetic construct
GGGSG

SEQ ID NO: 121 - protein, 5 aa, synthetic construct
GSSSG

SEQ ID NO: 122 - protein, 5 aa, synthetic construct
GGGGS

SEQ ID NO: 123 - protein, 15 aa, synthetic construct
GGGGSGGGGSGGGGS

SEQ ID NO: 124 - protein, 5 aa, synthetic construct
  REPEAT 1..5: note = repeat n times, where n is an integer of 1
GSGGS

SEQ ID NO: 125 - DNA, 45 bp, synthetic construct
  source 1..45: mol_type = other DNA
ggtggcggtggctcgggcggtggtgggtcgggtggcggcggatct

SEQ ID NO: 126 - DNA, 134 bp, synthetic construct
  source 1..134: mol_type = other DNA
accacgacgccagcgccgcgaccaccaacaccggcgcccaccatcgcgtcgcagcccctgtccctgcgcccagaggcgtgccggccagcggcggggggcgcagtgcacacgagggggctggacttcgcctgtga

(19) United States
(12) Patent Application Publication
(10) Pub. No.: US 2025/0025558 A1
(43) Pub. Date: Jan. 23, 2025

(54) Engineered Chimeric Antigen Receptor (CAR) Microglia-Like Cells for the Treatment of Neurodegenerative Disorders

(71) Applicant: The Trustees of the University of Pennsylvania, Philadelphia, PA (US)

(72) Inventors: Matias Porras-Paniagua, Philadelphia, PA (US); Saar Gill, Philadelphia, PA (US); Frederick Bennett, Philadelphia, PA (US)

(21) Appl. No.: 18/708,919

(22) PCT Filed: Nov. 10, 2022
(86) PCT No.: PCT/US2022/079659

§ 371 Date: May 9, 2024

Related U.S. Application Data
(60) Provisional application No. 63/277,886, filed on Nov. 10, 2021.

(51) Int. Cl. A61K 39/00; C07K 14/725; C07K 16/18

(52) U.S. Cl. CPC A61K 39/46432; A61K 39/4611; A61K 39/4614; A61K 39/4631; C07K 14/7051; C07K 16/18; C07K 2317/24

(57) ABSTRACT

The present disclosure provides compositions and methods comprising chimeric antigen receptors (CARs) specific for amyloid beta (Aβ) and/or Tau. In certain embodiments, the CARs do not comprise an intracellular domain. Methods of treatment are also disclosed herein.

### CROSS-REFERENCE TO RELATED APPLICATION

The present application is a 35 U.S.C. § 371 national phase application of, and claims priority to, International Application No. PCT/US2022/079659, filed Nov. 10, 2022, which is entitled to priority under 35 U.S.C. § 119(e) to U.S. Provisional Patent Application No. 63/277,886 filed Nov. 10, 2021, each of which applications is hereby incorporated by reference in its entirety herein.

### SEQUENCE LISTING

The present application contains a Sequence Listing which has been submitted in XML format via Patent Center and is hereby incorporated by reference in its entirety. Said XML file, created on Apr. 30, 2024, is named 046483-7351US1 Sequence Listing.xml and is 176,128 bytes in size.

### BACKGROUND OF THE INVENTION

Alzheimer's Disease (AD) is the leading cause of dementia and the incidence is expected to double by 2050 due to the increasing age of the US population. AD, an incurable neurodegenerative disorder is characterized by amyloid beta (Aβ) accumulation and subsequent deposition of tau, affecting downstream signaling cascades that lead to neurodegeneration and cognitive impairment. Preventing or reversing Aβ deposition is a long-standing therapeutic goal. Genome-wide association studies for sporadic late-onset AD implicate microglial genes, most notably genes linked to Aβ phagocytosis. Since microglia are brain-resident phagocytic cells with the capacity to uptake Aβ, these findings suggest that microglial dysfunction may play a crucial role in AD pathogenesis. To date, prevention of Aβ accumulation has been attempted via systemic administration of anti-Aβ monoclonal antibodies (mAbs), culminating in the recent FDA approval of aducanumab. Interestingly, this approval occurred despite poor efficacy and some toxicity in human studies, likely reflecting a profound unmet medical need. Poor efficacy may be attributed to 1) poor CNS penetration and 2) failure to recruit effector cells (microglia) to clear amyloid.

A need exists for a more direct and potent means of redirecting microglia to uptake Aβ, where traditional mAb-based therapeutics have failed. The present invention addresses this need.

### SUMMARY OF THE INVENTION

As described herein, the present invention relates to compositions and methods comprising chimeric antigen receptors (CARs) specific for amyloid beta (Aβ) and/or Tau.

In one aspect, the invention includes a chimeric antigen receptor (CAR) comprising an antigen-binding domain and a transmembrane domain, wherein the antigen-binding domain binds amyloid beta (Aβ).

In certain embodiments, the antigen-binding domain comprises a heavy chain variable region that comprises three heavy chain complementarity determining regions (HCDRs) and a light chain variable region that comprises three light chain complementarity determining regions (LCDRs), wherein at least one of the complementarity determining regions comprises any one of SEQ ID NOs: 1-6.

In certain embodiments, HCDR1 comprises the amino acid sequence SYGMH (SEQ ID NO: 1), HCDR2 comprises the amino acid sequence VIWFDGTKKYYTDSVKG (SEQ ID NO: 2), HCDR3 comprises the amino acid sequence DRGIGARRGPYYMDV (SEQ ID NO: 3), LCDR1 comprises the amino acid sequence RASQSISSYLN (SEQ ID NO: 4), LCDR2 comprises the amino acid sequence ASSLQS (SEQ ID NO: 5), and LCDR3 comprises the amino acid sequence QQSYSTPLT (SEQ ID NO: 6).

In certain embodiments, the heavy chain variable region (VH) of the antigen-binding domain comprises an amino acid sequence at least 80%, 85%, 90%, 95%, 96%, 97%, 98%, 99%, or 100% identical to SEQ ID NO: 7 and/or the light chain variable region (VL) comprises an amino acid sequence at least 80%, 85%, 90%, 95%, 96%, 97%, 98%, 99%, or 100% identical to SEQ ID NO: 8.

In certain embodiments, the VH of the antigen-binding domain is encoded by a nucleotide sequence at least 80%, 85%, 90%, 95%, 96%, 97%, 98%, 99%, or 100% identical to SEQ ID NO: 9 and/or the VL is encoded by a nucleotide sequence at least 80%, 85%, 90%, 95%, 96%, 97%, 98%, 99%, or 100% identical to SEQ ID NO: 10.

In certain embodiments, the antigen-binding domain is a single-chain variable fragment (scFv) comprising an amino acid sequence at least 80%, 85%, 90%, 95%, 96%, 97%, 98%, 99%, or 100% identical to SEQ ID NO: 11 or SEQ ID NO: 12.

In certain embodiments, the antigen-binding domain is a scFv encoded by a nucleotide sequence at least 80%, 85%, 90%, 95%, 96%, 97%, 98%, 99%, or 100% identical to SEQ ID NO: 13 or SEQ ID NO: 14.

In certain embodiments, the CAR comprises an amino acid sequence at least 80%, 85%, 90%, 95%, 96%, 97%, 98%, 99%, or 100% identical to SEQ ID NO: 15 or SEQ ID NO: 16.

In certain embodiments, the CAR is encoded by a nucleotide sequence at least 80%, 85%, 90%, 95%, 96%, 97%, 98%, 99%, or 100% identical to SEQ ID NO: 17 or SEQ ID NO: 18.

In certain embodiments, the antigen-binding domain comprises a heavy chain variable region that comprises three HCDRs and a light chain variable region that comprises three LCDRs, wherein at least one of the complementarity determining regions comprises any one of SEQ ID NOs: 19-24.

In certain embodiments, HCDR1 comprises the amino acid sequence GFTFSSYGMS (SEQ ID NO: 19), HCDR2 comprises the amino acid sequence SINSNGGSTYYPDSVK (SEQ ID NO: 20), HCDR3 comprises the amino acid sequence GDY (SEQ ID NO: 21), LCDR1 comprises the amino acid sequence RSSQSLVYSNGDTYLH (SEQ ID NO: 22), LCDR2 comprises the amino acid sequence KVSNRFS (SEQ ID NO: 23), and LCDR3 comprises the amino acid sequence SQSTHVPWT (SEQ ID NO: 24).

In certain embodiments, the VH of the antigen-binding domain comprises an amino acid sequence at least 80%, 85%, 90%, 95%, 96%, 97%, 98%, 99%, or 100% identical to SEQ ID NO: 25 and/or the VL comprises an amino acid sequence at least 80%, 85%, 90%, 95%, 96%, 97%, 98%, 99%, or 100% identical to SEQ ID NO: 26.

In certain embodiments, the VH of the antigen-binding domain is encoded by a nucleotide sequence at least 80%, 85%, 90%, 95%, 96%, 97%, 98%, 99%, or 100% identical to SEQ ID NO: 27 and/or the VL is encoded by a nucleotide sequence at least 80%, 85%, 90%, 95%, 96%, 97%, 98%, 99%, or 100% identical to SEQ ID NO: 28.

In certain embodiments, the antigen-binding domain is a scFv comprising an amino acid sequence at least 80%, 85%, 90%, 95%, 96%, 97%, 98%, 99%, or 100% identical to SEQ ID NO: 29 or SEQ ID NO: 30.

In certain embodiments, the antigen-binding domain is a scFv encoded by a nucleotide sequence at least 80%, 85%, 90%, 95%, 96%, 97%, 98%, 99%, or 100% identical to SEQ ID NO: 31 or SEQ ID NO: 32.

In certain embodiments, the CAR comprises an amino acid sequence at least 80%, 85%, 90%, 95%, 96%, 97%, 98%, 99%, or 100% identical to SEQ ID NO: 33 or SEQ ID NO: 34.

In certain embodiments, the CAR is encoded by a nucleotide sequence at least 80%, 85%, 90%, 95%, 96%, 97%, 98%, 99%, or 100% identical to SEQ ID NO: 35 or SEQ ID NO: 36.

In certain embodiments, the antigen-binding domain comprises a heavy chain variable region that comprises three HCDRs and a light chain variable region that comprises three LCDRs, wherein at least one of the complementarity determining regions comprises any one of SEQ ID NOs: 37-42.

In certain embodiments, HCDR1 comprises the amino acid sequence NYGMS (SEQ ID NO: 37), HCDR2 comprises the amino acid sequence IRSGGGRTYYSDNVKGR (SEQ ID NO: 38), HCDR3 comprises the amino acid sequence YDHYSGSSDY (SEQ ID NO: 39), LCDR1 comprises the amino acid sequence KSSQSLLDSDGKTYLN (SEQ ID NO: 40), LCDR2 comprises the amino acid sequence LVSKLD (SEQ ID NO: 41), and LCDR3 comprises the amino acid sequence WQGTHFPRT (SEQ ID NO: 42).

In certain embodiments, the VH of the antigen-binding domain comprises an amino acid sequence at least 80%, 85%, 90%, 95%, 96%, 97%, 98%, 99%, or 100% identical to SEQ ID NO: 43 and/or the VL comprises an amino acid sequence at least 80%, 85%, 90%, 95%, 96%, 97%, 98%, 99%, or 100% identical to SEQ ID NO: 44.

In certain embodiments, the VH of the antigen-binding domain is encoded by a nucleotide sequence at least 80%, 85%, 90%, 95%, 96%, 97%, 98%, 99%, or 100% identical to SEQ ID NO: 45 and/or the VL is encoded by a nucleotide sequence at least 80%, 85%, 90%, 95%, 96%, 97%, 98%, 99%, or 100% identical to SEQ ID NO: 46.

In certain embodiments, the antigen-binding domain is scFv comprising an amino acid sequence at least 80%, 85%, 90%, 95%, 96%, 97%, 98%, 99%, or 100% identical to SEQ ID NO: 47 or SEQ ID NO: 48.

In certain embodiments, the antigen-binding domain is a scFv encoded by a nucleotide sequence at least 80%, 85%, 90%, 95%, 96%, 97%, 98%, 99%, or 100% identical to SEQ ID NO: 49 or SEQ ID NO: 50.

In certain embodiments, the CAR comprises an amino acid sequence at least 80%, 85%, 90%, 95%, 96%, 97%, 98%, 99%, or 100% identical to SEQ ID NO: 51 or SEQ ID NO: 52.

In certain embodiments, the CAR is encoded by a nucleotide sequence at least 80%, 85%, 90%, 95%, 96%, 97%, 98%, 99%, or 100% identical to SEQ ID NO: 53 or SEQ ID NO: 54.

Another aspect of the invention includes a chimeric antigen receptor (CAR) comprising an antigen-binding domain and a transmembrane domain, wherein the antigen-binding domain binds Tau.

In certain embodiments, the antigen-binding domain comprises a heavy chain variable region that comprises three heavy chain complementarity determining regions (HCDRs) and a light chain variable region that comprises three light chain complementarity determining regions (LCDRs), wherein at least one of the complementarity determining regions comprises any one of SEQ ID NOs: 55-60.

In certain embodiments, HCDR1 comprises the amino acid sequence KYGMS (SEQ ID NO: 55), HCDR2 comprises the amino acid sequence ISSSGSRTYYPDSVKG (SEQ ID NO: 56), HCDR3 comprises the amino acid sequence WDGAMDY (SEQ ID NO: 57), LCDR1 comprises the amino acid sequence KSSQSIVHSNGNTYLE (SEQ ID NO: 58), LCDR2 comprises the amino acid sequence KVSNRF (SEQ ID NO: 59), and LCDR3 comprises the amino acid sequence FQGSLVPWA (SEQ ID NO: 60).

In certain embodiments, the VH of the antigen-binding domain comprises an amino acid sequence at least 80%, 85%, 90%, 95%, 96%, 97%, 98%, 99%, or 100% identical to SEQ ID NO: 61 and/or VL comprises an amino acid sequence at least 80%, 85%, 90%, 95%, 96%, 97%, 98%, 99%, or 100% identical to SEQ ID NO: 62.

In certain embodiments, the VH of the antigen-binding domain is encoded by a nucleotide sequence at least 80%, 85%, 90%, 95%, 96%, 97%, 98%, 99%, or 100% identical to SEQ ID NO: 63 and/or the VL is encoded by a nucleotide sequence at least 80%, 85%, 90%, 95%, 96%, 97%, 98%, 99%, or 100% identical to SEQ ID NO: 64.

In certain embodiments, the antigen-binding domain is a scFv comprising an amino acid sequence at least 80%, 85%, 90%, 95%, 96%, 97%, 98%, 99%, or 100% identical to SEQ ID NO: 65 or SEQ ID NO: 66.

In certain embodiments, the antigen-binding domain is a scFv encoded by a nucleotide sequence at least 80%, 85%, 90%, 95%, 96%, 97%, 98%, 99%, or 100% identical to SEQ ID NO: 67 or SEQ ID NO: 68.

In certain embodiments, the CAR comprises an amino acid sequence at least 80%, 85%, 90%, 95%, 96%, 97%, 98%, 99%, or 100% identical to SEQ ID NO: 69 or SEQ ID NO: 70.

In certain embodiments, the CAR is encoded by a nucleotide sequence at least 80%, 85%, 90%, 95%, 96%, 97%, 98%, 99%, or 100% identical to SEQ ID NO: 71 or SEQ ID NO: 72.

In certain embodiments, the CAR does not contain an intracellular domain.

In certain embodiments, the CAR further comprises an intracellular domain.

In certain embodiments, the CAR comprises an amino acid sequence at least 80%, 85%, 90%, 95%, 96%, 97%, 98%, 99%, or 100% identical to any one of SEQ ID NOs: 73, 74, 77, 78, 81, or 82.

In certain embodiments, the CAR is encoded by a nucleotide sequence at least 80%, 85%, 90%, 95%, 96%, 97%, 98%, 99%, or 100% identical to any one of SEQ ID NOs: 75, 76, 79, 80, 83, or 84.

Another aspect of the invention includes a modified immune cell comprising any of the CARs contemplated herein.

In certain embodiments, the modified immune cell further comprises a second CAR, wherein the second CAR comprises any of the CARs contemplated herein.

In certain embodiments, the cell is a monocyte, macrophage, B cell, T cell, NK cell, neutrophil, or stem cell.

Another aspect of the invention includes a pharmaceutical composition comprising any of the modified immune cells contemplated herein, and a pharmaceutically acceptable carrier.

Another aspect of the invention includes a method of treating a neurodegenerative disease in a subject in need thereof. The method comprises administering to the subject a therapeutically effective amount of any of the pharmaceutical compositions contemplated herein.

In certain embodiments, the neurodegenerative disease is Alzheimer's Disease (AD) or a tauopathy.

In certain embodiments, the method further comprises depleting the endogenous microglia in the subject prior to administering the pharmaceutical composition.

Another aspect of the invention includes a method of treating a neurodegenerative disease in a subject in need thereof. The method comprises administering to the subject a therapeutically effective amount of a composition comprising a cell comprising a chimeric antigen receptor (CAR), wherein the CAR comprises an antigen binding domain that binds amyloid beta, and wherein the CAR does not comprise an intracellular domain, and wherein the cell is a monocyte, macrophage, dendritic cell, or stem cell.

Another aspect of the invention includes a method of treating a neurodegenerative disease in a subject in need thereof. The method comprises depleting the endogenous microglia in the subject, and administering to the subject a therapeutically effective amount of a composition comprising a cell comprising a chimeric antigen receptor (CAR), wherein the CAR comprises an antigen binding domain that binds amyloid beta, and wherein the CAR does not comprise an intracellular domain, and wherein the cell is a monocyte, macrophage, dendritic cell, or stem cell.

In certain embodiments, the neurodegenerative disease is Alzheimer's Disease (AD) or a tauopathy.

In certain embodiments, the CAR delivers a payload.

In certain embodiments, the CAR is delivered via a lipid nanoparticle (LNP).

### BRIEF DESCRIPTION OF THE DRAWINGS

The foregoing and other features and advantages of the present invention will be more fully understood from the following detailed description of illustrative embodiments taken in conjunction with the accompanying drawings. It should be understood that the present invention is not limited to the precise arrangements and instrumentalities of the embodiments shown in the drawings.

FIG. 1: Schema illustrating the generation of a library of chimeric antigen receptor (CAR) constructs.

FIG. 2: CAR constructs used in the experiments herein.

FIG. 3: Gating strategy based on no Aβ control. The gating strategy was used to determine the percent of CAR+ cells that uptake Aβ (Cells→Single cells→CAR+ Cells→Aβ+ cells). CAR expression was defined by detection of the genetic reporter mCherry. Cells tested were HMC3, human fetal microglial cell line, at 10,000 cells per well in a 96 well plate. Cells were incubated with media containing fluorescently-labeled (AF488) amyloid beta for 1 hr at 37 degrees celcius. Amyloid beta concentration ranges were (733.33 nM, 244.4 nM, 81.5 nM, 27 nM, 9 nM, 3 nM, 1 nM, 0 nM).

FIG. 4: Gating Strategy (Ex: Aducanumab H2L CAR Uptaking Aβ). Same gating strategy as in FIG. 3 demonstrating uptake of Aβ via CAR-HMC3 cell.

FIG. 5: Anti-Aβ CAR Crenezumab HMC3 cells exhibit greater Aβ fluorescent signal compared to nonspecific CAR HMC3 cells. Evaluation of Aβ uptake by CAR expressing HMC3 cells (Crenezumab Constructs). Left: Evaluation by % Aβ+ cells, Right: Evaluation by weighted mean fluorescent intensity of fluorescently-labeled amyloid beta in CAR+ cells bearing amyloid beta. Weighted MFI was measured by multiplying fraction of CAR+ cells that were Aβ+ by the MFI of CAR+Aβ+ cells to determine Aβ content associated with the cell. Measurement: Flow cytometry.

FIG. 6: Anti-Aβ CAR Aducanumab HMC3 cells exhibit greater Aβ uptake signal compared to nonspecific CAR HMC3 cells. Evaluation of Aβ uptake by CAR expressing HMC3 cells (Aducanumab Constructs). Left: Evaluation by % Aβ+ cells, Right: Evaluation by weighted mean fluorescent intensity of fluorescently-labeled amyloid beta in CAR+ cells bearing amyloid beta. Measurement: Flow cytometry.

FIG. 7: Anti-Aβ CAR 3D6 HMC3 cells exhibit greater Aβ fluorescent signal compared to nonspecific CAR HMC3 cells. Evaluation of Aβ uptake by CAR expressing HMC3 cells (3D6 Constructs). Left: Evaluation by % Aβ+ cells, Right: Evaluation by weighted mean fluorescent intensity of fluorescently-labeled amyloid beta in CAR+ cells bearing amyloid beta. Measurement: Flow cytometry.

FIG. 8: Aβ fluorescence signal decreases after 24 hrs in Anti-Aβ CAR-HMC3 cells. Statistical Test: Two Way ANOVA.

FIGS. 9-11: Confocal microscopy imaging corroborates superior Aβ uptake by anti-Aβ CAR construct HMC3 cells. FIG. 9: Representative images 0 hr post Aβ removal. FIG. 10: Representative images 24 hr post Aβ removal. FIG. 11: Representative images 48 hr post Aβ removal. From left to right: CAR19 CD3z Construct, Aducanumab Construct, 3D6 Clone Construct, Crenezumab Construct.

FIG. 12: Confocal analysis supports trend observed on Flow Cytometry that Aβ decreases over time.

FIG. 13: Image analysis pipeline.

FIG. 14: CAR Aducanumab (H2L) HMC3 live cell imaging.

FIG. 15: CAR19 CD3z HMC3 live cell imaging.

FIG. 16: Schematic of Protocol to generate primary murine CAR macropahges.

FIG. 17: Gating Strategy (Based on No Aβ Control). Gating strategy demonstrating CAR gating and no Aβ uptake in no Aβ control, defining Aβ uptake threshold. (Cells→singlets→CAR+/CAR−→Aβ+(on CAR− cells)).

FIG. 18: Anti-Aβ CAR murine BMDMs have superior Aβ association compared to nonspecific CARs or untransduced cells after incubating cells in media containing fluorescently-labeled amyloid beta for 1 hr at 37° C.

FIG. 19: Anti-Aβ CAR murine BMDMs have superior Aβ MFI association compared to nonspecific CARs or untransduced cells after incubating cells in media containing fluorescently-labeled amyloid beta for 1 hr at 37° C.

FIG. 20: Confocal imaging showing that Anti-Aβ CAR murine BMDMs demonstrate superior Aβ internalization compared to nonspecific CAR murine BMDMs.

FIG. 21: Flow gating strategy (UTD No Aβ Control). (Cells→singlets→Live Cells→CAR+(mCherry)/Aβ+(AF488)).

FIG. 22: Flow gating strategy (CAR No Aβ Control). (Cells→singlets→Live Cells→CAR+(mCherry)/Aβ+(AF488)).

FIG. 23: Flow plots (CARAβ+Aβ) murine BMDMs. (Cells→singlets→Live Cells→CAR+(mCherry)/Aβ+(AF488)). Amyloid association is higher in CARAβ than untransduced cells.

FIG. 24: AB degradation curve in BMDMs showing that CARAβ bearing BMDMs are capable of uptaking more amyloid beta compared to untransduced cells and can degrade amyloid beta over time. Left: Evaluation of Aβ signal via % Aβ+ CAR BMDMs. Right: Evaluation of Aβ signal via weighted average of mean fluorescent intensity of fluorescently-labeled Aβ and % Aβ+ CAR bearing BMDMs.

FIG. 25: Fluorescent Aβ is not detectable in nonspecific CAR murine BMDMs via widefield microscopy.

FIG. 26: Aβ fluorescent signal is eliminated in 24 hrs by anti-Aβ CAR murine BMDMs as measured via widefield microscopy.

FIG. 27: Anti-Aβ CAR Murine BMDMs exhibit superior Aβ uptake compared to nonspecific CAR murine BMDMs as measured via widefield microscopy.

FIG. 28: Gating strategy (Cells→singlets→CAR+→Aβ+) based on UTD no Aβ control showing no amyloid beta association by untreated untransduced cells to set the detection limits for CAR expression and amyloid beta association.

FIG. 29: Example gating Aβ−CAR mBMDM (Cells→singlets→CAR+→Aβ+).

FIG. 30: Anti-Aβ CAR leads to higher Aβ uptake than non-specific CARs or to primary murine BMDMs redirected using aducanumab.

FIG. 31: Flow gating scheme (UTD no Aβ) negative control showing no CAR expression and no Aβ association.

FIG. 32: Flow gating scheme (CAR Aβ No Aβ) showing CAR expression in murine BMDMs but no amyloid beta association.

FIG. 33: Flow gating scheme (CAR Aβ No inhibitor) showing CAR expression and amyloid beta uptake.

FIG. 34: Dextran Sulfate is a potent inhibitor of the non-specific uptake of AB by UTDs, but exhibits minor inhibition of AB uptake by CAR-M.

FIG. 35: Anti-Tau CAR design and approach.

FIG. 36: Flow gating strategy (UTD No Tau) (cells→singlets→CAR (mCherry)/Tau (ATTO-488) showing no CAR expression and no Tau association.

FIG. 37: Flow gating strategy (CAR Tau+ Tau) showing Tau association specifically mediated by the anti-Tau CAR.

FIG. 38: Tau CARs outperform CARAβ (Aducanumab H2L CAR), CAR19 and UTDs in HMC3s showing that association is driven specifically by an anti-tau targeting CAR construct.

FIG. 39: Tau assay with BMDMs set-up.

FIG. 40: Flow gating scheme (UTD No Tau) (cells→singlets→CAR (mCherry)/Tau (ATTO-488)) showing no CAR expression and no Tau association.

FIG. 41: Flow gating scheme (H2L CAR-Tau No Tau) (cells→singlets→CAR (mCherry)/Tau (ATTO-488)) showing CAR expression but no Tau association.

FIG. 42: Flow plots (H2L CAR-Tau 250 nM Tau) (cells→singlets→CAR (mCherry)/Tau (ATTO-488)) showing CAR expression and Tau association.

FIG. 43: CARTau (H2L) (Gosuranemab H2L CAR) and CARTau (L2H) (Gosuranemab L2H CAR) outperform CAR19 and UTD at 250 nM.

FIG. 44: Gating Scheme UTD human macrophages no Aβ (cells→singlets→CAR (mCherry)/Aβ (AF488) showing no CAR expression and no Aβ association.

FIG. 45: Gating Scheme UTD human macrophages+Aβ (cells→singlets→CAR (mCherry)/Aβ (AF488)), showing no CAR expression and non-specific association with amyloid beta.

FIG. 46: Gating Scheme UTD human macrophages+Aducanumab 0.1 ug/mL (cells→singlets→CAR (mCherry)/Aβ (AF488)), showing no CAR expression and association with amyloid beta driven by aducanumab.

FIG. 47: Gating Scheme CAR-Aβ human macrophages+Aβ (cells→singlets→CAR (mCherry)/Aβ (AF488)) showing CAR expression and association with amyloid beta driven specifically by the anti-Aβ CAR.

FIG. 48: Evaluation of Aβ uptake in human macrophages demonstrating that anti-Aβ CAR mediated association is superior to amyloid beta association driven by aducanumab.

FIG. 49: Schema for quantification of Aβ degradation by CAR-BMDMs using ELISA.

FIG. 50: CAR-Aβ BMDMs cleared amyloid beta from supernatant more efficiently than UTDs.

FIG. 51: General CAR structure.

FIG. 52: Aducanumab H2L CAR diagram.

FIG. 53: Aducanumab L2H CAR diagram.

FIG. 54: Crenezumab H2L CAR diagram.

FIG. 55: Crenezumab L2H CAR diagram.

FIG. 56: mAb 3D6 H2L CAR diagram.

FIG. 57: mAb 3D6 L2H CAR diagram.

FIG. 58: Gosuranemab H2L diagram.

FIG. 59: Gosuranemab L2H diagram.

FIG. 60: Aducanumab H2L CD3z CAR diagram.

FIG. 61: Aducanumab L2H CD3z CAR diagram.

FIG. 62: 3D6 H2L CD3z CAR diagram.

FIG. 63: 3D6 L2H CD3z CAR diagram.

FIG. 64: Crenezumab H2L CD3z CAR diagram.

FIG. 65: Crenezumab L2H CD3z CAR diagram.

FIG. 66: Aducanumab H2L IL-10 CAR diagram.

FIG. 67: Aducanumab H2L Fcγ CAR diagram.

FIG. 68: Dual CAR (Aducanumab H2L, Gosuranemab H2L) diagram.

FIG. 69: Aducanumab H2L fused reporter CAR diagram.

FIG. 70: ELISA study showing HMC3 cells expressing CARAβ IL10 secrete IL10 compared to HMC3 cells expressing CARAβ demonstrating that CAR bearing cells can deliver a therapeutic payload.

FIG. 71: Representative flow (untreated) gating of HMC3 cell line expressing CARAβ, CARTau, both CARs, or neither (cells→singlets→CARAβ (BFP)/CARTau (mCherry)→Aβ (AF647)/Tau (ATTO-488)) showing no association with amyloid beta or Tau.

FIG. 72: Representative flow (exposure to Amyloid Beta) gating of HMC3 cell line expressing CARAβ, CARTau, both CARs, or neither (cells→singlets→CARAβ (BFP)/CARTau (mCherry)→Aβ (AF647)/Tau (ATTO-488)) showing association of amyloid beta driven specifically by the anti-Aβ CAR construct.

FIG. 73: Representative flow (exposure to Tau) gating of HMC3 cell line expressing CARAβ, CARTau, both CARs, or neither (cells→singlets→CARAβ (BFP)/CARTau (mCherry)→Aβ (AF647)/Tau (ATTO-488)) showing association of Tau driven specifically by the anti-Tau CAR construct.

FIG. 74: Representative flow (exposure to both Amyloid Beta & Tau) gating of HMC3 cell line expressing CARAβ, CARTau, both CARs, or neither (cells→singlets→CARAβ (BFP)/CARTau (mCherry)+Aβ (AF647)/Tau (ATTO-488)) showing that cells expressing both CARs are able to target multiple protein aggregates simultaneously in a specific manner.

FIG. 75: Cells expressing both CARs are able to target multiple protein aggregates simultaneously in a specific manner.

FIG. 76: Gating scheme for LNP CARAβ murine macrophages (untreated) to set GFP and CAR detection limits. (Cells→singlets→GFP/CAR (mCherry)).

FIG. 77: Gating scheme for LNP CARAβ murine macrophages (GFP LNP) (Cells→singlets→GFP/CAR (mCherry)) demonstrating that macrophages can express the mRNA GFP payload.

FIG. 78: Gating scheme for LNP CARAβ murine macrophages (CARAβ LNP) (Cells→singlets→GFP/CAR (mCherry)) demonstrating that macrophages can express the mRNA CARAβ payload.

FIG. 79: Gating scheme for LNP CARAβ murine macrophages (CARAβ FcY LNP) (Cells→singlets→GFP/CAR (mCherry)) demonstrating that macrophages are capable of expressing the mRNA CARAβ Fcγ payload.

FIG. 80: LNP CARAβ murine macrophages (summary). UTD expresses no GFP or CARAβ. UTD+GFP-LNPs express GFP but no CARAβ. UTD+CARAβ−mCherry & UTD+CARAβ FcY-mCherry express CARAβ but no GFP.

FIG. 81: Gating scheme for LNP human macrophages (UTD untreated) (Cells→singlets→Live cells→GFP/CAR (mCherry)) to set GFP and CAR detection limits.

FIG. 82: Gating scheme for LNP human macrophages (GFP LNP) (Cells→singlets→Live cells→GFP/CAR (mCherry)) showing that human macrophages can express the mRNA GFP payload.

FIG. 83: Gating scheme for LNP human macrophages (CARAβ mCherry LNP treated) (Cells→singlets→Live cells→GFP/CAR (mCherry)) showing that human macrophages can express the mRNA CARAβ payload.

FIG. 84: LNP macrophages (Summary). UTD expresses no GFP or CARAβ. UTD+GFP-LNPs express GFP but no CARAβ. UTD+CARAβ−mCherry express CARAβ but no GFP.

FIG. 85: Schematic of augmented HSC bone marrow transplant process to replace endogenous microglia with engineered surrogates.

FIG. 86: Gating strategy for peripheral engraftment wild type mice (Untreated) to set CAR (mCherry) detection limits.

FIG. 87: Gating strategy for peripheral engraftment wild type mice (CARAβ HSC treated) showing detectable CAR expressing cells in circulation.

FIG. 88: CARAβ Peripheral engraftment over time. Peripheral engraftment from retro-orbital bleeds. Showing CAR+ cells for 3 months post BMT.

FIG. 89: Gating scheme for lineage specific CAR+ at 3 months (untreated mouse) to set gating for donor derived cells.

FIG. 90: Gating scheme for lineage specific CAR+ at 3 months (CARAβ HSC treated mouse) showing engraftment of donor derived cells.

FIG. 91: Gating scheme for lineage specific CAR+ at 3 months (CARAβ HSC treated Mouse; Neutrophils). Gating continues from CD45.2 donor cells demonstrating CARAβ bearing neutrophils in circulation.

FIG. 92: Gating scheme for lineage specific CAR+ at 3 months (CARAβ HSC treated Mouse; B cells). Gating continues from CD45.2 non neutrophil showing CARAβ bearing B cells in circulation.

FIG. 93: Gating scheme for lineage specific CAR+ at 3 months (CARAβ HSC treated mouse; T cells). Gating continues from CD45.2 non neutrophil showing CARAβ bearing Tcells in circulation.

FIG. 94: Gating scheme for lineage specific CAR+ at 3 months (CARAβ HSC treated Mouse; Monocytes). Gating continues from CD45.2 non neutrophil showing CARAβ bearing monocytes in circulation.

FIG. 95: Gating scheme for lineage specific CAR+ at 3 months (CARAβ HSC treated mouse; NK cells). Gating continues from CD45.2 non neutrophil showing CARAβ bearing NK cells in circulation.

FIG. 96: CAR+ expression by lineage cells at 3 months post BMT. All HSC lineage cells have stable CAR expression 3 months post BMT.

FIG. 97: Representative image: untreated mice (no cell engraftment). Representative slice of the Cortex. 10× Magnification. Cells were stained for anti-mCherry primary with secondary 594 antibody.

FIG. 98: Representative image: CARAβ treated mice showing engraftment of CAR microglia. Representative slice of the cortex. 10× magnification. Cells were stained for anti-mCherry primary with secondary 594 antibody.

FIG. 99: Gating strategy for peripheral engraftment (untreated) in the 5×FAD Alzheimer's mouse model to set the gating for CAR detection.

FIG. 100: Gating strategy for peripheral engraftment (untreated, neutrophils) showing no expression of CARAβ in neutrophils of the untreated 5×FAD mice.

FIG. 101: Gating strategy for peripheral engraftment (untreated, B cells) showing no expression of CARAβ in B cells of the untreated 5×FAD mice.

FIG. 102: Gating strategy for peripheral engraftment (untreated, T cells) showing no expression of CARAβ in T cells of the untreated 5×FAD mice.

FIG. 103: Gating strategy for peripheral engraftment (untreated, monocytes) showing no expression of CARAβ in monocytes of the untreated 5×FAD mice.

FIG. 104: Gating strategy for peripheral engraftment (untreated, NK cells) showing no expression of CARAβ in NK cells of the untreated 5×FAD mice.

FIG. 105: Gating strategy for peripheral engraftment (CARAβ HSC treated) showing CARAβ expression of cells in circulation of CARAβ HSC treated 5×FAD mice.

FIG. 106: Gating strategy for peripheral engraftment (CARAβ HSCs, neutrophils) showing CARAβ expression of neutrophils in circulation of CARAβ HSC treated 5×FAD mice.

FIG. 107: Gating strategy for peripheral engraftment (CARAβ HSCs, B cells) showing CARAβ expression of B cells in circulation of CARAβ HSC treated 5×FAD mice.

FIG. 108: Gating strategy for peripheral engraftment (CARAβ HSCs, T cells) showing CARAβ expression of T cells in circulation of CARAβ HSC treated 5×FAD mice.

FIG. 109: Gating strategy for peripheral engraftment (CARAβ HSCs, monocytes) showing CARAβ expression of monocytes in circulation of CARAβ HSC treated 5×FAD mice.

FIG. 110: Gating strategy for peripheral engraftment (CARAβ HSCs, NK cells) showing CARAβ expression of NK cells in circulation of CARAβ HSC treated 5×FAD mice.

FIG. 111: Significant peripheral engraftment of CAR-HSC cells at 2-week timepoint. At 2 weeks post bone marrow transplant there is significant CAR engraftment of monocytes, NK cells and neutrophils in the 5×FAD mice.

FIG. 112: Aducanumab H2L P2A BFP CAR diagram.

FIG. 113: Representative image: CARP HSC treated mouse showing that CAR expressing cells that engraft in the brain express Iba1 (a macrophage/microglia specific marker) and adopt microglia-like morphology. Representative slice of the Midbrain at 40× Magnification. Cells were stained for anti-mCherry primary with secondary 594 antibody and cells were stained for Iba1 (macrophage/microglia marker) with an anti-Iba1 primary and a secondary 488 antibody.

FIG. 114: Representative CARAβ HSC treated mouse showing CAR expressing cells in the midbrain. Same image as FIG. 113 but isolated to the red channel (CAR expressing cells).

FIG. 115: Representative CARAβ HSC treated mouse showing CAR expressing cells in the midbrain. Same image as FIG. 113 but isolated to the green channel (Iba1 expressing cells).

### DETAILED DESCRIPTION OF THE INVENTION

### Definitions

Unless defined otherwise, all technical and scientific terms used herein have the same meaning as commonly understood by one of ordinary skill in the art to which the invention pertains. Although any methods and materials similar or equivalent to those described herein can be used in the practice for testing of the present invention, the preferred materials and methods are described herein. In describing and claiming the present invention, the following terminology will be used.

It is also to be understood that the terminology used herein is for the purpose of describing particular embodiments only, and is not intended to be limiting.

The articles “a” and “an” are used herein to refer to one or to more than one (i.e., to at least one) of the grammatical object of the article. By way of example, “an element” means one element or more than one element.

“About” as used herein when referring to a measurable value such as an amount, a temporal duration, and the like, is meant to encompass variations of ±20% or ±10%, more preferably ±5%, even more preferably ±1%, and still more preferably ±0.1% from the specified value, as such variations are appropriate to perform the disclosed methods. As used in this application, the terms “about” and “approximately” are used as equivalents. Any numerals used in this application with or without about/approximately are meant to cover any normal fluctuations appreciated by one of ordinary skill in the relevant art.

“Activation,” as used herein, refers to the state of a monocyte/macrophage/microglial cell that has been sufficiently stimulated to induce detectable cellular proliferation or has been stimulated to exert its effector function. Activation can also be associated with induced cytokine production, phagocytosis, cell signaling, target cell killing, or antigen processing and presentation.

The term “activated monocytes/macrophages/dendritic cells” refers to, among other things, monocyte/macrophage/dendritic cells that are undergoing cell division or exerting effector function. The term “activated monocytes/macrophages/dendritic cells” refers to, among others thing, cells that are performing an effector function or exerting any activity not seen in the resting state, including phagocytosis, cytokine secretion, proliferation, gene expression changes, metabolic changes, and other functions.

The term “agent,” or “biological agent” or “therapeutic agent” as used herein, refers to a molecule that may be expressed, released, secreted or delivered to a target by the modified cell described herein. The agent includes, but is not limited to, a nucleic acid, an antibiotic, an anti-inflammatory agent, an antibody, antibody agent or fragments thereof, a growth factor, a cytokine, an enzyme, a protein, a peptide, a fusion protein, a synthetic molecule, an organic molecule (e.g., a small molecule), a carbohydrate or the like, a lipid, a hormone, a microsome, a derivative or a variation thereof, and any combinations thereof. The agent may bind any cell moiety, such as a receptor, an antigenic determinant, or other binding site present on a target or target cell. The agent may diffuse or be transported into the cell, where it may act intracellularly.

The term “antibody,” as used herein, refers to a polypeptide that includes canonical immunoglobulin sequence elements sufficient to confer specific binding to a particular target antigen. As is known in the art, intact antibodies as produced in nature are approximately 150 kD tetrameric agents comprised of two identical heavy chain polypeptides (about 50 kD each) and two identical light chain polypeptides (about 25 kD each) that associate with each other into what is commonly referred to as a “Y-shaped” structure. Each heavy chain is comprised of at least four domains (each about 110 amino acids long)—an amino-terminal variable (VH) domain (located at the tips of the Y structure), followed by three constant domains: CH1, CH2, and the carboxy-terminal CH3 (located at the base of the Y's stem). A short region, known as the “switch”, connects the heavy chain variable and constant regions. The “hinge” connects CH2 and CH3 domains to the rest of the antibody. Two disulfide bonds in this hinge region connect the two heavy chain polypeptides to one another in an intact antibody. Each light chain is comprised of two domains—an amino-terminal variable (VL) domain, followed by a carboxy-terminal constant (CL) domain, separated from one another by another “switch”. Intact antibody tetramers are comprised of two heavy chain-light chain dimers in which the heavy and light chains are linked to one another by a single disulfide bond; two other disulfide bonds connect the heavy chain hinge regions to one another, so that the dimers are connected to one another and the tetramer is formed. Naturally-produced antibodies are also glycosylated, typically on the CH2 domain. Each domain in a natural antibody has a structure characterized by an “immunoglobulin fold” formed from two beta sheets (e.g., 3-, 4-, or 5-stranded sheets) packed against each other in a compressed antiparallel beta barrel. Each variable domain contains three hypervariable loops known as “complement determining regions” (CDR1, CDR2, and CDR3) and four somewhat invariant “framework” regions (FR1, FR2, FR3, and FR4). When natural antibodies fold, the FR regions form the beta sheets that provide the structural framework for the domains, and the CDR loop regions from both the heavy and light chains are brought together in three-dimensional space so that they create a single hypervariable antigen binding site located at the tip of the Y structure. The Fc region of naturally-occurring antibodies binds to elements of the complement system, and also to receptors on effector cells, including for example effector cells that mediate cytotoxicity. As is known in the art, affinity and/or other binding attributes of Fc regions for Fc receptors can be modulated through glycosylation or other modification. In some embodiments, antibodies produced and/or utilized in accordance with the present invention (e.g., as a component of a CAR) include glycosylated Fc domains, including Fc domains with modified or engineered such glycosylation. For purposes of the present invention, in certain embodiments, any polypeptide or complex of polypeptides that includes sufficient immunoglobulin domain sequences as found in natural antibodies can be referred to and/or used as an “antibody”, whether such polypeptide is naturally produced (e.g., generated by an organism reacting to an antigen), or produced by recombinant engineering, chemical synthesis, or other artificial system or methodology. In some embodiments, an antibody is polyclonal; in some embodiments, an antibody is monoclonal. In some embodiments, an antibody has constant region sequences that are characteristic of mouse, rabbit, primate, or human antibodies. In some embodiments, antibody sequence elements are humanized, primatized, chimeric, etc, as is known in the art. Moreover, the term “antibody” as used herein, can refer in appropriate embodiments (unless otherwise stated or clear from context) to any of the art-known or developed constructs or formats for utilizing antibody structural and functional features in alternative presentation. For example, embodiments, an antibody utilized in accordance with the present invention is in a format selected from, but not limited to, intact IgA, IgG, IgE or IgM antibodies; bi- or multi-specific antibodies (e.g., Zybodies®, etc); antibody fragments such as Fab fragments, Fab′ fragments, F(ab′)2 fragments, Fd′ fragments, Fd fragments, and isolated CDRs or sets thereof, single chain Fvs; polypeptide-Fc fusions; single domain antibodies (e.g., shark single domain antibodies such as IgNAR or fragments thereof); cameloid antibodies; masked antibodies (e.g., Probodies®); Small Modular ImmunoPharmaceuticals (“SMIPs™”); single chain or Tandem diabodies (TandAb®); VHHs; Anticalins®; Nanobodies® minibodies; BiTE®s; ankyrin repeat proteins or DARPINs®; Avimers®; DARTs; TCR-like antibodies; Adnectins®; Affilins®; Trans-bodies®; Affibodies®; TrimerX®; MicroProteins; Fynomers®, and Centyrins®. In some embodiments, an antibody may lack a covalent modification (e.g., attachment of a glycan) that it would have if produced naturally. In some embodiments, an antibody may contain a covalent modification (e.g., attachment of a glycan, a payload [e.g., a detectable moiety, a therapeutic moiety, a catalytic moiety, etc], or other pendant group [e.g., poly-ethylene glycol, etc.]

The term “antibody agent” refers to an agent that specifically binds to a particular antigen. In some embodiments, the term encompasses any polypeptide or polypeptide complex that includes immunoglobulin structural elements sufficient to confer specific binding. Exemplary antibody agents include, but are not limited to monoclonal antibodies or polyclonal antibodies. In some embodiments, an antibody agent may include one or more constant region sequences that are characteristic of mouse, rabbit, primate, or human antibodies. In some embodiments, an antibody agent may include one or more sequence elements are humanized, primatized, chimeric, etc., as is known in the art. In many embodiments, the term “antibody agent” is used to refer to one or more of the art-known or developed constructs or formats for utilizing antibody structural and functional features in alternative presentation. For example, in some embodiments, an antibody agent utilized in accordance with the present invention is in a format selected from, but not limited to, intact IgA, IgG, IgE or IgM antibodies; bi- or multi-specific antibodies (e.g., Zybodies®, etc); antibody fragments such as Fab fragments, Fab′ fragments, F(ab′)2 fragments, Fd′ fragments, Fd fragments, and isolated CDRs or sets thereof; single chain Fvs; polypeptide-Fc fusions; single domain antibodies (e.g., shark single domain antibodies such as IgNAR or fragments thereof); cameloid antibodies; masked antibodies (e.g., Probodies®); Small Modular ImmunoPharmaceuticals (“SMIPs™”); single chain or Tandem diabodies (TandAb®); VHHs; Anticalins®; Nanobodies® minibodies; BiTE®s; ankyrin repeat proteins or DARPINs®; Avimers®; DARTs; TCR-like antibodies; Adnectins®; Affilins®; Trans-bodies®; Affibodies®; TrimerX®; MicroProteins; Fynomers®; and Centyrins®. In some embodiments, an antibody may lack a covalent modification (e.g., attachment of a glycan) that it would have if produced naturally. In some embodiments, an antibody may contain a covalent modification (e.g., attachment of a glycan, a payload [e.g., a detectable moiety, a therapeutic moiety, a catalytic moiety, etc], or other pendant group [e.g., poly-ethylene glycol, etc.]. In many embodiments, an antibody agent is or comprises a polypeptide whose amino acid sequence includes one or more structural elements recognized by those skilled in the art as a complementarity determining region (CDR); in some embodiments an antibody agent is or comprises a polypeptide whose amino acid sequence includes at least one CDR (e.g., at least one heavy chain CDR and/or at least one light chain CDR) that is substantially identical to one found in a reference antibody. In some embodiments an included CDR is substantially identical to a reference CDR in that it is either identical in sequence or contains between 1-5 amino acid substitutions as compared with the reference CDR. In some embodiments an included CDR is substantially identical to a reference CDR in that it shows at least 85%, 86%, 87%, 88%, 89%, 90%, 91%, 92%, 93%, 94%, 95%, 96%, 97%, 98%, 99%, or 100% sequence identity with the reference CDR. In some embodiments an included CDR is substantially identical to a reference CDR in that it shows at least 96%, 96%, 97%, 98%, 99%, or 100% sequence identity with the reference CDR. In some embodiments an included CDR is substantially identical to a reference CDR in that at least one amino acid within the included CDR is deleted, added, or substituted as compared with the reference CDR but the included CDR has an amino acid sequence that is otherwise identical with that of the reference CDR. In some embodiments an included CDR is substantially identical to a reference CDR in that 1-5 amino acids within the included CDR are deleted, added, or substituted as compared with the reference CDR but the included CDR has an amino acid sequence that is otherwise identical to the reference CDR. In some embodiments an included CDR is substantially identical to a reference CDR in that at least one amino acid within the included CDR is substituted as compared with the reference CDR but the included CDR has an amino acid sequence that is otherwise identical with that of the reference CDR. In some embodiments an included CDR is substantially identical to a reference CDR in that 1-5 amino acids within the included CDR are deleted, added, or substituted as compared with the reference CDR but the included CDR has an amino acid sequence that is otherwise identical to the reference CDR. In some embodiments, an antibody agent is or comprises a polypeptide whose amino acid sequence includes structural elements recognized by those skilled in the art as an immunoglobulin variable domain. In some embodiments, an antibody agent is a polypeptide protein having a binding domain which is homologous or largely homologous to an immunoglobulin-binding domain. In some embodiments, an antibody agent is not and/or does not comprise a polypeptide whose amino acid sequence includes structural elements recognized by those skilled in the art as an immunoglobulin variable domain. In some embodiments, an antibody agent may be or comprise a molecule or composition which does not include immunoglobulin structural elements (e.g., a receptor or other naturally occurring molecule which includes at least one antigen binding domain).

The term “antibody fragment” refers to a portion of an intact antibody and refers to the antigenic determining variable regions of an intact antibody. Examples of antibody fragments include, but are not limited to, Fab, Fab′, F(ab′)2, and Fv fragments, linear antibodies, scFv antibodies, and multispecific antibodies formed from antibody fragments and human and humanized versions thereof.

An “antibody heavy chain,” as used herein, refers to the larger of the two types of polypeptide chains present in all antibody molecules in their naturally occurring conformations.

An “antibody light chain,” as used herein, refers to the smaller of the two types of polypeptide chains present in all antibody molecules in their naturally occurring conformations. a and 3 light chains refer to the two major antibody light chain isotypes.

By the term “synthetic antibody” as used herein, is meant an antibody which is generated using recombinant DNA technology, such as, for example, an antibody expressed by a bacteriophage as described herein. The term should also be construed to mean an antibody which has been generated by the synthesis of a DNA molecule encoding the antibody and which DNA molecule expresses an antibody protein, or an amino acid sequence specifying the antibody, wherein the DNA or amino acid sequence has been obtained using synthetic DNA or amino acid sequence technology which is available and well known in the art.

The term “antigen” or “Ag” as used herein is defined as a molecule that is capable of provoking an immune response. This immune response may involve either antibody production, or the activation of specific immunologically-competent cells, or both. The skilled artisan will understand that any macromolecule, including virtually all proteins or peptides, can serve as an antigen. Furthermore, antigens can be or be derived from recombinant or genomic DNA. A skilled artisan will understand that any DNA, which comprises a nucleotide sequences or a partial nucleotide sequence encoding a protein that elicits an immune response therefore encodes an “antigen” as that term is used herein. Furthermore, one skilled in the art will understand that an antigen need not be encoded solely by a full length nucleotide sequence of a gene. It is readily apparent that the present invention includes, but is not limited to, the use of partial nucleotide sequences of more than one gene and that these nucleotide sequences are arranged in various combinations to elicit the desired immune response. Moreover, a skilled artisan will understand that an antigen need not be encoded by a “gene” at all. It is readily apparent that an antigen can be generated synthesized or can be derived from a biological sample. Such a biological sample can include, but is not limited to a tissue sample, a tumor sample, a cell or a biological fluid.

The term “auto-antigen” means, in accordance with the present invention, any self-antigen which is recognized by the immune system as being foreign. Auto-antigens comprise, but are not limited to, cellular proteins, phosphoproteins, cellular surface proteins, cellular lipids, nucleic acids, glycoproteins, including cell surface receptors.

The term “autoimmune disease” as used herein is defined as a disorder that results from an autoimmune response. An autoimmune disease is the result of an inappropriate and excessive response to a self-antigen. Examples of autoimmune diseases include but are not limited to, Addison's disease, alopecia areata, ankylosing spondylitis, autoimmune hepatitis, autoimmune parotitis, Crohn's disease, diabetes (Type I), dystrophic epidermolysis bullosa, Epidermolysis Bullosa Simplex), epididymitis, glomerulonephritis, Graves' disease, Guillain-Barr syndrome, Hashimoto's disease, hemolytic anemia, systemic lupus erythematosus, multiple sclerosis, myasthenia gravis, pemphigus vulgaris, psoriasis, rheumatic fever, rheumatoid arthritis, sarcoidosis, scleroderma, Sjogren's syndrome, spondyloarthropathies, thyroiditis, vasculitis, vitiligo, myxedema, pernicious anemia, ulcerative colitis, among others.

As used herein, the term “autologous” is meant to refer to any material derived from the same individual to which it is later to be re-introduced into the individual.

“Allogeneic” refers to a graft derived from a different animal of the same species.

“Xenogeneic” refers to a graft derived from an animal of a different species.

The term “chimeric antigen receptor” or “CAR,” as used herein, refers to an artificial T cell surface receptor that is engineered to be expressed on an immune effector cell and specifically targets a cell and/or binds an antigen. CARs may be used as a therapy with adoptive cell transfer. Monocytes, macrophages and/or dendritic cells are removed from a patient (e.g., via blood or ascites fluid) and modified so that they express the receptors specific to a particular form of antigen. In some embodiments, the CARs have been expressed with specificity for amyloid protein antigens, for example. CARs may also comprise an extracellular domain comprising, for example, an amyloid protein antigen binding region. In some aspects, CARs comprise fusions of single-chain variable fragments (scFv) derived monoclonal antibodies. The specificity of CAR designs may be derived from ligands of receptors (e.g., peptides). In some embodiments, a CAR can target a neurodegenerative, inflammatory, cardiovascular, fibrotic or other disease/disorder by redirecting a monocyte, macrophage, or stem cell expressing the CAR specific for protein aggregates, associated with the disease/disorder.

As used herein, the term “conservative sequence modifications” is intended to refer to amino acid modifications that do not significantly affect or alter the binding characteristics of the antibody containing the amino acid sequence. Such conservative modifications include amino acid substitutions, additions and deletions. Modifications can be introduced into an antibody of the invention by standard techniques known in the art, such as site-directed mutagenesis and PCR-mediated mutagenesis. Conservative amino acid substitutions are ones in which the amino acid residue is replaced with an amino acid residue having a similar side chain. Families of amino acid residues having similar side chains have been defined in the art. These families include amino acids with basic side chains (e.g., lysine, arginine, histidine), acidic side chains (e.g., aspartic acid, glutamic acid), uncharged polar side chains (e.g., glycine, asparagine, glutamine, serine, threonine, tyrosine, cysteine, tryptophan), nonpolar side chains (e.g., alanine, valine, leucine, isoleucine, proline, phenylalanine, methionine), beta-branched side chains (e.g., threonine, valine, isoleucine) and aromatic side chains (e.g., tyrosine, phenylalanine, tryptophan, histidine). Thus, one or more amino acid residues within the CDR regions of an antibody can be replaced with other amino acid residues from the same side chain family and the altered antibody can be tested for the ability to bind antigens using the functional assays described herein.

“Co-stimulatory ligand,” as the term is used herein, includes a molecule on an antigen presenting cell (e.g., an aAPC, dendritic cell, B cell, and the like) that specifically binds a cognate co-stimulatory molecule on a monocyte/macrophage/dendritic cell, thereby providing a signal which mediates a monocyte/macrophage/dendritic cell response, including, but not limited to, proliferation, activation, differentiation, and the like. A co-stimulatory ligand can include, but is not limited to, CD7, B7-1 (CD80), B7-2 (CD86), PD-L1, PD-L2, 4-1BBL, OX40L, inducible costimulatory ligand (ICOS-L), intercellular adhesion molecule (ICAM), CD30L, CD40, CD70, CD83, HLA-G, MICA, MICB, HVEM, lymphotoxin beta receptor, 3/TR6, ILT3, ILT4, HVEM, an agonist or antibody that binds Toll ligand receptor and a ligand that specifically binds with B7-H3. A co-stimulatory ligand also encompasses, inter alia, an antibody that specifically binds with a co-stimulatory molecule present on a monocyte/macrophage/dendritic cell, such as, but not limited to, CD27, CD28, 4-1BB, OX40, CD30, CD40, PD-1, ICOS, lymphocyte function-associated antigen-1 (LFA-1), CD2, CD7, LIGHT, NKG2C, B7-H3, and a ligand that specifically binds with CD83.

A “co-stimulatory molecule” or co-stimulatory domain” refers to a molecule on an innate immune cell that is used to heighten or dampen the initial stimulus. For example, pathogen-associated pattern recognition receptors, such as TLR (heighten) or the CD47/SIRPα axis (dampen), are molecules on innate immune cells. Co-stimulatory molecules include, but are not limited to TCR, CD3 zeta, CD3 gamma, CD3 delta, CD3 epsilon, CD86, common FcR gamma, FcR beta (Fc Epsilon Rib), CD79a, CD79b, Fcgamma RIIa, DAP10, DAP12, T cell receptor (TCR), CD27, CD28, 4-1BB (CD137), OX40, CD30, CD40, PD-1, ICOS, lymphocyte function-associated antigen-1 (LFA-1), CD2, CD7, LIGHT, NKG2C, B7-H3, a ligand that specifically binds with CD83, CDS, ICAM-1, GITR, BAFFR, HVEM (LIGHTR), SLAMF7, NKp80 (KLRF1), CD127, CD160, CD19, CD4, CD8alpha, CD8beta, IL2R beta, IL2R gamma, IL7R alpha, ITGA4, VLA1, CD49a, ITGA4, IA4, CD49D, ITGA6, VLA-6, CD49f, ITGAD, CD11d, ITGAE, CD103, ITGAL, CD11a, LFA-1, ITGAM, CD11b, ITGAX, CD11c, ITGB1, CD29, ITGB2, CD18, LFA-1, ITGB7, TNFR2, TRANCE/RANKL, DNAM1 (CD226), SLAMF4 (CD244, 2B4), CD84, CD96 (Tactile), CEACAM1, CRTAM, Ly9 (CD229), CD160 (BY55), PSGL1, CD100 (SEMA4D), CD69, SLAMF6 (NTB-A, Ly108), SLAM (SLAMF1, CD150, IPO-3), BLAME (SLAMF8), SELPLG (CD162), LTBR, LAT, GADS, SLP-76, PAG/Cbp, NKp44, NKp30, NKp46, NKG2D, other co-stimulatory molecules described herein, any derivative, variant, or fragment thereof, any synthetic sequence of a co-stimulatory molecule that has the same functional capability, and any combinations thereof.

A “co-stimulatory signal”, as used herein, refers to a signal, which in combination with a primary signal, such as activation of the CAR on a macrophage, leads to activation of the macrophage.

The term “cytotoxic” or “cytotoxicity” refers to killing or damaging cells. In one embodiment, cytotoxicity of the metabolically enhanced cells is improved, e.g. increased cytolytic activity of macrophages.

A “disease” is a state of health of an animal wherein the animal cannot maintain homeostasis, and wherein if the disease is not ameliorated then the animal's health continues to deteriorate. In contrast, a “disorder” in an animal is a state of health in which the animal is able to maintain homeostasis, but in which the animal's state of health is less favorable than it would be in the absence of the disorder. Left untreated, a disorder does not necessarily cause a further decrease in the animal's state of health.

The term “neurodegenerative disease” as used herein, refers to a neurological disease characterized by loss or degeneration of neurons and/or by the presence of misfolded protein aggregates in the cytoplasm and/or nucleus of nerve cells or in the extracellular space (Forman et al., Nat. Med. 10, 1055 (2004)). Neurodegenerative diseases include neurodegenerative movement disorders and neurodegenerative conditions relating to memory loss and/or dementia. Neurodegenerative diseases include tauopathies and α-synucleopathies. Examples of neurodegenerative diseases include, but are not limited to, presenile dementia, senile dementia, Alzheimer's disease, Parkinsonism linked to chromosome 17 (FTDP-17), progressive supranuclear palsy (PSP), Pick's disease, primary progressive aphasia, frontotemporal dementia, corticobasal dementia, Parkinson's disease, Parkinson's disease with dementia, dementia with Lewy bodies, Down's syndrome, multiple system atrophy, amyotrophic lateral sclerosis (ALS) and Hallervorden-Spatz syndrome.

“Effective amount” or “therapeutically effective amount” are used interchangeably herein, and refer to an amount of a compound, formulation, material, or composition, as described herein effective to achieve a particular biological result or provides a therapeutic or prophylactic benefit.

“Encoding” refers to the inherent property of specific sequences of nucleotides in a polynucleotide, such as a gene, a cDNA, or an mRNA, to serve as templates for synthesis of other polymers and macromolecules in biological processes having either a defined sequence of nucleotides (i.e., rRNA, tRNA and mRNA) or a defined sequence of amino acids and the biological properties resulting therefrom. Thus, a gene encodes a protein if transcription and translation of mRNA corresponding to that gene produces the protein in a cell or other biological system. Both the coding strand, the nucleotide sequence of which is identical to the mRNA sequence and is usually provided in sequence listings, and the non-coding strand, used as the template for transcription of a gene or cDNA, can be referred to as encoding the protein or other product of that gene or cDNA.

As used herein “endogenous” refers to any material from or produced inside an organism, cell, tissue or system.

As used herein, the term “exogenous” refers to any material introduced from or produced outside an organism, cell, tissue or system.

The term “expand” as used herein refers to increasing in number, as in an increase in the number of monocytes, macrophages, or dendritic cells. In one embodiment, the monocytes, macrophages, or dendritic cells that are expanded ex vivo increase in number relative to the number originally present in the culture. In another embodiment, the monocytes, macrophages, or dendritic cells that are expanded ex vivo increase in number relative to other cell types in the culture. The term “ex vivo,” as used herein, refers to cells that have been removed from a living organism, (e.g., a human) and propagated outside the organism (e.g., in a culture dish, test tube, or bioreactor).

The term “expression” as used herein is defined as the transcription and/or translation of a particular nucleotide sequence driven by its promoter.

“Expression vector” refers to a vector comprising a recombinant polynucleotide comprising expression control sequences operatively linked to a nucleotide sequence to be expressed. An expression vector comprises sufficient cis-acting elements for expression; other elements for expression can be supplied by the host cell or in an in vitro expression system. Expression vectors include all those known in the art, such as cosmids, plasmids (e.g., naked or contained in liposomes) and viruses (e.g., lentiviruses, retroviruses, adenoviruses (e.g., Ad5F35), and adeno-associated viruses) that incorporate the recombinant polynucleotide.

“Homologous” as used herein, refers to the subunit sequence identity between two polymeric molecules, e.g., between two nucleic acid molecules, such as, two DNA molecules or two RNA molecules, or between two polypeptide molecules. When a subunit position in both of the two molecules is occupied by the same monomeric subunit; e.g., if a position in each of two DNA molecules is occupied by adenine, then they are homologous at that position. The homology between two sequences is a direct function of the number of matching or homologous positions; e.g., if half (e.g., five positions in a polymer ten subunits in length) of the positions in two sequences are homologous, the two sequences are 50% homologous; if 90% of the positions (e.g., 9 of 10), are matched or homologous, the two sequences are 90% homologous. As applied to the nucleic acid or protein, “homologous” as used herein refers to a sequence that has about 50% sequence identity. More preferably, the homologous sequence has about 75% sequence identity, even more preferably, has at least about 80%, 85%, 90%, 91%, 92%, 93%, 94%, 95%, 96%, 97%, 98%, 99% sequence identity.

“Humanized” forms of non-human (e.g., murine) antibodies are chimeric immunoglobulins, immunoglobulin chains or fragments thereof (such as Fv, scFv, Fab, Fab′, F(ab′)2 or other antigen-binding subsequences of antibodies) which contain minimal sequence derived from non-human immunoglobulin. For the most part, humanized antibodies are human immunoglobulins (recipient antibody) in which residues from a complementary-determining region (CDR) of the recipient are replaced by residues from a CDR of a non-human species (donor antibody) such as mouse, rat or rabbit having the desired specificity, affinity, and capacity. In some instances, Fv framework region (FR) residues of the human immunoglobulin are replaced by corresponding non-human residues. Furthermore, humanized antibodies can comprise residues which are found neither in the recipient antibody nor in the imported CDR or framework sequences. These modifications are made to further refine and optimize antibody performance. In general, the humanized antibody will comprise substantially all of at least one, and typically two, variable domains, in which all or substantially all of the CDR regions correspond to those of a non-human immunoglobulin and all or substantially all of the FR regions are those of a human immunoglobulin sequence. The humanized antibody optimally also will comprise at least a portion of an immunoglobulin constant region (Fc), typically that of a human immunoglobulin. For further details, see Jones et al., Nature, 321: 522-525, 1986; Reichmann et al., Nature, 332: 323-329, 1988; Presta, Curr. Op. Struct. Biol., 2: 593-596, 1992.

“Fully human” refers to an immunoglobulin, such as an antibody, where the whole molecule is of human origin or consists of an amino acid sequence identical to a human form of the antibody.

“Identity” as used herein refers to the subunit sequence identity between two polymeric molecules particularly between two amino acid molecules, such as, between two polypeptide molecules. When two amino acid sequences have the same residues at the same positions; e.g., if a position in each of two polypeptide molecules is occupied by an Arginine, then they are identical at that position. The identity or extent to which two amino acid sequences have the same residues at the same positions in an alignment is often expressed as a percentage. The identity between two amino acid sequences is a direct function of the number of matching or identical positions; e.g., if half (e.g., five positions in a polymer ten amino acids in length) of the positions in two sequences are identical, the two sequences are 50% identical; if 90% of the positions (e.g., 9 of 10), are matched or identical, the two amino acids sequences are 90% identical.

By “substantially identical” is meant a polypeptide or nucleic acid molecule exhibiting at least 50% identity to a reference amino acid sequence (for example, any one of the amino acid sequences described herein) or nucleic acid sequence (for example, any one of the nucleic acid sequences described herein). Preferably, such a sequence is at least 60%, more preferably 80% or 85%, and more preferably 90%, 95% or even 99% identical at the amino acid level or nucleic acid to the sequence used for comparison.

The guide nucleic acid sequence may be complementary to one strand (nucleotide sequence) of a double stranded DNA target site. The percentage of complementation between the guide nucleic acid sequence and the target sequence can be at least 50%, 51%, 52%, 53%, 54%, 55%, 56%, 57%, 58%, 59%, 60%, 61%, 62%, 63%, 63%, 65%, 66%, 67%, 68%, 69%, 70%, 71%, 72%, 73%, 74%, 75%, 76%, 77%, 78%, 79%, 80%, 81%, 82%, 83%, 84%, 85%, 86%, 87%, 88%, 89%, 90%, 91%, 92%, 93%, 94%, 95%, 96%, 97%, 98%, 99% or 100%. The guide nucleic acid sequence can be at least 10, 11, 12, 13, 14, 15, 16, 17, 18, 19, 20, 21, 22, 23, 24, 25, 26, 27, 28, 29, 30, 31, 32, 33, 34, 35 or more nucleotides in length. In some embodiments, the guide nucleic acid sequence comprises a contiguous stretch of 10 to 40 nucleotides. The variable targeting domain can be composed of a DNA sequence, a RNA sequence, a modified DNA sequence, a modified RNA sequence (see for example modifications described herein), or any combinations thereof.

Sequence identity is typically measured using sequence analysis software (for example, Sequence Analysis Software Package of the Genetics Computer Group, University of Wisconsin Biotechnology Center, 1710 University Avenue, Madison, Wis. 53705, BLAST, BESTFIT, GAP, or PILEUP/PRETTYBOX programs). Such software matches identical or similar sequences by assigning degrees of homology to various substitutions, deletions, and/or other modifications. Conservative substitutions typically include substitutions within the following groups: glycine, alanine; valine, isoleucine, leucine; aspartic acid, glutamic acid, asparagine, glutamine; serine, threonine; lysine, arginine; and phenylalanine, tyrosine. In an exemplary approach to determining the degree of identity, a BLAST program may be used, with a probability score between e⁻³ and e⁻¹⁰⁰ indicating a closely related sequence.

The term “immunoglobulin” or “Ig,” as used herein is defined as a class of proteins, which function as antibodies. Antibodies expressed by B cells are sometimes referred to as the BCR (B cell receptor) or antigen receptor. The five members included in this class of proteins are IgA, IgG, IgM, IgD, and IgE. IgA is the primary antibody that is present in body secretions, such as saliva, tears, breast milk, gastrointestinal secretions and mucus secretions of the respiratory and genitourinary tracts. IgG is the most common circulating antibody. IgM is the main immunoglobulin produced in the primary immune response in most subjects. It is the most efficient immunoglobulin in agglutination, complement fixation, and other antibody responses, and is important in defense against bacteria and viruses. IgD is the immunoglobulin that has no known antibody function, but may serve as an antigen receptor. IgE is the immunoglobulin that mediates immediate hypersensitivity by causing release of mediators from mast cells and basophils upon exposure to allergen.

The term “immune response” as used herein is defined as a cellular response to an antigen that occurs when lymphocytes identify antigenic molecules as foreign and induce the formation of antibodies and/or activate lymphocytes to remove the antigen.

As used herein, an “instructional material” includes a publication, a recording, a diagram, or any other medium of expression which can be used to communicate the usefulness of the compositions and methods of the invention. The instructional material of the kit of the invention may, for example, be affixed to a container which contains the nucleic acid, peptide, and/or composition of the invention or be shipped together with a container which contains the nucleic acid, peptide, and/or composition. Alternatively, the instructional material may be shipped separately from the container with the intention that the instructional material and the compound be used cooperatively by the recipient.

“Isolated” means altered or removed from the natural state. For example, a nucleic acid or a peptide naturally present in a living animal is not “isolated,” but the same nucleic acid or peptide partially or completely separated from the coexisting materials of its natural state is “isolated.” An isolated nucleic acid or protein can exist in substantially purified form, or can exist in a non-native environment such as, for example, a host cell.

A “lentivirus” as used herein refers to a genus of the Retroviridae family. Lentiviruses are unique among the retroviruses in being able to infect non-dividing cells; they can deliver a significant amount of genetic information into the DNA of the host cell, so they are one of the most efficient methods of a gene delivery vector. HIV, SIV, and FIV are all examples of lentiviruses. Vectors derived from lentiviruses offer the means to achieve significant levels of gene transfer in vivo.

The terms “Lewy body”, “Lewy bodies”, and “Lewy neurites”, refer to abnormal aggregates of protein that develop in nerve cells.

By the term “modified” as used herein, is meant a changed state or structure of a molecule or cell of the invention. Molecules may be modified in many ways, including chemically, structurally, and functionally. Cells may be modified through the introduction of nucleic acids.

By the term “modulating,” as used herein, is meant mediating a detectable increase or decrease in the level of a response in a subject compared with the level of a response in the subject in the absence of a treatment or compound, and/or compared with the level of a response in an otherwise identical but untreated subject. The term encompasses perturbing and/or affecting a native signal or response thereby mediating a beneficial therapeutic response in a subject, preferably, a human.

In the context of the present invention, the following abbreviations for the commonly occurring nucleic acid bases are used. “A” refers to adenosine, “C” refers to cytosine, “G” refers to guanosine, “T” refers to thymidine, and “U” refers to uridine.

Unless otherwise specified, a “nucleotide sequence encoding an amino acid sequence” includes all nucleotide sequences that are degenerate versions of each other and that encode the same amino acid sequence. The phrase nucleotide sequence that encodes a protein or an RNA may also include introns to the extent that the nucleotide sequence encoding the protein may in some version contain an intron(s).

The term “operably linked” refers to functional linkage between a regulatory sequence and a heterologous nucleic acid sequence resulting in expression of the latter. For example, a first nucleic acid sequence is operably linked with a second nucleic acid sequence when the first nucleic acid sequence is placed in a functional relationship with the second nucleic acid sequence. For instance, a promoter is operably linked to a coding sequence if the promoter affects the transcription or expression of the coding sequence. Generally, operably linked DNA sequences are contiguous and, where necessary to join two protein coding regions, in the same reading frame.

“Parenteral” administration of an immunogenic composition includes, e.g., subcutaneous (s.c.), intravenous (i.v.), intramuscular (i.m.), intratumoral (i.t.) or intra-peritoneal (i.p.), or intrasternal injection, or infusion techniques.

The term “polynucleotide” as used herein is defined as a chain of nucleotides. Furthermore, nucleic acids are polymers of nucleotides. Thus, nucleic acids and polynucleotides as used herein are interchangeable. One skilled in the art has the general knowledge that nucleic acids are polynucleotides, which can be hydrolyzed into the monomeric “nucleotides.” The monomeric nucleotides can be hydrolyzed into nucleosides. As used herein polynucleotides include, but are not limited to, all nucleic acid sequences which are obtained by any means available in the art, including, without limitation, recombinant means, i.e., the cloning of nucleic acid sequences from a recombinant library or a cell genome, using ordinary cloning technology and PCR™, and the like, and by synthetic means.

As used herein, the terms “peptide,” “polypeptide,” and “protein” are used interchangeably, and refer to a compound comprised of amino acid residues covalently linked by peptide bonds. A protein or peptide must contain at least two amino acids, and no limitation is placed on the maximum number of amino acids that can comprise a protein's or peptide's sequence. Polypeptides include any peptide or protein comprising two or more amino acids joined to each other by peptide bonds. As used herein, the term refers to both short chains, which also commonly are referred to in the art as peptides, oligopeptides and oligomers, for example, and to longer chains, which generally are referred to in the art as proteins, of which there are many types. “Polypeptides” include, for example, biologically active fragments, substantially homologous polypeptides, oligopeptides, homodimers, heterodimers, variants of polypeptides, modified polypeptides, derivatives, analogs, fusion proteins, among others. The polypeptides include natural peptides, recombinant peptides, synthetic peptides, or any combinations thereof.

The term “protein aggregate” as used herein means two or more proteins (e.g., two or more of the same protein, two or more different proteins, etc) that have aggregated together in a tissue in a subject to give rise to a pathological condition, or which places the subject at risk for a pathological condition. In some embodiments, a protein aggregate may be or comprise one or more of: misfolded protein(s), otherwise improperly formed/malformed protein(s) (e.g., as a result of a mutation which may not affect folding but does affect function), and/or an aggregation of protein and non-protein components (e.g., nucleic acids, small molecules, etc). Non-limiting examples of such protein aggregates include aggregates of amyloid protein, aggregates of tau protein, aggregates of TDP-43 protein, aggregates of immunoglobulin light chains or transthyretin protein, aggregates of prion protein and the like.

The term “promoter” as used herein is defined as a DNA sequence recognized by the synthetic machinery of the cell, or introduced synthetic machinery, required to initiate the specific transcription of a polynucleotide sequence.

As used herein, the term “promoter/regulatory sequence” means a nucleic acid sequence which is required for expression of a gene product operably linked to the promoter/regulatory sequence. In some instances, this sequence may be the core promoter sequence and in other instances, this sequence may also include an enhancer sequence and other regulatory elements which are required for expression of the gene product. The promoter/regulatory sequence may, for example, be one which expresses the gene product in a tissue specific manner.

A “constitutive” promoter is a nucleotide sequence which, when operably linked with a polynucleotide which encodes or specifies a gene product, causes the gene product to be produced in a cell under most or all physiological conditions of the cell.

An “inducible” promoter is a nucleotide sequence which, when operably linked with a polynucleotide which encodes or specifies a gene product, causes the gene product to be produced in a cell substantially only when an inducer which corresponds to the promoter is present in the cell.

A “tissue-specific” promoter is a nucleotide sequence which, when operably linked with a polynucleotide encodes or specified by a gene, causes the gene product to be produced in a cell substantially only if the cell is a cell of the tissue type corresponding to the promoter.

The term “resistance to immunosuppression” refers to lack of suppression or reduced suppression of an immune system activity or activation.

A “signal transduction pathway” refers to the biochemical relationship between a variety of signal transduction molecules that play a role in the transmission of a signal from one portion of a cell to another portion of a cell. The phrase “cell surface receptor” includes molecules and complexes of molecules capable of receiving a signal and transmitting signal across the plasma membrane of a cell.

“Single chain antibodies” refer to antibodies formed by recombinant DNA techniques in which immunoglobulin heavy and light chain fragments are linked to the Fv region via an engineered span of amino acids. Various methods of generating single chain antibodies are known, including those described in U.S. Pat. No. 4,694,778; Bird, 1988, Science 242:423-442; Huston et al., 1988, Proc. Natl. Acad. Sci. USA 85:5879-5883; Ward et al., 1989, Nature 334:54454; Skerra et al., 1988, Science 242:1038-1041.

By the term “specifically binds,” as used herein with respect to an antigen binding domain, such as an antibody agent, is meant an antigen binding domain or antibody agent which recognizes a specific antigen, but does not substantially recognize or bind other molecules in a sample. For example, an antigen binding domain or antibody agent that specifically binds to an antigen from one species may also bind to that antigen from one or more species. But, such cross-species reactivity does not itself alter the classification of an antigen binding domain or antibody agent as specific. In another example, an antigen binding domain or antibody agent that specifically binds to an antigen may also bind to different allelic forms of the antigen. However, such cross reactivity does not itself alter the classification of an antigen binding domain or antibody agent as specific. In some instances, the terms “specific binding” or “specifically binding,” can be used in reference to the interaction of an antigen binding domain or antibody agent, a protein, or a peptide with a second chemical species, to mean that the interaction is dependent upon the presence of a particular structure (e.g., an antigenic determinant or epitope) on the chemical species; for example, an antigen binding domain or antibody agent recognizes and binds to a specific protein structure rather than to proteins generally. If an antigen binding domain or antibody agent is specific for epitope “A”, the presence of a molecule containing epitope A (or free, unlabeled A), in a reaction containing labeled “A” and the antigen binding domain or antibody agent, will reduce the amount of labeled A bound to the antibody.

By the term “stimulation,” is meant a primary response induced by binding of a stimulatory molecule (e.g., a TCR/CD3 complex) with its cognate ligand thereby mediating a signal transduction event, such as, but not limited to, signal transduction via the Fc receptor machinery or via the synthetic CAR. Stimulation can mediate altered expression of certain molecules, such as downregulation of TGF-beta, and/or reorganization of cytoskeletal structures, and the like.

A “stimulatory molecule,” as the term is used herein, means a molecule of a monocyte, macrophage, or dendritic cell that specifically binds with a cognate stimulatory ligand present on an antigen presenting cell.

A “stimulatory ligand,” as used herein, means a ligand that when present on an antigen presenting cell (e.g., an aAPC, a dendritic cell, a B-cell, and the like) or tumor cell can specifically bind with a cognate binding partner (referred to herein as a “stimulatory molecule”) on a monocyte, macrophage, or dendritic cell, thereby mediating a response by the immune cell, including, but not limited to, activation, initiation of an immune response, proliferation, and the like. Stimulatory ligands are well-known in the art and encompass, inter alia, Toll-like receptor (TLR) ligand, an anti-toll-like receptor antibody, an agonist, and an antibody for a monocyte/macrophage receptor. In addition, cytokines, such as interferon-gamma, are potent stimulants of macrophages.

The term “subject” is intended to include living organisms in which an immune response can be elicited (e.g., mammals). A “subject” or “patient,” as used therein, may be a human or non-human mammal. Non-human mammals include, for example, livestock and pets, such as ovine, bovine, porcine, canine, feline and murine mammals. Preferably, the subject is human.

As used herein, a “substantially purified” cell is a cell that is essentially free of other cell types. A substantially purified cell also refers to a cell which has been separated from other cell types with which it is normally associated in its naturally occurring state. In some instances, a population of substantially purified cells refers to a homogenous population of cells. In other instances, this term refers simply to cell that have been separated from the cells with which they are naturally associated in their natural state. In some embodiments, the cells are cultured in vitro. In other embodiments, the cells are not cultured in vitro.

A “target site” or “target sequence” refers to a genomic nucleic acid sequence that defines a portion of a nucleic acid to which a binding molecule may specifically bind under conditions sufficient for binding to occur.

By “target” is meant a cell, organ, tissue or site within the body (e.g., a protein aggregate) that is in need of treatment.

As used herein, the term “T cell receptor” or “TCR” refers to a complex of membrane proteins that participate in the activation of T cells in response to the presentation of antigen. The TCR is responsible for recognizing antigens bound to major histocompatibility complex molecules. TCR is composed of a heterodimer of an alpha (α) and beta (β) chain, although in some cells the TCR consists of gamma and delta (γ/δ) chains. TCRs may exist in alpha/beta and gamma/delta forms, which are structurally similar but have distinct anatomical locations and functions. Each chain is composed of two extracellular domains, a variable and constant domain. In some embodiments, the TCR may be modified on any cell comprising a TCR, including, for example, a helper T cell, a cytotoxic T cell, a memory T cell, regulatory T cell, natural killer T cell, and gamma delta T cell.

The term “therapeutic” as used herein means a treatment and/or prophylaxis. A therapeutic effect is obtained by suppression, remission, or eradication of a disease state.

The term “transfected” or “transformed” or “transduced” as used herein refers to a process by which exogenous nucleic acid is transferred or introduced into the host cell. A “transfected” or “transformed” or “transduced” cell is one which has been transfected, transformed or transduced with exogenous nucleic acid. The cell includes the primary subject cell and its progeny.

To “treat” a disease as the term is used herein, means to reduce the frequency or severity of at least one sign or symptom of a disease or disorder experienced by a subject. Treating a disease can also include a reversal of the symptoms and/or prevention or reduction of disease progression.

The phrase “under transcriptional control” or “operatively linked” as used herein means that the promoter is in the correct location and orientation in relation to a polynucleotide to control the initiation of transcription by RNA polymerase and expression of the polynucleotide.

A “vector” is a composition of matter which comprises an isolated nucleic acid and which can be used to deliver the isolated nucleic acid to the interior of a cell. Numerous vectors are known in the art including, but not limited to, linear polynucleotides, polynucleotides associated with ionic or amphiphilic compounds, plasmids, and viruses. Thus, the term “vector” includes an autonomously replicating plasmid or a virus. The term should also be construed to include non-plasmid and non-viral compounds which facilitate transfer of nucleic acid into cells, such as, for example, polylysine compounds, liposomes, and the like. Examples of viral vectors include, but are not limited to, adenoviral vectors, adeno-associated virus vectors, retroviral vectors, lentiviral vectors, and the like.

Ranges: throughout this disclosure, various aspects of the invention can be presented in a range format. It should be understood that the description in range format is merely for convenience and brevity and should not be construed as an inflexible limitation on the scope of the invention. Accordingly, the description of a range should be considered to have specifically disclosed all the possible subranges as well as individual numerical values within that range. For example, description of a range such as from 1 to 6 should be considered to have specifically disclosed subranges such as from 1 to 3, from 1 to 4, from 1 to 5, from 2 to 4, from 2 to 6, from 3 to 6 etc., as well as individual numbers within that range, for example, 1, 2, 2.7, 3, 4, 5, 5.3, and 6. This applies regardless of the breadth of the range.

### DESCRIPTION

The present disclosure provides, among other things, compositions comprising chimeric antigen receptors (CARs) specific for amyloid beta (Aβ) and/or Tau, and methods of using modified immune cells described herein comprising CARs specific for Aβ and/or Tau. In certain embodiments, CARs described herein do not comprise an intracellular domain. Methods of treatment are also disclosed herein.

Chimeric antigen receptor (CAR)-redirected T cells are generally more potent as immunotherapies than their corresponding mAbs, and CAR macrophages are now being tested clinically (PMID 32361713; https://clinicaltrials.gov/ct2/show/NCT04660929). Regardless of underlying potency of antibody-dependent phagocytosis (the putative mechanism of action of mAbs in AD), monoclonal antibodies engagement cannot directly change the underlying physiology of aged/Alzheimer's Disease (AD) microglia. In some embodiments, approaches disclosed herein replace endogenous microglia with anti-amyloid beta CAR-macrophages to ameliorate disease pathology by lowering extracellular amyloid beta burden and/or replacing dysfunctional microglia. Since extracellular and intracellular signaling domains of the CAR can be modularized, use of CAR constructs to redirect macrophage function in the presence of a specific pathological agent paves the way for novel cellular-based therapies for other disorders driven by accumulation of misfolded proteins.

This innovation differs, at least in part, from other CAR technologies in that an intracellular signaling/activation domain is not required for Aβ uptake. In contrast, all other CAR T, CAR NK, or CAR macrophage technologies appear to require at least a CD3 zeta (or similar) signaling domain (see e.g. WO 2019/152,781 A1).

In accordance with several embodiments, provided cells and compositions may exhibit any of several beneficial activities (e.g., in a subject or patient). In some embodiments, an immune exhibits one or more activities selected from the group consisting of phagocytosis, targeted cellular cytotoxicity, antigen presentation, cytokine secretion, and any combination thereof. In addition, in some embodiments, one or more activities of a provided immune cell may be enhanced or otherwise modulated using any of a variety of methods described herein. By way of specific example, in some embodiments, an activity of a provided immune cell is enhanced by inhibition of CD47 and/or SIRPα activity.

It is specifically contemplated that, in some embodiments, provided immune cells and/or compositions may be used as a component of a combination therapy. In some embodiments, provided immune cell(s) and/or compositions may further include at least one agent selected from the group consisting of a nucleic acid, an antibiotic, an anti-inflammatory agent, an antibody or antibody fragments thereof, a growth factor, a cytokine, an enzyme, a protein, a peptide, a fusion protein, a synthetic molecule, an organic molecule, a carbohydrate, a lipid, a hormone, a microsome, and any combinations thereof.

It is specifically contemplated that, in some embodiments, provided cells immune and/or compositions may be used in the manufacture of a medicament for the treatment of a neurodegenerative disease, in a subject in need thereof.

In accordance with various embodiments, the present disclosure provides methods of treating a neurodegenerative disease in a subject in need thereof, methods comprising administering to a subject a therapeutically effective amount of a pharmaceutical composition described herein.

In accordance with various embodiments, the present invention provides methods of modifying an immune cell, the methods comprising introducing into an immune cell (e.g., a monocyte, macrophage and/or stem cell (e.g. a hematopoietic stem cell with the potential to become a myeloid cell such as a monocyte, macrophage or microglia-like cell) a chimeric antigen receptor (CAR), wherein the CAR comprises an antigen binding domain and a transmembrane domain, wherein the antigen binding domain is capable of binding (Aβ) and/or Tau.

In some embodiments, introducing a CAR into an immune cell described herein comprises introducing a nucleic acid sequence encoding the CAR into the immune cell. In some embodiments, introducing the nucleic acid sequence into the immune cell comprises electroporating an mRNA encoding the CAR into the immune cell. In some embodiments, introducing the nucleic acid sequence into the cell comprises at least one procedure selected from the group consisting of electroporation, a lentiviral transduction, adenoviral transduction, retroviral transduction, chemical-based transfection, and any combination thereof. In some embodiments, the method further comprises modifying an immune cell to deliver to a target an agent selected from the group consisting of a nucleic acid, an antibiotic, an anti-inflammatory agent, an antibody, a growth factor, a cytokine, an enzyme, a protein, a peptide, a fusion protein, a synthetic molecule, an organic molecule, a carbohydrate or the like, a lipid, a hormone, a microsome, and any combinations thereof. In some embodiments, the disclosure provides compositions comprising an immune cell made by a method described herein.

Other features, objects, and advantages of the present invention are apparent in the detailed description that follows. It should be understood, however, that the detailed description, while indicating embodiments of the present invention, is given by way of illustration only, not limitation. Various changes and modifications within the scope of the invention will become apparent to those skilled in the art from the detailed description.

### Chimeric Antigen Receptors (CARs)

The present disclosure provides compositions and methods comprising modified immune cells or precursors thereof, e.g., modified macrophages, comprising a chimeric antigen receptor (CAR). Thus, in some embodiments, immune cells have been genetically modified to express a CAR described herein. CARs of the disclosure comprise an antigen binding domain and a transmembrane domain. In certain embodiments, the CAR comprises an intracellular domain. In certain embodiments the CAR does not comprise an intracellular domain.

In one aspect, a modified immune cell (e.g., monocyte, macrophage, or stem cell; e.g. hematopoietic stem cell with the potential to become a myeloid cell such as a monocyte, macrophage or microglia-like cell), is generated by expressing a CAR therein. Thus, the present disclosure encompasses provided CARs, as well as nucleic acid constructs encoding provided CARs, wherein the CAR includes an antigen binding domain and a transmembrane domain. In certain embodiments the CAR comprises an intracellular domain. In certain embodiments the CAR does not comprise an intracellular domain. In certain instances, an immune cell (e.g., a monocyte, macrophage or stem cell; e.g., hematopoietic stem cell with the potential to become a myeloid cell such as a monocyte, macrophage or microglia-like cell), comprising a CAR is referred to herein as a CAR-Macrophage.

In one aspect, the disclosure includes a modified immune cell including a chimeric antigen receptor (CAR), wherein the CAR comprises an antigen binding domain and a transmembrane domain, wherein the antigen binding domain is capable of binding to amyloid beta (Aβ), and wherein the immune cell is a monocyte, macrophage or stem cell (e.g. hematopoietic stem cell with the potential to become a myeloid cell such as a monocyte, macrophage or microglia-like cell), that expresses the CAR.

In one aspect, the disclosure includes a modified immune cell including a chimeric antigen receptor (CAR), wherein the CAR comprises an antigen binding domain and a transmembrane domain, wherein the antigen binding domain is capable of binding to Tau, and wherein the cell is a monocyte, macrophage or stem cell (e.g., hematopoietic stem cell with the potential to become a myeloid cell such as a monocyte, macrophage or microglia-like cell), that expresses the CAR.

In another aspect, the present disclosure provides modified immune cells including a nucleic acid sequence (e.g., an isolated nucleic acid sequence) encoding a chimeric antigen receptor (CAR), wherein the nucleic acid sequence comprises a nucleic acid sequence encoding an antigen binding domain and a nucleic acid sequence encoding a transmembrane domain, wherein the antigen binding domain is capable of binding to an antigen amyloid beta (Aβ) and/or Tau, and wherein the immune cell is a monocyte, macrophage and/or stem cell (e.g., hematopoietic stem cell with the potential to become a myeloid cell such as a monocyte, macrophage or microglia-like cell), that expresses the CAR

### Antigen-Binding Domain

The antigen-binding domain of a CAR is an extracellular region of the CAR for binding to a specific target antigen including proteins, carbohydrates, and glycolipids. A subject CAR of the disclosure comprises an antigen-binding domain that is capable of binding amyloid beta (Aβ) or Tau.

The antigen-binding domain can include any domain that binds to the antigen and may include, but is not limited to, a monoclonal antibody, a polyclonal antibody, a synthetic antibody, a human antibody, a humanized antibody, a non-human antibody, and any fragment thereof. In some embodiments, the antigen-binding domain portion comprises a mammalian antibody or a fragment thereof. The choice of antigen-binding domain may depend upon the type and number of antigens that are present on the surface of a target cell.

In some embodiments, the antigen-binding domain is selected from the group consisting of a full-length antibody, an antigen-binding fragment, a Fab, a single-chain variable fragment (scFv), or a single-domain antibody. In some embodiments, an Aβ binding domain of the present disclosure is selected from the group consisting of an Aβ-specific antibody, an Aβ-specific Fab, and an Aβ-specific scFv. In one embodiment, an Aβ binding domain is an Aβ-specific antibody. In one embodiment, an Aβ binding domain is an Aβ-specific Fab. In one embodiment, an Aβ binding domain is an Aβ-specific scFv.

As used herein, the term “single-chain variable fragment” or “scFv” is a fusion protein of the variable regions of the heavy (VH) and light chains (VL) of an immunoglobulin (e.g., mouse or human) covalently linked to form a VH::VL heterodimer. The heavy (VH) and light chains (VL) are either joined directly or joined by a peptide-encoding linker, which connects the N-terminus of the VH with the C-terminus of the VL, or the C-terminus of the VH with the N-terminus of the VL. In some embodiments, the antigen-binding domain (e.g., FAP binding domain) comprises an scFv having the configuration from N-terminus to C-terminus, VH-linker-VL. In some embodiments, the antigen-binding domain comprises an scFv having the configuration from N-terminus to C-terminus, VL-linker-VH. Those of skill in the art would be able to select the appropriate configuration for use in the present invention.

A linker is usually rich in glycine for flexibility, as well as serine or threonine for solubility. In some embodiments, a linker can link the heavy chain variable region and the light chain variable region of the extracellular antigen-binding domain. Non-limiting examples of linkers are disclosed in Shen et al., Anal. Chem. 80(6):1910-1917 (2008) and WO 2014/087010, the contents of which are hereby incorporated by reference in their entireties. Various linker sequences are known in the art, including, without limitation, glycine serine (GS) linkers such as (GS)ₙ, (GSGGS)ₙ (SEQ ID NO: 124), (GGGS)ₙ (SEQ ID NO: 114), and (GGGGS)ₙ (SEQ ID NO: 115), where n represents an integer of at least 1. Exemplary linker sequences can comprise amino acid sequences including, without limitation, GGSG (SEQ ID NO: 116), GGSGG (SEQ ID NO: 117), GSGSG (SEQ ID NO: 118), GSGGG (SEQ ID NO: 119), GGGSG (SEQ ID NO: 120), GSSSG (SEQ ID NO: 121), GGGGS (SEQ ID NO: 122), GGGGSGGGGSGGGGS (SEQ ID NO: 123) and the like. Those of skill in the art would be able to select the appropriate linker sequence for use in the present invention. In one embodiment, an antigen-binding domain of the present invention comprises a heavy chain variable region (VH) and a light chain variable region (VL), wherein the VH and VL is separated by the linker sequence having the amino acid sequence GGGGSGGGGSGGGGS (SEQ ID NO: 123), which may be encoded by the nucleic acid sequence GGTGGCGGTGGCTCGGGCGGTGGTGGGTCGGGTGGCGGCGGATCT (SEQ ID NO: 125).

Despite removal of the constant regions and the introduction of a linker, scFv proteins retain the specificity of the original immunoglobulin. Single chain Fv polypeptide antibodies can be expressed from a nucleic acid comprising VH- and VL-encoding sequences as described by Huston, et al. (Proc. Nat. Acad. Sci. USA, 85:5879-5883, 1988). See, also, U.S. Pat. Nos. 5,091,513, 5,132,405 and 4,956,778; and U.S. Patent Publication Nos. 20050196754 and 20050196754. Antagonistic scFvs having inhibitory activity have been described (see, e.g., Zhao et al., Hyrbidoma (Larchmt) 2008 27(6):455-51; Peter et al., J Cachexia Sarcopenia Muscle 2012 Aug. 12; Shieh et al., J Imunol 2009 183(4):2277-85; Giomarelli et al., Thromb Haemost 2007 97(6):955-63; Fife eta., J Clin Invst 2006 116(8):2252-61; Brocks et al., Immunotechnology 1997 3(3):173-84; Moosmayer et al., Ther Immunol 1995 2(10:31-40), each of which are hereby incorporated by reference in their entirety. Agonistic scFvs having stimulatory activity have been described (see, e.g., Peter et al., J Bioi Chem 2003 25278(38):36740-7; Xie et al., Nat Biotech 1997 15(8):768-71; Ledbetter et al., Crit Rev Immunol 1997 17(5-6):427-55; Ho et al., BioChim Biophys Acta 2003 1638(3):257-66), each of which are hereby incorporated by reference in their entirety.

As used herein, “Fab” refers to a fragment of an antibody structure that binds to an antigen but is monovalent and does not have a Fc portion, for example, an antibody digested by the enzyme papain yields two Fab fragments and an Fc fragment (e.g., a heavy (H) chain constant region; Fc region that does not bind to an antigen).

As used herein, “F(ab′)2” refers to an antibody fragment generated by pepsin digestion of whole IgG antibodies, wherein this fragment has two antigen-binding (ab′) (bivalent) regions, wherein each (ab′) region comprises two separate amino acid chains, a part of a H chain and a light (L) chain linked by an S—S bond for binding an antigen and where the remaining H chain portions are linked together. A “F(ab′)2” fragment can be split into two individual Fab′ fragments.

In some embodiments, an antigen-binding domain may be derived from the same species in which a CAR described herein will ultimately be used. For example, for use in humans, an antigen-binding domain of a CAR described herein may comprise a human antibody or a fragment thereof. In some embodiments, an antigen-binding domain may be derived from a different species in which a CAR described herein will ultimately be used. For example, for use in humans, an antigen-binding domain of a CAR described herein may comprise a murine antibody, or a canine antibody, or a fragment thereof.

Antigen-Binding Domain Derived from Aducanumab Antibody

In certain embodiments, an antigen-binding domain of a CAR described herein is capable of binding amyloid beta (Aβ). In certain embodiments, the antigen-binding domain is derived from the Aducanumab antibody. Aducanumab, sold under the brand name Aduhelm™, is an amyloid beta-directed antibody. Aducanumab is described in WO 2021/081101 A1, the contents of which is incorporated herein by reference in its entirety.

In certain embodiments, the antigen-binding domain comprises a heavy chain variable region that comprises three heavy chain complementarity determining regions (HCDRs). In certain embodiments, HCDR1 comprises the amino acid sequence SYGMH (SEQ ID NO: 1), and/or HCDR2 comprises the amino acid sequence VIWFDGTKKYYTDSVKG (SEQ ID NO: 2), and/or HCDR3 comprises the amino acid sequence DRGIGARRGPYYMDV (SEQ ID NO: 3). The antigen-binding domain also comprises a light chain variable region that comprises three light chain complementarity determining regions (LCDRs). In certain embodiments, LCDR1 comprises the amino acid sequence RASQSISSYLN (SEQ ID NO: 4), and/or LCDR2 comprises the amino acid sequence ASSLQS (SEQ ID NO: 5), and/or LCDR3 comprises the amino acid sequence QQSYSTPLT (SEQ ID NO: 6).

In certain embodiments, the antigen-binding domain comprises any one of SEQ ID NOs: 1-6.

In certain embodiments, the heavy chain variable region (VH) of the antigen-binding domain comprises an amino acid sequence at least 80%, 85%, 90%, 95%, 96%, 97%, 98%, 99%, or 100% identical to SEQ ID NO: 7 and/or the light chain variable region (VL) comprises an amino acid sequence at least 80%, 85%, 90%, 95%, 96%, 97%, 98%, 99%, or 100% identical to SEQ ID NO: 8.

In certain embodiments, the heavy chain variable region (VH) of the antigen-binding domain is encoded by a nucleotide sequence at least 80%, 85%, 90%, 95%, 96%, 97%, 98%, 99%, or 100% identical to SEQ ID NO: 9 and/or the light chain variable region (VL) is encoded by a nucleotide sequence at least 80%, 85%, 90%, 95%, 96%, 97%, 98%, 99%, or 100% identical to SEQ ID NO: 10.

In certain embodiments, the antigen-binding domain is a single-chain variable fragment (scFv) comprising an amino acid sequence at least 80%, 85%, 90%, 95%, 96%, 97%, 98%, 99%, or 100% identical to SEQ ID NO: 11 or SEQ ID NO: 12.

In certain embodiments, the antigen-binding domain is a single-chain variable fragment (scFv) encoded by a nucleotide sequence at least 80%, 85%, 90%, 95%, 96%, 97%, 98%, 99%, or 100% identical to SEQ ID NO: 13 or SEQ ID NO: 14.

In certain embodiments the CAR comprises an antigen binding domain capable of binding Aβ (e.g derived from Aducanumab) and does not contain an intracellular domain. In certain embodiments the CAR comprises an antigen binding domain capable of binding Aβ, a CD8 alpha hinge domain, and a CD28 transmembrane domain. In certain embodiments, the CAR comprises an amino acid sequence at least 80%, 85%, 90%, 95%, 96%, 97%, 98%, 99%, or 100% identical to SEQ ID NO: 15 or SEQ ID NO: 16. In certain embodiments, the CAR is encoded by a nucleotide sequence at least 80%, 85%, 90%, 95%, 96%, 97%, 98%, 99%, or 100% identical to SEQ ID NO: 17 or SEQ ID NO: 18.

In certain embodiments the CAR comprises an antigen binding domain capable of binding Aβ (e.g. derived from Aducanumab), a transmembrane domain, and an intracellular domain. The CAR can comprise any intracellular domain known in the art and/or disclosed herein. In certain embodiments, the intracellular domain comprises CD3 zeta. In certain embodiments the CAR comprises an antigen binding domain capable of binding Aβ (e.g. derived from Aducanumab), a CD8 alpha hinge domain, a CD28 transmembrane domain, and a CD3 zeta intracellular domain. In certain embodiments, the CAR comprises an amino acid sequence at least 80%, 85%, 90%, 95%, 96%, 97%, 98%, 99%, or 100% identical to SEQ ID NO: 73 or SEQ ID NO: 74. In certain embodiments, the CAR is encoded by a nucleotide sequence at least 80%, 85%, 90%, 95%, 96%, 97%, 98%, 99%, or 100% identical to SEQ ID NO: 75 or SEQ ID NO: 76.

Antigen-Binding Domain Derived from Crenezumab Antibody

In certain embodiments, the antigen-binding domain of the CAR is capable of binding amyloid beta (Aβ). In certain embodiments, the antigen-binding domain is derived from the Crenezumab antibody. Crenezumab is a humanized monoclonal antibody against human 1-40 and 1-42 Beta amyloid. Crenezumab is described in U.S. Pat. No. 10,494,429, the contents of which is incorporated herein by reference in its entirety.

In certain embodiments, the antigen-binding domain comprises a heavy chain variable region that comprises three heavy chain complementarity determining regions (HCDRs). In certain embodiments, HCDR1 comprises the amino acid sequence GFTFSSYGMS (SEQ ID NO: 19), and/or HCDR2 comprises the amino acid sequence SINSNGGSTYYPDSVK (SEQ ID NO: 20), and/or HCDR3 comprises the amino acid sequence GDY (SEQ ID NO: 21). The antigen-binding domain also comprises a light chain variable region that comprises three light chain complementarity determining regions (LCDRs). In certain embodiments, LCDR1 comprises the amino acid sequence RSSQSLVYSNGDTYLH (SEQ ID NO: 22), and/or LCDR2 comprises the amino acid sequence KVSNRFS (SEQ ID NO: 23), and/or LCDR3 comprises the amino acid sequence SQSTHVPWT (SEQ ID NO: 24).

In certain embodiments, the antigen-binding domain comprises any one of SEQ ID NOs: 19-24.

In certain embodiments, the heavy chain variable region (VH) of the antigen-binding domain comprises an amino acid sequence at least 80%, 85%, 90%, 95%, 96%, 97%, 98%, 99%, or 100% identical to SEQ ID NO: 25 and/or the light chain variable region (VL) comprises an amino acid sequence at least 80%, 85%, 90%, 95%, 96%, 97%, 98%, 99%, or 100% identical to SEQ ID NO: 26.

In certain embodiments, the heavy chain variable region (VH) of the antigen-binding domain is encoded by a nucleotide sequence at least 80%, 85%, 90%, 95%, 96%, 97%, 98%, 99%, or 100% identical to SEQ ID NO: 27 and/or the light chain variable region (VL) is encoded by a nucleotide sequence at least 80%, 85%, 90%, 95%, 96%, 97%, 98%, 99%, or 100% identical to SEQ ID NO: 28.

In certain embodiments, the antigen-binding domain is a single-chain variable fragment (scFv) comprising an amino acid sequence at least 80%, 85%, 90%, 95%, 96%, 97%, 98%, 99%, or 100% identical to SEQ ID NO: 29 or SEQ ID NO: 30.

In certain embodiments, the antigen-binding domain is a single-chain variable fragment (scFv) encoded by a nucleotide sequence at least 80%, 85%, 90%, 95%, 96%, 97%, 98%, 99%, or 100% identical to SEQ ID NO: 31 or SEQ ID NO: 32.

In certain embodiments, the CAR comprises an antigen binding domain capable of binding Aβ (e.g derived from Crenezumab) and does not contain an intracellular domain. In certain embodiments the CAR comprises an antigen binding domain capable of binding Aβ, a CD8 alpha hinge domain, and a CD28 transmembrane domain. In certain embodiments, the CAR comprises an amino acid sequence at least 80%, 85%, 90%, 95%, 96%, 97%, 98%, 99%, or 100% identical to SEQ ID NO: 33 or SEQ ID NO: 34. In certain embodiments, the CAR is encoded by a nucleotide sequence at least 80%, 85%, 90%, 95%, 96%, 97%, 98%, 99%, or 100% identical to SEQ ID NO: 35 or SEQ ID NO: 36.

In certain embodiments, the CAR comprises an antigen binding domain capable of binding Aβ (e.g. derived from Crenezumab), a transmembrane domain, and an intracellular domain. The CAR can comprise any intracellular domain known in the art and/or disclosed herein. In certain embodiments, the intracellular domain comprises CD3 zeta. In certain embodiments the CAR comprises an antigen binding domain capable of binding Aβ (e.g. derived from Crenezumab), a CD8 alpha hinge domain, a CD28 transmembrane domain, and a CD3 zeta intracellular domain. In certain embodiments, the CAR comprises an amino acid sequence at least 80%, 85%, 90%, 95%, 96%, 97%, 98%, 99%, or 100% identical to SEQ ID NO: 77 or SEQ ID NO: 78. In certain embodiments, the CAR is encoded by a nucleotide sequence at least 80%, 85%, 90%, 95%, 96%, 97%, 98%, 99%, or 100% identical to SEQ ID NO: 79 or SEQ ID NO: 80.

Antigen-Binding Domain Derived from Mouse Anti-APP Antibody (Clone 3d6)

In certain embodiments, the antigen-binding domain of the CAR is capable of binding amyloid beta (Aβ) and is derived from mouse anti-amyloid beta antibody (clone 3d6), known as ‘3D6’. 3D6 is described in U.S. Pat. No. 8,614,308 B2 and WO 2006/083689 Aβ, contents of which are incorporated herein by reference in their entireties.

In certain embodiments, the antigen-binding domain comprises a heavy chain variable region that comprises three heavy chain complementarity determining regions (HCDRs). In certain embodiments, HCDR1 comprises the amino acid sequence NYGMS (SEQ ID NO: 37), and/or HCDR2 comprises the amino acid sequence IRSGGGRTYYSDNVKGR (SEQ ID NO: 38), and/or HCDR3 comprises the amino acid sequence YDHYSGSSDY (SEQ ID NO: 39). The antigen-binding domain also comprises a light chain variable region that comprises three light chain complementarity determining regions (LCDRs). In certain embodiments, LCDR1 comprises the amino acid sequence KSSQSLLDSDGKTYLN (SEQ ID NO: 40), and/or LCDR2 comprises the amino acid sequence LVSKLD (SEQ ID NO: 41), and/or LCDR3 comprises the amino acid sequence WQGTHFPRT (SEQ ID NO: 42).

In certain embodiments, the antigen-binding domain comprises any one of SEQ ID NOs: 37-42.

In certain embodiments, the heavy chain variable region (VH) of the antigen-binding domain comprises an amino acid sequence at least 80%, 85%, 90%, 95%, 96%, 97%, 98%, 99%, or 100% identical to SEQ ID NO: 43 and/or the light chain variable region (VL) comprises an amino acid sequence at least 80%, 85%, 90%, 95%, 96%, 97%, 98%, 99%, or 100% identical to SEQ ID NO: 44.

In certain embodiments, the heavy chain variable region (VH) of the antigen-binding domain is encoded by a nucleotide sequence at least 80%, 85%, 90%, 95%, 96%, 97%, 98%, 99%, or 100% identical to SEQ ID NO: 45 and/or the light chain variable region (VL) is encoded by a nucleotide sequence at least 80%, 85%, 90%, 95%, 96%, 97%, 98%, 99%, or 100% identical to SEQ ID NO: 46.

In certain embodiments, the antigen-binding domain is a single-chain variable fragment (scFv) comprising an amino acid sequence at least 80%, 85%, 90%, 95%, 96%, 97%, 98%, 99%, or 100% identical to SEQ ID NO: 47 or SEQ ID NO: 48.

In certain embodiments, the antigen-binding domain is a single-chain variable fragment (scFv) encoded by a nucleotide sequence at least 80%, 85%, 90%, 95%, 96%, 97%, 98%, 99%, or 100% identical to SEQ ID NO: 49 or SEQ ID NO: 50.

In certain embodiments, the CAR comprises an antigen binding domain capable of binding Aβ (e.g. derived from mAb 3D6) and does not contain an intracellular domain. In certain embodiments the CAR comprises an antigen binding domain capable of binding Aβ (e.g. derived from mAb 3D6), a CD8 alpha hinge domain, and a CD28 transmembrane domain. In certain embodiments, the CAR comprises an amino acid sequence at least 80%, 85%, 90%, 95%, 96%, 97%, 98%, 99%, or 100% identical to SEQ ID NO: 51 or SEQ ID NO: 52. In certain embodiments, the CAR is encoded by a nucleotide sequence at least 80%, 85%, 90%, 95%, 96%, 97%, 98%, 99%, or 100% identical to SEQ ID NO: 53 or SEQ ID NO: 54.

In certain embodiments, the CAR comprises an antigen binding domain capable of binding Aβ (e.g. derived from mAb 3D6), a transmembrane domain, and an intracellular domain. The CAR can comprise any intracellular domain known in the art and/or disclosed herein. In certain embodiments, the intracellular domain comprises CD3 zeta. In certain embodiments the CAR comprises an antigen binding domain capable of binding Aβ (e.g. derived from mAb 3D6), a CD8 alpha hinge domain, a CD28 transmembrane domain, and a CD3 zeta intracellular domain. In certain embodiments, the CAR comprises an amino acid sequence at least 80%, 85%, 90%, 95%, 96%, 97%, 98%, 99%, or 100% identical to SEQ ID NO: 81 or SEQ ID NO: 82. In certain embodiments, the CAR is encoded by a nucleotide sequence at least 80%, 85%, 90%, 95%, 96%, 97%, 98%, 99%, or 100% identical to SEQ ID NO: 83 or SEQ ID NO: 84.

Antigen-Binding Domain Derived from Gosuranemab Antibody

In certain embodiments, the antigen-binding domain of the CAR is capable of binding Tau and is derived from the Gosuranemab antibody. Gosuranemab is a humanized IgG4 monoclonal anti-tau antibody (Synonyms: BIIB092, BMS-986168, IPN007) and is described in WO 2018/231254 A1, contents of which is incorporated herein by reference in its entirety.

In certain embodiments, the antigen-binding domain comprises a heavy chain variable region that comprises three heavy chain complementarity determining regions (HCDRs). In certain embodiments, HCDR1 comprises the amino acid sequence KYGMS (SEQ ID NO: 55), and/or HCDR2 comprises the amino acid sequence ISSSGSRTYYPDSVKG (SEQ ID NO: 56), and/or HCDR3 comprises the amino acid sequence WDGAMDY (SEQ ID NO: 57). The antigen-binding domain also comprises a light chain variable region that comprises three light chain complementarity determining regions (LCDRs). In certain embodiments, LCDR1 comprises the amino acid sequence KSSQSIVHSNGNTYLE (SEQ ID NO: 58), and/or LCDR2 comprises the amino acid sequence KVSNRF (SEQ ID NO: 59), and/or LCDR3 comprises the amino acid sequence FQGSLVPWA (SEQ ID NO: 60).

In certain embodiments, the antigen-binding domain comprises any one of SEQ ID NOs: 55-60.

In certain embodiments, the heavy chain variable region (VH) of the antigen-binding domain comprises an amino acid sequence at least 80%, 85%, 90%, 95%, 96%, 97%, 98%, 99%, or 100% identical to SEQ ID NO: 61 and/or the light chain variable region (VL) comprises an amino acid sequence at least 80%, 85%, 90%, 95%, 96%, 97%, 98%, 99%, or 100% identical to SEQ ID NO: 62.

In certain embodiments, the heavy chain variable region (VH) of the antigen-binding domain is encoded by a nucleotide sequence at least 80%, 85%, 90%, 95%, 96%, 97%, 98%, 99%, or 100% identical to SEQ ID NO: 63 and/or the light chain variable region (VL) is encoded by a nucleotide sequence at least 80%, 85%, 90%, 95%, 96%, 97%, 98%, 99%, or 100% identical to SEQ ID NO: 64.

In certain embodiments, the antigen-binding domain is a single-chain variable fragment (scFv) comprising an amino acid sequence at least 80%, 85%, 90%, 95%, 96%, 97%, 98%, 99%, or 100% identical to SEQ ID NO: 65 or SEQ ID NO: 66.

In certain embodiments, the antigen-binding domain is a single-chain variable fragment (scFv) encoded by a nucleotide sequence at least 80%, 85%, 90%, 95%, 96%, 97%, 98%, 99%, or 100% identical to SEQ ID NO: 67 or SEQ ID NO: 68.

In certain embodiments, the CAR comprises an antigen binding domain capable of binding Tau (e.g. derived from Gosuranemab) and does not contain an intracellular domain.

In certain embodiments the CAR comprises an antigen binding domain capable of binding Tau (e.g. derived from Gosuranemab), a CD8 alpha hinge domain, and a CD28 transmembrane domain. In certain embodiments, the CAR comprises an amino acid sequence at least 80%, 85%, 90%, 95%, 96%, 97%, 98%, 99%, or 100% identical to SEQ ID NO: 69 or SEQ ID NO: 70. In certain embodiments, the CAR is encoded by a nucleotide sequence at least 80%, 85%, 90%, 95%, 96%, 97%, 98%, 99%, or 100% identical to SEQ ID NO: 71 or SEQ ID NO: 72.

In certain embodiments, the antigen-binding domain comprises a heavy chain variable region that comprises any of the three heavy chain complementarity determining regions, HCDR1, HCDR2, and HCDR3, as described herein. In certain embodiments, the antigen-binding domain comprises a light chain variable region that comprises any of the three light chain complementarity determining regions, LCDR1, LCDR2, and LCDR3, as described herein. In certain embodiments, the antigen-binding domain comprises any combination of HCDR1, HCDR2, HCDR3, LCDR1, LCDR2, and LCDR3, and described herein. The skilled artisan would readily be able to determine the relevant complementarity determining regions based on amino acid numbering in view of the heavy and light chain variable region sequences provided herein.

In some aspects of the disclosure, an antigen binding domain is operably linked to another domain of a provided CAR, such as the transmembrane domain for expression in the immune cell. In some embodiments, a nucleic acid encoding an antigen binding domain is operably linked to a nucleic acid encoding a transmembrane domain and the transmembrane domain is operably linked to a nucleic acid encoding an intracellular domain. In some embodiments, a modified immune cell (e.g., a modified monocyte, macrophage, or stem cell; e.g., hematopoietic stem cell with the potential to become a myeloid cell such as a monocyte, macrophage or microglia-like cell) comprising a CAR further comprises an additional antigen-binding domain that is required for activation (e.g., a bispecific CAR or bispecific modified immune cell). In some embodiments, a bispecific modified cell can reduce off-target and/or on-target off-tissue effects by requiring that two antigens are present. In some embodiments, a CAR and an additional antigen-binding domain provide distinct signals that in isolation are insufficient to mediate activation of the modified immune cell, but are synergistic together, stimulating activation of the modified immune cell. In some embodiments, such a construct may be referred to as an ‘AND’ logic gate.

In certain embodiments the CAR comprises an antigen binding domain capable of binding Aβ (e.g. derived from Aducanumab, Crenezumab, or mAb 3D6) or Tau (e.g. derived from Gosuranemab), and is capable of delivering a payload. The CAR can comprise any of the antigen-binding domains disclosed herein. The CAR can comprise any type of payload known in the art and/or disclosed herein. In certain embodiments, the payload comprises IL-10. In certain embodiments the CAR comprises an antigen binding domain capable of binding Aβ (e.g. derived from Aducanumab, Crenezumab, or mAb 3D6), a CD8 alpha hinge domain, a CD28 transmembrane domain, and an IL-10 sequence (e.g. SEQ ID NO: 108). In certain embodiments, the CAR comprises an amino acid sequence at least 80%, 85%, 90%, 95%, 96%, 97%, 98%, 99%, or 100% identical to SEQ ID NO: 85. In certain embodiments, the CAR is encoded by a nucleotide sequence at least 80%, 85%, 90%, 95%, 96%, 97%, 98%, 99%, or 100% identical to SEQ ID NO: 86.

In certain embodiments the CAR comprises an antigen binding domain capable of binding Aβ (e.g. derived from Aducanumab, Crenezumab, or mAb 3D6) or Tau (e.g. derived from Gosuranemab), and an Fcγ intracellular signaling domain. The CAR can comprise any of the antigen-binding domains disclosed herein. In certain embodiments the CAR comprises an antigen binding domain capable of binding Aβ (e.g. derived from Aducanumab, Crenezumab, or mAb 3D6), a CD8 alpha hinge domain, a CD28 transmembrane domain, and an Fcγ sequence (e.g. SEQ ID NO: 110). In certain embodiments, the CAR comprises an amino acid sequence at least 80%, 85%, 90%, 95%, 96%, 97%, 98%, 99%, or 100% identical to SEQ ID NO: 87. In certain embodiments, the CAR is encoded by a nucleotide sequence at least 80%, 85%, 90%, 95%, 96%, 97%, 98%, 99%, or 100% identical to SEQ ID NO: 88.

In certain embodiments a composition provided herein comprises a Dual CAR. Dual CAR can comprise any combination of any two antigen binding domains disclosed herein. In certain embodiments, the Dual CAR comprises an antigen binding domain capable of binding Aβ (e.g. derived from Aducanumab, Crenezumab, or mAb 3D6) and an antigen binding domain capable of binding Tau (e.g. derived from Gosuranemab). In certain embodiments the Dual CAR comprises an antigen binding domain derived from Gosuranemab and an antigen binding domain derived from Aducanumab. In certain embodiments, the Dual CAR comprises an amino acid sequence at least 80%, 85%, 90%, 95%, 96%, 97%, 98%, 99%, or 100% identical to SEQ ID NO: 89. In certain embodiments, the Dual CAR is encoded by a nucleotide sequence at least 80%, 85%, 90%, 95%, 96%, 97%, 98%, 99%, or 100% identical to SEQ ID NO: 90.

In certain embodiments the CAR comprises an antigen binding domain capable of binding Aβ (e.g. derived from Aducanumab, Crenezumab, or mAb 3D6) or Tau (e.g. derived from Gosuranemab), and a fused reporter. The CAR can comprise any of the antigen-binding domains disclosed herein. In certain embodiments the CAR comprises an antigen binding domain capable of binding Aβ (e.g. derived from Aducanumab, Crenezumab, or mAb 3D6), a CD8 alpha hinge domain, a CD28 transmembrane domain, and mCherry sequence (e.g. SEQ ID NO: 106). In certain embodiments, the CAR comprises an amino acid sequence at least 80%, 85%, 90%, 95%, 96%, 97%, 98%, 99%, or 100% identical to SEQ ID NO: 91. In certain embodiments, the CAR is encoded by a nucleotide sequence at least 80%, 85%, 90%, 95%, 96%, 97%, 98%, 99%, or 100% identical to SEQ ID NO: 92.

TABLE 1
Amino Acid and Nucleotide Sequences

| SEQ | | |
|---|---|---|
| ID | | |
| NO: | Name | Sequence |
| 1 | Aducanumab | SYGMH |
| | HCDR1 | |
| 2 | Aducanumab | VIWFDGTKKYYTDSVKG |
| | HCDR2 | |
| 3 | Aducanumab | DRGIGARRGPYYMDV |
| | HCDR3 | |
| 4 | Aducanumab | RASQSISSYLN |
| | LCDR1 | |
| 5 | Aducanumab | ASSLQS |
| | LCDR2 | |
| 6 | Aducanumab | QQSYSTPLT |
| | LCDR3 | |
| 7 | Aducanumab | QVQLVESGGGVVQPGRSLRLSCAASGFAFSSYGMHWVRQAPGK |
| | Heavy Chain (VH) | GLEWVAVIWFDGTKKYYTDSVKGRFTISRDNSKNTLYLQMNTL |
| | | RAEDTAVYYCARDRGIGARRGPYYMDVWGKGTTVTVSS |
| 8 | Aducanumab | DIQMTQSPSSLSASVGDRVTITCRASQSISSYLNWYQQKPGKAPK |
| | Light Chain (VL) | LLIYAASSLQSGVPSRFSGSGSGTDFTLTISSLQPEDFATYYCQQSY |
| | | STPLTFGGGTKVEIKRTV |
| 9 | Aducanumab | caggtgcagctggtggaaagcggcggcggcgtggtgcagccgggccgcagcctgcgcctgagctg |
| | Heavy Chain (VH) | cgcggcgagcggctttgcgtttagcagctatggcatgcattgggtgcgccaggcgccgggcaaaggc |
| | | ctggaatgggtggcggtgatttggtttgatggcaccaaaaaatattataccgatagcgtgaaaggccgct |
| | | ttaccattagccgcgataacagcaaaaacaccctgtatctgcagatgaacaccctgcgcgcggaagata |
| | | ccgcggtgtattattgcgcgcgcgatcgcggcattggcgcgcgccgcggcccgtattatatggatgtgt |
| | | ggggcaaaggcaccaccgtgaccgtgagcagc |
| 10 | Aducanumab | gatattcagatgacccagagcccgagcagcctgagcgcgagcgtgggcgatcgcgtgaccattacct |
| | Light Chain (VL) | gccgcgcgagccagagcattagcagctatctgaactggtatcagcagaaaccgggcaaagcgccga |
| | | aactgctgatttatgcggcgagcagcctgcagagcggcgtgccgagccgctttagcggcagcggcag |
| | | cggcaccgattttaccctgaccattagcagcctgcagccggaagattttgcgacctattattgccagcag |
| | | agctatagcaccccgctgacctttggcggcggcaccaaagtggaaattaaacgc |
| 11 | Aducanumab H2L | QVQLVESGGGVVQPGRSLRLSCAASGFAFSSYGMHWVRQAPGK |
| | scFv | GLEWVAVIWFDGTKKYYTDSVKGRFTISRDNSKNTLYLQMNTL |
| | | RAEDTAVYYCARDRGIGARRGPYYMDVWGKGTTVTVSSGGGG |
| | | SGGGGSGGGGSGGGGSDIQMTQSPSSLSASVGDRVTITCRASQSIS |
| | | SYLNWYQQKPGKAPKLLIYAASSLQSGVPSRFSGSGSGTDFTLTIS |
| | | SLQPEDFATYYCQQSYSTPLTFGGGTKVEIKRTV |
| 12 | Aducanumab L2H | DIQMTQSPSSLSASVGDRVTITCRASQSISSYLNWYQQKPGKAPK |
| | scFv | LLIYAASSLQSGVPSRFSGSGSGTDFTLTISSLQPEDFATYYCQQSY |
| | | STPLTFGGGTKVEIKRTVGGGGSGGGGSGGGGSGGGGSQVQLVE |
| | | SGGGVVQPGRSLRLSCAASGFAFSSYGMHWVRQAPGKGLEWVA |
| | | VIWFDGTKKYYTDSVKGRFTISRDNSKNTLYLQMNTLRAEDTAV |
| | | YYCARDRGIGARRGPYYMDVWGKGTTVTVSS |
| 13 | Aducanumab H2L | caggtgcagctggtggaaagcggcggcggcgtggtgcagccgggccgcagcctgcgcctgagctg |
| | scFv | cgcggcgagcggctttgcgtttagcagctatggcatgcattgggtgcgccaggcgccgggcaaaggc |
| | | ctggaatgggtggcggtgatttggtttgatggcaccaaaaaatattataccgatagcgtgaaaggccgct |
| | | ttaccattagccgcgataacagcaaaaacaccctgtatctgcagatgaacaccctgcgcgcggaagata |
| | | ccgcggtgtattattgcgcgcgcgatcgcggcattggcgcgcgccgcggcccgtattatatggatgtgt |
| | | ggggcaaaggcaccaccgtgaccgtgagcagcggcggaggtggaagcggaggcggaggatcagg |
| | | tggcggtggatctggcggtggcggatctgatattcagatgacccagagcccgagcagcctgagcgcg |
| | | agcgtgggcgatcgcgtgaccattacctgccgcgcgagccagagcattagcagctatctgaactggtat |
| | | cagcagaaaccgggcaaagcgccgaaactgctgatttatgcggcgagcagcctgcagagcggcgtg |
| | | ccgagccgctttagcggcagcggcagcggcaccgattttaccctgaccattagcagcctgcagccgga |
| | | agattttgcgacctattattgccagcagagctatagcaccccgctgacctttggcggcggcaccaaagtg |
| | | gaaattaaacgc |
| 14 | Aducanumab L2H | gatatccagatgactcagagccccagcagcctgtctgcctctgtgggagacagagtgaccatcacctgt |
| | scFv | agagccagccagagcatcagcagctacctgaactggtatcagcagaagcccggcaaggcccctaaa |
| | | ctgctgatctatgccgcctccagtctgcagagcggagtgccttctagattttccggcagcggctccggca |
| | | ccgatttcaccctgaccatatctagcctgcagcctgaggacttcgccacctactactgccagcagagcta |
| | | cagcacccctctgacttttggcggaggcaccaaggtggaaatcaagcggacagtgtggcggaggtgg |
| | | aagcggaggcggaggatcaggtggcggtggatctggcggtggcggatctcaggtgcagctggtgga |
| | | aagcggcggcggcgtggtgcagccgggccgcagcctgcgcctgagctgcgcggcgagcggctttg |
| | | cgtttagcagctatggcatgcattgggtgcgccaggcgccgggcaaaggcctggaatgggtggcggt |
| | | gatttggtttgatggcaccaaaaaatattataccgatagcgtgaaaggccgctttaccattagccgcgata |
| | | acagcaaaaacaccctgtatctgcagatgaacaccctgcgcgcggaagataccgcggtgtattattgcg |
| | | cgcgcgatcgcggcattggcgcgcgccgcggcccgtattatatggatgtgtggggcaaaggcaccac |
| | | cgtgaccgtgagcagc |
| 15 | Aducanumab H2L | MALPVTALLLPLALLLHAARPGSQVQLVESGGGVVQPGRSLRLS |
| | CAR | CAASGFAFSSYGMHWVRQAPGKGLEWVAVIWFDGTKKYYTDS |
| | | VKGRFTISRDNSKNTLYLQMNTLRAEDTAVYYCARDRGIGARRG |
| | | PYYMDVWGKGTTVTVSSGGGGSGGGGSGGGGSGGGGSDIQMT |
| | | QSPSSLSASVGDRVTITCRASQSISSYLNWYQQKPGKAPKLLIYAA |
| | | SSLQSGVPSRFSGSGSGTDFTLTISSLQPEDFATYYCQQSYSTPLTF |
| | | GGGTKVEIKRTVSGTTTPAPRPPTPAPTIASQPLSLRPEACRPAAG |
| | | GAVHTRGLDFACDFWVLVVVGGVLACYSLLVTVAFIIFWVRSKR |
| | | STSGSGATNFSLLKQAGDVEENPGPASMVSKGEEDNMAIIKEFM |
| | | RFKVHMEGSVNGHEFEIEGEGEGRPYEGTQTAKLKVTKGGPLPF |
| | | AWDILSPQFMYGSKAYVKHPADIPDYLKLSFPEGFKWERVMNFE |
| | | DGGVVTVTQDSSLQDGEFIYKVKLRGTNFPSDGPVMQKKTMGW |
| | | EASSERMYPEDGALKGEIKQRLKLKDGGHYDAEVKTTYKAKKP |
| | | VQLPGAYNVNIKLDITSHNEDYTIVEQYERAEGRHSTGGMDELY |
| | | K |
| 16 | Aducanumab L2H | MALPVTALLLPLALLLHAARPGSDIQMTQSPSSLSASVGDRVTIT |
| | CAR | CRASQSISSYLNWYQQKPGKAPKLLIYAASSLQSGVPSRFSGSGS |
| | | GTDFTLTISSLQPEDFATYYCQQSYSTPLTFGGGTKVEIKRTVGGG |
| | | GSGGGGSGGGGSGGGGSQVQLVESGGGVVQPGRSLRLSCAASG |
| | | FAFSSYGMHWVRQAPGKGLEWVAVIWFDGTKKYYTDSVKGRF |
| | | TISRDNSKNTLYLQMNTLRAEDTAVYYCARDRGIGARRGPYYM |
| | | DVWGKGTTVTVSSSGTTTPAPRPPTPAPTIASQPLSLRPEACRPAA |
| | | GGAVHTRGLDFACDFWVLVVVGGVLACYSLLVTVAFIIFWVRSK |
| | | RSTSGSGATNFSLLKQAGDVEENPGPASMVSKGEEDNMAIIKEF |
| | | MRFKVHMEGSVNGHEFEIEGEGEGRPYEGTQTAKLKVTKGGPLP |
| | | FAWDILSPQFMYGSKAYVKHPADIPDYLKLSFPEGFKWERVMNF |
| | | EDGGVVTVTQDSSLQDGEFIYKVKLRGTNFPSDGPVMQKKTMG |
| | | WEASSERMYPEDGALKGEIKQRLKLKDGGHYDAEVKTTYKAKK |
| | | PVQLPGAYNVNIKLDITSHNEDYTIVEQYERAEGRHSTGGMDEL |
| | | YK |
| 17 | Aducanumab H2L | atggccctgcctgtgacagccctgctgctgcctctggctctgctgctgcatgccgctagacccggatccc |
| | CAR | aggtgcagctggtggaaagcggcggcggcgtggtgcagccgggccgcagcctgcgcctgagctgc |
| | | gcggcgagcggctttgcgtttagcagctatggcatgcattgggtgcgccaggcgccgggcaaaggcc |
| | | tggaatgggtggcggtgatttggtttgatggcaccaaaaaatattataccgatagcgtgaaaggccgcttt |
| | | accattagccgcgataacagcaaaaacaccctgtatctgcagatgaacaccctgcgcgcggaagatac |
| | | cgcggtgtattattgcgcgcgcgatcgcggcattggcgcgcgccgcggcccgtattatatggatgtgtg |
| | | gggcaaaggcaccaccgtgaccgtgagcagcggcggaggtggaagcggaggcggaggatcaggt |
| | | ggcggtggatctggcggtggcggatctgatattcagatgacccagagcccgagcagcctgagcgcga |
| | | gcgtgggcgatcgcgtgaccattacctgccgcgcgagccagagcattagcagctatctgaactggtatc |
| | | agcagaaaccgggcaaagcgccgaaactgctgatttatgcggcgagcagcctgcagagcggcgtgc |
| | | cgagccgctttagcggcagcggcagcggcaccgattttaccctgaccattagcagcctgcagccggaa |
| | | gattttgcgacctattattgccagcagagctatagcaccccgctgacctttggcggcggcaccaaagtgg |
| | | aaattaaacgctccggaaccacgacgccagcgccgcgaccaccaacaccggcgcccaccatcgcgt |
| | | cgcagcccctgtccctgcgcccagaggcgtgccggccagcggcggggggcgcagtgcacacgag |
| | | ggggctggacttcgcctgtgatttttgggtgctggtggtggttggtggagtcctggcttgctatagcttgc |
| | | tagtaacagtggcctttattattttctgggtgaggagtaagaggagcactagtggaagcggagctactaac |
| | | ttcagcctgctgaagcaggctggagacgtggaggagaaccctggacctgctagcatggtgagcaagg |
| | | gcgaggaggataacatggccatcatcaaggagttcatgcgcttcaaggtgcacatggagggctccgtg |
| | | aacggccacgagttcgagatcgagggcgagggcgagggccgcccctacgagggcacccagaccg |
| | | ccaagctgaaggtgaccaagggtggccccctgcccttcgcctgggacatcctgtcccctcagttcatgt |
| | | acggctccaaggcctacgtgaagcaccccgccgacatccccgactacttgaagctgtccttccccgag |
| | | ggcttcaagtgggagcgcgtgatgaacttcgaggacggcggcgtggtgaccgtgacccaggactcct |
| | | ccctgcaggacggcgagttcatctacaaggtgaagctgcgcggcaccaacttcccctccgacggccc |
| | | cgtaatgcagaagaagaccatgggctgggaggcctcctccgagcggatgtaccccgaggacggcgc |
| | | cctgaagggcgagatcaagcagaggctgaagctgaaggacggcggccactacgacgctgaggtcaa |
| | | gaccacctacaaggccaagaagcccgtgcagctgcccggcgcctacaacgtcaacatcaagttggac |
| | | atcacctcccacaacgaggactacaccatcgtggaacagtacgaacgcgccgagggccgccactcca |
| | | ccggcggcatggacgagctgtacaagtaa |
| 18 | Aducanumab L2H | atggccctgcctgtgacagccctgctgctgcctctggctctgctgctgcatgccgctagacccggatcc |
| | CAR | gatatccagatgactcagagccccagcagcctgtctgcctctgtgggagacagagtgaccatcacctgt |
| | | agagccagccagagcatcagcagctacctgaactggtatcagcagaagcccggcaaggcccctaaa |
| | | ctgctgatctatgccgcctccagtctgcagagcggagtgccttctagattttccggcagcggctccggca |
| | | ccgatttcaccctgaccatatctagcctgcagcctgaggacttcgccacctactactgccagcagagcta |
| | | cagcacccctctgacttttggcggaggcaccaaggtggaaatcaagcggacagtgtggcggaggtgg |
| | | aagcggaggcggaggatcaggtggcggtggatctggcggtggcggatctcaggtgcagctggtgga |
| | | aagcggcggcggcgtggtgcagccgggccgcagcctgcgcctgagctgcgcggcgagcggctttg |
| | | cgtttagcagctatggcatgcattgggtgcgccaggcgccgggcaaaggcctggaatgggtggcggt |
| | | gatttggtttgatggcaccaaaaaatattataccgatagcgtgaaaggccgctttaccattagccgcgata |
| | | acagcaaaaacaccctgtatctgcagatgaacaccctgcgcgcggaagataccgcggtgtattattgcg |
| | | cgcgcgatcgcggcattggcgcgcgccgcggcccgtattatatggatgtgtggggcaaaggcaccac |
| | | cgtgaccgtgagcagcccggaaccacgacgccagcgccgcgaccaccaacaccggcgcccaccat |
| | | cgcgtcgcagcccctgtccctgcgcccagaggcgtgccggccagcggggggggcgcagtgcaca |
| | | cgagggggctggacttcgcctgtgatttttgggtgctggtggtggttggtggagtcctggcttgctatagc |
| | | ttgctagtaacagtggcctttattattttctgggtgaggagtaagaggagcactagtggaagcggagctac |
| | | taacttcagcctgctgaagcaggctggagacgtggaggagaaccctggacctgctagcatggtgagca |
| | | agggcgaggaggataacatggccatcatcaaggagttcatgcgcttcaaggtgcacatggagggctcc |
| | | gtgaacggccacgagttcgagatcgagggcgagggcgagggccgcccctacgagggcacccagac |
| | | cgccaagctgaaggtgaccaagggtggccccctgcccttcgcctgggacatcctgtcccctcagttcat |
| | | gtacggctccaaggcctacgtgaagcaccccgccgacatccccgactacttgaagctgtccttccccga |
| | | gggcttcaagtgggagcgcgtgatgaacttcgaggacggcggcgtggtgaccgtgacccaggactcc |
| | | tccctgcaggacggcgagttcatctacaaggtgaagctgcgcggcaccaacttcccctccgacggccc |
| | | cgtaatgcagaagaagaccatgggctgggaggcctcctccgagcggatgtaccccgaggacggcgc |
| | | cctgaagggcgagatcaagcagaggctgaagctgaaggacggcggccactacgacgctgaggtcaa |
| | | gaccacctacaaggccaagaagcccgtgcagctgcccggcgcctacaacgtcaacatcaagttggac |
| | | atcacctcccacaacgaggactacaccatcgtggaacagtacgaacgcgccgagggccgccactcca |
| | | ccggcggcatggacgagctgtacaagtaa |
| 19 | Crenezumab | GFTFSSYGMS |
| | HCDR1 | |
| 20 | Crenezumab | SINSNGGSTYYPDSVK |
| | HCDR2 | |
| 21 | Crenezumab | GDY |
| | HCDR3 | |
| 22 | Crenezumab | RSSQSLVYSNGDTYLH |
| | LCDR1 | |
| 23 | Crenezumab | KVSNRFS |
| | LCDR2 | |
| 24 | Crenezumab | SQSTHVPWT |
| | LCDR3 | |
| 25 | Crenezumab | EVQLVESGGGLVQPGGSLRLSCAASGFTFSSYGMSWVRQAPGKG |
| | Heavy Chain (VH) | LELVASINSNGGSTYYPDSVKGRFTISRDNAKNSLYLQMNSLRAE |
| | | DTAVYYCASGDYWGQGTTVTVSS |
| 26 | Crenezumab | DIVMTQSPLSLPVTPGEPASISCRSSQSLVYSNGDTYLHWYLQKP |
| | Light Chain (VL) | GQSPQLLIYKVSNRFSGVPDRFSGSGSGTDFTLKISRVEAEDVGV |
| | | YYCSQSTHVPWTFGQGTKVEIK |
| 27 | Crenezumab | gaggtgcagctggttgaatctggcggaggactggttcagcctggcggatctctgagactgtcttgtgcc |
| | Heavy Chain (VH) | gccagcggcttcacctttagcagctacggcatgagctgggtccgacaggctcctggcaaaggactgga |
| | | actggtggccagcatcaacagcaatggcggcagcacctactatcccgacagcgtgaagggcagattc |
| | | accatcagccgggacaacgccaagaacagcctgtacctgcagatgaactccctgagagccgaggac |
| | | accgccgtgtactattgtgccagcggcgattattggggccagggcacaacagtgacagtgtctagc |
| 28 | Crenezumab | gacattgtgatgacacagagccctctgagcctgccagtgacacctggcgaacctgccagcatctcctgt |
| | Light Chain (VL) | agaagcagccagagcctggtgtacagcaacggcgatacctacctgcactggtatctgcagaagcccg |
| | | gccagtctcctcagctgctgatctacaaggtgtccaaccggttcagcggcgtgcccgatagattttctgg |
| | | cagcggctctggcaccgacttcaccctgaagatctccagagtggaagccgaggatgtgggcgtgtact |
| | | actgcagccagtctacccacgtgccatggacatttggacagggcaccaaggtggaaatcaagtccgga |
| 29 | Crenezumab H2L | EVQLVESGGGLVQPGGSLRLSCAASGFTFSSYGMSWVRQAPGKG |
| | scFv | LELVASINSNGGSTYYPDSVKGRFTISRDNAKNSLYLQMNSLRAE |
| | | DTAVYYCASGDYWGQGTTVTVSSGGGGSGGGGSGGGGSGGGG |
| | | SDIVMTQSPLSLPVTPGEPASISCRSSQSLVYSNGDTYLHWYLQKP |
| | | GQSPQLLIYKVSNRFSGVPDRFSGSGSGTDFTLKISRVEAEDVGV |
| | | YYCSQSTHVPWTFGQGTKVEIK |
| 30 | Crenezumab L2H | DIVMTQSPLSLPVTPGEPASISCRSSQSLVYSNGDTYLHWYLQKP |
| | scFv | GQSPQLLIYKVSNRFSGVPDRFSGSGSGTDFTLKISRVEAEDVGV |
| | | YYCSQSTHVPWTFGQGTKVEIKGGGGSGGGGSGGGGSGGGGSE |
| | | VQLVESGGGLVQPGGSLRLSCAASGFTFSSYGMSWVRQAPGKGL |
| | | ELVASINSNGGSTYYPDSVKGRFTISRDNAKNSLYLQMNSLRAED |
| | | TAVYYCASGDYWGQGTTVTVSS |
| 31 | Crenezumab H2L | gaggtgcagctggttgaatctggcggaggactggttcagcctggcggatctctgagactgtcttgtgcc |
| | scFv | gccagcggcttcacctttagcagctacggcatgagctgggtccgacaggctcctggcaaaggactgga |
| | | actggtggccagcatcaacagcaatggcggcagcacctactatcccgacagcgtgaagggcagattc |
| | | accatcagccgggacaacgccaagaacagcctgtacctgcagatgaactccctgagagccgaggac |
| | | accgccgtgtactattgtgccagcggcgattattggggccagggcacaacagtgacagtgtctagcgg |
| | | cggaggtggaagcggaggcggaggatcaggtggcggtggatctggcggtggcggatctgacattgt |
| | | gatgacacagagccctctgagcctgccagtgacacctggcgaacctgccagcatctcctgtagaagca |
| | | gccagagcctggtgtacagcaacggcgatacctacctgcactggtatctgcagaagcccggccagtct |
| | | cctcagctgctgatctacaaggtgtccaaccggttcagcggcgtgcccgatagattttctggcagcggct |
| | | ctggcaccgacttcaccctgaagatctccagagtggaagccgaggatgtgggcgtgtactactgcagc |
| | | cagtctacccacgtgccatggacatttggacagggcaccaaggtggaaatcaagtccgga |
| 32 | Crenezumab L2H | gacattgtgatgacacagagccctctgagcctgccagtgacacctggcgaacctgccagcatctcctgt |
| | scFv | agaagcagccagagcctggtgtacagcaacggcgatacctacctgcactggtatctgcagaagcccg |
| | | gccagtctcctcagctgctgatctacaaggtgtccaaccggttcagcggcgtgcccgatagattttctgg |
| | | cagcggctctggcaccgacttcaccctgaagatctccagagtggaagccgaggatgtgggcgtgtact |
| | | actgcagccagtctacccacgtgccatggacatttggacagggcaccaaggtggaaatcaagtccgga |
| | | ggcggaggtggaagcggaggcggaggatcaggtggcggtggatctggcggtggcggatctgaggt |
| | | gcagctggttgaatctggcggaggactggttcagcctggcggatctctgagactgtcttgtgccgccag |
| | | cggcttcacctttagcagctacggcatgagctgggtccgacaggctcctggcaaaggactggaactgg |
| | | tggccagcatcaacagcaatggcggcagcacctactatcccgacagcgtgaagggcagattcaccatc |
| | | agccgggacaacgccaagaacagcctgtacctgcagatgaactccctgagagccgaggacaccgcc |
| | | gtgtactattgtgccagcggcgattattggggccagggcacaacagtgacagtgtctagc |
| 33 | Crenezumab H2L | MALPVTALLLPLALLLHAARPGSEVQLVESGGGLVQPGGSLRLS |
| | CAR | CAASGFTFSSYGMSWVRQAPGKGLELVASINSNGGSTYYPDSVK |
| | | GRFTISRDNAKNSLYLQMNSLRAEDTAVYYCASGDYWGQGTTV |
| | | TVSSGGGGSGGGGSGGGGSGGGGSDIVMTQSPLSLPVTPGEPASI |
| | | SCRSSQSLVYSNGDTYLHWYLQKPGQSPQLLIYKVSNRFSGVPDR |
| | | FSGSGSGTDFTLKISRVEAEDVGVYYCSQSTHVPWTFGQGTKVEI |
| | | KSGTTTPAPRPPTPAPTIASQPLSLRPEACRPAAGGAVHTRGLDFA |
| | | CDFWVLVVVGGVLACYSLLVTVAFIIFWVRSKRSTSGSGATNFSL |
| | | LKQAGDVEENPGPASMVSKGEEDNMAIIKEFMRFKVHMEGSVN |
| | | GHEFEIEGEGEGRPYEGTQTAKLKVTKGGPLPFAWDILSPQFMYG |
| | | SKAYVKHPADIPDYLKLSFPEGFKWERVMNFEDGGVVTVTQDSS |
| | | LQDGEFIYKVKLRGTNFPSDGPVMQKKTMGWEASSERMYPEDG |
| | | ALKGEIKQRLKLKDGGHYDAEVKTTYKAKKPVQLPGAYNVNIK |
| | | LDITSHNEDYTIVEQYERAEGRHSTGGMDELYK |
| 34 | Crenezumab L2H | MALPVTALLLPLALLLHAARPGSDIVMTQSPLSLPVTPGEPASISC |
| | CAR | RSSQSLVYSNGDTYLHWYLQKPGQSPQLLIYKVSNRFSGVPDRFS |
| | | GSGSGTDFTLKISRVEAEDVGVYYCSQSTHVPWTFGQGTKVEIK |
| | | GGGGSGGGGSGGGGSGGGGSEVQLVESGGGLVQPGGSLRLSCA |
| | | ASGFTFSSYGMSWVRQAPGKGLELVASINSNGGSTYYPDSVKGR |
| | | FTISRDNAKNSLYLQMNSLRAEDTAVYYCASGDYWGQGTTVTV |
| | | SSSGTTTPAPRPPTPAPTIASQPLSLRPEACRPAAGGAVHTRGLDF |
| | | ACDFWVLVVVGGVLACYSLLVTVAFIIFWVRSKRSTSGSGATNF |
| | | SLLKQAGDVEENPGPASMVSKGEEDNMAIIKEFMRFKVHMEGSV |
| | | NGHEFEIEGEGEGRPYEGTQTAKLKVTKGGPLPFAWDILSPQFMY |
| | | GSKAYVKHPADIPDYLKLSFPEGFKWERVMNFEDGGVVTVTQDS |
| | | SLQDGEFIYKVKLRGTNFPSDGPVMQKKTMGWEASSERMYPED |
| | | GALKGEIKQRLKLKDGGHYDAEVKTTYKAKKPVQLPGAYNVNI |
| | | KLDITSHNEDYTIVEQYERAEGRHSTGGMDELYK |
| 35 | Crenezumab H2L | atggccctgcctgtgacagccctgctgctgcctctggctctgctgctgcatgccgctagacccggatcc |
| | CAR | gaggtgcagctggttgaatctggcggaggactggttcagcctggcggatctctgagactgtcttgtgcc |
| | | gccagcggcttcacctttagcagctacggcatgagctgggtccgacaggctcctggcaaaggactgga |
| | | actggtggccagcatcaacagcaatggcggcagcacctactatcccgacagcgtgaagggcagattc |
| | | accatcagccgggacaacgccaagaacagcctgtacctgcagatgaactccctgagagccgaggac |
| | | accgccgtgtactattgtgccagcggcgattattggggccagggcacaacagtgacagtgtctagcgg |
| | | cggaggtggaagcggaggcggaggatcaggtggcggtggatctggcggtggcggatctgacattgt |
| | | gatgacacagagccctctgagcctgccagtgacacctggcgaacctgccagcatctcctgtagaagca |
| | | gccagagcctggtgtacagcaacggcgatacctacctgcactggtatctgcagaagcccggccagtct |
| | | cctcagctgctgatctacaaggtgtccaaccggttcagcggcgtgcccgatagattttctggcagcggct |
| | | ctggcaccgacttcaccctgaagatctccagagtggaagccgaggatgtgggcgtgtactactgcagc |
| | | cagtctacccacgtgccatggacatttggacagggcaccaaggtggaaatcaagtccggaccggaac |
| | | cacgacgccagcgccgcgaccaccaacaccggcgcccaccatcgcgtcgcagcccctgtccctgcg |
| | | cccagaggcgtgccggccagcggcggggggcgcagtgcacacgagggggctggacttcgcctgtg |
| | | atttttgggtgctggtggtggttggtggagtcctggcttgctatagcttgctagtaacagtggcctttatt |
| | | attttctgggtgaggagtaagaggagcactagtggaagcggagctactaacttcagcctgctgaagcaggct |
| | | ggagacgtggaggagaaccctggacctgctagcatggtgagcaagggcgaggaggataacatggcc |
| | | atcatcaaggagttcatgcgcttcaaggtgcacatggagggctccgtgaacggccacgagttcgagatc |
| | | gagggcgagggcgagggccgcccctacgagggcacccagaccgccaagctgaaggtgaccaagg |
| | | gtggccccctgcccttcgcctgggacatcctgtcccctcagttcatgtacggctccaaggcctacgtgaa |
| | | gcaccccgccgacatccccgactacttgaagctgtccttccccgagggcttcaagtgggagcgcgtga |
| | | tgaacttcgaggacggcggcgtggtgaccgtgacccaggactcctccctgcaggacggcgagttcatc |
| | | tacaaggtgaagctgcgcggcaccaacttcccctccgacggccccgtaatgcagaagaagaccatgg |
| | | gctgggaggcctcctccgagcggatgtaccccgaggacggcgccctgaagggcgagatcaagcag |
| | | aggctgaagctgaaggacggcggccactacgacgctgaggtcaagaccacctacaaggccaagaag |
| | | cccgtgcagctgcccggcgcctacaacgtcaacatcaagttggacatcacctcccacaacgaggacta |
| | | caccatcgtggaacagtacgaacgcgccgagggccgccactccaccggcggcatggacgagctgta |
| | | caagtaa |
| 36 | Crenezumab L2H | atggccctgcctgtgacagccctgctgctgcctctggctctgctgctgcatgccgctagacccggatcc |
| | CAR | gacattgtgatgacacagagccctctgagcctgccagtgacacctggcgaacctgccagcatctcctgt |
| | | agaagcagccagagcctggtgtacagcaacggcgatacctacctgcactggtatctgcagaagcccg |
| | | gccagtctcctcagctgctgatctacaaggtgtccaaccggttcagcggcgtgcccgatagattttctgg |
| | | cagcggctctggcaccgacttcaccctgaagatctccagagtggaagccgaggatgtgggcgtgtact |
| | | actgcagccagtctacccacgtgccatggacatttggacagggcaccaaggtggaaatcaagtccgga |
| | | ggcggaggtggaagcggaggcggaggatcaggtggcggtggatctggcggtggcggatctgaggt |
| | | gcagctggttgaatctggcggaggactggttcagcctggcggatctctgagactgtcttgtgccgccag |
| | | cggcttcacctttagcagctacggcatgagctgggtccgacaggctcctggcaaaggactggaactgg |
| | | tggccagcatcaacagcaatggcggcagcacctactatcccgacagcgtgaagggcagattcaccatc |
| | | agccgggacaacgccaagaacagcctgtacctgcagatgaactccctgagagccgaggacaccgcc |
| | | gtgtactattgtgccagcggcgattattggggccagggcacaacagtgacagtgtctagcccggaacc |
| | | acgacgccagcgccgcgaccaccaacaccggcgcccaccatcgcgtcgcagcccctgtccctgcgc |
| | | ccagaggcgtgccggccagcggggggggcgcagtgcacacgagggggctggacttcgcctgtga |
| | | tttttgggtgctggtggtggttggtggagtcctggcttgctatagcttgctagtaacagtggcctttatta |
| | | ttttctgggtgaggagtaagaggagcactagtggaagcggagctactaacttcagcctgctgaagcaggct |
| | | ggagacgtggaggagaaccctggacctgctagcatggtgagcaagggcgaggaggataacatggcc |
| | | atcatcaaggagttcatgcgcttcaaggtgcacatggagggctccgtgaacggccacgagttcgagatc |
| | | gagggcgagggcgagggccgcccctacgagggcacccagaccgccaagctgaaggtgaccaagg |
| | | gtggccccctgcccttcgcctgggacatcctgtcccctcagttcatgtacggctccaaggcctacgtgaa |
| | | gcaccccgccgacatccccgactacttgaagctgtccttccccgagggcttcaagtgggagcgcgtga |
| | | tgaacttcgaggacggcggcgtggtgaccgtgacccaggactcctccctgcaggacggcgagttcatc |
| | | tacaaggtgaagctgcgcggcaccaacttcccctccgacggccccgtaatgcagaagaagaccatgg |
| | | gctgggaggcctcctccgagcggatgtaccccgaggacggcgccctgaagggcgagatcaagcag |
| | | aggctgaagctgaaggacggcggccactacgacgctgaggtcaagaccacctacaaggccaagaag |
| | | cccgtgcagctgcccggcgcctacaacgtcaacatcaagttggacatcacctcccacaacgaggacta |
| | | caccatcgtggaacagtacgaacgcgccgagggccgccactccaccggcggcatggacgagctgta |
| | | caagtaa |
| 37 | mAb 3d6 HCDR1 | NYGMS |
| 38 | mAb 3d6 HCDR2 | IRSGGGRTYYSDNVKGR |
| 39 | mAb 3d6 HCDR3 | YDHYSGSSDY |
| 40 | mAb 3d6 LCDR1 | KSSQSLLDSDGKTYLN |
| 41 | mAb 3d6 LCDR2 | LVSKLD |
| 42 | mAb 3d6 LCDR3 | WQGTHFPRT |
| 43 | mAb 3d6 Heavy | EVKLVESGGGLVKPGASLKLSCAASGFTFSNYGMSWVRQNSDK |
| | Chain (VH) | RLEWVASIRSGGGRTYYSDNVKGRFTISRENAKNTLYLQMSSLK |
| | | SEDTALYYCVRYDHYSGSSDYWGQGTTVTVSSSG |
| 44 | mAb 3d6 Light | YVVMTQTPLTLSVTIGQPASISCKSSQSLLDSDGKTYLNWLLQRP |
| | Chain (VL) | GQSPKRLIYLVSKLDSGVPDRFTGSGSGTDFTLKISRIEAEDLGLY |
| | | YCWQGTHFPRTFGGGTKLEIK |
| 45 | mAb 3d6 Heavy | gaagtgaagctggttgaaagtggcggaggcctggttaagcctggcgcctctctgaaactgtcttgtgcc |
| | Chain (VH) | gccagcggcttcaccttcagcaactacggcatgagctgggtccgacagaacagcgacaagcggcttg |
| | | agtgggtcgcctctattagatccggcggaggcagaacctactactccgacaacgtgaagggcagattc |
| | | accatcagcagagagaacgccaagaacaccctgtacctgcagatgagcagcctgaagtccgaggac |
| | | accgctctgtactattgcgtcagatacgaccactacagcggcagcagcgattattggggccagggcac |
| | | aaccgtgaccgtgtctagctccgga |
| 46 | mAb 3d6 Light | tacgtggtcatgacacagacccctctgacactgagcgtgaccattggacagcctgccagcatcagctgc |
| | Chain (VL) | aagagcagtcagagcctgctggactccgacggcaagacctacctgaattggctgctgcagaggcccg |
| | | gacagagccccaagagactgatctacctggtgtccaagctggacagcggcgtgcccgatagattcaca |
| | | ggctctggcagcggcaccgacttcaccctgaagatctctagaatcgaggccgaggacctgggcctgta |
| | | ctactgttggcagggcacacacttccccagaacctttggcggcggaacaaagctggaaatcaaa |
| 47 | mAb 3d6 H2L | EVKLVESGGGLVKPGASLKLSCAASGFTFSNYGMSWVRQNSDK |
| | scFv | RLEWVASIRSGGGRTYYSDNVKGRFTISRENAKNTLYLQMSSLK |
| | | SEDTALYYCVRYDHYSGSSDYWGQGTTVTVSSSGGGGGSGGGG |
| | | SGGGGSGGGGSYVVMTQTPLTLSVTIGQPASISCKSSQSLLDSDG |
| | | KTYLNWLLQRPGQSPKRLIYLVSKLDSGVPDRFTGSGSGTDFTLK |
| | | ISRIEAEDLGLYYCWQGTHFPRTFGGGTKLEIK |
| 48 | mAb 3d6 L2H | YVVMTQTPLTLSVTIGQPASISCKSSQSLLDSDGKTYLNWLLQRP |
| | scFv | GQSPKRLIYLVSKLDSGVPDRFTGSGSGTDFTLKISRIEAEDLGLY |
| | | YCWQGTHFPRTFGGGTKLEIKGGGGSGGGGSGGGGSGGGGSEV |
| | | KLVESGGGLVKPGASLKLSCAASGFTFSNYGMSWVRQNSDKRLE |
| | | WVASIRSGGGRTYYSDNVKGRFTISRENAKNTLYLQMSSLKSED |
| | | TALYYCVRYDHYSGSSDYWGQGTTVTVSSSG |
| 49 | mAb 3d6 H2L | gaagtgaagctggttgaaagtggcggaggcctggttaagcctggcgcctctctgaaactgtcttgtgcc |
| | scFv | gccagcggcttcaccttcagcaactacggcatgagctgggtccgacagaacagcgacaagcggcttg |
| | | agtgggtcgcctctattagatccggcggaggcagaacctactactccgacaacgtgaagggcagattc |
| | | accatcagcagagagaacgccaagaacaccctgtacctgcagatgagcagcctgaagtccgaggac |
| | | accgctctgtactattgcgtcagatacgaccactacagcggcagcagcgattattggggccagggcac |
| | | aaccgtgaccgtgtctagctccggaggcggaggtggaagcggaggcggaggatcaggtggcggtg |
| | | gatctggcggtggcggatcttacgtggtcatgacacagacccctctgacactgagcgtgaccattggac |
| | | agcctgccagcatcagctgcaagagcagtcagagcctgctggactccgacggcaagacctacctgaa |
| | | ttggctgctgcagaggcccggacagagccccaagagactgatctacctggtgtccaagctggacagc |
| | | ggcgtgcccgatagattcacaggctctggcagcggcaccgacttcaccctgaagatctctagaatcga |
| | | ggccgaggacctgggcctgtactactgttggcagggcacacacttccccagaacctttggcggcggaa |
| | | caaagctggaaatcaaa |
| 50 | mAb 3d6 L2H | tacgtggtcatgacacagacccctctgacactgagcgtgaccattggacagcctgccagcatcagctgc |
| | scFv | aagagcagtcagagcctgctggactccgacggcaagacctacctgaattggctgctgcagaggcccg |
| | | gacagagccccaagagactgatctacctggtgtccaagctggacagcggcgtgcccgatagattcaca |
| | | ggctctggcagcggcaccgacttcaccctgaagatctctagaatcgaggccgaggacctgggcctgta |
| | | ctactgttggcagggcacacacttccccagaacctttggcggcggaacaaagctggaaatcaaaggcg |
| | | gaggtggaagcggaggcggaggatcaggtggcggtggatctggcggtggcggatctgaagtgaag |
| | | ctggttgaaagtggcggaggcctggttaagcctggcgcctctctgaaactgtcttgtgccgccagcggc |
| | | ttcaccttcagcaactacggcatgagctgggtccgacagaacagcgacaagcggcttgagtgggtcgc |
| | | ctctattagatccggcggaggcagaacctactactccgacaacgtgaagggcagattcaccatcagca |
| | | gagagaacgccaagaacaccctgtacctgcagatgagcagcctgaagtccgaggacaccgctctgta |
| | | ctattgcgtcagatacgaccactacagcggcagcagcgattattggggccagggcacaaccgtgaccg |
| | | tgtctagctccgga |
| 51 | mAb 3d6 H2L | MALPVTALLLPLALLLHAARPGSEVKLVESGGGLVKPGASLKLS |
| | CAR | CAASGFTFSNYGMSWVRQNSDKRLEWVASIRSGGGRTYYSDNV |
| | | KGRFTISRENAKNTLYLQMSSLKSEDTALYYCVRYDHYSGSSDY |
| | | WGQGTTVTVSSSGGGGGSGGGGSGGGGSGGGGSYVVMTQTPLT |
| | | LSVTIGQPASISCKSSQSLLDSDGKTYLNWLLQRPGQSPKRLIYLV |
| | | SKLDSGVPDRFTGSGSGTDFTLKISRIEAEDLGLYYCWQGTHFPR |
| | | TFGGGTKLEIKSGTTTPAPRPPTPAPTIASQPLSLRPEACRPAAGGA |
| | | VHTRGLDFACDFWVLVVVGGVLACYSLLVTVAFIIFWVRSKRST |
| | | SGSGATNFSLLKQAGDVEENPGPASMVSKGEEDNMAIIKEFMRF |
| | | KVHMEGSVNGHEFEIEGEGEGRPYEGTQTAKLKVTKGGPLPFAW |
| | | DILSPQFMYGSKAYVKHPADIPDYLKLSFPEGFKWERVMNFEDG |
| | | GVVTVTQDSSLQDGEFIYKVKLRGTNFPSDGPVMQKKTMGWEA |
| | | SSERMYPEDGALKGEIKQRLKLKDGGHYDAEVKTTYKAKKPVQ |
| | | LPGAYNVNIKLDITSHNEDYTIVEQYERAEGRHSTGGMDELYK |
| 52 | mAb 3d6 L2H | MALPVTALLLPLALLLHAARPGSYVVMTQTPLTLSVTIGQPASIS |
| | CAR | CKSSQSLLDSDGKTYLNWLLQRPGQSPKRLIYLVSKLDSGVPDRF |
| | | TGSGSGTDFTLKISRIEAEDLGLYYCWQGTHFPRTFGGGTKLEIK |
| | | GGGGSGGGGSGGGGSGGGGSEVKLVESGGGLVKPGASLKLSCA |
| | | ASGFTFSNYGMSWVRQNSDKRLEWVASIRSGGGRTYYSDNVKG |
| | | RFTISRENAKNTLYLQMSSLKSEDTALYYCVRYDHYSGSSDYWG |
| | | QGTTVTVSSSGSGTTTPAPRPPTPAPTIASQPLSLRPEACRPAAGG |
| | | AVHTRGLDFACDFWVLVVVGGVLACYSLLVTVAFIIFWVRSKRS |
| | | TSGSGATNFSLLKQAGDVEENPGPASMVSKGEEDNMAIIKEFMR |
| | | FKVHMEGSVNGHEFEIEGEGEGRPYEGTQTAKLKVTKGGPLPFA |
| | | WDILSPQFMYGSKAYVKHPADIPDYLKLSFPEGFKWERVMNFED |
| | | GGVVTVTQDSSLQDGEFIYKVKLRGTNFPSDGPVMQKKTMGWE |
| | | ASSERMYPEDGALKGEIKQRLKLKDGGHYDAEVKTTYKAKKPV |
| | | QLPGAYNVNIKLDITSHNEDYTIVEQYERAEGRHSTGGMDELYK |
| 53 | mAb 3d6 H2L | atggccctgcctgtgacagccctgctgctgcctctggctctgctgctgcatgccgctagacccggatcc |
| | CAR | gaagtgaagctggttgaaagtggcggaggcctggttaagcctggcgcctctctgaaactgtcttgtgcc |
| | | gccagcggcttcaccttcagcaactacggcatgagctgggtccgacagaacagcgacaagcggcttg |
| | | agtgggtcgcctctattagatccggcggaggcagaacctactactccgacaacgtgaagggcagattc |
| | | accatcagcagagagaacgccaagaacaccctgtacctgcagatgagcagcctgaagtccgaggac |
| | | accgctctgtactattgcgtcagatacgaccactacagcggcagcagcgattattggggccagggcac |
| | | aaccgtgaccgtgtctagctccggaggcggaggtggaagcggaggcggaggatcaggtggcggtg |
| | | gatctggcggtggcggatcttacgtggtcatgacacagacccctctgacactgagcgtgaccattggac |
| | | agcctgccagcatcagctgcaagagcagtcagagcctgctggactccgacggcaagacctacctgaa |
| | | ttggctgctgcagaggcccggacagagccccaagagactgatctacctggtgtccaagctggacagc |
| | | ggcgtgcccgatagattcacaggctctggcagcggcaccgacttcaccctgaagatctctagaatcga |
| | | ggccgaggacctgggcctgtactactgttggcagggcacacacttccccagaacctttggcggcggaa |
| | | caaagctggaaatcaaaccggaaccacgacgccagcgccgcgaccaccaacaccggcgcccacca |
| | | tcgcgtcgcagcccctgtccctgcgcccagaggcgtgccggccagcggggggggcgcagtgcac |
| | | acgagggggctggacttcgcctgtgatttttgggtgctggtggtggttggtggagtcctggcttgctatag |
| | | cttgctagtaacagtggcctttattattttctgggtgaggagtaagaggagcactagtggaagcggagcta |
| | | ctaacttcagcctgctgaagcaggctggagacgtggaggagaaccctggacctgctagcatggtgagc |
| | | aagggcgaggaggataacatggccatcatcaaggagttcatgcgcttcaaggtgcacatggagggctc |
| | | cgtgaacggccacgagttcgagatcgagggcgagggcgagggccgcccctacgagggcacccaga |
| | | ccgccaagctgaaggtgaccaagggtggccccctgcccttcgcctgggacatcctgtcccctcagttca |
| | | tgtacggctccaaggcctacgtgaagcaccccgccgacatccccgactacttgaagctgtccttccccg |
| | | agggcttcaagtgggagcgcgtgatgaacttcgaggacggcggcgtggtgaccgtgacccaggactc |
| | | ctccctgcaggacggcgagttcatctacaaggtgaagctgcgcggcaccaacttcccctccgacggcc |
| | | ccgtaatgcagaagaagaccatgggctgggaggcctcctccgagcggatgtaccccgaggacggcg |
| | | ccctgaagggcgagatcaagcagaggctgaagctgaaggacggcggccactacgacgctgaggtca |
| | | agaccacctacaaggccaagaagcccgtgcagctgcccggcgcctacaacgtcaacatcaagttgga |
| | | catcacctcccacaacgaggactacaccatcgtggaacagtacgaacgcgccgagggccgccactcc |
| | | accggcggcatggacgagctgtacaagtaa |
| 54 | mAb 3d6 L2H | atggccctgcctgtgacagccctgctgctgcctctggctctgctgctgcatgccgctagacccggatcct |
| | CAR | acgtggtcatgacacagacccctctgacactgagcgtgaccattggacagcctgccagcatcagctgc |
| | | aagagcagtcagagcctgctggactccgacggcaagacctacctgaattggctgctgcagaggcccg |
| | | gacagagccccaagagactgatctacctggtgtccaagctggacagcggcgtgcccgatagattcaca |
| | | ggctctggcagcggcaccgacttcaccctgaagatctctagaatcgaggccgaggacctgggcctgta |
| | | ctactgttggcagggcacacacttccccagaacctttggcggcggaacaaagctggaaatcaaaggcg |
| | | gaggtggaagcggaggcggaggatcaggtggcggtggatctggcggtggcggatctgaagtgaag |
| | | ctggttgaaagtggcggaggcctggttaagcctggcgcctctctgaaactgtcttgtgccgccagcggc |
| | | ttcaccttcagcaactacggcatgagctgggtccgacagaacagcgacaagcggcttgagtgggtcgc |
| | | ctctattagatccggcggaggcagaacctactactccgacaacgtgaagggcagattcaccatcagca |
| | | gagagaacgccaagaacaccctgtacctgcagatgagcagcctgaagtccgaggacaccgctctgta |
| | | ctattgcgtcagatacgaccactacagcggcagcagcgattattggggccagggcacaaccgtgaccg |
| | | tgtctagctccggaccggaaccacgacgccagcgccgcgaccaccaacaccggcgcccaccatcgc |
| | | gtcgcagcccctgtccctgcgcccagaggcgtgccggccagcggcggggggcgcagtgcacacga |
| | | gggggctggacttcgcctgtgatttttgggtgctggtggtggttggtggagtcctggcttgctatagcttg |
| | | ctagtaacagtggcctttattattttctgggtgaggagtaagaggagcactagtggaagcggagctactaa |
| | | cttcagcctgctgaagcaggctggagacgtggaggagaaccctggacctgctagcatggtgagcaag |
| | | ggcgaggaggataacatggccatcatcaaggagttcatgcgcttcaaggtgcacatggagggctccgt |
| | | gaacggccacgagttcgagatcgagggcgagggcgagggccgcccctacgagggcacccagacc |
| | | gccaagctgaaggtgaccaagggtggccccctgcccttcgcctgggacatcctgtcccctcagttcatg |
| | | tacggctccaaggcctacgtgaagcaccccgccgacatccccgactacttgaagctgtccttccccgag |
| | | ggcttcaagtgggagcgcgtgatgaacttcgaggacggcggcgtggtgaccgtgacccaggactcct |
| | | ccctgcaggacggcgagttcatctacaaggtgaagctgcgcggcaccaacttcccctccgacggccc |
| | | cgtaatgcagaagaagaccatgggctgggaggcctcctccgagcggatgtaccccgaggacggcgc |
| | | cctgaagggcgagatcaagcagaggctgaagctgaaggacggcggccactacgacgctgaggtcaa |
| | | gaccacctacaaggccaagaagcccgtgcagctgcccggcgcctacaacgtcaacatcaagttggac |
| | | atcacctcccacaacgaggactacaccatcgtggaacagtacgaacgcgccgagggccgccactcca |
| | | ccggcggcatggacgagctgtacaagtaa |
| 55 | Gosuranemab | KYGMS |
| | HCDR1 | |
| 56 | Gosuranemab | ISSSGSRTYYPDSVKG |
| | HCDR2 | |
| 57 | Gosuranemab | WDGAMDY |
| | HCDR3 | |
| 58 | Gosuranemab | KSSQSIVHSNGNTYLE |
| | LCDR1 | |
| 59 | Gosuranemab | KVSNRF |
| | LCDR2 | |
| 60 | Gosuranemab | FQGSLVPWA |
| | LCDR3 | |
| 61 | Gosuranemab | EVHLVESGGALVKPGGSLRLSCAASGFSFSKYGMSWVRQAPGK |
| | Heavy Chain (VH) | GLEWVATISSSGSRTYYPDSVKGRFTISRDNAKNTLYLQMNSLRA |
| | | EDTAMYYCSISWDGAMDYWGQGTTVTVSS |
| 62 | Gosuranemab | DVVMTQSPLSLPVTLGQPASISCKSSQSIVHSNGNTYLEWYLQKP |
| | Light Chain (VL) | GQSPQLLVYKVSNRFSGVPDRFSGSGSGTDFTLKISRVEAEDVGT |
| | | YYCFQGSLVPWAFGGGTKVEIK |
| 63 | Gosuranemab | gaagtgcatctggtggaaagcggcggcgcgctggtgaaaccgggcggcagcctgcgcctgagctgc |
| | Heavy Chain (VH) | gcggcgagcggctttagctttagcaaatatggcatgagctgggtgcgccaggcgccgggcaaaggcc |
| | | tggaatgggtggcgaccattagcagcagcggcagccgcacctattatccggatagcgtgaaaggccg |
| | | ctttaccattagccgcgataacgcgaaaaacaccctgtatctgcagatgaacagcctgcgcgcggaag |
| | | ataccgcgatgtattattgcagcattagctgggatggcgcgatggattattggggccagggcaccaccg |
| | | tgaccgtgagcagc |
| 64 | Gosuranemab | gatgtggtgatgacccagagcccgctgagcctgccggtgaccctgggccagccggcgagcattagct |
| | Light Chain (VL) | gcaaaagcagccagagcattgtgcatagcaacggcaacacctatctggaatggtatctgcagaaaccg |
| | | ggccagagcccgcagctgctggtgtataaagtgagcaaccgctttagcggcgtgccggatcgctttag |
| | | cggcagcggcagcggcaccgattttaccctgaaaattagccgcgtggaagcggaagatgtgggcacc |
| | | tattattgctttcagggcagcctggtgccgtgggcgtttggcggcggcaccaaagtggaaattaaa |
| 65 | Gosuranemab | EVHLVESGGALVKPGGSLRLSCAASGFSFSKYGMSWVRQAPGK |
| | H2L scFv | GLEWVATISSSGSRTYYPDSVKGRFTISRDNAKNTLYLQMNSLRA |
| | | EDTAMYYCSISWDGAMDYWGQGTTVTVSSGGGGSGGGGSGGG |
| | | GSGGGGSDVVMTQSPLSLPVTLGQPASISCKSSQSIVHSNGNTYL |
| | | EWYLQKPGQSPQLLVYKVSNRFSGVPDRFSGSGSGTDFTLKISRV |
| | | EAEDVGTYYCFQGSLVPWAFGGGTKVEIK |
| 66 | Gosuranemab | DVVMTQSPLSLPVTLGQPASISCKSSQSIVHSNGNTYLEWYLQKP |
| | L2H scFv | GQSPQLLVYKVSNRFSGVPDRFSGSGSGTDFTLKISRVEAEDVGT |
| | | YYCFQGSLVPWAFGGGTKVEIKGGGGSGGGGSGGGGSGGGGSE |
| | | VHLVESGGALVKPGGSLRLSCAASGFSFSKYGMSWVRQAPGKG |
| | | LEWVATISSSGSRTYYPDSVKGRFTISRDNAKNTLYLQMNSLRAE |
| | | DTAMYYCSISWDGAMDYWGQGTTVTVSS |
| 67 | Gosuranemab | gaagtgcatctggtggaaagcggcggcgcgctggtgaaaccgggcggcagcctgcgcctgagctgc |
| | H2L scFv | gcggcgagcggctttagctttagcaaatatggcatgagctgggtgcgccaggcgccgggcaaaggcc |
| | | tggaatgggtggcgaccattagcagcagcggcagccgcacctattatccggatagcgtgaaaggccg |
| | | ctttaccattagccgcgataacgcgaaaaacaccctgtatctgcagatgaacagcctgcgcgcggaag |
| | | ataccgcgatgtattattgcagcattagctgggatggcgcgatggattattggggccagggcaccaccg |
| | | tgaccgtgagcagcggcggaggtggaagcggaggcggaggatcaggtggcggtggatctggcggt |
| | | ggcggatctgatgtggtgatgacccagagcccgctgagcctgccggtgaccctgggccagccggcg |
| | | agcattagctgcaaaagcagccagagcattgtgcatagcaacggcaacacctatctggaatggtatctg |
| | | cagaaaccgggccagagcccgcagctgctggtgtataaagtgagcaaccgctttagcggcgtgccgg |
| | | atcgctttagcggcagcggcagcggcaccgattttaccctgaaaattagccgcgtggaagcggaagat |
| | | gtgggcacctattattgctttcagggcagcctggtgccgtgggcgtttggcggcggcaccaaagtggaa |
| | | attaaa |
| 68 | Gosuranemab | gatgtggtgatgacccagagcccgctgagcctgccggtgaccctgggccagccggcgagcattagct |
| | L2H scFv | gcaaaagcagccagagcattgtgcatagcaacggcaacacctatctggaatggtatctgcagaaaccg |
| | | ggccagagcccgcagctgctggtgtataaagtgagcaaccgctttagcggcgtgccggatcgctttag |
| | | cggcagcggcagcggcaccgattttaccctgaaaattagccgcgtggaagcggaagatgtgggcacc |
| | | tattattgctttcagggcagcctggtgccgtgggcgtttggcggcggcaccaaagtggaaattaaaggc |
| | | ggaggtggaagcggaggcggaggatcaggtggcggtggatctggcggtggcggatctgaagtgcat |
| | | ctggtggaaagcggcggcgcgctggtgaaaccgggcggcagcctgcgcctgagctgcgcggcgag |
| | | cggctttagctttagcaaatatggcatgagctgggtgcgccaggcgccgggcaaaggcctggaatggg |
| | | tggcgaccattagcagcagcggcagccgcacctattatccggatagcgtgaaaggccgctttaccatta |
| | | gccgcgataacgcgaaaaacaccctgtatctgcagatgaacagcctgcgcgcggaagataccgcgat |
| | | gtattattgcagcattagctgggatggcgcgatggattattggggccagggcaccaccgtgaccgtgag |
| | | cagc |
| 69 | Gosuranemab | MALPVTALLLPLALLLHAARPGSEVHLVESGGALVKPGGSLRLS |
| | H2L CAR | CAASGFSFSKYGMSWVRQAPGKGLEWVATISSSGSRTYYPDSVK |
| | | GRFTISRDNAKNTLYLQMNSLRAEDTAMYYCSISWDGAMDYWG |
| | | QGTTVTVSSGGGGSGGGGSGGGGSGGGGSDVVMTQSPLSLPVTL |
| | | GQPASISCKSSQSIVHSNGNTYLEWYLQKPGQSPQLLVYKVSNRF |
| | | SGVPDRFSGSGSGTDFTLKISRVEAEDVGTYYCFQGSLVPWAFGG |
| | | GTKVEIKSGTTTPAPRPPTPAPTIASQPLSLRPEACRPAAGGAVHT |
| | | RGLDFACDFWVLVVVGGVLACYSLLVTVAFIIFWVRSKRSTSGS |
| | | GATNFSLLKQAGDVEENPGPASMVSKGEEDNMAIIKEFMRFKVH |
| | | MEGSVNGHEFEIEGEGEGRPYEGTQTAKLKVTKGGPLPFAWDILS |
| | | PQFMYGSKAYVKHPADIPDYLKLSFPEGFKWERVMNFEDGGVV |
| | | TVTQDSSLQDGEFIYKVKLRGTNFPSDGPVMQKKTMGWEASSER |
| | | MYPEDGALKGEIKQRLKLKDGGHYDAEVKTTYKAKKPVQLPGA |
| | | YNVNIKLDITSHNEDYTIVEQYERAEGRHSTGGMDELYK |
| 70 | Gosuranemab | MALPVTALLLPLALLLHAARPGSDVVMTQSPLSLPVTLGQPASIS |
| | L2H CAR | CKSSQSIVHSNGNTYLEWYLQKPGQSPQLLVYKVSNRFSGVPDR |
| | | FSGSGSGTDFTLKISRVEAEDVGTYYCFQGSLVPWAFGGGTKVEI |
| | | KGGGGSGGGGSGGGGSGGGGSEVHLVESGGALVKPGGSLRLSC |
| | | AASGFSFSKYGMSWVRQAPGKGLEWVATISSSGSRTYYPDSVKG |
| | | RFTISRDNAKNTLYLQMNSLRAEDTAMYYCSISWDGAMDYWGQ |
| | | GTTVTVSSSGTTTPAPRPPTPAPTIASQPLSLRPEACRPAAGGAVH |
| | | TRGLDFACDFWVLVVVGGVLACYSLLVTVAFIIFWVRSKRSTSG |
| | | SGATNFSLLKQAGDVEENPGPASMVSKGEEDNMAIIKEFMRFKV |
| | | HMEGSVNGHEFEIEGEGEGRPYEGTQTAKLKVTKGGPLPFAWDI |
| | | LSPQFMYGSKAYVKHPADIPDYLKLSFPEGFKWERVMNFEDGGV |
| | | VTVTQDSSLQDGEFIYKVKLRGTNFPSDGPVMQKKTMGWEASSE |
| | | RMYPEDGALKGEIKQRLKLKDGGHYDAEVKTTYKAKKPVQLPG |
| | | AYNVNIKLDITSHNEDYTIVEQYERAEGRHSTGGMDELYK |
| 71 | Gosuranemab | atggccctgcctgtgacagccctgctgctgcctctggctctgctgctgcatgccgctagacccggatcc |
| | H2L CAR | gaagtgcatctggtggaaagcggcggcgcgctggtgaaaccgggcggcagcctgcgcctgagctgc |
| | | gcggcgagcggctttagctttagcaaatatggcatgagctgggtgcgccaggcgccgggcaaaggcc |
| | | tggaatgggtggcgaccattagcagcagcggcagccgcacctattatccggatagcgtgaaaggccg |
| | | ctttaccattagccgcgataacgcgaaaaacaccctgtatctgcagatgaacagcctgcgcgcggaag |
| | | ataccgcgatgtattattgcagcattagctgggatggcgcgatggattattggggccagggcaccaccg |
| | | tgaccgtgagcagcggcggaggtggaagcggaggcggaggatcaggtggcggtggatctggcggt |
| | | ggcggatctgatgtggtgatgacccagagcccgctgagcctgccggtgaccctgggccagccggcg |
| | | agcattagctgcaaaagcagccagagcattgtgcatagcaacggcaacacctatctggaatggtatctg |
| | | cagaaaccgggccagagcccgcagctgctggtgtataaagtgagcaaccgctttagcggcgtgccgg |
| | | atcgctttagcggcagcggcagcggcaccgattttaccctgaaaattagccgcgtggaagcggaagat |
| | | gtgggcacctattattgctttcagggcagcctggtgccgtgggcgtttggcggcggcaccaaagtggaa |
| | | attaaaccggaaccacgacgccagcgccgcgaccaccaacaccggcgcccaccatcgcgtcgcagc |
| | | ccctgtccctgcgcccagaggcgtgccggccagcggcggggggcgcagtgcacacgagggggctg |
| | | gacttcgcctgtgatttttgggtgctggtggtggttggtggagtcctggcttgctatagcttgctagtaaca |
| | | gtggcctttattattttctgggtgaggagtaagaggagcactagtggaagcggagctactaacttcagcct |
| | | gctgaagcaggctggagacgtggaggagaaccctggacctgctagcatggtgagcaagggcgagg |
| | | aggataacatggccatcatcaaggagttcatgcgcttcaaggtgcacatggagggctccgtgaacggc |
| | | cacgagttcgagatcgagggcgagggcgagggccgcccctacgagggcacccagaccgccaagct |
| | | gaaggtgaccaagggtggccccctgcccttcgcctgggacatcctgtcccctcagttcatgtacggctc |
| | | caaggcctacgtgaagcaccccgccgacatccccgactacttgaagctgtccttccccgagggcttcaa |
| | | gtgggagcgcgtgatgaacttcgaggacggcggcgtggtgaccgtgacccaggactcctccctgcag |
| | | gacggcgagttcatctacaaggtgaagctgcgcggcaccaacttcccctccgacggccccgtaatgca |
| | | gaagaagaccatgggctgggaggcctcctccgagcggatgtaccccgaggacggcgccctgaagg |
| | | gcgagatcaagcagaggctgaagctgaaggacggcggccactacgacgctgaggtcaagaccacct |
| | | acaaggccaagaagcccgtgcagctgcccggcgcctacaacgtcaacatcaagttggacatcacctc |
| | | ccacaacgaggactacaccatcgtggaacagtacgaacgcgccgagggccgccactccaccggcg |
| | | gcatggacgagctgtacaagtaa |
| 72 | Gosuranemab | atggccctgcctgtgacagccctgctgctgcctctggctctgctgctgcatgccgctagacccggatcc |
| | L2H CAR | gatgtggtgatgacccagagcccgctgagcctgccggtgaccctgggccagccggcgagcattagct |
| | | gcaaaagcagccagagcattgtgcatagcaacggcaacacctatctggaatggtatctgcagaaaccg |
| | | ggccagagcccgcagctgctggtgtataaagtgagcaaccgctttagcggcgtgccggatcgctttag |
| | | cggcagcggcagcggcaccgattttaccctgaaaattagccgcgtggaagcggaagatgtgggcacc |
| | | tattattgctttcagggcagcctggtgccgtgggcgtttggcggcggcaccaaagtggaaattaaaggc |
| | | ggaggtggaagcggaggcggaggatcaggtggcggtggatctggcggtggcggatctgaagtgcat |
| | | ctggtggaaagcggcggcgcgctggtgaaaccgggcggcagcctgcgcctgagctgcgcggcgag |
| | | cggctttagctttagcaaatatggcatgagctgggtgcgccaggcgccgggcaaaggcctggaatggg |
| | | tggcgaccattagcagcagcggcagccgcacctattatccggatagcgtgaaaggccgctttaccatta |
| | | gccgcgataacgcgaaaaacaccctgtatctgcagatgaacagcctgcgcgcggaagataccgcgat |
| | | gtattattgcagcattagctgggatggcgcgatggattattggggccagggcaccaccgtgaccgtgag |
| | | cagcccggaaccacgacgccagcgccgcgaccaccaacaccggcgcccaccatcgcgtcgcagcc |
| | | cctgtccctgcgcccagaggcgtgccggccagcggcggggggcgcagtgcacacgagggggctg |
| | | gacttcgcctgtgatttttgggtgctggtggtggttggtggagtcctggcttgctatagcttgctagtaac |
| | | agtggcctttattattttctgggtgaggagtaagaggagcactagtggaagcggagctactaacttcagcc |
| | | tgctgaagcaggctggagacgtggaggagaaccctggacctgctagcatggtgagcaagggcgagg |
| | | aggataacatggccatcatcaaggagttcatgcgcttcaaggtgcacatggagggctccgtgaacggc |
| | | cacgagttcgagatcgagggcgagggcgagggccgcccctacgagggcacccagaccgccaagct |
| | | gaaggtgaccaagggtggccccctgcccttcgcctgggacatcctgtcccctcagttcatgtacggctc |
| | | caaggcctacgtgaagcaccccgccgacatccccgactacttgaagctgtccttccccgagggcttcaa |
| | | gtgggagcgcgtgatgaacttcgaggacggcggcgtggtgaccgtgacccaggactcctccctgcag |
| | | gacggcgagttcatctacaaggtgaagctgcgcggcaccaacttcccctccgacggccccgtaatgca |
| | | gaagaagaccatgggctgggaggcctcctccgagcggatgtaccccgaggacggcgccctgaagg |
| | | gcgagatcaagcagaggctgaagctgaaggacggcggccactacgacgctgaggtcaagaccacct |
| | | acaaggccaagaagcccgtgcagctgcccggcgcctacaacgtcaacatcaagttggacatcacctc |
| | | ccacaacgaggactacaccatcgtggaacagtacgaacgcgccgagggccgccactccaccggcg |
| | | gcatggacgagctgtacaagtaa |
| 73 | Aducanumab H2L | MALPVTALLLPLALLLHAARPGSQVQLVESGGGVVQPGRSLRLS |
| | CD3z CAR | CAASGFAFSSYGMHWVRQAPGKGLEWVAVIWFDGTKKYYTDS |
| | | VKGRFTISRDNSKNTLYLQMNTLRAEDTAVYYCARDRGIGARRG |
| | | PYYMDVWGKGTTVTVSSGGGGSGGGGSGGGGSGGGGSDIQMT |
| | | QSPSSLSASVGDRVTITCRASQSISSYLNWYQQKPGKAPKLLIYAA |
| | | SSLQSGVPSRFSGSGSGTDFTLTISSLQPEDFATYYCQQSYSTPLTF |
| | | GGGTKVEIKRTVSGTTTPAPRPPTPAPTIASQPLSLRPEACRPAAG |
| | | GAVHTRGLDFACDFWVLVVVGGVLACYSLLVTVAFIIFWVRVKF |
| | | SRSADAPAYKQGQNQLYNELNLGRREEYDVLDKRRGRDPEMGG |
| | | KPRRKNPQEGLYNELQKDKMAEAYSEIGMKGERRRGKGHDGLY |
| | | QGLSTATKDTYDALHMQALPPRTSGSGATNFSLLKQAGDVEENP |
| | | GPASMVSKGEEDNMAIIKEFMRFKVHMEGSVNGHEFEIEGEGEG |
| | | RPYEGTQTAKLKVTKGGPLPFAWDILSPQFMYGSKAYVKHPADI |
| | | PDYLKLSFPEGFKWERVMNFEDGGVVTVTQDSSLQDGEFIYKVK |
| | | LRGTNFPSDGPVMQKKTMGWEASSERMYPEDGALKGEIKQRLK |
| | | LKDGGHYDAEVKTTYKAKKPVQLPGAYNVNIKLDITSHNEDYTI |
| | | VEQYERAEGRHSTGGMDELYK |
| 74 | Aducanumab L2H | MALPVTALLLPLALLLHAARPGSDIQMTQSPSSLSASVGDRVTIT |
| | CD3z CAR | CRASQSISSYLNWYQQKPGKAPKLLIYAASSLQSGVPSRFSGSGS |
| | | GTDFTLTISSLQPEDFATYYCQQSYSTPLTFGGGTKVEIKRTVGGG |
| | | GSGGGGSGGGGSGGGGSQVQLVESGGGVVQPGRSLRLSCAASG |
| | | FAFSSYGMHWVRQAPGKGLEWVAVIWFDGTKKYYTDSVKGRF |
| | | TISRDNSKNTLYLQMNTLRAEDTAVYYCARDRGIGARRGPYYM |
| | | DVWGKGTTVTVSSSGTTTPAPRPPTPAPTIASQPLSLRPEACRPAA |
| | | GGAVHTRGLDFACDFWVLVVVGGVLACYSLLVTVAFIIFWVRV |
| | | KFSRSADAPAYKQGQNQLYNELNLGRREEYDVLDKRRGRDPEM |
| | | GGKPRRKNPQEGLYNELQKDKMAEAYSEIGMKGERRRGKGHDG |
| | | LYQGLSTATKDTYDALHMQALPPRTSGSGATNFSLLKQAGDVEE |
| | | NPGPASMVSKGEEDNMAIIKEFMRFKVHMEGSVNGHEFEIEGEG |
| | | EGRPYEGTQTAKLKVTKGGPLPFAWDILSPQFMYGSKAYVKHPA |
| | | DIPDYLKLSFPEGFKWERVMNFEDGGVVTVTQDSSLQDGEFIYK |
| | | VKLRGTNFPSDGPVMQKKTMGWEASSERMYPEDGALKGEIKQR |
| | | LKLKDGGHYDAEVKTTYKAKKPVQLPGAYNVNIKLDITSHNED |
| | | YTIVEQYERAEGRHSTGGMDELYK |
| 75 | Aducanumab H2L | atggccctgcctgtgacagccctgctgctgcctctggctctgctgctgcatgccgctagacccggatccc |
| | CD3z CAR | aggtgcagctggtggaaagcggcggcggcgtggtgcagccgggccgcagcctgcgcctgagctgc |
| | | gcggcgagcggctttgcgtttagcagctatggcatgcattgggtgcgccaggcgccgggcaaaggcc |
| | | tggaatgggtggcggtgatttggtttgatggcaccaaaaaatattataccgatagcgtgaaaggccgcttt |
| | | accattagccgcgataacagcaaaaacaccctgtatctgcagatgaacaccctgcgcgcggaagatac |
| | | cgcggtgtattattgcgcgcgcgatcgcggcattggcgcgcgccgcggcccgtattatatggatgtgtg |
| | | gggcaaaggcaccaccgtgaccgtgagcagcggcggaggtggaagcggaggcggaggatcaggt |
| | | ggcggtggatctggcggtggcggatctgatattcagatgacccagagcccgagcagcctgagcgcga |
| | | gcgtgggcgatcgcgtgaccattacctgccgcgcgagccagagcattagcagctatctgaactggtatc |
| | | agcagaaaccgggcaaagcgccgaaactgctgatttatgcggcgagcagcctgcagagcggcgtgc |
| | | cgagccgctttagcggcagcggcagcggcaccgattttaccctgaccattagcagcctgcagccggaa |
| | | gattttgcgacctattattgccagcagagctatagcaccccgctgacctttggcggcggcaccaaagtgg |
| | | aaattaaacgctccggaaccacgacgccagcgccgcgaccaccaacaccggcgcccaccatcgcgt |
| | | cgcagcccctgtccctgcgcccagaggcgtgccggccagcggcggggggcgcagtgcacacgag |
| | | ggggctggacttcgcctgtgatttttgggtgctggtggtggttggtggagtcctggcttgctatagcttgc |
| | | tagtaacagtggcctttattattttctgggtgagagtgaagttcagcaggagcgcagacgcccccgcgtac |
| | | aagcagggccagaaccagctctataacgagctcaatctaggacgaagagaggagtacgatgttttgga |
| | | caagagacgtggccgggaccctgagatggggggaaagccgagaaggaagaaccctcaggaaggc |
| | | ctgtacaatgaactgcagaaagataagatggcggaggcctacagtgagattgggatgaaaggcgagc |
| | | gccggaggggcaaggggcacgatggcctttaccagggtctcagtacagccaccaaggacacctacg |
| | | acgcccttcacatgcaggccctgccccctcgcactagtggaagcggagctactaacttcagcctgctga |
| | | agcaggctggagacgtggaggagaaccctggacctgctagcatggtgagcaagggcgaggaggat |
| | | aacatggccatcatcaaggagttcatgcgcttcaaggtgcacatggagggctccgtgaacggccacga |
| | | gttcgagatcgagggcgagggcgagggccgcccctacgagggcacccagaccgccaagctgaagg |
| | | tgaccaagggtggccccctgcccttcgcctgggacatcctgtcccctcagttcatgtacggctccaagg |
| | | cctacgtgaagcaccccgccgacatccccgactacttgaagctgtccttccccgagggcttcaagtggg |
| | | agcgcgtgatgaacttcgaggacggcggcgtggtgaccgtgacccaggactcctccctgcaggacgg |
| | | cgagttcatctacaaggtgaagctgcgcggcaccaacttcccctccgacggccccgtaatgcagaaga |
| | | agaccatgggctgggaggcctcctccgagcggatgtaccccgaggacggcgccctgaagggcgag |
| | | atcaagcagaggctgaagctgaaggacggcggccactacgacgctgaggtcaagaccacctacaag |
| | | gccaagaagcccgtgcagctgcccggcgcctacaacgtcaacatcaagttggacatcacctcccacaa |
| | | cgaggactacaccatcgtggaacagtacgaacgcgccgagggccgccactccaccggcggcatgga |
| | | cgagctgtacaagtaa |
| 76 | Aducanumab L2H | atggccctgcctgtgacagccctgctgctgcctctggctctgctgctgcatgccgctagacccggatcc |
| | CD3z CAR | gatatccagatgactcagagccccagcagcctgtctgcctctgtgggagacagagtgaccatcacctgt |
| | | agagccagccagagcatcagcagctacctgaactggtatcagcagaagcccggcaaggcccctaaa |
| | | ctgctgatctatgccgcctccagtctgcagagcggagtgccttctagattttccggcagcggctccggca |
| | | ccgatttcaccctgaccatatctagcctgcagcctgaggacttcgccacctactactgccagcagagcta |
| | | cagcacccctctgacttttggcggaggcaccaaggtggaaatcaagcggacagtgtggcggaggtgg |
| | | aagcggaggcggaggatcaggtggcggtggatctggcggtggcggatctcaggtgcagctggtgga |
| | | aagcggcggcggcgtggtgcagccgggccgcagcctgcgcctgagctgcgcggcgagcggctttg |
| | | cgtttagcagctatggcatgcattgggtgcgccaggcgccgggcaaaggcctggaatgggtggcggt |
| | | gatttggtttgatggcaccaaaaaatattataccgatagcgtgaaaggccgctttaccattagccgcgata |
| | | acagcaaaaacaccctgtatctgcagatgaacaccctgcgcgcggaagataccgcggtgtattattgcg |
| | | cgcgcgatcgcggcattggcgcgcgccgcggcccgtattatatggatgtgtggggcaaaggcaccac |
| | | cgtgaccgtgagcagcccggaaccacgacgccagcgccgcgaccaccaacaccggcgcccaccat |
| | | cgcgtcgcagcccctgtccctgcgcccagaggcgtgccggccagcggcggggggcgcagtgcaca |
| | | cgagggggctggacttcgcctgtgatttttgggtgctggtggtggttggtggagtcctggcttgctatagc |
| | | ttgctagtaacagtggcctttattattttctgggtgagagtgaagttcagcaggagcgcagacgcccccg |
| | | cgtacaagcagggccagaaccagctctataacgagctcaatctaggacgaagagaggagtacgatgtt |
| | | ttggacaagagacgtggccgggaccctgagatggggggaaagccgagaaggaagaaccctcagga |
| | | aggcctgtacaatgaactgcagaaagataagatggcggaggcctacagtgagattgggatgaaaggc |
| | | gagcgccggaggggcaaggggcacgatggcctttaccagggtctcagtacagccaccaaggacacc |
| | | tacgacgcccttcacatgcaggccctgccccctcgcctagtggaagcggagctactaacttcagcctgc |
| | | tgaagcaggctggagacgtggaggagaaccctggacctgctagcatggtgagcaagggcgaggag |
| | | gataacatggccatcatcaaggagttcatgcgcttcaaggtgcacatggagggctccgtgaacggcca |
| | | cgagttcgagatcgagggcgagggcgagggccgcccctacgagggcacccagaccgccaagctga |
| | | aggtgaccaagggtggccccctgcccttcgcctgggacatcctgtcccctcagttcatgtacggctcca |
| | | aggcctacgtgaagcaccccgccgacatccccgactacttgaagctgtccttccccgagggcttcaagt |
| | | gggagcgcgtgatgaacttcgaggacggcggcgtggtgaccgtgacccaggactcctccctgcagg |
| | | acggcgagttcatctacaaggtgaagctgcgcggcaccaacttcccctccgacggccccgtaatgcag |
| | | aagaagaccatgggctgggaggcctcctccgagcggatgtaccccgaggacggcgccctgaaggg |
| | | cgagatcaagcagaggctgaagctgaaggacggcggccactacgacgctgaggtcaagaccaccta |
| | | caaggccaagaagcccgtgcagctgcccggcgcctacaacgtcaacatcaagttggacatcacctcc |
| | | cacaacgaggactacaccatcgtggaacagtacgaacgcgccgagggccgccactccaccggcgg |
| | | catggacgagctgtacaagtaa |
| 77 | Crenezumab H2L | MALPVTALLLPLALLLHAARPGSEVQLVESGGGLVQPGGSLRLS |
| | CD3z CAR | CAASGFTFSSYGMSWVRQAPGKGLELVASINSNGGSTYYPDSVK |
| | | GRFTISRDNAKNSLYLQMNSLRAEDTAVYYCASGDYWGQGTTV |
| | | TVSSGGGGSGGGGSGGGGSGGGGSDIVMTQSPLSLPVTPGEPASI |
| | | SCRSSQSLVYSNGDTYLHWYLQKPGQSPQLLIYKVSNRFSGVPDR |
| | | FSGSGSGTDFTLKISRVEAEDVGVYYCSQSTHVPWTFGQGTKVEI |
| | | KSGTTTPAPRPPTPAPTIASQPLSLRPEACRPAAGGAVHTRGLDFA |
| | | CDFWVLVVVGGVLACYSLLVTVAFIIFWVRVKFSRSADAPAYKQ |
| | | GQNQLYNELNLGRREEYDVLDKRRGRDPEMGGKPRRKNPQEGL |
| | | YNELQKDKMAEAYSEIGMKGERRRGKGHDGLYQGLSTATKDTY |
| | | DALHMQALPPRTSGSGATNFSLLKQAGDVEENPGPASMVSKGEE |
| | | DNMAIIKEFMRFKVHMEGSVNGHEFEIEGEGEGRPYEGTQTAKL |
| | | KVTKGGPLPFAWDILSPQFMYGSKAYVKHPADIPDYLKLSFPEGF |
| | | KWERVMNFEDGGVVTVTQDSSLQDGEFIYKVKLRGTNFPSDGPV |
| | | MQKKTMGWEASSERMYPEDGALKGEIKQRLKLKDGGHYDAEV |
| | | KTTYKAKKPVQLPGAYNVNIKLDITSHNEDYTIVEQYERAEGRHS |
| | | TGGMDELYK |
| 78 | Crenezumab L2H | MALPVTALLLPLALLLHAARPGSDIVMTQSPLSLPVTPGEPASISC |
| | CD3z CAR | RSSQSLVYSNGDTYLHWYLQKPGQSPQLLIYKVSNRFSGVPDRES |
| | | GSGSGTDFTLKISRVEAEDVGVYYCSQSTHVPWTFGQGTKVEIK |
| | | GGGGSGGGGSGGGGSGGGGSEVQLVESGGGLVQPGGSLRLSCA |
| | | ASGFTFSSYGMSWVRQAPGKGLELVASINSNGGSTYYPDSVKGR |
| | | FTISRDNAKNSLYLQMNSLRAEDTAVYYCASGDYWGQGTTVTV |
| | | SSSGTTTPAPRPPTPAPTIASQPLSLRPEACRPAAGGAVHTRGLDF |
| | | ACDFWVLVVVGGVLACYSLLVTVAFIIFWVRVKFSRSADAPAYK |
| | | QGQNQLYNELNLGRREEYDVLDKRRGRDPEMGGKPRRKNPQEG |
| | | LYNELQKDKMAEAYSEIGMKGERRRGKGHDGLYQGLSTATKDT |
| | | YDALHMQALPPRTSGSGATNFSLLKQAGDVEENPGPASMVSKGE |
| | | EDNMAIIKEFMRFKVHMEGSVNGHEFEIEGEGEGRPYEGTQTAK |
| | | LKVTKGGPLPFAWDILSPQFMYGSKAYVKHPADIPDYLKLSFPEG |
| | | FKWERVMNFEDGGVVTVTQDSSLQDGEFIYKVKLRGTNFPSDGP |
| | | VMQKKTMGWEASSERMYPEDGALKGEIKQRLKLKDGGHYDAE |
| | | VKTTYKAKKPVQLPGAYNVNIKLDITSHNEDYTIVEQYERAEGR |
| | | HSTGGMDELYK |
| 79 | Crenezumab H2L | atggccctgcctgtgacagccctgctgctgcctctggctctgctgctgcatgccgctagacccggatcc |
| | CD3z CAR | gaggtgcagctggttgaatctggcggaggactggttcagcctggcggatctctgagactgtcttgtgcc |
| | | gccagcggcttcacctttagcagctacggcatgagctgggtccgacaggctcctggcaaaggactgga |
| | | actggtggccagcatcaacagcaatggcggcagcacctactatcccgacagcgtgaagggcagattc |
| | | accatcagccgggacaacgccaagaacagcctgtacctgcagatgaactccctgagagccgaggac |
| | | accgccgtgtactattgtgccagcggcgattattggggccagggcacaacagtgacagtgtctagcgg |
| | | cggaggtggaagcggaggcggaggatcaggtggcggtggatctggcggtggcggatctgacattgt |
| | | gatgacacagagccctctgagcctgccagtgacacctggcgaacctgccagcatctcctgtagaagca |
| | | gccagagcctggtgtacagcaacggcgatacctacctgcactggtatctgcagaagcccggccagtct |
| | | cctcagctgctgatctacaaggtgtccaaccggttcagcggcgtgcccgatagattttctggcagcggct |
| | | ctggcaccgacttcaccctgaagatctccagagtggaagccgaggatgtgggcgtgtactactgcagc |
| | | cagtctacccacgtgccatggacatttggacagggcaccaaggtggaaatcaagtccggaccggaac |
| | | cacgacgccagcgccgcgaccaccaacaccggcgcccaccatcgcgtcgcagcccctgtccctgcg |
| | | cccagaggcgtgccggccagcggggggggcgcagtgcacacgagggggctggacttcgcctgtg |
| | | atttttgggtgctggtggtggttggtggagtcctggcttgctatagcttgctagtaacagtggcctttat |
| | | tattttctgggtgagagtgaagttcagcaggagcgcagacgcccccgcgtacaagcagggccagaaccagc |
| | | tctataacgagctcaatctaggacgaagagaggagtacgatgttttggacaagagacgtggccgggac |
| | | cctgagatggggggaaagccgagaaggaagaaccctcaggaaggcctgtacaatgaactgcagaaa |
| | | gataagatggcggaggcctacagtgagattgggatgaaaggcgagcgccggaggggcaaggggca |
| | | cgatggcctttaccagggtctcagtacagccaccaaggacacctacgacgcccttcacatgcaggccct |
| | | gccccctcgcctagtggaagcggagctactaacttcagcctgctgaagcaggctggagacgtggagg |
| | | agaaccctggacctgctagcatggtgagcaagggcgaggaggataacatggccatcatcaaggagtt |
| | | catgcgcttcaaggtgcacatggagggctccgtgaacggccacgagttcgagatcgagggcgaggg |
| | | cgagggccgcccctacgagggcacccagaccgccaagctgaaggtgaccaagggtggccccctgc |
| | | ccttcgcctgggacatcctgtcccctcagttcatgtacggctccaaggcctacgtgaagcaccccgccg |
| | | acatccccgactacttgaagctgtccttccccgagggcttcaagtgggagcgcgtgatgaacttcgagg |
| | | acggcggcgtggtgaccgtgacccaggactcctccctgcaggacggcgagttcatctacaaggtgaa |
| | | gctgcgcggcaccaacttcccctccgacggccccgtaatgcagaagaagaccatgggctgggaggc |
| | | ctcctccgagcggatgtaccccgaggacggcgccctgaagggcgagatcaagcagaggctgaagct |
| | | gaaggacggcggccactacgacgctgaggtcaagaccacctacaaggccaagaagcccgtgcagct |
| | | gcccggcgcctacaacgtcaacatcaagttggacatcacctcccacaacgaggactacaccatcgtgg |
| | | aacagtacgaacgcgccgagggccgccactccaccggcggcatggacgagctgtacaagtaa |
| 80 | Crenezumab L2H | atggccctgcctgtgacagccctgctgctgcctctggctctgctgctgcatgccgctagacccggatcc |
| | CD3z CAR | gacattgtgatgacacagagccctctgagcctgccagtgacacctggcgaacctgccagcatctcctgt |
| | | agaagcagccagagcctggtgtacagcaacggcgatacctacctgcactggtatctgcagaagcccg |
| | | gccagtctcctcagctgctgatctacaaggtgtccaaccggttcagcggcgtgcccgatagattttctgg |
| | | cagcggctctggcaccgacttcaccctgaagatctccagagtggaagccgaggatgtgggcgtgtact |
| | | actgcagccagtctacccacgtgccatggacatttggacagggcaccaaggtggaaatcaagtccgga |
| | | ggcggaggtggaagcggaggcggaggatcaggtggcggtggatctggcggtggcggatctgaggt |
| | | gcagctggttgaatctggcggaggactggttcagcctggcggatctctgagactgtcttgtgccgccag |
| | | cggcttcacctttagcagctacggcatgagctgggtccgacaggctcctggcaaaggactggaactgg |
| | | tggccagcatcaacagcaatggcggcagcacctactatcccgacagcgtgaagggcagattcaccatc |
| | | agccgggacaacgccaagaacagcctgtacctgcagatgaactccctgagagccgaggacaccgcc |
| | | gtgtactattgtgccagcggcgattattggggccagggcacaacagtgacagtgtctagcccggaacc |
| | | acgacgccagcgccgcgaccaccaacaccggcgcccaccatcgcgtcgcagcccctgtccctgcgc |
| | | ccagaggcgtgccggccagcggggggggcgcagtgcacacgagggggctggacttcgcctgtga |
| | | tttttgggtgctggtggtggttggtggagtcctggcttgctatagcttgctagtaacagtggcctttatt |
| | | attttctgggtgagagtgaagttcagcaggagcgcagacgcccccgcgtacaagcagggccagaaccagct |
| | | ctataacgagctcaatctaggacgaagagaggagtacgatgttttggacaagagacgtggccgggacc |
| | | ctgagatggggggaaagccgagaaggaagaaccctcaggaaggcctgtacaatgaactgcagaaag |
| | | ataagatggcggaggcctacagtgagattgggatgaaaggcgagcgccggaggggcaaggggcac |
| | | gatggcctttaccagggtctcagtacagccaccaaggacacctacgacgcccttcacatgcaggccct |
| | | gccccctcgcctagtggaagcggagctactaacttcagcctgctgaagcaggctggagacgtggagg |
| | | agaaccctggacctgctagcatggtgagcaagggcgaggaggataacatggccatcatcaaggagtt |
| | | catgcgcttcaaggtgcacatggagggctccgtgaacggccacgagttcgagatcgagggcgaggg |
| | | cgagggccgcccctacgagggcacccagaccgccaagctgaaggtgaccaagggtggccccctgc |
| | | ccttcgcctgggacatcctgtcccctcagttcatgtacggctccaaggcctacgtgaagcaccccgccg |
| | | acatccccgactacttgaagctgtccttccccgagggcttcaagtgggagcgcgtgatgaacttcgagg |
| | | acggcggcgtggtgaccgtgacccaggactcctccctgcaggacggcgagttcatctacaaggtgaa |
| | | gctgcgcggcaccaacttcccctccgacggccccgtaatgcagaagaagaccatgggctgggaggc |
| | | ctcctccgagcggatgtaccccgaggacggcgccctgaagggcgagatcaagcagaggctgaagct |
| | | gaaggacggcggccactacgacgctgaggtcaagaccacctacaaggccaagaagcccgtgcagct |
| | | gcccggcgcctacaacgtcaacatcaagttggacatcacctcccacaacgaggactacaccatcgtgg |
| | | aacagtacgaacgcgccgagggccgccactccaccggcggcatggacgagctgtacaagtaa |
| 81 | mAb 3D6 H2L | MALPVTALLLPLALLLHAARPGSEVKLVESGGGLVKPGASLKLS |
| | CAR | CAASGFTFSNYGMSWVRQNSDKRLEWVASIRSGGGRTYYSDNV |
| | | KGRFTISRENAKNTLYLQMSSLKSEDTALYYCVRYDHYSGSSDY |
| | | WGQGTTVTVSSSGGGGGSGGGGSGGGGSGGGGSYVVMTQTPLT |
| | | LSVTIGQPASISCKSSQSLLDSDGKTYLNWLLQRPGQSPKRLIYLV |
| | | SKLDSGVPDRFTGSGSGTDFTLKISRIEAEDLGLYYCWQGTHFPR |
| | | TFGGGTKLEIKSGTTTPAPRPPTPAPTIASQPLSLRPEACRPAAGGA |
| | | VHTRGLDFACDFWVLVVVGGVLACYSLLVTVAFIIFWVRVKFSR |
| | | SADAPAYKQGQNQLYNELNLGRREEYDVLDKRRGRDPEMGGKP |
| | | RRKNPQEGLYNELQKDKMAEAYSEIGMKGERRRGKGHDGLYQ |
| | | GLSTATKDTYDALHMQALPPRTSGSGATNFSLLKQAGDVEENPG |
| | | PASMVSKGEEDNMAIIKEFMRFKVHMEGSVNGHEFEIEGEGEGR |
| | | PYEGTQTAKLKVTKGGPLPFAWDILSPQFMYGSKAYVKHPADIP |
| | | DYLKLSFPEGFKWERVMNFEDGGVVTVTQDSSLQDGEFIYKVKL |
| | | RGTNFPSDGPVMQKKTMGWEASSERMYPEDGALKGEIKQRLKL |
| | | KDGGHYDAEVKTTYKAKKPVQLPGAYNVNIKLDITSHNEDYTIV |
| | | EQYERAEGRHSTGGMDELYK |
| 82 | mAb 3D6 L2H | MALPVTALLLPLALLLHAARPGSYVVMTQTPLTLSVTIGQPASIS |
| | CAR | CKSSQSLLDSDGKTYLNWLLQRPGQSPKRLIYLVSKLDSGVPDRF |
| | | TGSGSGTDFTLKISRIEAEDLGLYYCWQGTHFPRTFGGGTKLEIK |
| | | GGGGSGGGGSGGGGSGGGGSEVKLVESGGGLVKPGASLKLSCA |
| | | ASGFTFSNYGMSWVRQNSDKRLEWVASIRSGGGRTYYSDNVKG |
| | | RFTISRENAKNTLYLQMSSLKSEDTALYYCVRYDHYSGSSDYWG |
| | | QGTTVTVSSSGSGTTTPAPRPPTPAPTIASQPLSLRPEACRPAAGG |
| | | AVHTRGLDFACDFWVLVVVGGVLACYSLLVTVAFIIFWVRVKFS |
| | | RSADAPAYKQGQNQLYNELNLGRREEYDVLDKRRGRDPEMGG |
| | | KPRRKNPQEGLYNELQKDKMAEAYSEIGMKGERRRGKGHDGLY |
| | | QGLSTATKDTYDALHMQALPPRTSGSGATNFSLLKQAGDVEENP |
| | | GPASMVSKGEEDNMAIIKEFMRFKVHMEGSVNGHEFEIEGEGEG |
| | | RPYEGTQTAKLKVTKGGPLPFAWDILSPQFMYGSKAYVKHPADI |
| | | PDYLKLSFPEGFKWERVMNFEDGGVVTVTQDSSLQDGEFIYKVK |
| | | LRGTNFPSDGPVMQKKTMGWEASSERMYPEDGALKGEIKQRLK |
| | | LKDGGHYDAEVKTTYKAKKPVQLPGAYNVNIKLDITSHNEDYTI |
| | | VEQYERAEGRHSTGGMDELYK |
| 83 | mAb 3D6 H2L | atggccctgcctgtgacagccctgctgctgcctctggctctgctgctgcatgccgctagacccggatcc |
| | CAR | gaagtgaagctggttgaaagtggcggaggcctggttaagcctggcgcctctctgaaactgtcttgtgcc |
| | | gccagcggcttcaccttcagcaactacggcatgagctgggtccgacagaacagcgacaagcggcttg |
| | | agtgggtcgcctctattagatccggcggaggcagaacctactactccgacaacgtgaagggcagattc |
| | | accatcagcagagagaacgccaagaacaccctgtacctgcagatgagcagcctgaagtccgaggac |
| | | accgctctgtactattgcgtcagatacgaccactacagcggcagcagcgattattggggccagggcac |
| | | aaccgtgaccgtgtctagctccggaggcggaggtggaagcggaggcggaggatcaggtggcggtg |
| | | gatctggcggtggcggatcttacgtggtcatgacacagacccctctgacactgagcgtgaccattggac |
| | | agcctgccagcatcagctgcaagagcagtcagagcctgctggactccgacggcaagacctacctgaa |
| | | ttggctgctgcagaggcccggacagagccccaagagactgatctacctggtgtccaagctggacagc |
| | | ggcgtgcccgatagattcacaggctctggcagcggcaccgacttcaccctgaagatctctagaatcga |
| | | ggccgaggacctgggcctgtactactgttggcagggcacacacttccccagaacctttggcggcggaa |
| | | caaagctggaaatcaaaccggaaccacgacgccagcgccgcgaccaccaacaccggcgcccacca |
| | | tcgcgtcgcagcccctgtccctgcgcccagaggcgtgccggccagcggggggggcgcagtgcac |
| | | acgagggggctggacttcgcctgtgatttttgggtgctggtggtggttggtggagtcctggcttgctatag |
| | | cttgctagtaacagtggcctttattattttctgggtgagagtgaagttcagcaggagcgcagacgccccc |
| | | gcgtacaagcagggccagaaccagctctataacgagctcaatctaggacgaagagaggagtacgatg |
| | | ttttggacaagagacgtggccgggaccctgagatggggggaaagccgagaaggaagaaccctcagg |
| | | aaggcctgtacaatgaactgcagaaagataagatggcggaggcctacagtgagattgggatgaaagg |
| | | cgagcgccggaggggcaaggggcacgatggcctttaccagggtctcagtacagccaccaaggacac |
| | | ctacgacgcccttcacatgcaggccctgccccctcgcctagtggaagcggagctactaacttcagcctg |
| | | ctgaagcaggctggagacgtggaggagaaccctggacctgctagcatggtgagcaagggcgagga |
| | | ggataacatggccatcatcaaggagttcatgcgcttcaaggtgcacatggagggctccgtgaacggcc |
| | | acgagttcgagatcgagggcgagggcgagggccgcccctacgagggcacccagaccgccaagctg |
| | | aaggtgaccaagggtggccccctgcccttcgcctgggacatcctgtcccctcagttcatgtacggctcc |
| | | aaggcctacgtgaagcaccccgccgacatccccgactacttgaagctgtccttccccgagggcttcaa |
| | | gtgggagcgcgtgatgaacttcgaggacggcggcgtggtgaccgtgacccaggactcctccctgcag |
| | | gacggcgagttcatctacaaggtgaagctgcgcggcaccaacttcccctccgacggccccgtaatgca |
| | | gaagaagaccatgggctgggaggcctcctccgagcggatgtaccccgaggacggcgccctgaagg |
| | | gcgagatcaagcagaggctgaagctgaaggacggcggccactacgacgctgaggtcaagaccacct |
| | | acaaggccaagaagcccgtgcagctgcccggcgcctacaacgtcaacatcaagttggacatcacctc |
| | | ccacaacgaggactacaccatcgtggaacagtacgaacgcgccgagggccgccactccaccggcg |
| | | gcatggacgagctgtacaagtaa |
| 84 | mAb 3D6 L2H | atggccctgcctgtgacagccctgctgctgcctctggctctgctgctgcatgccgctagacccggatcct |
| | CAR | acgtggtcatgacacagacccctctgacactgagcgtgaccattggacagcctgccagcatcagctgc |
| | | aagagcagtcagagcctgctggactccgacggcaagacctacctgaattggctgctgcagaggcccg |
| | | gacagagccccaagagactgatctacctggtgtccaagctggacagcggcgtgcccgatagattcaca |
| | | ggctctggcagcggcaccgacttcaccctgaagatctctagaatcgaggccgaggacctgggcctgta |
| | | ctactgttggcagggcacacacttccccagaacctttggcggcggaacaaagctggaaatcaaaggcg |
| | | gaggtggaagcggaggcggaggatcaggtggcggtggatctggcggtggcggatctgaagtgaag |
| | | ctggttgaaagtggcggaggcctggttaagcctggcgcctctctgaaactgtcttgtgccgccagcggc |
| | | ttcaccttcagcaactacggcatgagctgggtccgacagaacagcgacaagcggcttgagtgggtcgc |
| | | ctctattagatccggcggaggcagaacctactactccgacaacgtgaagggcagattcaccatcagca |
| | | gagagaacgccaagaacaccctgtacctgcagatgagcagcctgaagtccgaggacaccgctctgta |
| | | ctattgcgtcagatacgaccactacagcggcagcagcgattattggggccagggcacaaccgtgaccg |
| | | tgtctagctccggaccggaaccacgacgccagcgccgcgaccaccaacaccggcgcccaccatcgc |
| | | gtcgcagcccctgtccctgcgcccagaggcgtgccggccagcggcggggggcgcagtgcacacga |
| | | gggggctggacttcgcctgtgatttttgggtgctggtggtggttggtggagtcctggcttgctatagcttg |
| | | ctagtaacagtggcctttattattttctgggtgagagtgaagttcagcaggagcgcagacgcccccgcgta |
| | | caagcagggccagaaccagctctataacgagctcaatctaggacgaagagaggagtacgatgttttgg |
| | | acaagagacgtggccgggaccctgagatggggggaaagccgagaaggaagaaccctcaggaagg |
| | | cctgtacaatgaactgcagaaagataagatggcggaggcctacagtgagattgggatgaaaggcgag |
| | | cgccggaggggcaaggggcacgatggcctttaccagggtctcagtacagccaccaaggacacctac |
| | | gacgcccttcacatgcaggccctgccccctcgcctagtggaagcggagctactaacttcagcctgctga |
| | | agcaggctggagacgtggaggagaaccctggacctgctagcatggtgagcaagggcgaggaggat |
| | | aacatggccatcatcaaggagttcatgcgcttcaaggtgcacatggagggctccgtgaacggccacga |
| | | gttcgagatcgagggcgagggcgagggccgcccctacgagggcacccagaccgccaagctgaagg |
| | | tgaccaagggtggccccctgcccttcgcctgggacatcctgtcccctcagttcatgtacggctccaagg |
| | | cctacgtgaagcaccccgccgacatccccgactacttgaagctgtccttccccgagggcttcaagtggg |
| | | agcgcgtgatgaacttcgaggacggcggcgtggtgaccgtgacccaggactcctccctgcaggacgg |
| | | cgagttcatctacaaggtgaagctgcgcggcaccaacttcccctccgacggccccgtaatgcagaaga |
| | | agaccatgggctgggaggcctcctccgagcggatgtaccccgaggacggcgccctgaagggcgag |
| | | atcaagcagaggctgaagctgaaggacggcggccactacgacgctgaggtcaagaccacctacaag |
| | | gccaagaagcccgtgcagctgcccggcgcctacaacgtcaacatcaagttggacatcacctcccacaa |
| | | cgaggactacaccatcgtggaacagtacgaacgcgccgagggccgccactccaccggcggcatgga |
| | | cgagctgtacaagtaa |
| 85 | Aducanumab H2L | MALPVTALLLPLALLLHAARPGSQVQLVESGGGVVQPGRSLRLS |
| | IL-10 CAR | CAASGFAFSSYGMHWVRQAPGKGLEWVAVIWFDGTKKYYTDS |
| | | VKGRFTISRDNSKNTLYLQMNTLRAEDTAVYYCARDRGIGARRG |
| | | PYYMDVWGKGTTVTVSSGGGGSGGGGSGGGGSGGGGSDIQMT |
| | | QSPSSLSASVGDRVTITCRASQSISSYLNWYQQKPGKAPKLLIYAA |
| | | SSLQSGVPSRFSGSGSGTDFTLTISSLQPEDFATYYCQQSYSTPLTF |
| | | GGGTKVEIKRTVSGTTTPAPRPPTPAPTIASQPLSLRPEACRPAAG |
| | | GAVHTRGLDFACDFWVLVVVGGVLACYSLLVTVAFIIFWVRSKR |
| | | SMVSKGEEDNMAIIKEFMRFKVHMEGSVNGHEFEIEGEGEGRPY |
| | | EGTQTAKLKVTKGGPLPFAWDILSPQFMYGSKAYVKHPADIPDY |
| | | LKLSFPEGFKWERVMNFEDGGVVTVTQDSSLQDGEFIYKVKLRG |
| | | TNFPSDGPVMQKKTMGWEASSERMYPEDGALKGEIKQRLKLKD |
| | | GGHYDAEVKTTYKAKKPVQLPGAYNVNIKLDITSHNEDYTIVEQ |
| | | YERAEGRHSTGGMDELYKASGSGATNFSLLKQAGDVEENPGPAS |
| | | MPGSALLCCLLLLTGMRISRGQYSREDNNCTHFPVGQSHMLLEL |
| | | RTAFSQVKTFFQTKDQLDNILLTDSLMQDFKGYLGCQALSEMIQF |
| | | YLVEVMPQAEKHGPEIKEHLNSLGEKLKTLRMRLRRCHRFLPCE |
| | | NKSKAVEQVKSDFNKLQDQGVYKAMNEFDIFINCIEAYMMIKM |
| | | KS |
| 86 | Aducanumab H2L | Atggccctgcctgtgacagccctgctgctgcctctggctctgctgctgcatgccgctagacccggatcc |
| | IL-10 CAR | caggtgcagctggtggaaagcggcggcggcgtggtgcagccgggccgcagcctgcgcctgagctg |
| | | cgcggcgagcggctttgcgtttagcagctatggcatgcattgggtgcgccaggcgccgggcaaaggc |
| | | ctggaatgggtggcggtgatttggtttgatggcaccaaaaaatattataccgatagcgtgaaaggccgct |
| | | ttaccattagccgcgataacagcaaaaacaccctgtatctgcagatgaacaccctgcgcgcggaagata |
| | | ccgcggtgtattattgcgcgcgcgatcgcggcattggcgcgcgccgcggcccgtattatatggatgtgt |
| | | ggggcaaaggcaccaccgtgaccgtgagcagcggcggaggtggaagcggaggcggaggatcagg |
| | | tggcggtggatctggcggtggcggatctgatattcagatgacccagagcccgagcagcctgagcgcg |
| | | agcgtgggcgatcgcgtgaccattacctgccgcgcgagccagagcattagcagctatctgaactggtat |
| | | cagcagaaaccgggcaaagcgccgaaactgctgatttatgcggcgagcagcctgcagagcggcgtg |
| | | ccgagccgctttagcggcagcggcagcggcaccgattttaccctgaccattagcagcctgcagccgga |
| | | agattttgcgacctattattgccagcagagctatagcaccccgctgacctttggcggcggcaccaaagtg |
| | | gaaattaaacgctccggaaccacgacgccagcgccgcgaccaccaacaccggcgcccaccatcgcg |
| | | tcgcagcccctgtccctgcgcccagaggcgtgccggccagcggcggggggcgcagtgcacacgag |
| | | ggggctggacttcgcctgtgatttttgggtgctggtggtggttggtggagtcctggcttgctatagcttgc |
| | | tagtaacagtggcctttattattttctgggtgaggagtaagaggagctcaaccgaagaacagttcaaatca |
| | | gtcttggagattcagaagactggaaaatacaagaaagttgaaaccgaactcctaaccctaggtggaagc |
| | | ggagctactaacttcagcctgctgaagcaggctggagacgtggaggagaaccctggacctgctagcat |
| | | ggtgagcaagggcgaggaggataacatggccatcatcaaggagttcatgcgcttcaaggtgcacatg |
| | | gagggctccgtgaacggccacgagttcgagatcgagggcgagggcgagggccgcccctacgaggg |
| | | cacccagaccgccaagctgaaggtgaccaagggtggccccctgcccttcgcctgggacatcctgtcc |
| | | cctcagttcatgtacggctccaaggcctacgtgaagcaccccgccgacatccccgactacttgaagctg |
| | | tccttccccgagggcttcaagtgggagcgcgtgatgaacttcgaggacggcggcgtggtgaccgtgac |
| | | ccaggactcctccctgcaggacggcgagttcatctacaaggtgaagctgcgcggcaccaacttcccct |
| | | ccgacggccccgtaatgcagaagaagaccatgggctgggaggcctcctccgagcggatgtaccccg |
| | | aggacggcgccctgaagggcgagatcaagcagaggctgaagctgaaggacggcggccactacgac |
| | | gctgaggtcaagaccacctacaaggccaagaagcccgtgcagctgcccggcgcctacaacgtcaac |
| | | atcaagttggacatcacctcccacaacgaggactacaccatcgtggaacagtacgaacgcgccgagg |
| | | gccgccactccaccggcggcatggacgagctgtacaagtaa |
| 87 | Aducanumab H2L | MALPVTALLLPLALLLHAARPGSQVQLVESGGGVVQPGRSLRLS |
| | FcV CAR | CAASGFAFSSYGMHWVRQAPGKGLEWVAVIWFDGTKKYYTDS |
| | | VKGRFTISRDNSKNTLYLQMNTLRAEDTAVYYCARDRGIGARRG |
| | | PYYMDVWGKGTTVTVSSGGGGSGGGGSGGGGSGGGGSDIQMT |
| | | QSPSSLSASVGDRVTITCRASQSISSYLNWYQQKPGKAPKLLIYAA |
| | | SSLQSGVPSRFSGSGSGTDFTLTISSLQPEDFATYYCQQSYSTPLTF |
| | | GGGTKVEIKRTVSGTTTPAPRPPTPAPTIASQPLSLRPEACRPAAG |
| | | GAVHTRGLDFACDFWVLVVVGGVLACYSLLVTVAFIIFWVRSKR |
| | | SSTEEQFKSVLEIQKTGKYKKVETELLTLGGSGATNFSLLKQAGD |
| | | VEENPGPASMVSKGEEDNMAIIKEFMRFKVHMEGSVNGHEFEIE |
| | | GEGEGRPYEGTQTAKLKVTKGGPLPFAWDILSPQFMYGSKAYVK |
| | | HPADIPDYLKLSFPEGFKWERVMNFEDGGVVTVTQDSSLQDGEFI |
| | | YKVKLRGTNFPSDGPVMQKKTMGWEASSERMYPEDGALKGEIK |
| | | QRLKLKDGGHYDAEVKTTYKAKKPVQLPGAYNVNIKLDITSHNE |
| | | DYTIVEQYERAEGRHSTGGMDELYK |
| 88 | Aducanumab H2L | Atggccctgcctgtgacagccctgctgctgcctctggctctgctgctgcatgccgctagacccggatcc |
| | Fcγ CAR | caggtgcagctggtggaaagcggcggcggcgtggtgcagccgggccgcagcctgcgcctgagctg |
| | | cgcggcgagcggctttgcgtttagcagctatggcatgcattgggtgcgccaggcgccgggcaaaggc |
| | | ctggaatgggtggcggtgatttggtttgatggcaccaaaaaatattataccgatagcgtgaaaggccgct |
| | | ttaccattagccgcgataacagcaaaaacaccctgtatctgcagatgaacaccctgcgcgcggaagata |
| | | ccgcggtgtattattgcgcgcgcgatcgcggcattggcgcgcgccgcggcccgtattatatggatgtgt |
| | | ggggcaaaggcaccaccgtgaccgtgagcagcggcggaggtggaagcggaggcggaggatcagg |
| | | tggcggtggatctggcggtggcggatctgatattcagatgacccagagcccgagcagcctgagcgcg |
| | | agcgtgggcgatcgcgtgaccattacctgccgcgcgagccagagcattagcagctatctgaactggtat |
| | | cagcagaaaccgggcaaagcgccgaaactgctgatttatgcggcgagcagcctgcagagcggcgtg |
| | | ccgagccgctttagcggcagcggcagcggcaccgattttaccctgaccattagcagcctgcagccgga |
| | | agattttgcgacctattattgccagcagagctatagcaccccgctgacctttggcggcggcaccaaagtg |
| | | gaaattaaacgctccggaaccacgacgccagcgccgcgaccaccaacaccggcgcccaccatcgcg |
| | | tcgcagcccctgtccctgcgcccagaggcgtgccggccagcggcggggggcgcagtgcacacgag |
| | | ggggctggacttcgcctgtgatttttgggtgctggtggtggttggtggagtcctggcttgctatagcttgc |
| | | tagtaacagtggcctttattattttctgggtgaggagtaagaggagctcaaccgaagaacagttcaaatca |
| | | gtcttggagattcagaagactggaaaatacaagaaagttgaaaccgaactcctaaccctaggtggaagc |
| | | ggagctactaacttcagcctgctgaagcaggctggagacgtggaggagaaccctggacctgctagcat |
| | | ggtgagcaagggcgaggaggataacatggccatcatcaaggagttcatgcgcttcaaggtgcacatg |
| | | gagggctccgtgaacggccacgagttcgagatcgagggcgagggcgagggccgcccctacgaggg |
| | | cacccagaccgccaagctgaaggtgaccaagggggccccctgcccttcgcctgggacatcctgtcc |
| | | cctcagttcatgtacggctccaaggcctacgtgaagcaccccgccgacatccccgactacttgaagctg |
| | | tccttccccgagggcttcaagtgggagcgcgtgatgaacttcgaggacggcggcgtggtgaccgtgac |
| | | ccaggactcctccctgcaggacggcgagttcatctacaaggtgaagctgcgcggcaccaacttcccct |
| | | ccgacggccccgtaatgcagaagaagaccatgggctgggaggcctcctccgagcggatgtaccccg |
| | | aggacggcgccctgaagggcgagatcaagcagaggctgaagctgaaggacggggccactacgac |
| | | gctgaggtcaagaccacctacaaggccaagaagcccgtgcagctgcccggcgcctacaacgtcaac |
| | | atcaagttggacatcacctcccacaacgaggactacaccatcgtggaacagtacgaacgcgccgagg |
| | | gccgccactccaccggcggcatggacgagctgtacaagtaa |
| 89 | Dual CAR | MALPVTALLLPLALLLHAARPGSEVHLVESGGALVKPGGSLRLS |
| | (Aducanumab | CAASGFSFSKYGMSWVRQAPGKGLEWVATISSSGSRTYYPDSVK |
| | H2L, | GRFTISRDNAKNTLYLQMNSLRAEDTAMYYCSISWDGAMDYWG |
| | Gosuranemab | QGTTVTVSSGGGGSGGGGSGGGGSGGGGSDVVMTQSPLSLPVTL |
| | H2L) | GQPASISCKSSQSIVHSNGNTYLEWYLQKPGQSPQLLVYKVSNRF |
| | | SGVPDRFSGSGSGTDFTLKISRVEAEDVGTYYCFQGSLVPWAFGG |
| | | GTKVEIKSGTTTPAPRPPTPAPTIASQPLSLRPEACRPAAGGAVHT |
| | | RGLDFACDGGGGSEQKLISEEDLSGFWVLVVVGGVLACYSLLVT |
| | | VAFIIFWVRSKRSASGSGATNFSLLKQAGDVEENPGPASMALPVT |
| | | ALLLPLALLLHAARPGSQVQLVESGGGVVQPGRSLRLSCAASGF |
| | | AFSSYGMHWVRQAPGKGLEWVAVIWFDGTKKYYTDSVKGRFTI |
| | | SRDNSKNTLYLQMNTLRAEDTAVYYCARDRGIGARRGPYYMDV |
| | | WGKGTTVTVSSGGGGSGGGGSGGGGSGGGGSDIQMTQSPSSLSA |
| | | SVGDRVTITCRASQSISSYLNWYQQKPGKAPKLLIYAASSLQSGV |
| | | PSRFSGSGSGTDFTLTISSLQPEDFATYYCQQSYSTPLTFGGGTKV |
| | | EIKRSGTTTPAPRPPTPAPTIASQPLSLRPEACRPAAGGAVHTRGL |
| | | DFACDGGGGSEQKLISEEDLSGFWVLVVVGGVLACYSLLVTVAF |
| | | IIFWVRSKRS |
| 90 | Dual CAR | atggccctgcctgtgacagccctgctgctgcctctggctctgctgctgcatgccgctagacccggatcc |
| | (Aducanumab | gaggtgcagctggttgaatctggcggaggactggttcagcctggcggatctctgagactgtcttgtgcc |
| | H2L, | gccagcggcttcacctttagcagctacggcatgagctgggtccgacaggctcctggcaaaggactgga |
| | Gosuranemab | actggtggccagcatcaacagcaatggcggcagcacctactatcccgacagcgtgaagggcagattc |
| | H2L) | accatcagccgggacaacgccaagaacagcctgtacctgcagatgaactccctgagagccgaggac |
| | | accgccgtgtactattgtgccagcggcgattattggggccagggcacaacagtgacagtgtctagcgg |
| | | cggaggtggaagcggaggcggaggatcaggtggcggtggatctggcggtggcggatctgatgtggt |
| | | gatgacccagagcccgctgagcctgccggtgaccctgggccagccggcgagcattagctgcaaaag |
| | | cagccagagcattgtgcatagcaacggcaacacctatctggaatggtatctgcagaaaccgggccaga |
| | | gcccgcagctgctggtgtataaagtgagcaaccgctttagcggcgtgccggatcgctttagcggcagc |
| | | ggcagcggcaccgattttaccctgaaaattagccgcgtggaagcggaagatgtgggcacctattattgc |
| | | tttcagggcagcctggtgccgtgggcgtttggcggcggcaccaaagtggaaattaaatccggaaccac |
| | | gacgccagcgccgcgaccaccaacaccggcgcccaccatcgcgtcgcagcccctgtccctgcgccc |
| | | agaggcgtgccggccagcggcggggggcgcagtgcacacgagggggctggacttcgcctgtgagg |
| | | ggggggggggagcgaacaaaaactcatctcagaagaggatctgtccggatttttgggtgctggtggtg |
| | | gttggtggagtcctggcttgctatagcttgctagtaacagtggcctttattattttctgggtgaggagtaa |
| | | gaggagcgctagcggaagcggagctactaacttcagcctgctgaagcaggctggagacgtggaggaga |
| | | accctggacctgctagcatggccctgcctgtgacagccctgctgctgcctctggctctgctgctgcatgc |
| | | cgctagacccggatcccaggtgcagctggtggaaagcggcggcggcgtggtgcagccgggccgca |
| | | gcctgcgcctgagctgcgcggcgagcggctttgcgtttagcagctatggcatgcattgggtgcgccag |
| | | gcgccgggcaaaggcctggaatgggggcggtgatttggtttgatggcaccaaaaaatattataccgat |
| | | agcgtgaaaggccgctttaccattagccgcgataacagcaaaaacaccctgtatctgcagatgaacacc |
| | | ctgcgcgcggaagataccgcggtgtattattgcgcgcgcgatcgcggcattggcgcgcgccgcggcc |
| | | cgtattatatggatgtgtggggcaaaggcaccaccgtgaccgtgagcagcggcggaggtggaagcgg |
| | | aggcggaggatcaggtggcggtggatctggcggtggcggatctgatattcagatgacccagagcccg |
| | | agcagcctgagcgcgagcgtgggcgatcgcgtgaccattacctgccgcgcgagccagagcattagc |
| | | agctatctgaactggtatcagcagaaaccgggcaaagcgccgaaactgctgatttatgcggcgagcag |
| | | cctgcagagcggcgtgccgagccgctttagcggcagcggcagcggcaccgattttaccctgaccatta |
| | | gcagcctgcagccggaagattttgcgacctattattgccagcagagctatagcaccccgctgacctttgg |
| | | cggcggcaccaaagtggaaattaaacgctccggaaccacgacgccagcgccgcgaccaccaacac |
| | | cggcgcccaccatcgcgtcgcagcccctgtccctgcgcccagaggcgtgccggccagcggcgggg |
| | | ggcgcagtgcacacgagggggctggacttcgcctgtgaggggggggggggggggggagctacccatacgat |
| | | gttccagattacgcttccggatttttgggtgctggtggtggttggtggagtcctggcttgctatagcttgct |
| | | agtaacagtggcctttattattttctgggtgaggagtaagaggagctaa |
| 91 | Aducanumab H2L | MALPVTALLLPLALLLHAARPGSQVQLVESGGGVVQPGRSLRLS |
| | Fused Reporter | CAASGFAFSSYGMHWVRQAPGKGLEWVAVIWFDGTKKYYTDS |
| | CAR | VKGRFTISRDNSKNTLYLQMNTLRAEDTAVYYCARDRGIGARRG |
| | | PYYMDVWGKGTTVTVSSGGGGSGGGGSGGGGSGGGGSDIQMT |
| | | QSPSSLSASVGDRVTITCRASQSISSYLNWYQQKPGKAPKLLIYAA |
| | | SSLQSGVPSRFSGSGSGTDFTLTISSLQPEDFATYYCQQSYSTPLTF |
| | | GGGTKVEIKRTVSGTTTPAPRPPTPAPTIASQPLSLRPEACRPAAG |
| | | GAVHTRGLDFACDFWVLVVVGGVLACYSLLVTVAFIIFWVRSKR |
| | | SMVSKGEEDNMAIIKEFMRFKVHMEGSVNGHEFEIEGEGEGRPY |
| | | EGTQTAKLKVTKGGPLPFAWDILSPQFMYGSKAYVKHPADIPDY |
| | | LKLSFPEGFKWERVMNFEDGGVVTVTQDSSLQDGEFIYKVKLRG |
| | | TNFPSDGPVMQKKTMGWEASSERMYPEDGALKGEIKQRLKLKD |
| | | GGHYDAEVKTTYKAKKPVQLPGAYNVNIKLDITSHNEDYTIVEQ |
| | | YERAEGRHSTGGMDELYK |
| 92 | Aducanumab H2L | atggccctgcctgtgacagccctgctgctgcctctggctctgctgctgcatgccgctagacccggatccc |
| | Fused Reporter | aggtgcagctggtggaaagcggcggcggcgtggtgcagccgggccgcagcctgcgcctgagctgc |
| | CAR | gcggcgagcggctttgcgtttagcagctatggcatgcattgggtgcgccaggcgccgggcaaaggcc |
| | | tggaatgggtggcggtgatttggtttgatggcaccaaaaaatattataccgatagcgtgaaaggccgcttt |
| | | accattagccgcgataacagcaaaaacaccctgtatctgcagatgaacaccctgcgcgcggaagatac |
| | | cgcggtgtattattgcgcgcgcgatcgcggcattggcgcgcgccgcggcccgtattatatggatgtgtg |
| | | gggcaaaggcaccaccgtgaccgtgagcagcggcggaggtggaagcggaggcggaggatcaggt |
| | | ggcggtggatctggcggtggcggatctgatattcagatgacccagagcccgagcagcctgagcgcga |
| | | gcgtgggcgatcgcgtgaccattacctgccgcgcgagccagagcattagcagctatctgaactggtatc |
| | | agcagaaaccgggcaaagcgccgaaactgctgatttatgcggcgagcagcctgcagagcggcgtgc |
| | | cgagccgctttagcggcagcggcagcggcaccgattttaccctgaccattagcagcctgcagccggaa |
| | | gattttgcgacctattattgccagcagagctatagcaccccgctgacctttggcggcggcaccaaagtgg |
| | | aaattaaacgctccggaaccacgacgccagcgccgcgaccaccaacaccggcgcccaccatcgcgt |
| | | cgcagcccctgtccctgcgcccagaggcgtgccggccagcggggggggcgcagtgcacacgag |
| | | ggggctggacttcgcctgtgatttttgggtgctggtggtggttggtggagtcctggcttgctatagcttgc |
| | | tagtaacagtggcctttattattttctgggtgaggagtaagaggagcatggtgagcaagggcgaggagga |
| | | taacatggccatcatcaaggagttcatgcgcttcaaggtgcacatggagggctccgtgaacggccacg |
| | | agttcgagatcgagggcgagggcgagggccgcccctacgagggcacccagaccgccaagctgaag |
| | | gtgaccaagggggccccctgcccttcgcctgggacatcctgtcccctcagttcatgtacggctccaag |
| | | gcctacgtgaagcaccccgccgacatccccgactacttgaagctgtccttccccgagggcttcaagtgg |
| | | gagcgcgtgatgaacttcgaggacggcggcgtggtgaccgtgacccaggactcctccctgcaggacg |
| | | gcgagttcatctacaaggtgaagctgcgcggcaccaacttcccctccgacggccccgtaatgcagaag |
| | | aagaccatgggctgggaggcctcctccgagcggatgtaccccgaggacggcgccctgaagggcga |
| | | gatcaagcagaggctgaagctgaaggacggcggccactacgacgctgaggtcaagaccacctacaa |
| | | ggccaagaagcccgtgcagctgcccggcgcctacaacgtcaacatcaagttggacatcacctcccac |
| | | aacgaggactacaccatcgtggaacagtacgaacgcgccgagggccgccactccaccggcggcatg |
| | | gacgagctgtacaagtaa |
| 93 | CD8 Leader | atggccctgcctgtgacagccctgctgctgcctctggctctgctgctgcatgccgctagaccc |
| 94 | Linker | ggcggaggtggaagcggaggcggaggatcaggtggcggtggatctggcggtggcggatct |
| 126 | CD8a/hinge | accacgacgccagcgccgcgaccaccaacaccggcgcccaccatcgcgtcgcagcccctgtccctg |
| | | cgcccagaggcgtgccggccagcggcggggggcgcagtgcacacgagggggctggacttcgcct |
| | | gtga |
| 95 | CD28/TM | tttttgggtgctggtggtggttggtggagtcctggcttgctatagcttgctagtaacagtggcctttatt |
| | | attttctgggtg |
| 96 | CD28 tail | aggagtaagaggagc |
| 97 | P2A | ggaagcggagctactaacttcagcctgctgaagcaggctggagacgtggaggagaaccctggacct |
| 98 | mCherry | atggtgagcaagggcgaggaggataacatggccatcatcaaggagttcatgcgcttcaaggtgcacat |
| | | ggagggctccgtgaacggccacgagttcgagatcgagggcgagggcgagggccgcccctacgagg |
| | | gcacccagaccgccaagctgaaggtgaccaagggtggccccctgcccttcgcctgggacatcctgtc |
| | | ccctcagttcatgtacggctccaaggcctacgtgaagcaccccgccgacatccccgactacttgaagct |
| | | gtccttccccgagggcttcaagtgggagcgcgtgatgaacttcgaggacggcggcgtggtgaccgtg |
| | | acccaggactcctccctgcaggacggcgagttcatctacaaggtgaagctgcgcggcaccaacttccc |
| | | ctccgacggccccgtaatgcagaagaagaccatgggctgggaggcctcctccgagcggatgtacccc |
| | | gaggacggcgccctgaagggcgagatcaagcagaggctgaagctgaaggacggcggccactacga |
| | | cgctgaggtcaagaccacctacaaggccaagaagcccgtgcagctgcccggcgcctacaacgtcaa |
| | | catcaagttggacatcacctcccacaacgaggactacaccatcgtggaacagtacgaacgcgccgag |
| | | ggccgccactccaccggcggcatggacgagctgtacaag |
| 99 | CD3z ICD | agagtgaagttcagcaggagcgcagacgcccccgcgtacaagcagggccagaaccagctctataac |
| | | gagctcaatctaggacgaagagaggagtacgatgttttggacaagagacgtggccgggaccctgaga |
| | | tggggggaaagccgagaaggaagaaccctcaggaaggcctgtacaatgaactgcagaaagataaga |
| | | tggcggaggcctacagtgagattgggatgaaaggcgagcgccggaggggcaaggggcacgatggc |
| | | ctttaccagggtctcagtacagccaccaaggacacctacgacgcccttcacatgcaggccctgccccct |
| | | cgc |
| 100 | CD8 Leader | MALPVTALLLPLALLLHAARP |
| 101 | Linker | GGGGSGGGGSGGGGSGGGGS |
| 102 | CD8a/hinge | TTTPAPRPPTPAPTIASQPLSLRPEACRPAAGGAVHTRGLDFACD |
| 103 | CD28/TM | WVLVVVGGVLACYSLLVTVAFIIFWV |
| 104 | CD28 tail | RSKRS |
| 105 | P2A | GSGATNFSLLKQAGDVEENPGP |
| 106 | mCherry | MVSKGEEDNMAIIKEFMRFKVHMEGSVNGHEFEIEGEGEGRPYE |
| | | GTQTAKLKVTKGGPLPFAWDILSPQFMYGSKAYVKHPADIPDYL |
| | | KLSFPEGFKWERVMNFEDGGVVTVTQDSSLQDGEFIYKVKLRGT |
| | | NFPSDGPVMQKKTMGWEASSERMYPEDGALKGEIKQRLKLKDG |
| | | GHYDAEVKTTYKAKKPVQLPGAYNVNIKLDITSHNEDYTIVEQY |
| | | ERAEGRHSTGGMDELY |
| 107 | CD3z ICD | RVKFSRSADAPAYKQGQNQLYNELNLGRREEYDVLDKRRGRDP |
| | | EMGGKPRRKNPQEGLYNELQKDKMAEAYSEIGMKGERRRGKG |
| | | HDGLYQGLSTATKDTYDALHMQALPPR |
| 108 | Mouse IL-10 | MPGSALLCCLLLLTGMRISRGQYSREDNNCTHFPVGQSHMLLEL |
| | | RTAFSQVKTFFQTKDQLDNILLTDSLMQDFKGYLGCQALSEMIQF |
| | | YLVEVMPQAEKHGPEIKEHLNSLGEKLKTLRMRLRRCHRFLPCE |
| | | NKSKAVEQVKSDFNKLQDQGVYKAMNEFDIFINCIEAYMMIKM |
| | | KS |
| 109 | Mouse IL-10 | atgcctggctcagcactgctatgctgcctgctcttactgactggcatgaggatcagcaggggccagtac |
| | | agccgggaagacaataactgcacccacttcccagtcggccagagccacatgctcctagagctgcgga |
| | | ctgccttcagccaggtgaagactttctttcaaacaaaggaccagctggacaacatactgctaaccgactc |
| | | cttaatgcaggactttaagggttacttgggttgccaagccttatcggaaatgatccagttttacctggtag |
| | | aagtgatgccccaggcagagaagcatggcccagaaatcaaggagcatttgaattccctgggtgagaag |
| | | ctgaagaccctcaggatgcggctgaggcgctgtcatcgatttctcccctgtgaaaataagagcaaggca |
| | | gtggagcaggtgaagagtgattttaataagctccaagaccaaggtgtctacaaggccatgaatgaatttg |
| | | acatcttcatcaactgcatagaagcatacatgatgatcaaaatgaaaagc |
| 110 | Fcγ ICD | STEEQFKSVLEIQKTGKYKKVETELLT |
| 111 | Fcγ ICD | tcaaccgaagaacagttcaaatcagtcttggagattcagaagactggaaaatacaagaaagttgaaacc |
| | | gaactcctaacc |
| 112 | BFP | atgagcgagctgattaaggagaacatgcacatgaagctgtacatggagggcaccgtggacaaccatca |
| | | cttcaagtgcacatccgagggcgaaggcaagccctacgagggcacccagaccatgagaatcaaggt |
| | | ggtcgagggcggccctctccccttcgccttcgacatcctggctactagcttcctctacggcagcaagac |
| | | cttcatcaaccacacccagggcatccccgacttcttcaagcagtccttccctgagggcttcacatgggag |
| | | agagtcaccacatacgaagacgggggcgtgctgaccgctacccaggacaccagcctccaggacggc |
| | | tgcctcatctacaacgtcaagatcagaggggtgaacttcacatccaacggccctgtgatgcagaagaaa |
| | | acactcggctgggaggccttcaccgagacgctgtaccccgctgacggcggcctggaaggcagaaac |
| | | gacatggccctgaagctcgtggggggagccatctgatcgcaaacgccaagaccacatatagatcca |
| | | agaaacccgctaagaacctcaagatgcctggcgtctactatgtggactacagactggaaagaatcaag |
| | | gaggccaacaacgagacctacgtcgagcagcacgaggtggcagtggccagatactgcgacctccct |
| | | agcaaactggggcacaagcttaattaa |
| 113 | BFP | MSELIKENMHMKLYMEGTVDNHHFKCTSEGEGKPYEGTQTMRI |
| | | KVVEGGPLPFAFDILATSFLYGSKTFINHTQGIPDFFKQSFPEGFT |
| | | WERVTTYEDGGVLTATQDTSLQDGCLIYNVKIRGVNFTSNGPVM |
| | | QKKTLGWEAFTETLYPADGGLEGRNDMALKLVGGSHLIANAKT |
| | | TYRSKKPAKNLKMPGVYYVDYRLERIKEANNETYVEQHEVAVA |
| | | RYCDLPSKLGHKLN |

### Transmembrane Domain

With respect to the transmembrane domain, a provided CAR can be designed to comprise a transmembrane domain that: a) connects the antigen binding domain of the CAR to an intracellular domain, and/or b) anchors the antigen binding domain in the membrane of a cell (e.g., an immune cell). In some embodiments, a transmembrane domain may be naturally associated with one or more of the domains in a CAR. In some instances, a transmembrane domain can be selected or modified by amino acid substitution to avoid binding of such domain(s) to the transmembrane domains of the same or different surface membrane proteins to minimize interactions with other members of the receptor complex.

In some embodiments, a transmembrane domain may be derived either from a natural or from a synthetic source. Where the source is natural, the domain may be derived from any membrane-bound or transmembrane protein. In some embodiments, transmembrane regions of particular use may be derived from (i.e. comprise at least the transmembrane region(s) of) the alpha, beta or zeta chain of the T-cell receptor, CD28, CD3 epsilon, CD45, CD4, CD5, CD8, CD9, CD16, CD22, CD33, CD37, CD64, CD80, CD86, CD134, CD137, CD154, Toll-like receptor 1 (TLR1), TLR2, TLR3, TLR4, TLR5, TLR6, TLR7, TLR8, and/or TLR9. In some embodiments, a transmembrane region may comprise one or more hinge regions. In some instances, any of a variety of human hinge regions can be employed as well (e.g., a CD28 or CD8 hinge region) including the human Ig (immunoglobulin) hinge region.

In some embodiments, the transmembrane domain may be synthetic, in which case it will comprise predominantly hydrophobic residues such as leucine and valine. In some embodiments, a triplet of phenylalanine, tryptophan and valine will be found at each end of a synthetic transmembrane domain.

In some embodiments, between the antigen binding domain and the transmembrane domain of a provided CAR, a spacer domain may be incorporated. As used herein, the term “spacer domain” generally means any oligo- or polypeptide that functions to link the transmembrane domain to either the antigen binding domain or to the intracellular domain in the polypeptide chain. In some embodiments, the spacer domain may comprise up to 300 amino acids, preferably 10 to 100 amino acids and most preferably 25 to 50 amino acids. In some embodiments, a short oligo- or polypeptide linker, preferably between 2 and 10 amino acids in length may form the linkage between the transmembrane domain and the intracellular domain of the CAR. An example of a linker includes a glycine-serine doublet.

### Intracellular Signaling Domain

CARs of the present disclosure may optionally include an intracellular signaling domain. In certain embodiments, however the intracellular signaling domain is absent from the CAR. The terms “intracellular signaling domain” and “intracellular domain” are used interchangeably herein. In some embodiments, the intracellular signaling domain of the CAR is responsible for activation of at least one of the effector functions of the cell in which the CAR is expressed (e.g., immune cell). In some embodiments, the intracellular signaling domain transduces the effector function signal and directs the cell (e.g., immune cell) to perform its specialized function, e.g., harming and/or destroying a target cell.

Examples of an intracellular domain for use in the invention include, but are not limited to, the cytoplasmic portion of a surface receptor, co-stimulatory molecule, and any molecule that acts in concert to initiate signal transduction in the cell, as well as any derivative or variant of these elements and any synthetic sequence that has the same functional capability.

Examples of the intracellular signaling domain include, without limitation, the ζ chain of the T cell receptor complex or any of its homologs, e.g., 1 chain, FcsRIγ and β chains, MB 1 (Iga) chain, B29 (Ig) chain, etc., human CD3 zeta chain, CD3 polypeptides (Δ, δ and ε), syk family tyrosine kinases (Syk, ZAP 70, etc.), src family tyrosine kinases (Lck, Fyn, Lyn, etc.), and other molecules involved in T cell transduction, such as CD2, CD5 and CD28. In one embodiment, the intracellular signaling domain may be human CD3 zeta chain, FcγRIII, FesRI, cytoplasmic tails of Fc receptors, an immunoreceptor tyrosine-based activation motif (ITAM) bearing cytoplasmic receptors, and combinations thereof.

In some embodiments, the intracellular signaling domain of the CAR includes any portion of one or more co-stimulatory molecules, such as at least one signaling domain from CD2, CD3, CD8, CD27, CD28, ICOS, 4-1BB, PD-1, any derivative or variant thereof, any synthetic sequence thereof that has the same functional capability, and any combination thereof.

Other examples of the intracellular domain include a fragment or domain from one or more molecules or receptors including, but not limited to, TCR, CD3 zeta, CD3 gamma, CD3 delta, CD3 epsilon, CD86, common FcR gamma, FcR beta (Fc Epsilon RIb), CD79a, CD79b, Fcgamma RIIa, DAP10, DAP12, T cell receptor (TCR), CD8, CD27, CD28, 4-1BB (CD137), OX9, OX40, CD30, CD40, PD-1, ICOS, a KIR family protein, lymphocyte function-associated antigen-1 (LFA-1), CD2, CD7, LIGHT, NKG2C, B7-H3, a ligand that specifically binds with CD83, CDS, ICAM-1, GITR, BAFFR, HVEM (LIGHTR), SLAMF7, NKp80 (KLRF1), CD127, CD160, CD19, CD4, CD8alpha, CD8beta, IL2R beta, IL2R gamma, IL7R alpha, ITGA4, VLA1, CD49a, ITGA4, IA4, CD49D, ITGA6, VLA-6, CD49f, ITGAD, CD11d, ITGAE, CD103, ITGAL, CD11a, LFA-1, ITGAM, CD11b, ITGAX, CD11c, ITGB1, CD29, ITGB2, CD18, LFA-1, ITGB7, TNFR2, TRANCE/RANKL, DNAM1 (CD226), SLAMF4 (CD244, 2B4), CD84, CD96 (Tactile), CEACAM1, CRT AM, Ly9 (CD229), CD160 (BY55), PSGL1, CD100 (SEMA4D), CD69, SLAMF6 (NTB-A, Ly108), SLAM (SLAMF1, CD150, IPO-3), BLAME (SLAMF8), SELPLG (CD162), LTBR, LAT, GADS, SLP-76, PAG/Cbp, NKp44, NKp30, NKp46, NKG2D, Toll-like receptor 1 (TLR1), TLR2, TLR3, TLR4, TLR5, TLR6, TLR7, TLR8, TLR9, other co-stimulatory molecules described herein, any derivative, variant, or fragment thereof, any synthetic sequence of a co-stimulatory molecule that has the same functional capability, and any combination thereof.

Additional examples of intracellular domains include, without limitation, intracellular signaling domains of several types of various other immune signaling receptors, including, but not limited to, first, second, and third generation cell signaling proteins including CD3, B7 family costimulatory, and Tumor Necrosis Factor Receptor (TNFR) superfamily receptors (see, e.g., Park and Brentjens, J. Clin. Oncol. (2015) 33(6): 651-653). Additionally, intracellular signaling domains may include signaling domains used by NK and NKT cells (see, e.g., Hermanson and Kaufman, Front. Immunol. (2015) 6: 195) such as signaling domains of NKp30 (B7-H6) (see, e.g., Zhang et al., J. Immunol. (2012) 189(5): 2290-2299), and DAP 12 (see, e.g., Topfer et al., J. Immunol. (2015) 194(7): 3201-3212), NKG2D, NKp44, NKp46, DAP10, and CD3z.

Intracellular signaling domains suitable for use in a subject CAR of the present disclosure include any desired signaling domain that provides a distinct and detectable signal (e.g., increased production of one or more cytokines by the cell; change in transcription of a target gene; change in activity of a protein; change in cell behavior, e.g., cell death; cellular proliferation; cellular differentiation; cell survival; modulation of cellular signaling responses; etc.) in response to activation of the CAR (i.e., activated by antigen and dimerizing agent). In some embodiments, the intracellular signaling domain includes at least one (e.g., one, two, three, four, five, six, etc.) ITAM motifs as described below. In some embodiments, the intracellular signaling domain includes DAP10/CD28 type signaling chains. In some embodiments, the intracellular signaling domain is not covalently attached to the membrane bound CAR, but is instead diffused in the cytoplasm.

Intracellular signaling domains suitable for use in a subject CAR of the present invention include immunoreceptor tyrosine-based activation motif (ITAM)-containing intracellular signaling polypeptides. In some embodiments, an ITAM motif is repeated twice in an intracellular signaling domain, where the first and second instances of the ITAM motif are separated from one another by 6 to 8 amino acids. In one embodiment, the intracellular signaling domain of a subject CAR comprises 3 ITAM motifs.

In some embodiments, intracellular signaling domains includes the signaling domains of human immunoglobulin receptors that contain immunoreceptor tyrosine based activation motifs (ITAMs) such as, but not limited to, FcgammaRI, FcgammaRIIA, FcgammaRIIC, FcgammaRIIIA, FcRL5 (see, e.g., Gillis et al., Front. Immunol. (2014) 5:254).

A suitable intracellular signaling domain can be an ITAM motif-containing portion that is derived from a polypeptide that contains an ITAM motif. For example, a suitable intracellular signaling domain can be an ITAM motif-containing domain from any ITAM motif-containing protein. Thus, a suitable intracellular signaling domain need not contain the entire sequence of the entire protein from which it is derived. Examples of suitable ITAM motif-containing polypeptides include, but are not limited to: DAP12, FCER1G (Fc epsilon receptor I gamma chain), CD3D (CD3 delta), CD3E (CD3 epsilon), CD3G (CD3 gamma), CD3Z (CD3 zeta), and CD79A (antigen receptor complex-associated protein alpha chain).

In one embodiment, the intracellular signaling domain is derived from DAP12 (also known as TYROBP; TYRO protein tyrosine kinase binding protein; KARAP; PLOSL; DNAX-activation protein 12; KAR-associated protein; TYRO protein tyrosine kinase-binding protein; killer activating receptor associated protein; killer-activating receptor-associated protein; etc.). In one embodiment, the intracellular signaling domain is derived from FCER1G (also known as FCRG; Fc epsilon receptor I gamma chain; Fc receptor gamma-chain; fc-epsilon RI-gamma; fcRgamma; fceR1 gamma; high affinity immunoglobulin epsilon receptor subunit gamma; immunoglobulin E receptor, high affinity, gamma chain; etc.). In one embodiment, the intracellular signaling domain is derived from T-cell surface glycoprotein CD3 delta chain (also known as CD3D; CD3-DELTA; T3D; CD3 antigen, delta subunit; CD3 delta; CD3d antigen, delta polypeptide (TiT3 complex); OKT3, delta chain; T-cell receptor T3 delta chain; T-cell surface glycoprotein CD3 delta chain; etc.). In one embodiment, the intracellular signaling domain is derived from T-cell surface glycoprotein CD3 epsilon chain (also known as CD3e, T-cell surface antigen T3/Leu-4 epsilon chain, T-cell surface glycoprotein CD3 epsilon chain, AI504783, CD3, CD3epsilon, T3e, etc.). In one embodiment, the intracellular signaling domain is derived from T-cell surface glycoprotein CD3 gamma chain (also known as CD3G, T-cell receptor T3 gamma chain, CD3-GAMMA, T3G, gamma polypeptide (TiT3 complex), etc.). In one embodiment, the intracellular signaling domain is derived from T-cell surface glycoprotein CD3 zeta chain (also known as CD3Z, T-cell receptor T3 zeta chain, CD247, CD3-ZETA, CD3H, CD3Q, T3Z, TCRZ, etc.). In one embodiment, the intracellular signaling domain is derived from CD79A (also known as B-cell antigen receptor complex-associated protein alpha chain; CD79a antigen (immunoglobulin-associated alpha); MB-1 membrane glycoprotein; ig-alpha; membrane-bound immunoglobulin-associated protein; surface IgM-associated protein; etc.). In one embodiment, an intracellular signaling domain suitable for use in an FN3 CAR of the present disclosure includes a DAP10/CD28 type signaling chain. In one embodiment, an intracellular signaling domain suitable for use in an FN3 CAR of the present disclosure includes a ZAP70 polypeptide. In some embodiments, the intracellular signaling domain includes a cytoplasmic signaling domain of TCR zeta, FcR gamma, FcR beta, CD3 gamma, CD3 delta, CD3 epsilon, CD5, CD22, CD79a, CD79b, or CD66d. In one embodiment, the intracellular signaling domain in the CAR includes a cytoplasmic signaling domain of human CD3 zeta.

While usually the entire intracellular signaling domain can be employed, in many cases it is not necessary to use the entire chain. To the extent that a truncated portion of the intracellular signaling domain is used, such truncated portion may be used in place of the intact chain as long as it transduces the effector function signal. The intracellular signaling domain includes any truncated portion of the intracellular signaling domain sufficient to transduce the effector function signal.

The intracellular signaling domains described herein can be combined with any of the antigen binding domains described herein, any of the transmembrane domains described herein, or any of the other domains described herein that may be included in the CAR.

### Modified Immune Cells

The present disclosure provides modified immune cells or precursors thereof (e.g., monocyte, macrophage, B cell, T cell, NK cell, neutrophil, stem cell) for use in immunotherapy (e.g. CAR T cells). In certain aspects, the disclosure provides a modified immune cell or precursor cell thereof comprising an anti-amyloid beta (Aβ) chimeric antigen receptor (CAR). In certain aspects, the disclosure provides a modified immune cell or precursor cell thereof comprising an anti-Tau CAR. In certain aspects, the disclosure provides a modified immune cell or precursor cell thereof comprising an anti-Tau CAR and an anti-amyloid beta (Aβ) CAR. The invention should be construed to include any cell comprising one or more of any of the CARs disclosed herein. CARs of the present disclosure comprise an antigen binding domain and a transmembrane domain. In certain embodiments, the CAR does not comprise an intracellular domain.

In certain embodiments, the immune cell or precursor cell thereof is a monocyte, macrophage, B cell, NK cell, neutrophil, or stem cell. In certain embodiments, the immune cell or precursor cell thereof is a T cell. In certain embodiments, the cell is a human cell. In certain embodiments, the cell is an autologous cell (e.g. an autologous T cell, monocyte, macrophage, B cell, NK cell, neutrophil, or stem cell).

Thus, provided are cells, compositions and methods that enhance immune cell function in adoptive cell therapy, including those offering improved efficacy, such as by increasing activity and potency of administered genetically engineered cells, while maintaining persistence or exposure to the transferred cells over time.

In some embodiments, the degree or extent of persistence of administered cells can be detected or quantified after administration to a subject. For example, in some aspects, quantitative PCR (qPCR) is used to assess the quantity of cells expressing the CAR in the blood or serum or organ or tissue (e.g., disease site) of the subject. In some aspects, persistence is quantified as copies of DNA or plasmid encoding the exogenous receptor per microgram of DNA, or as the number of receptor-expressing cells per microliter of the sample, e.g., of blood or serum, or per total number of peripheral blood mononuclear cells (PBMCs) or white blood cells or T cells per microliter of the sample. In some embodiments, flow cytometric assays detecting cells expressing the receptor generally using antibodies specific for the receptors also can be performed. Cell-based assays may also be used to detect the number or percentage of functional cells, such as cells capable of binding to and/or neutralizing and/or inducing responses, e.g., cytotoxic responses, against cells of the disease or condition or expressing the antigen recognized by the receptor. In any of such embodiments, the extent or level of expression of another marker associated with the modified cell can be used to distinguish the administered cells from endogenous cells in a subject.

### Vectors

In some embodiments, a vector may be used to introduce a CAR into an immune cell (e.g., a monocyte, macrophage or stem cell; e.g. a hematopoietic stem cell with the potential to become a myeloid cell such as a monocyte, macrophage or microglia-like cell), as described elsewhere herein. In one aspect, the disclosure includes a vector comprising a nucleic acid sequence encoding a CAR as described herein. In some embodiments, the vector comprises a plasmid vector, viral vector, retrotransposon (e.g. piggyback, sleeping beauty), site directed insertion vector (e.g. CRISPR, Zn finger nucleases, TALEN), or suicide expression vector, or other known vector in the art.

In some embodiments, constructs mentioned above are capable of use with 3rd generation lentiviral vector plasmids, other viral vectors, or RNA approved for use in human cells. In some embodiments, the vector is a viral vector, such as a lentiviral vector. In some embodiments, a lentiviral vector is packaged with a Vpx protein. In some embodiments, a Vpx protein is provided to a population of cells separately from a lentiviral vector (e.g., before administration of a vector, substantially at the same time as a vector, or after administration of a vector). In some embodiments, the vector is a RNA vector.

The production of any of the molecules described herein can be verified by sequencing. Expression of the full length proteins may be verified using immunoblot, immunohistochemistry, flow cytometry or other technology well known and available in the art.

In some embodiments, the present disclosure also provides vectors in which DNA of the present invention is inserted. Vectors, including those derived from retroviruses such as lentivirus, are suitable tools to achieve long-term gene transfer, at least in part, since they allow long-term, stable integration of a transgene and its propagation in daughter cells. Lentiviral vectors have the added advantage over vectors derived from onco-retroviruses, such as murine leukemia viruses, in that they can transduce non-proliferating cells, such as hepatocytes. They also have the added advantage of resulting in low immunogenicity in the subject into which they are introduced.

The expression of natural or synthetic nucleic acids is typically achieved by operably linking a nucleic acid or portions thereof to a promoter, and incorporating the construct into an expression vector. The vector is one generally capable of replication in a mammalian cell, and/or also capable of integration into the cellular genome of the mammal. Typical vectors contain transcription and translation terminators, initiation sequences, and promoters useful for regulation of the expression of the desired nucleic acid sequence.

In accordance with various embodiments, a nucleic acid can be cloned into any number of different types of vectors. For example, in some embodiments, a nucleic acid can be cloned into a vector including, but not limited to a plasmid, a phagemid, a phage derivative, an animal virus, and a cosmid. Vectors of particular interest in some embodiments include expression vectors, replication vectors, probe generation vectors, and sequencing vectors.

In some embodiments, an expression vector may be provided to a cell in the form of a viral vector. Viral vector technology is well known in the art and is described, for example, in Sambrook et al., 2012, MOLECULAR CLONING: A LABORATORY MANUAL, volumes 1-4, Cold Spring Harbor Press, NY, and in other virology and molecular biology manuals. Viruses, which are useful as vectors include, but are not limited to, retroviruses, adenoviruses, adeno-associated viruses, herpes viruses, and lentiviruses. In general, a suitable vector contains an origin of replication functional in at least one organism, a promoter sequence, convenient restriction endonuclease sites, and one or more selectable markers, (e.g., WO 01/96584; WO 01/29058; and U.S. Pat. No. 6,326,193, the contents of which are incorporated herein by reference in their entireties).

Additional promoter elements, e.g., enhancers, regulate the frequency of transcriptional initiation and may be useful in some embodiments. Typically, these are located in the region 30-110 bp upstream of the start site, although a number of promoters have recently been shown to contain functional elements downstream of the start site as well. The spacing between promoter elements frequently is flexible, so that promoter function is preserved when elements are inverted or moved relative to one another. In the thymidine kinase (tk) promoter, the spacing between promoter elements can be increased to 50 bp apart before activity begins to decline. Depending on the promoter, it appears that individual elements can function either cooperatively or independently to activate transcription.

An example of a promoter is the immediate early cytomegalovirus (CMV) promoter sequence. This promoter sequence is a strong constitutive promoter sequence capable of driving high levels of expression of any polynucleotide sequence operatively linked thereto. However, other constitutive promoter sequences may also be used in accordance with various embodiments, including, but not limited to the simian virus 40 (SV40) early promoter, mouse mammary tumor virus (MMTV), human immunodeficiency virus (HIV) long terminal repeat (LTR) promoter, MoMuLV promoter, an avian leukemia virus promoter, an Epstein-Barr virus immediate early promoter, a Rous sarcoma virus promoter, the elongation factor-1α promoter, as well as human gene promoters such as, but not limited to, the actin promoter, the myosin promoter, the hemoglobin promoter, and the creatine kinase promoter. Further, the invention should not be limited to the use of constitutive promoters. Inducible promoters are also contemplated as part of the invention. The use of an inducible promoter provides a molecular switch capable of turning on expression of the polynucleotide sequence which it is operatively linked when such expression is desired, or turning off the expression when expression is not desired. Examples of inducible promoters include, but are not limited to a metallothionine promoter, a glucocorticoid promoter, a progesterone promoter, and a tetracycline promoter.

In order to assess expression of a polypeptide or portions thereof, the expression vector to be introduced into a cell can also contain either a selectable marker gene or a reporter gene or both to facilitate identification and selection of expressing cells from the population of cells sought to be transfected or infected through viral vectors. In other aspects, the selectable marker may be carried on a separate piece of DNA and used in a co-transfection procedure. Both selectable markers and reporter genes may be flanked with appropriate regulatory sequences to enable expression in the host cells. Useful selectable markers include, for example, antibiotic-resistance genes, such as neo and the like.

In some embodiments, reporter genes are used for identifying potentially transfected cells and for evaluating the functionality of regulatory sequences. In general, a reporter gene is a gene that is not present in or expressed by the recipient organism or tissue and that encodes a polypeptide whose expression is manifested by some easily detectable property, e.g., enzymatic activity. Expression of a reporter gene is assessed at a suitable time after the DNA has been introduced into the recipient cells. Suitable reporter genes may include genes encoding luciferase, beta-galactosidase, chloramphenicol acetyl transferase, secreted alkaline phosphatase, or the green fluorescent protein gene (e.g., Ui-Tei et al., 2000 FEBS Letters 479: 79-82, which is incorporated herein by reference in its entirety). Suitable expression systems are well known and may be prepared using known techniques or obtained commercially. In general, a construct with the minimal 5′ flanking region showing the highest level of expression of reporter gene is identified as the promoter. Such promoter regions may be linked to a reporter gene and used to evaluate agents for the ability to modulate promoter-driven transcription.

### Introduction of Nucleic Acids

In some embodiments, the disclosure provides methods for modifying cells comprising introducing a nucleic acid sequence encoding some or all of a chimeric antigen receptor (CAR) into an immune cell (e.g., a monocyte, macrophage, or stem cell; e.g., a hematopoietic stem cell with the potential to become a myeloid cell such as a monocyte, macrophage or microglia-like cell), wherein the CAR comprises an antigen binding domain and a transmembrane domain. In certain embodiments, the CAR comprises an intracellular domain. In certain embodiments, the CAR does not comprise an intracellular domain. In certain embodiments, the antigen binding domain is capable of binding to amyloid beta (Aβ) or Tau, and the cell is a monocyte, macrophage and/or a hematopoietic stem cell with the potential to become a myeloid cell such as a monocyte, macrophage or microglia-like cell that expresses the CAR.

In some embodiments, the disclosure provides methods for modifying a cell comprising introducing a nucleic acid sequence (e.g., an isolated or non-native nucleic acid sequence) encoding a chimeric antigen receptor (CAR) into an immune cell (e.g., a monocyte, macrophage, or stem cell; e.g., a hematopoietic stem cell with the potential to become a myeloid cell such as a monocyte, macrophage or microglia-like cell), wherein the isolated nucleic acid sequence comprises a nucleic acid sequence encoding an antigen binding domain and a nucleic acid sequence encoding a transmembrane domain. In some embodiments, and a nucleic acid sequence encoding an intracellular domain is also introduced. In certain embodiments, the antigen binding domain is capable of binding to amyloid beta (Aβ) or Tau, and the cell is a monocyte, macrophage, or stem cell (e.g., a hematopoietic stem cell with the potential to become a myeloid cell such as a monocyte, macrophage or microglia-like cell), that expresses the CAR. In some embodiments, one or more of the antigen binding domain, transmembrane domain, and the intracellular domain are encoded by separate nucleic acid molecules.

In some embodiments, the modified immune cell expresses the CAR and possesses targeted effector activity. In some embodiments, introducing the CAR into the immune cell comprises introducing a nucleic acid sequence encoding the CAR (e.g., some components or all of the CAR). In some embodiments, introducing the nucleic acid sequence comprises electroporating DNA or a mRNA encoding the CAR into a cell.

Methods of introducing and expressing genes, such as those that encode a CAR, into a cell are known in the art. In the context of an expression vector, the vector can be readily introduced into a host cell, e.g., mammalian, bacterial, yeast, or insect cell by any method in the art. For example, in some embodiments, an expression vector can be transferred into a host cell by physical, chemical, or biological means.

Physical methods for introducing a polynucleotide into a host cell include calcium phosphate precipitation, lipofection, particle bombardment, microinjection, electroporation, squeeze technology, and the like. Methods for producing cells comprising vectors and/or exogenous nucleic acids are well-known in the art. See, for example, Sambrook et al., 2012, MOLECULAR CLONING: A LABORATORY MANUAL, volumes 1-4, Cold Spring Harbor Press, NY). Nucleic acids can be introduced into target cells using commercially available methods which include electroporation (Amaxa Nucleofector-II (Amaxa Biosystems, Cologne, Germany)), (ECM 830 (BTX) (Harvard Instruments, Boston, Mass.) or the Gene Pulser II (BioRad, Denver, Colo.), Multiporator (Eppendort, Hamburg Germany). Nucleic acids can also be introduced into cells using cationic liposome mediated transfection, using lipofection, using polymer encapsulation, using peptide mediated transfection, or using biolistic particle delivery systems such as “gene guns” (see, for example, Nishikawa, et al. Hum Gene Ther., 12(8):861-70 (2001).

In some embodiments, biological methods for introducing a polynucleotide of interest into a host cell may be or include the use of DNA and RNA vectors. RNA vectors include vectors having a RNA promoter and/or other relevant domains for production of a RNA transcript. Viral vectors, and especially retroviral vectors, have become the most widely used method for inserting genes into mammalian, e.g., human, cells. Other viral vectors may be derived from lentivirus, poxviruses, herpes simplex virus, adenoviruses (e.g. Ad5F35) and adeno-associated viruses, and the like. See, for example, U.S. Pat. Nos. 5,350,674 and 5,585,362.

In some embodiments, introducing a nucleic acid sequence into the cell comprises adenoviral transduction. In some embodiments, adenoviral transduction comprises use of an Ad5F35 adenovirus vector. In some embodiments, an Ad5F35 adenovirus vector is a helper-dependent Ad5F35 adenovirus vector. In some embodiments, an AD5F35 adenovirus vector is an integrating, CD46-targeted, helper-dependent adenovirus HDAd5/35++ vector system.

Chemical means for introducing a polynucleotide into a host cell include colloidal dispersion systems, such as macromolecule complexes, nanocapsules, microspheres, beads, and lipid-based systems including oil-in-water emulsions, micelles, mixed micelles, and liposomes. An exemplary colloidal system for use as a delivery vehicle in vitro and in vivo is a liposome (e.g., an artificial membrane vesicle).

In some embodiments, where a non-viral delivery system is utilized, an exemplary delivery vehicle may be or comprise a liposome. The use of lipid formulations is contemplated for the introduction of the nucleic acids into a host cell (in vitro, ex vivo or in vivo). In another aspect, a nucleic acid may be associated with a lipid. In some embodiments, a nucleic acid associated with a lipid may be encapsulated in the aqueous interior of a liposome, interspersed within the lipid bilayer of a liposome, attached to a liposome via a linking molecule that is associated with both the liposome and the oligonucleotide, entrapped in a liposome, complexed with a liposome, dispersed in a solution containing a lipid, mixed with a lipid, combined with a lipid, contained as a suspension in a lipid, contained or complexed with a micelle, or otherwise associated with a lipid. Lipid, lipid/DNA or lipid/expression vector associated compositions are not limited to any particular structure in solution. For example, they may be present in a bilayer structure, as micelles, or with a “collapsed” structure. They may also simply be interspersed in a solution, possibly forming aggregates that are not uniform in size or shape. Lipids are fatty substances which may be naturally occurring or synthetic lipids. For example, lipids include the fatty droplets that naturally occur in the cytoplasm as well as the class of compounds which contain long-chain aliphatic hydrocarbons and their derivatives, such as fatty acids, alcohols, amines, amino alcohols, and aldehydes.

Lipids suitable for use can be obtained from commercial sources. For example, dimyristyl phosphatidylcholine (“DMPC”) can be obtained from Sigma, St. Louis, MO; dicetyl phosphate (“DCP”) can be obtained from K & K Laboratories (Plainview, NY); cholesterol (“Choi”) can be obtained from Calbiochem-Behring; dimyristyl phosphatidylglycerol (“DMPG”) and other lipids may be obtained from Avanti Polar Lipids, Inc. (Birmingham, AL.). Stock solutions of lipids in chloroform or chloroform/methanol can be stored at about −20° C. Chloroform is used as the only solvent since it is more readily evaporated than methanol. “Liposome” is a generic term encompassing a variety of single and multilamellar lipid vehicles formed by the generation of enclosed lipid bilayers or aggregates. Liposomes can be characterized as having vesicular structures with a phospholipid bilayer membrane and an inner aqueous medium. Multilamellar liposomes have multiple lipid layers separated by aqueous medium. They form spontaneously when phospholipids are suspended in an excess of aqueous solution. The lipid components undergo self-rearrangement before the formation of closed structures and entrap water and dissolved solutes between the lipid bilayers (Ghosh et al., 1991 Glycobiology 5: 505-10). However, compositions that have different structures in solution than the normal vesicular structure are also encompassed. For example, the lipids may assume a micellar structure or merely exist as nonuniform aggregates of lipid molecules. Also contemplated are lipofectamine-nucleic acid complexes.

Lipid-based nanoparticles (LNPs) may also be used to deliver nucleic acids (i.e. nucleic acids encoding CARs) to cells. LNPs are one of the most effective non-viral transfection strategies for in vivo delivery of nucleic acid-based therapeutics, including RNA-based therapeutics. LNPs are typically composed of four main lipid types: an ionizable lipid, a neutral helper lipid, cholesterol for structural integrity, and sterically stabilizing lipid. Ionizable lipids contain an amine group that can be positively charged at low pH values. Sterically stabilizing lipids are usually the PEG-lipid conjugates (e.g., PEG-DMG), which cover the surface of the LNPs and shield overall surface charges (positive or negative), making the surface hydrophilic. In vivo applications of sterically stabilizing lipids prevent opsonization and increase the longevity of the LNPs in the blood.

Regardless of the method used to introduce exogenous nucleic acids into a host cell or otherwise expose a cell to the molecules described herein, in order to confirm the presence of the nucleic acids in the host cell, a variety of assays may be performed. Such assays include, for example, “molecular biological” assays well known to those of skill in the art, such as Southern and Northern blotting, RT-PCR and PCR; “biochemical” assays, such as detecting the presence or absence of a particular peptide, e.g., by immunological means (ELISAs and Western blots) or by assays described herein to identify agents falling within the scope of the invention.

In some embodiments, one or more nucleic acid sequences are introduced by a method selected from the group consisting of transducing the population of cells, transfecting the population of cells, and electroporating the population of cells. In some embodiments, a population of cells comprises one or more of the nucleic acid sequences described herein. In some embodiments, one or more nucleic acids are transfected, transduced and/or electroporated with one or more nuclease enzymes (e.g. Cas9, Cas12a, or C2c2, for example).

In some embodiments, nucleic acids introduced into the cell are or comprise RNA. In some embodiments, RNA is mRNA that comprises in vitro transcribed RNA or synthetic RNA. In some embodiments, RNA is produced by in vitro transcription using a polymerase chain reaction (PCR)-generated template. DNA of interest from any source can be directly converted by PCR into a template for in vitro mRNA synthesis using appropriate primers and RNA polymerase. In some embodiments, the source of the DNA can be, for example, genomic DNA, plasmid DNA, phage DNA, cDNA, synthetic DNA sequence or any other appropriate source of DNA. In some embodiments, a desired template for in vitro transcription is or comprises a CAR.

In some embodiments, PCR can be used to generate a template for in vitro transcription of mRNA which is then introduced into cells. Methods for performing PCR are well known in the art. Primers for use in PCR are designed to have regions that are substantially complementary to regions of the DNA to be used as a template for the PCR. “Substantially complementary”, as used herein, refers to sequences of nucleotides where a majority or all of the bases in the primer sequence are complementary, or one or more bases are non-complementary, or mismatched. Substantially complementary sequences are able to anneal or hybridize with the intended DNA target under annealing conditions used for PCR. The primers can be designed to be substantially complementary to any portion of the DNA template. For example, the primers can be designed to amplify the portion of a gene that is normally transcribed in cells (the open reading frame), including 5′ and 3′ UTRs. The primers can also be designed to amplify a portion of a gene that encodes a particular domain of interest. In one embodiment, the primers are designed to amplify the coding region of a human cDNA, including all or portions of the 5′ and 3′ UTRs. Primers useful for PCR are generated by synthetic methods that are well known in the art. “Forward primers” are primers that contain a region of nucleotides that are substantially complementary to nucleotides on the DNA template that are upstream of the DNA sequence that is to be amplified. “Upstream” is used herein to refer to a location 5, to the DNA sequence to be amplified relative to the coding strand. “Reverse primers” are primers that contain a region of nucleotides that are substantially complementary to a double-stranded DNA template that are downstream of the DNA sequence that is to be amplified. “Downstream” is used herein to refer to a location 3′ to the DNA sequence to be amplified relative to the coding strand.

Chemical structures that have the ability to promote stability and/or translation efficiency of the RNA may also be used. The RNA preferably has 5′ and 3′ UTRs. In one embodiment, the 5′ UTR is between zero and 3000 nucleotides in length. The length of 5′ and 3′ UTR sequences to be added to the coding region can be altered by different methods, including, but not limited to, designing primers for PCR that anneal to different regions of the UTRs. Using this approach, one of ordinary skill in the art can modify the 5′ and 3′ UTR lengths required to achieve optimal translation efficiency following transfection of the transcribed RNA.

The 5′ and 3′ UTRs can be the naturally occurring, endogenous 5′ and 3′ UTRs for the gene of interest. Alternatively, UTR sequences that are not endogenous to the gene of interest can be added by incorporating the UTR sequences into the forward and reverse primers or by any other modifications of the template. The use of UTR sequences that are not endogenous to the gene of interest can be useful for modifying the stability and/or translation efficiency of the RNA. For example, it is known that AU-rich elements in 3′ UTR sequences can decrease the stability of mRNA. Therefore, 3′ UTRs can be selected or designed to increase the stability of the transcribed RNA based on properties of UTRs that are well known in the art.

In some embodiments, the 5′ UTR can contain the Kozak sequence of the endogenous gene. Alternatively, when a 5′ UTR that is not endogenous to the gene of interest is being added by PCR as described above, a consensus Kozak sequence can be redesigned by adding the 5′ UTR sequence. Kozak sequences can increase the efficiency of translation of some RNA transcripts, but does not appear to be required for all RNAs to enable efficient translation. The requirement for Kozak sequences for many mRNAs is known in the art. In other embodiments the 5′ UTR can be derived from an RNA virus whose RNA genome is stable in cells. In other embodiments various nucleotide analogues can be used in the 3′ or 5′ UTR to impede exonuclease degradation of the mRNA.

To enable synthesis of RNA from a DNA template without the need for gene cloning, a promoter of transcription should be attached to the DNA template upstream of the sequence to be transcribed. When a sequence that functions as a promoter for an RNA polymerase is added to the 5′ end of the forward primer, the RNA polymerase promoter becomes incorporated into the PCR product upstream of the open reading frame that is to be transcribed. In one embodiment, the promoter is a T7 polymerase promoter, as described elsewhere herein. Other useful promoters include, but are not limited to, T3 and SP6 RNA polymerase promoters. Consensus nucleotide sequences for T7, T3 and SP6 promoters are known in the art.

In some embodiments, the mRNA has both a cap on the 5′ end and a 3′ poly(A) tail which determine ribosome binding, initiation of translation and stability mRNA in the cell. On a circular DNA template, for instance, plasmid DNA, RNA polymerase produces a long concatameric product which is not suitable for expression in eukaryotic cells. The transcription of plasmid DNA linearized at the end of the 3′ UTR results in normal sized mRNA which is not effective in eukaryotic transfection even if it is polyadenylated after transcription.

On a linear DNA template, phage T7 RNA polymerase can extend the 3′ end of the transcript beyond the last base of the template (Schenborn and Mierendorf, Nuc Acids Res., 13:6223-36 (1985); Nacheva and Berzal-Herranz, Eur. J. Biochem., 270:1485-65 (2003).

The conventional method of integration of polyA/T stretches into a DNA template is molecular cloning. However, polyA/T sequence integrated into plasmid DNA can cause plasmid instability, which is why plasmid DNA templates obtained from bacterial cells are often highly contaminated with deletions and other aberrations. This makes cloning procedures not only laborious and time consuming but often not reliable. That is why a method which allows construction of DNA templates with polyA/T 3′ stretch without cloning highly desirable.

The polyA/T segment of the transcriptional DNA template can be produced during PCR by using a reverse primer containing a polyT tail, such as 100 T tail (size can be 50-5000 T), or after PCR by any other method, including, but not limited to, DNA ligation or in vitro recombination. Poly(A) tails also provide stability to RNAs and reduce their degradation. Generally, the length of a poly(A) tail positively correlates with the stability of the transcribed RNA. In one embodiment, the poly(A) tail is between 100 and 5000 adenosines.

Poly(A) tails of RNAs can be further extended following in vitro transcription with the use of a poly(A) polymerase, such as E. coli polyA polymerase (E-PAP). In one embodiment, increasing the length of a poly(A) tail from 100 nucleotides to between 300 and 400 nucleotides results in about a two-fold increase in the translation efficiency of the RNA. Additionally, the attachment of different chemical groups to the 3′ end can increase mRNA stability. Such attachment can contain modified/artificial nucleotides, aptamers and other compounds. For example, ATP analogs can be incorporated into the poly(A) tail using poly(A) polymerase. ATP analogs can further increase the stability of the RNA.

5′ caps can also provide stability to RNA molecules. In a preferred embodiment, RNAs produced by the methods disclosed herein include a 5′ cap. The 5′ cap is provided using techniques known in the art and described herein (Cougot, et al., Trends in Biochem. Sci., 29:436-444 (2001); Stepinski, et al., RNA, 7:1468-95 (2001); Elango, et al., Biochim. Biophys. Res. Commun., 330:958-966 (2005)).

In some embodiments, RNAs produced by the methods disclosed herein can also contain an internal ribosome entry site (IRES) sequence. The IRES sequence may be any viral, chromosomal or artificially designed sequence which initiates cap-independent ribosome binding to mRNA and facilitates the initiation of translation. Any solutes suitable for cell electroporation, which can contain factors facilitating cellular permeability and viability such as sugars, peptides, lipids, proteins, antioxidants, and surfactants can be included.

Some in vitro-transcribed RNA (IVT-RNA) vectors are known in the literature which are utilized in a standardized manner as template for in vitro transcription and which have been genetically modified in such a way that stabilized RNA transcripts are produced. Currently protocols used in the art are based on a plasmid vector with the following structure: a 5′ RNA polymerase promoter enabling RNA transcription, followed by a gene of interest which is flanked either 3′ and/or 5′ by untranslated regions (UTR), and a 3′ polyadenyl cassette containing 50-70 A nucleotides. Prior to in vitro transcription, the circular plasmid is linearized downstream of the polyadenyl cassette by type II restriction enzymes (recognition sequence corresponds to cleavage site). The polyadenyl cassette thus corresponds to the later poly(A) sequence in the transcript. As a result of this procedure, some nucleotides remain as part of the enzyme cleavage site after linearization and extend or mask the poly(A) sequence at the 3′ end. It is not clear, whether this nonphysiological overhang affects the amount of protein produced intracellularly from such a construct.

In some embodiments, an RNA construct is delivered into the cells by electroporation. See, e.g., the formulations and methodology of electroporation of nucleic acid constructs into mammalian cells as taught in US 2004/0014645, US 2005/0052630A1, US 2005/0070841A1, US 2004/0059285A1, US 2004/0092907A1, each of which is hereby incorporated by reference in its entirety. The various parameters including electric field strength required for electroporation of any known cell type are generally known in the relevant research literature as well as numerous patents and applications in the field. See e.g., U.S. Pat. Nos. 6,678,556, 7,171,264, and 7,173,116, each of which is hereby incorporated by reference in its entirety. Apparatus for therapeutic application of electroporation are available commercially, e.g., the MedPulser™ DNA Electroporation Therapy System (Inovio/Genetronics, San Diego, Calif.), and are described in patents such as U.S. Pat. Nos. 6,567,694; 6,516,223, 5,993,434, 6,181,964, 6,241,701, and 6,233,482, each of which is hereby incorporated by reference in its entirety; electroporation may also be used for transfection of cells in vitro as described e.g. in US20070128708A1. Electroporation may also be utilized to deliver nucleic acids into cells in vitro. Accordingly, electroporation-mediated administration into cells of nucleic acids including expression constructs utilizing any of the many available devices and electroporation systems known to those of skill in the art presents an exciting new means for delivering an RNA of interest to a target cell.

### Sources of Cells

In some embodiments, phagocytic cells are used in the compositions and methods described herein. In some embodiments, a source of phagocytic cells, such as monocytes, macrophages and/or hematopoietic stem cells with the potential to become a myeloid cells such as a monocytes, macrophages or microglia-like cells, is obtained from a subject. Non-limiting examples of subjects include humans, dogs, cats, mice, rats, and transgenic species thereof. Preferably, the subject is a human. In some embodiments, cells can be obtained from a number of sources, including peripheral blood mononuclear cells, bone marrow, lymph node tissue, spleen tissue, umbilical cord, and induced pluripotent stem cells. In certain embodiments, any number of monocyte, macrophage, dendritic cell or progenitor cell lines available in the art, may be used. In certain embodiments, cells can be obtained from a unit of blood collected from a subject using any number of techniques known to the skilled artisan, such as Ficoll separation. In some embodiments, cells from the circulating blood of an individual are obtained by apheresis or leukapheresis. The apheresis product typically contains lymphocytes, including T cells, monocytes, granulocytes, B cells, other nucleated white blood cells, red blood cells, and platelets. The cells collected by apheresis may be washed to remove the plasma fraction and to place the cells in an appropriate buffer or media, such as phosphate buffered saline (PBS) or wash solution lacks calcium and may lack magnesium or may lack many if not all divalent cations, for subsequent processing steps. After washing, the cells may be resuspended in a variety of biocompatible buffers, such as, for example, Ca-free, Mg-free PBS. Alternatively, the undesirable components of the apheresis sample may be removed and the cells directly resuspended in culture media.

In some embodiments, precursors to monocytes, macrophages, or dendritic cells may be used. Non-limiting examples include, hematopoietic stem cells, common myeloid progenitors, myeloblasts, monoblasts, promonocytes, and intermediates. In another embodiment, induced pluripotent stem cells may be used as a source of generating monocytes, macrophages, and/or dendritic cells.

If myeloid precursors are used, such as hematopoietic stem cells, they may be ex vivo differentiated into monocytes, macrophages, and/or microglia-like cells, or precursors of said pathway. In addition, precursors (such as but not limited to hematopoietic stem cells) may be used as the therapeutic cell, such that the myeloid differentiation occurs in vivo. Cells may be autologous or sourced from allogeneic or universal donors. In some embodiments, myeloid progenitors or hematopoietic stem cells may be engineered such that expression of the CAR is under the control of a cell type specific promoter, such as a known myeloid, macrophage, monocyte, dendritic cell, microglial cell, M1 specific, or M2 specific promoter.

In some embodiments, monocytes or precursors may be ex vivo differentiated into microglial cells prior to infusion with cytokines known to those in the art. In some embodiments, differentiation of monocytes into microglial cells may improve activity in the central nervous system.

In some embodiments, cells are isolated from peripheral blood by lysing the red blood cells and depleting the lymphocytes and red blood cells, for example, by centrifugation through a PERCOLL™ gradient. Alternatively, cells can be isolated from umbilical cord. In any event, a specific subpopulation of the monocytes, macrophages and/or dendritic cells can be further isolated by positive or negative selection techniques.

The mononuclear cells so isolated can be depleted of cells expressing certain antigens, including, but not limited to, CD34, CD3, CD4, CD8, CD19 or CD20. Depletion of these cells can be accomplished using an isolated antibody, a biological sample comprising an antibody, such as ascites fluid, an antibody bound to a physical support, and a cell bound antibody.

Enrichment of a monocyte, macrophage and/or dendritic cell population by negative selection can be accomplished using a combination of antibodies directed to surface markers unique to the negatively selected cells. A preferred method is cell sorting and/or selection via negative magnetic immunoadherence or flow cytometry that uses a cocktail of monoclonal antibodies directed to cell surface markers present on the cells negatively selected. For example, enrich of a cell population for monocytes, macrophages and/or dendritic cells by negative selection can be accomplished using a monoclonal antibody cocktail that typically includes antibodies to CD34, CD3, CD4, CD8, CD14, CD19 or CD20.

During isolation of a desired population of cells by positive or negative selection, the concentration of cells and surface (e.g., particles such as beads) can be varied. In certain embodiments, it may be desirable to significantly decrease the volume in which beads and cells are mixed together (i.e., increase the concentration of cells), to ensure maximum contact of cells and beads. For example, in one embodiment, a concentration of 2 billion cells/ml is used. In one embodiment, a concentration of 1 billion cells/ml is used. In a further embodiment, greater than 100 million cells/ml is used. In a further embodiment, a concentration of cells of 10, 15, 20, 25, 30, 35, 40, 45, or 50 million cells/ml is used. In yet another embodiment, a concentration of cells from 75, 80, 85, 90, 95, or 100 million cells/ml is used. In further embodiments, concentrations of 125 or 150 million cells/ml can be used. The use of high concentrations of cells can result in increased cell yield, cell activation, and cell expansion.

In some embodiments, a population of cells comprises the monocytes, macrophages, or hematopoietic stem cells with the potential to become myeloid cells such as a monocytes, macrophages or microglia-like cells of the present invention. Examples of a population of cells include, but are not limited to, peripheral blood mononuclear cells, cord blood cells, a purified population of monocytes, macrophages, or stem cells, and a cell line. In some embodiments, peripheral blood mononuclear cells comprise the population of monocytes, macrophages, or hematopoietic stem cells with the potential to become myeloid cells such as monocytes, macrophages or microglia-like cells. In some embodiments, purified cells comprise the population of monocytes, macrophages, or hematopoietic stem cells with the potential to become myeloid cells such as monocytes, macrophages or microglia-like cells.

In some embodiments, cells may have upregulated M1 markers and/or downregulated M2 markers. For example, in some embodiments, at least one M1 marker, such as HLA DR, CD86, CD80, and PDL1, is upregulated in the phagocytic cell. In another example, in some embodiments, at least one M2 marker, such as CD206, CD163, is downregulated in the phagocytic cell. In one embodiment, the cell has at least one upregulated M1 marker and at least one downregulated M2 marker.

In some embodiments, targeted effector activity in the phagocytic cell is enhanced by inhibition of either CD47 or SIRPα activity. CD47 and/or SIRPα activity may be inhibited by treating the phagocytic cell with an anti-CD47 or anti-SIRPα antibody. Alternatively, CD47 or SIRPα activity may be inhibited by any method known to those skilled in the art.

### Expansion of Cells

In some embodiments, cells or population of cells comprising monocytes or macrophages are cultured for expansion. In some embodiments, cells or population of cells comprising progenitor cells (e.g. stem cells; e.g. hematopoietic stem cells with the potential to become myeloid cells such as a monocytes, macrophages or microglia-like cells) are cultured for differentiation and expansion of monocytes or macrophages. In some embodiments, the methods provided herein comprise expanding a population of monocytes, macrophages, or stem cells comprising a chimeric antigen receptor as described herein.

Expanding the immune cells by the methods disclosed herein can be multiplied by about 10 fold, 20 fold, 30 fold, 40 fold, 50 fold, 60 fold, 70 fold, 80 fold, 90 fold, 100 fold, 200 fold, 300 fold, 400 fold, 500 fold, 600 fold, 700 fold, 800 fold, 900 fold, 1000 fold, 2000 fold, 3000 fold, 4000 fold, 5000 fold, 6000 fold, 7000 fold, 8000 fold, 9000 fold, 10,000 fold, 100,000 fold, 1,000,000 fold, 10,000,000 fold, or greater, and any and all whole or partial integers therebetween. In one embodiment, the cells expand in the range of about 20 fold to about 50 fold.

Following culturing, cells can be incubated in cell medium in a culture apparatus for a period of time or until the cells reach confluency or high cell density for optimal passage before passing the cells to another culture apparatus. In some embodiments, a culturing apparatus can be any culture apparatus commonly used for culturing cells in vitro. Preferably, the level of confluence is 70% or greater before passing the cells to another culture apparatus. More preferably, the level of confluence is 90% or greater. A period of time can be any time suitable for the culture of cells in vitro. The culture medium may be replaced during the culture of the cells at any time. Preferably, the culture medium is replaced about every 2 to 3 days. The cells are then harvested from the culture apparatus whereupon the cells can be used immediately or stored for use at a later time

The culturing step as described herein (contact with agents as described herein) can be very short, for example less than 24 hours such as 1, 2, 3, 4, 5, 6, 7, 8, 9, 10, 11, 12, 13, 14, 15, 16, 17, 18, 19, 20, 21, 22, or 23 hours. The culturing step as described further herein (contact with agents as described herein) can be longer, for example 1, 2, 3, 4, 5, 6, 7, 8, 9, 10, 11, 12, 13, 14, or more days.

In some embodiments, cells may be cultured for several hours (about 3 hours) to about 14 days or any hourly integer value in between. Conditions appropriate for cell culture include an appropriate media (e.g., macrophage complete medium, DMEM/F12, DMEM/F12-10 (Invitrogen)) that may contain factors necessary for proliferation and viability, including serum (e.g., fetal bovine or human serum), L-glutamine, insulin, M-CSF, GM-CSF, IL-10, IL-12, IL-15, TGF-beta, and TNF-α or any other additives for the growth of cells known to the skilled artisan. Other additives for the growth of cells include, but are not limited to, surfactant, plasmanate, and reducing agents such as N-acetyl-cysteine and 2-mercaptoethanol. Media can include RPMI 1640, AIM-V, DMEM, MEM, α-MEM, F-12, X-Vivo 15, and X-Vivo 20, Optimizer, with added amino acids, sodium pyruvate, and vitamins, either serum-free or supplemented with an appropriate amount of serum (or plasma) or a defined set of hormones, and/or an amount of cytokine(s) sufficient for the growth and expansion of the cells. Antibiotics, e.g., penicillin and streptomycin, are included only in experimental cultures, not in cultures of cells that are to be infused into a subject. The target cells are maintained under conditions necessary to support growth, for example, an appropriate temperature (e.g., 37° C.) and atmosphere (e.g., air plus 5% CO₂).

The medium used to culture the cells may include an agent that can activate the cells. For example, an agent that is known in the art to activate the monocyte or macrophage is included in the culture medium.

### Therapy

In some embodiments, modified immune cells described herein may be included in a composition for treatment of a subject. In one aspect, the composition comprises the modified cell comprising the chimeric antigen receptor described herein. In some embodiments, a provided composition may include a pharmaceutical composition and further include a pharmaceutically acceptable carrier. In some embodiments, a therapeutically effective amount of the pharmaceutical composition comprising the modified immune cells may be administered.

In one aspect, the disclosure provides methods of treating a disease, disorder, or condition associated with a neurodegenerative disease/disorder, an inflammatory disease/disorder, a cardiovascular disease/disorder, a fibrotic disease/disorder or amyloidosis in a subject comprising administering to the subject a therapeutically effective amount of a pharmaceutical composition comprising modified immune cells as described herein. In another aspect, the disclosure provides methods for stimulating an immune response to a target a diseased/disordered cell or tissue in a subject comprising administering to a subject a therapeutically effective amount of a pharmaceutical composition comprising modified immune cells as described herein. In yet another aspect, the disclosure includes use of provided modified immune cells as described herein in the manufacture of a medicament for the treatment of an immune response in a subject in need thereof. In still another aspect, the disclosure includes use of provided modified immune cells as described herein in the manufacture of a medicament for the treatment of a neurodegenerative disease/disorder, an inflammatory disease/disorder, a cardiovascular disease/disorder, a fibrotic disease/disorder or amyloidosis in a subject in need thereof.

In one aspect, the disclosure provides a method of treating Alzheimer's Disease (AD) comprising administering to the subject a therapeutically effective amount of a pharmaceutical composition comprising modified immune cells as described herein.

In one aspect, the disclosure provides a method of treating Parkinson's Disease (AD) comprising administering to the subject a therapeutically effective amount of a pharmaceutical composition comprising modified immune cells as described herein.

In one aspect, the disclosure provides a method of treating a Tauopathy (e.g. Frontotemporal Dementia (FTLD-Tau), progressive supranuclear palsy, corticobasal degeneration) comprising administering to the subject a therapeutically effective amount of a pharmaceutical composition comprising modified immune cells as described herein.

In some embodiments, provided modified immune cells generated as described herein possess targeted effector activity. In some embodiments, provided modified immune cells have targeted effector activity directed against an antigen on a target cell, such as through specific binding to an antigen binding domain of a CAR. In some embodiments, targeted effector activity includes, but is not limited to, phagocytosis, targeted cellular cytotoxicity, antigen presentation, and cytokine secretion.

In some embodiments, modified immune cells described herein have the capacity to deliver an agent, for example, a biological agent or a therapeutic agent to the target. In some embodiments, an immune cell may be modified or engineered to deliver an agent to a target, wherein the agent is selected from the group consisting of a nucleic acid, an antibiotic, an anti-inflammatory agent, an antibody or antibody fragments thereof, a growth factor, a cytokine, an enzyme, a protein, a peptide, a fusion protein, a synthetic molecule, an organic molecule, a carbohydrate or the like, a lipid, a hormone, a microsome, a derivative or a variation thereof, and any combinations thereof. As a non-limiting example, a macrophage modified with a CAR that targets an antigen is capable of secreting an agent, such as a cytokine or antibody, to aid in macrophage function. Antibodies, such as anti-CD47/antiSIRPα mAb, may also aid in macrophage function. In yet another example, a macrophage modified with a CAR that targets an antigen (such as a protein in a protein aggregate such as α-synuclein or β-amyloid) is engineered to encode a siRNA that aids macrophage function by downregulating inhibitory genes (i.e. SIRPα). Another example, the CAR macrophage is engineered to express a dominant negative (or otherwise mutated) version of a receptor or enzyme that aids in macrophage function.

In some embodiments, a macrophage is modified with multiple genes, wherein at least one gene includes a CAR and at least one other gene comprises a genetic element that enhances CAR macrophage function. In some embodiments, a macrophage is modified with multiple genes, wherein at least one gene includes a CAR and at least one other gene aids or reprograms the function of other immune cells (such as T cells). In some embodiments, a macrophage is modified with multiple genes, wherein at least one gene includes a CAR and at least one other gene comprises a genetic element that enhances therapeutic efficacy. Therapeutic efficacy may be enhanced by a variety of genetic elements including, but not limited to, proteins that act by blocking checkpoint receptors, proteins that have immunostimulatory activity, proteins that have immunosuppressive/anti-inflammatory activity, and proteins that destabilize protein plaques.

Further, in some embodiments, modified immune cells can be administered to an animal, preferably a mammal, even more preferably a human, to treat a neurodegenerative disease/disorder, an inflammatory disease/disorder, a cardiovascular disease/disorder, a fibrotic disease, amyloidosis or any disease/disorder known in the art to be related to protein misfolding or protein aggregation. In some embodiments, the neurodegenerative disease/disorder comprises tauopathy, α-synucleopathy, presenile dementia, senile dementia, Alzheimer's disease, Parkinsonism linked to chromosome 17 (FTDP-17), progressive supranuclear palsy (PSP), Pick's disease, primary progressive aphasia, frontotemporal dementia, corticobasal dementia, Parkinson's disease, Parkinson's disease with dementia, dementia with Lewy bodies, Down's syndrome, multiple system atrophy, amyotrophic lateral sclerosis (ALS), Hallervorden-Spatz syndrome, polyglutamine disease, trinucleotide repeat disease, and/or prion disease. In addition, in some embodiments, cells of the present invention can be used for the treatment of any condition in which a diminished or otherwise inhibited immune response, especially a cell-mediated immune response, is desirable to treat or alleviate the disease/disorder. In one aspect, the invention includes treating a condition, such as a neurodegenerative disease/disorder, an inflammatory disease/disorder, a cardiovascular disease/disorder, a fibrotic disease, amyloidosis or any disease/disorder known in the art to be related to protein misfolding and/or protein aggregation, in a subject, comprising administering to the subject a therapeutically effective amount of a pharmaceutical composition comprising a population of the immune cells described herein. In addition, in some embodiments, modified immune cells described herein can be administered as pre-treatment or conditioning prior to treatment.

In some embodiments, provided modified immune cells can also be used to treat inflammatory diseases/disorders that comprises a protein in a protein aggregate, in a tissue of a subject. Examples of such inflammatory diseases/disorders include but are not limited to fibrotic diseases.

In some embodiments, modified immune cells can be administered in dosages and routes and at times to be determined in appropriate pre-clinical and clinical experimentation and trials. Cell compositions may be administered multiple times at dosages within these ranges. Administration of the modified immune cells may be combined with other methods useful to treat the desired disease/disorder or condition as determined by those of skill in the art.

In accordance with various embodiments, modified immune cells to be administered may be autologous, allogeneic, xenogeneic, or universal donor with respect to the subject undergoing therapy.

Administration of modified immune cells may be carried out in any convenient application-appropriate manner known to those of skill in the art. The modified immune cells may be administered to a subject by aerosol inhalation, injection, ingestion, transfusion, implantation or transplantation. The compositions described herein may be administered to a patient transarterially, subcutaneously, intradermally, intratumorally, intranodally, intramedullary, intramuscularly, by intravenous (i.v.) injection, intraperitoneally, or intracranially. In some embodiments, modified immune cells may be injected directly into a site of inflammation in the subject, a local disease site in the subject, a lymph node, an organ, a tumor, and the like.

### Alzheimer's Disease

In one aspect, the invention provides a method of treating Alzheimer's Disease (AD) comprising administering to the subject a therapeutically effective amount of a pharmaceutical composition comprising modified immune cells as described herein.

Alzheimer's Disease (AD) is the leading cause of dementia worldwide. Hallmarks of the disease include accumulation of protein aggregates (Amyloid Beta, Tau), cognitive decline, chronic inflammation, and brain atrophy. There is currently no curative treatment for AD and incidence of the disease is expected to double by 2050. Age and genetics are the major risk factors for AD.

AD is the sixth leading cause of death in the US. There is no current therapy to treat the underlying pathology of AD. The main pathology observed in the brains of AD patients is the accumulation of extracellular aggregates/plaques, composed of amyloid beta protein, including Aβ₄₂ (Sadigh-Eteghad et al. 2014. Medical Principles and Practice. 24 (1): 1-10). Beta amyloid protein can form plaques in other disease conditions such as other dementias (Lewy body) or muscle diseases.

While AD sufferers may exhibit any of a variety of signs or symptoms, common signs or symptoms include loss of memory (e.g., short term memory or long-term memory), inhibition of reasoning capacity, inhibition or loss of ability to make decisions, impaired planning ability, changes to personality, and/or altered behavior patterns (e.g., depression, mood swings, loss of inhibition, apathy, and withdrawal).

The symptoms of AD worsen over time, although the rate at which the disease progresses varies. On average, a subject with AD lives four to eight years after diagnosis, but can live as long as 20 years, depending on other factors. Changes in the brain related to AD begin years before any signs of the disease. This time period, which can last for years, is referred to as preclinical AD.

A subject in the early stage of AD (mild AD) may function independently, for example by driving, working and participating in social activities. In some embodiments, a subject suffering from early AD may feel as if he or she is having memory lapses, such as forgetting familiar words or the location of everyday objects. In some embodiments, friends, family or others close to a subject with early AD begin to notice difficulties. In some embodiments, a doctor performing a detailed medical interview with a subject suffering from early AD may be able to detect problems in memory or concentration. In some embodiments, a subject suffering from early stage AD experiences one or more difficulties selected from a group consisting of: problems coming up with the right word or name, trouble remembering names when introduced to new people, challenges performing tasks in social or work settings, forgetting material that one has just read, losing or misplacing a valuable object, and increasing trouble with planning or organizing.

The middle stage of AD (moderate AD) is typically the longest stage and can last for many years. As the disease progresses, a subject with AD will require a greater level of care. In some embodiments, a subject suffering from the middle stage of AD can experience symptoms selected from the group consisting of: confusing words, getting frustrated or angry, and acting in unexpected ways (e.g., refusing to bathe or other personality changes). Neurodegeneration in the brain of a subject with moderate AD can make it difficult for the subject to express thoughts and perform routine tasks. In some embodiments, symptoms in a subject suffering from the middle stage of AD will be noticeable to others outside of close family. In some embodiments, a subject suffering from the middle stage of AD experiences one or more difficulties selected from a group consisting of; and may include: forgetfulness of events or about the subject's own personal history, feeling moody or withdrawn, especially in socially or mentally challenging situations, being unable to recall the subject's own address or telephone number or the high school or college from which the subject graduated, confusion about where the subject is or what day it is, the need for help choosing proper clothing for the season or the occasion, trouble controlling bladder and bowels, changes in sleep patterns (e.g., sleeping during the day and becoming restless at night), an increased risk of wandering and becoming lost, personality and behavioral changes (e.g., suspiciousness and delusions) and compulsive, repetitive behavior (e.g., hand-wringing or tissue shredding).

In the final stage of AD (severe AD), a subject loses the ability to respond to his or her environment, to carry on a conversation and, eventually, to control movement. In some embodiments, a subject suffering from the final stage of AD may still say words or phrases, but communicating pain becomes difficult. In some embodiments, a subject suffering from the final stage of AD experiences significant personality changes. In some embodiments, a subject suffering from the final stage of AD needs extensive help with daily activities. In some embodiments, a subject suffering from the final stage of AD experiences one or more difficulties selected from a group consisting of: requiring round-the-clock assistance with daily activities and personal care, losing awareness of recent experiences as well as of surroundings, experiencing changes in physical abilities (e.g., the ability to walk, sit and, eventually, swallow), having increasing difficulty communicating, and becoming vulnerable to infections (e.g., pneumonia).

In some embodiments, a composition described herein is administered to a subject suffering from the early stage of AD. In some embodiments, a composition described herein is administered to a subject suffering from the middle stage of AD. In some embodiments, a composition described herein is administered to a subject suffering from the final stage of AD.

Microglia play a vital role in AD pathogenesis. Aging microglia exhibit reduced Aβ phagocytosis, reduced surveillance activity, increased reactivity, enhanced release of cytokines, and decreased secretion of neurotrophic factors. Microglia in AD exhibit reduced phagocytosis and digestion of Aβ (redistribution), and an induction of senescent-like phenotype.

The Amyloid Hypothesis presents Aβ as a therapeutic target. Impairment of amyloid metabolism and processing leads to Aβ accumulation. Aβ amyloidosis leads to neuroinflammation. Increased Aβ deposition may impact deposition of neurotoxic Tau leading to cognitive decline. Familial AD mutations in amyloid processing pathway support a role for Aβ in disease pathogenesis. Aducanumab, an amyloid beta-directed antibody, was recently approved to treat AD. However, monoclonal antibodies do not change the underlying physiology of aged or AD microglia. Other treatments for AD include acetyl cholinesterase inhibitors and the N-methyl-D-aspartate receptor antagonist Memantine, but offer symptomatic rather than disease-modifying benefits (Malik and Robertson. 2017. J Neurol 264:416-418).

Embodiments of the methods disclosed herein utilize microglia replacement as a therapeutic modality. Microglial surrogates (e.g. CAR macrophages) are developed that are safe for introduction into humans. Host microglia are depleted and microglial surrogates are introduced in a safe and timely fashion. Microglia surrogates (e.g. CAR macrophages) adopt microglia-like properties. This therapeutic replacement allows recovery of healthy functions, elimination of ‘toxic’ microglia, and cross-correction of lost enzymes.

In certain embodiments, endogenous microglia are replaced with anti-amyloid beta CAR macrophages. Disease pathology can be ameliorated by lowering amyloid beta burden and/or replacing dysfunctional microglia.

In some embodiments, methods described herein comprise treating a subject suffering from AD with a composition comprising modified immune cells comprising chimeric antigen receptors (CARs) as described herein. In some embodiments, treating a subject suffering from AD comprises administering a CAR-based therapeutic composition as described herein alone or in combination with an additional (non-CAR) therapeutic composition. In some embodiments, treating a subject suffering from AD with only a CAR-based therapeutic composition as described herein has a greater effect on the AD symptoms and/or pathology of the subject than treating a subject suffering from AD with only the additional (non-CAR) therapeutic composition. In some embodiments, treating a subject suffering from AD with a combination of a CAR-based therapeutic composition as described herein and an additional therapeutic composition has a synergistic effect on the AD symptoms and/or pathology of the subject. In some embodiments, the additional therapeutic composition comprises a human monoclonal antibody that selectively targets aggregated Aβ. In some embodiments, the human monoclonal antibody that selectively targets aggregated Aβ is aducanumab. In some embodiments, the additional therapeutic composition comprises a selective inhibitor of tau protein aggregation. In some embodiments, the selective inhibitor of tau protein aggregation is Leuco-methylthioninium bishydromethanesulfonate (LMTM).

In some embodiments, the effect of an AD treatment on AD symptoms in a subject is evaluated using the Alzheimer's Disease Assessment Scale-Cognitive Subscale (ADAS-Cog) and/or the AD Co-operative Study-Activities of Daily Living Inventory (ADCS-ADL). In some embodiments, the effect of an AD treatment on AD pathology in a subject is evaluated by measuring the level of protein aggregates in the brain of the subject. In some embodiments, protein aggregates are aggregated Aβ (e.g., Aβ₄₂). In some embodiments, protein aggregates are tau protein aggregates.

### Amyloid/Amyloidosis

Amyloidosis is a term used to describe a group of diseases with the common pathological feature of abnormal build-up of incorrectly folded proteins known as amyloid or amyloid fibrils (Chiti and Dobson. 2006. Ann Review Biochem, 75: 333-366; Sipe et al., Amyloid 21(4): 221-224). An amyloid fibril is an insoluble protein complex deposited extracellularly as a result of misfolding of a soluble precursor protein (Nienhuis et al., Kidney Dis (Basel). 2016 April; 2(1): 10-19). The formation of the amyloid fibrils is the result of oligomerization and aggregation of the defective proteins. Amyloidosis can be localized or systemic and can be classified, according to one approach, in 6 groups: Primary Amyloidosis (AL), in which the amyloid fibrils are made up of immunoglobulin light chain proteins; Secondary Amyloidosis (AA), in which the source of amyloid is the serum amyloid A (SAA) as a result of inflammation; Familial Amyloidosis (ATTR), typically a result of a mutated transthyretin protein; Other Familial Amyloidoses with misfolding of different proteins leading to the disease pathology; Beta-2 Microglobulin Amyloidosis, where pathologic aggregates are made up of beta-2 microglobulin protein; and Localized Amyloidosis, associated with a variety of proteins in different tissues and organs (Boston University Amyloidosis Center).

Immunoglobulin light chain amyloidosis (AL) is a multisystem, lethal disorder characterized by organ dysfunction caused by the deposition of amyloid fibrils derived from an underlying plasma cell neoplasm. AL is a rare condition diagnosed in ˜3,000 patients in the US annually. The current treatment approach is chemotherapy directed to plasma cells and therefore is non-specific. The side-effects of this kind of treatment are associated with high early mortality (about one-third die within the first year) as well as delayed and incomplete clearance of the amyloid deposits from organs. This leaves patients with chronic morbidity from heart failure, nephrotic syndrome and disabling neuropathies along with ongoing risk of death. In order to improve outcomes in AL, therapies beyond the plasma cell are needed, in particular, clearance of deposited amyloid light chains. Herein, a treatment platform was developed based on macrophages expressing a chimeric antigen receptor (CAR macrophages) that identifies organ deposits of amyloid and clears the deposits through phagocytosis, which may lead to improvement in organ function, reduced morbidity and improved survival in AL. This treatment platform can be extended to additional types of amyloidosis as well as other diseases associated with extracellular deposits of misfolded protein.

Types of amyloidosis include but are not limited to Heavy Chain Amyloidosis (AH), primary systemic amyloidosis, ApoAI Amyloidosis, ApoAII Amyloidosis, ApoAIV Amyloidosis, Apolipoprotein C2 Amyloidosis, and Apolipoprotein C3 Amyloidosis, Corneal lactoferrin amyloidosis, Transthyretin-Related Amyloidosis, Dialysis amyloidosis, Fibrinogen amyloidosis, Lect2 amyloidosis (ALECT2), and Lysozyme amyloidosis.

Examples of amyloid-associated disease include, but are not limited to, Alzheimer's disease, where an aggregation of Tau protein and beta-amyloid is observed; spongiform encephalopathies (prion diseases), where mutated prion proteins make up the toxic aggregates; cataracts, caused by aggregation of the protein crystallin; type 2 diabetes, with aggregates made from amylin and others (Caughey and Lansbury. 2003. Annu Rev Neurosci. 26:267-98; Valastyan and Lindquist, Disease Models & Mechanisms (2014) 7, 9-14).

Proteins implicated in amyloidosis include, but are not limited to, serum amyloid A (SAA) protein, monoclonal immunoglobulin light proteins (kappa or lambda), immunoglobulin heavy chain proteins, transthyretin protein, apolipoprotein A-I (AApoAI), apolipoprotein A-II (AApoAII), apolipoprotein A-IV (AApoAIV), apolipoprotein C2 (ApoC2), apolipoprotein C3 (ApoC3), keratin, amyloid Dan (ADan), lactoferrin, gelsolin (AGel or GSN), fibrinogen (AFib), fibrinogen alpha chain (FGA), lysozyme (ALys or LYZ), Lect2, beta-2 microglobulin, amyloid beta, crystallin, amylin (islet amyloid peptide), prion protein (PrP), leukocyte cell derived chemotaxin 2 (LECT2), cystatin C (CST3), oncostatin M receptor (OSMR), integral membrane protein 2B (ITM2b), prolactin (PRL), keratoepithelin and atrial natriuretic factor (ANF). Amyloidosis may present via any of a variety of signs or symptoms including, but not limited to, swelling of extremities, especially ankles and/or legs, fatigue, shortness of breath, weight loss, irregular heartbeat, numbness in hands or feet, tingling or pain in hands or feet, and/or shortness of breath. These clinical manifestations reflect involvement of most major organ systems but particularly heart, kidneys, and/or peripheral nerves.

In some embodiments, provided compositions may be used with one or more other treatments for amyloidosis including, but not limited to chemotherapy, stem cell therapy, anti-inflammatory agents, or myeloma-directed therapy such as proteasome inhibitors among others.

The compositions and methods disclosed herein may comprise an antibody, antibody agent, and/or other antigen binding domain against common epitopes of amyloid aggregates or epitopes that are distinct for different misfolded proteins contributing to the formation of an amyloid fibril, herein referred to as “amyloid”. The term amyloid includes naturally occurring human amino acid sequences both wild type and mutated as well as fragments, analogs including allelic, species and induced variants. Amino acids of analogs are assigned the same numbers as corresponding amino acids in the natural human sequence when the analog and human sequence are maximally aligned. Analogs typically differ from naturally occurring peptides at one, two or a few positions, often by virtue of conservative substitutions. The term “allelic variant” is used to refer to variations between genes of different individuals in the same species and corresponding variations in proteins encoded by the genes. Anti-amyloid antibodies, their fragments, and analogs can be synthesized by solid phase peptide synthesis or recombinant expression, or can be obtained from natural sources. Automatic peptide synthesizers are commercially available from numerous suppliers, such as Applied Biosystems, Foster City, Calif.

### Pharmaceutical Compositions

In some embodiments, pharmaceutical compositions described herein may comprise modified immune cells as described herein, in combination with one or more pharmaceutically or physiologically acceptable carriers, diluents or excipients. Such compositions may comprise buffers such as neutral buffered saline, phosphate buffered saline and the like; carbohydrates such as glucose, mannose, sucrose or dextrans, mannitol; proteins; polypeptides or amino acids such as glycine; antioxidants; chelating agents such as EDTA or glutathione; adjuvants (e.g., aluminum hydroxide); and preservatives. Compositions of the present invention are preferably formulated for intravenous administration.

Pharmaceutical compositions described herein may be administered in a manner appropriate to the disease/disorder to be treated (or prevented). The quantity and frequency of administration will be determined by such factors as the condition of the patient, and the type and severity of the patient's disease/disorder, although appropriate dosages may be determined by clinical trials.

When “an immunologically effective amount”, “an anti-immune response effective amount”, “an immune response-inhibiting effective amount”, or “therapeutic amount” is indicated, the precise amount of the compositions of the present invention to be administered can be determined by a physician with consideration of individual differences in age, weight, immune response, and condition of the patient (subject). It can generally be stated that a pharmaceutical composition comprising the cells described herein may be administered at a dosage of 10⁴ to 10⁹ cells/kg body weight, preferably 10⁵ to 10⁶ cells/kg body weight, including all integer values within those ranges. The cell compositions described herein may also be administered multiple times at these dosages. The cells can be administered by using infusion techniques that are commonly known in immunotherapy (see, e.g., Rosenberg et al., New Eng. J. of Med. 319:1676, 1988). The optimal dosage and treatment regime for a particular patient can readily be determined by one skilled in the art of medicine by monitoring the patient for signs of disease/disorder and adjusting the treatment accordingly.

In certain embodiments, it may be desired to administer monocytes, macrophages, or dendritic cells to a subject and then subsequently redraw blood (or have an apheresis performed), activate the monocytes, macrophages, or dendritic cells therefrom according to the present invention, and reinfuse the patient with these activated cells. This process can be carried out multiple times every few weeks. In certain embodiments, the cells can be activated from blood draws of from 10 ml to 400 ml. In certain embodiments, the cells are activated from blood draws of 20 ml, 30 ml, 40 ml, 50 ml, 60 ml, 70 ml, 80 ml, 90 ml, or 100 ml. Not to be bound by theory, using this multiple blood draw/multiple reinfusion protocol, may select out certain populations of cells.

In certain embodiments, cells are modified using the methods described herein, or other methods known in the art where the cells are expanded to therapeutic levels, are administered to a patient in conjunction with (e.g., before, simultaneously or following) any number of relevant treatment modalities, In an additional embodiment, the cells may be administered before or following a surgery.

The dosage of the above treatments to be administered to a subject will vary with the precise nature of the condition being treated and the recipient of the treatment. The scaling of dosages for human administration can be performed according to art-accepted practices. The dose for CAMPATH antibody, for example, will generally be in the range 1 to about 100 mg for an adult patient, usually administered daily for a period between 1 and 30 days. The preferred daily dose is 1 to 10 mg per day although in some instances larger doses of up to 40 mg per day may be used (described in U.S. Pat. No. 6,120,766).

It should be understood that the method and compositions that would be useful in the present invention are not limited to the particular formulations set forth in the examples. The following examples are put forth so as to provide those of ordinary skill in the art with a complete disclosure and description of how to make and use the cells, expansion and culture methods, and therapeutic methods of the invention, and are not intended to limit the scope of what the inventors regard as their invention.

The practice of the present invention employs, unless otherwise indicated, conventional techniques of molecular biology (including recombinant techniques), microbiology, cell biology, biochemistry and immunology, which are well within the purview of the skilled artisan. Such techniques are explained fully in the literature, such as, “Molecular Cloning: A Laboratory Manual”, fourth edition (Sambrook, 2012); “Oligonucleotide Synthesis” (Gait, 1984); “Culture of Animal Cells” (Freshney, 2010); “Methods in Enzymology” “Handbook of Experimental Immunology” (Weir, 1997); “Gene Transfer Vectors for Mammalian Cells” (Miller and Calos, 1987); “Short Protocols in Molecular Biology” (Ausubel, 2002); “Polymerase Chain Reaction: Principles, Applications and Troubleshooting”, (Babar, 2011); “Current Protocols in Immunology” (Coligan, 2002). These techniques are applicable to the production of the polynucleotides and polypeptides of the invention, and, as such, may be considered in making and practicing the invention. Particularly useful techniques for particular embodiments will be discussed in the sections that follow.

### EXPERIMENTAL EXAMPLES

The invention is further described in detail by reference to the following experimental examples. These examples are provided for purposes of illustration only, and are not intended to be limiting unless otherwise specified. Thus, the invention should in no way be construed as being limited to the following examples, but rather, should be construed to encompass any and all variations which become evident as a result of the teaching provided herein.

Without further description, it is believed that one of ordinary skill in the art can, using the preceding description and the following illustrative examples, make and utilize the compounds/cells of the present invention and practice the claimed methods. The following examples therefore, specifically point out the preferred embodiments of the present invention, and are not to be construed as limiting in any way the remainder of the disclosure.

### Example 1: Replacement of Endogenous Microglia with Chimeric Antigen Receptor (CAR) Microglia-Like Cells can Recognize and Internalize Neurodegenerative Pathology

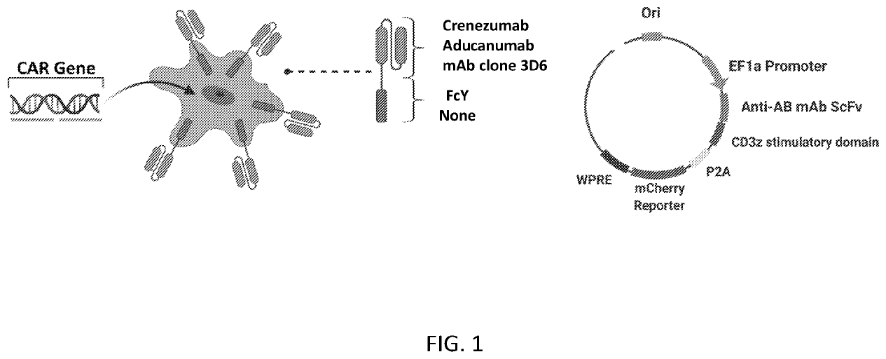

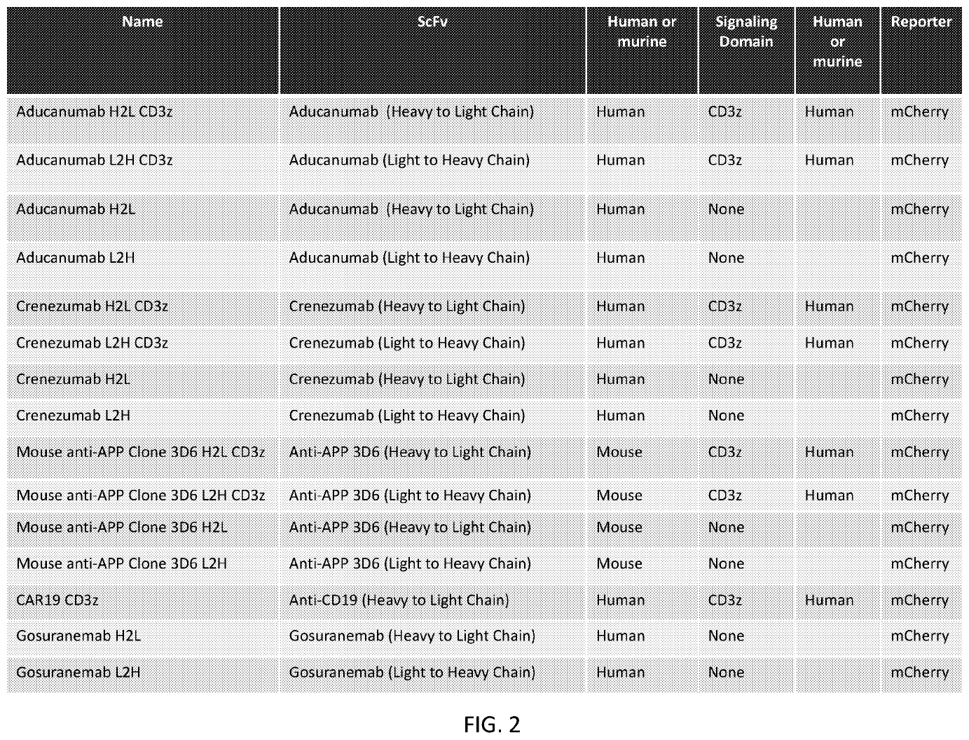

Microglia-like cell-based therapies were developed herein. Chimeric antigen receptors (CARs) were designed to contain antigen binding domains specific for Amyloid Beta (Aβ) or Tau. Anti-Aβ or anti-Tau scFvs generated using commercially available monoclonal antibodies (Aducanumab, Crenezumab, and Gosuranemab) were cloned into a lentiviral vector (FIGS. 1-2). A portion of the CAR constructs were designed to contain a CD3 zeta costimulatory domain, while in other CAR constructs, the costimulatory domain was absent (FIGS. 1-2).

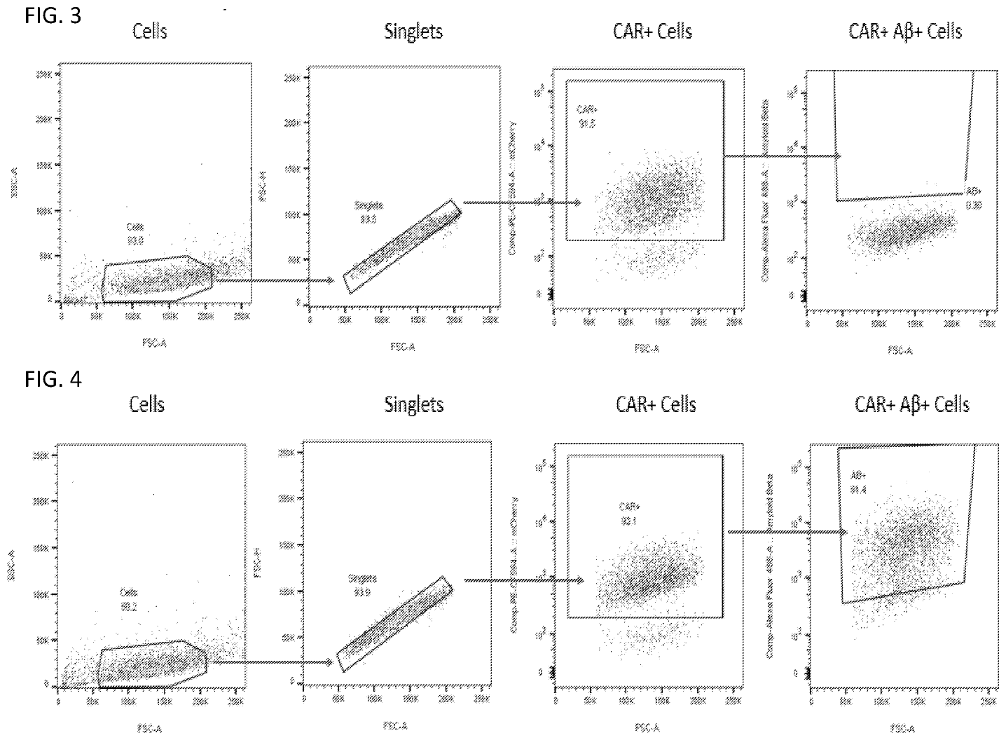

Human Microglial Fetal Cell Lines (HMC3) were developed by transducing cells with lentivirus containing various CAR constructs and sorting cells for CAR expression. mCherry is a reporter gene in the CAR constructs. 10,000 CAR expressing HMC3 cells were incubated in media containing fluorescently-labeled (AF488) amyloid beta (Aβ 1-42) at various concentrations for 1 hour at 37° C. Media was washed off and Aβ uptake was determined via flow cytometry (AF488 positivity) (FIGS. 3-4). Aβ uptake was measured in two ways: 1.) % CAR+ cells with Aβ (AF488+) and 2.) weighted average of Aβ (AF488+) intensity was calculated by multiplying the fraction of CAR+ cells that were Aβ+ with the MFI of CAR+ cells at the AF488 channel.

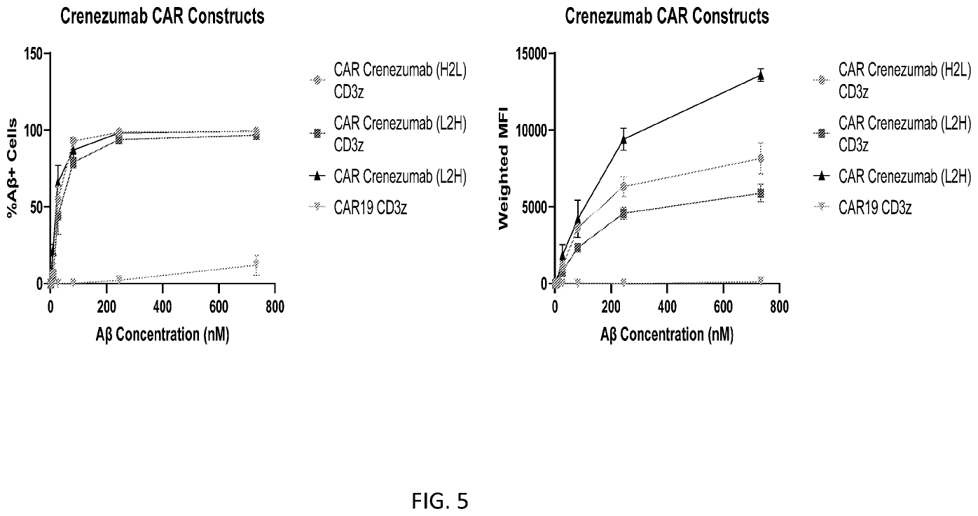

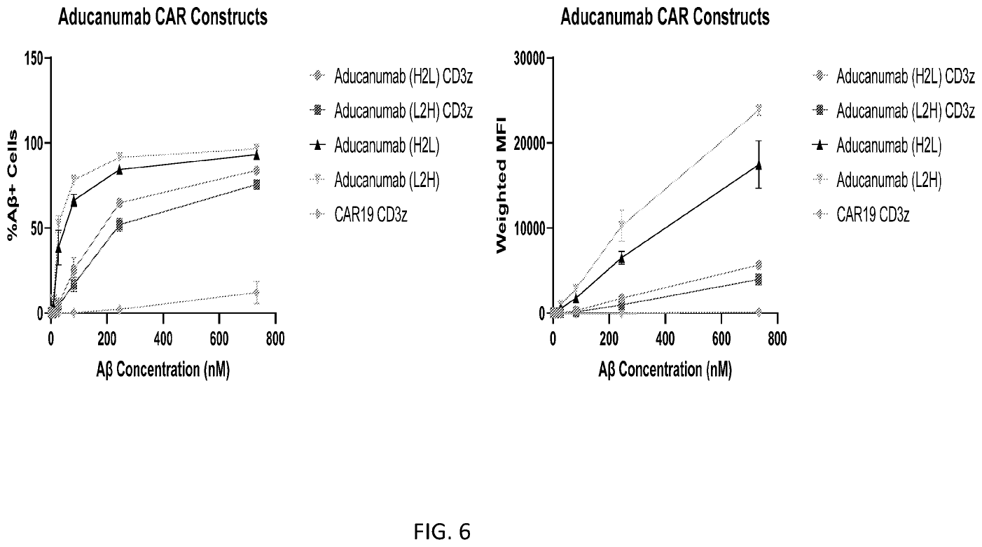

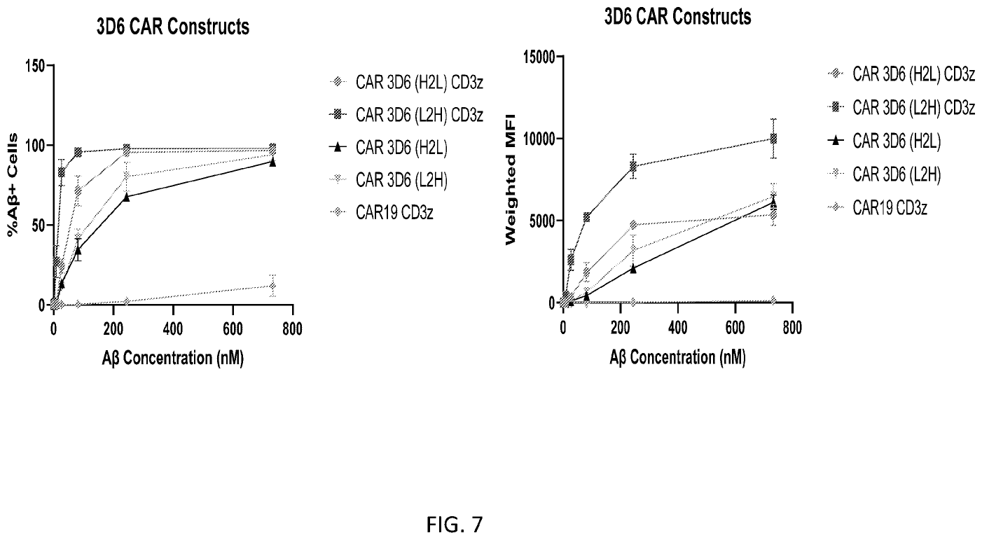

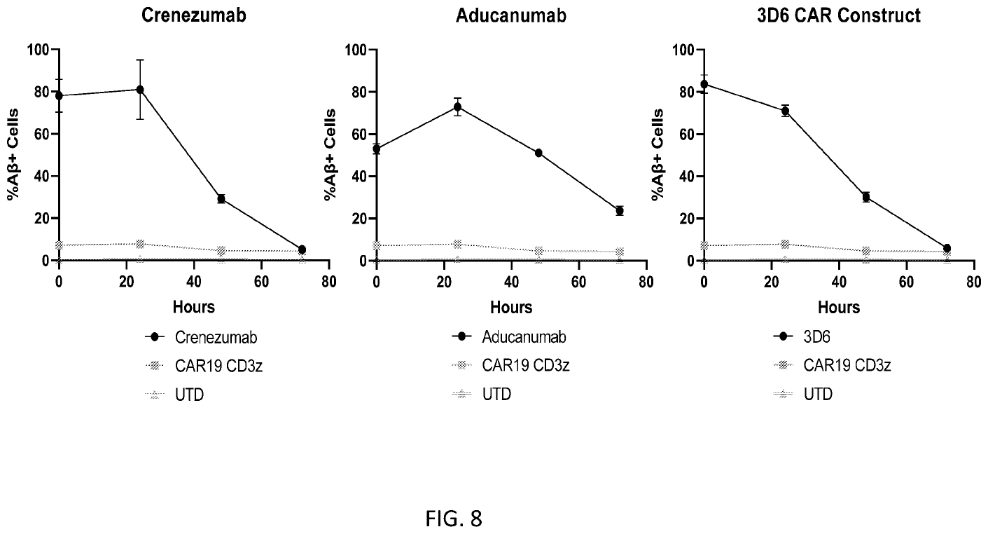

Results showed that anti-Aβ Crenezumab constructs allowed for specific uptake of amyloid beta compared to nonspecific CAR19 CD3z (FIG. 5). Lack of signaling domain in Aβ uptake occurred in the presence and absence of intracellular signaling domain in anti-Aβ crenezumab constructs (FIG. 5). Anti-Aβ Aducanumab constructs also allowed for specific uptake of amyloid beta compared to nonspecific CAR19 CD3z (FIG. 6). Lack of signaling domain in Aβ uptake occurred in the presence and absence of intracellular signaling domain in anti-Aβ Aducanumab constructs (FIG. 6). Anti-Aβ clone 3D6 constructs also allowed for specific uptake of amyloid beta compared to nonspecific CAR19 CD3z (FIG. 7). Lack of signaling domain in Aβ uptake occurred in the presence and absence of intracellular signaling domain in anti-Aβ clone 3D6 constructs (FIG. 7). 100,000 CAR expressing HMC3 cells were incubated in media containing fluorescently-labeled (AF488) amyloid beta (Aβ 1-42) at a concentration of 82 nM for 1 hour at 37° C. Media was washed off and Aβ uptake was determined via flow cytometry (AF488 positivity) at the following timepoints (0 hr, 24 hr, 48 hr, 72 hr). Aβ signal was measured via gating on % CAR+ cells with Aβ (AF488+). The same gating strategy as in previous experiments was used (FIGS. 3-4). Results showed Aβ fluorescence signal decreases after 24 hrs in Anti-Aβ CAR-HMC3 cells (FIG. 8). Anti-Aβ CAR constructs are significantly different From UTD and CAR19 CD3z at all concentrations (except Crenezumab & 3D6 at 72 hrs) (FIG. 8).

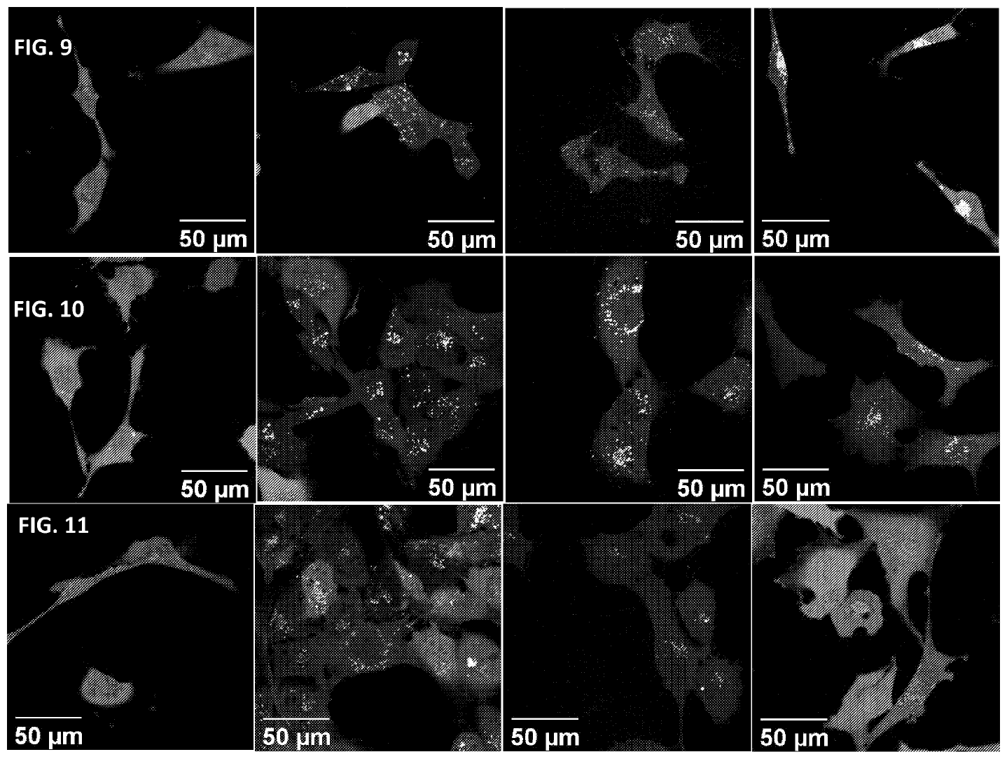

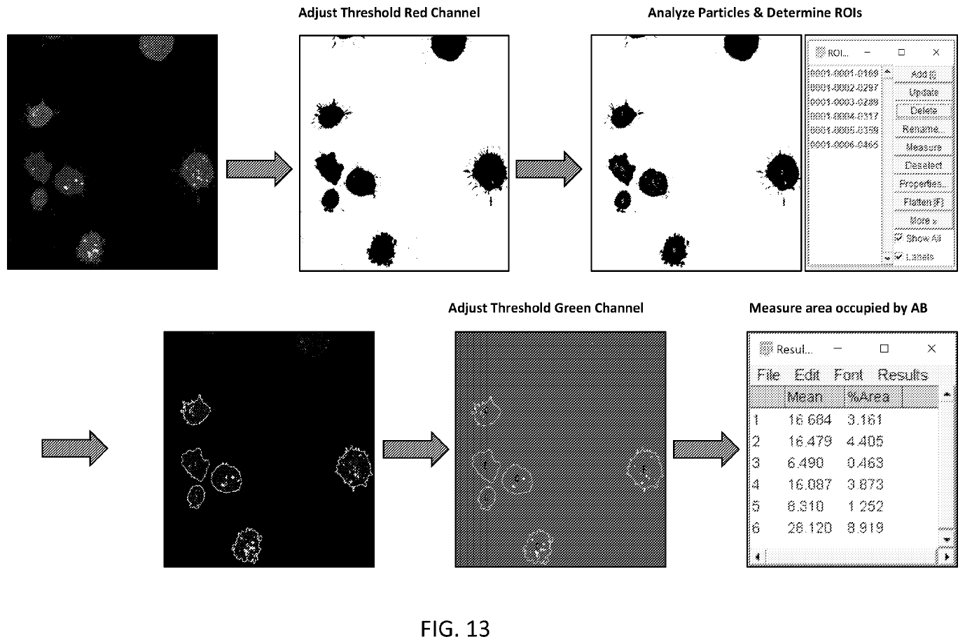

20,000 CAR-HMC3 cells were plated in a 27 mm2 Nunc dish. Cells were exposed to media with fluorescently-labeled amyloid beta at a concentration of 82 nM for 1 hr at 37° C. Amyloid beta media was washed off and cells were visualized via confocal microscopy at the following timepoints post-Aβ media removal: 0 hr, 24 hr, 48 hr. Analysis was conducted from 10 pictures per dish on the middle slice of the z-sections via segmentation algorithm on FIJI to determine % Area covered by fluorescent Aβ particles (FIG. 13). Confocal Data corroborates superior Aβ uptake by anti-Aβ CAR construct HMC3 cells (FIGS. 9-11). All images were obtained at the same laser settings at all time points. Representative Images (0 hr post Aβ Removal)). Representative images 0, 24, and 48 hours post Aβ removal are shown in FIG. 9, FIG. 10, and FIG. 11, respectively.

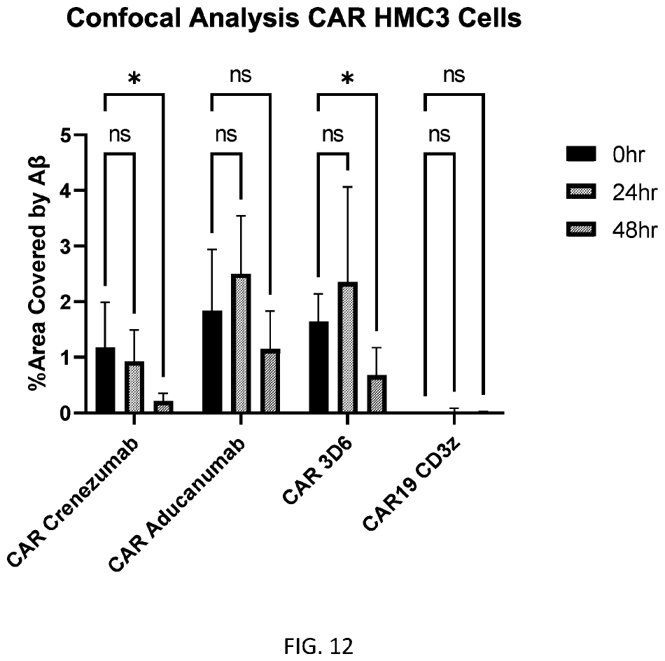

Confocal analysis supports the trend observed by flow cytometry that Aβ decreases over time (FIG. 12). The trend observed was that amyloid beta signal increases or stays unchanged during first 24 hours due to potential redistribution during cell division. At the 48 hour time point statistically significant differences were observed compared to the 24 hour timepoint suggesting Aβ degradation by cells expressing anti-Aβ CARs lacking the signaling domain (FIG. 12). Statistical analysis performed was a Two Way ANOVA.

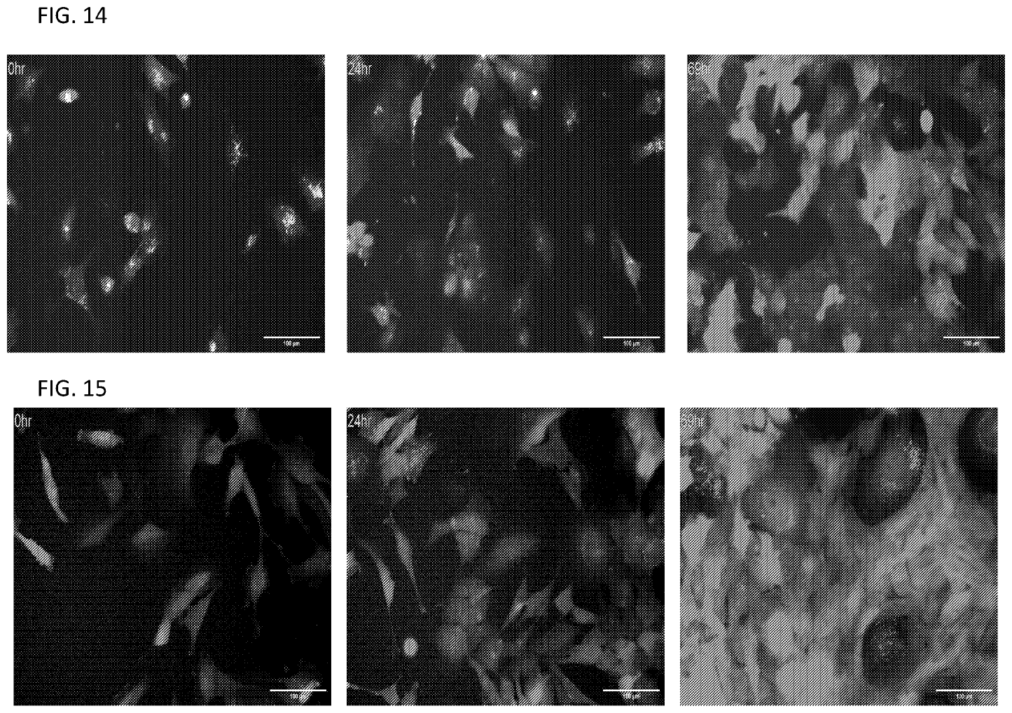

Live cell imaging was performed. 100,000 cells per well were incubated with Aβ media @ 37° C. for 1 hour, and amyloid media removed afterwards. Widefield microscopy was performed using 30 mins picture intervals (red & green channels) at 10× magnification. Constructs tested were Aducanumab H2L and CAR19 CD3z. Results showed Anti-Aβ Aducanumab (H2L) CAR HMC3 cells uptake fluorescently-labeled Aβ (FIG. 14). Aβ signal decreased over the 48 hour time period (FIG. 14). In contrast, no significant Aβ uptake by CAR19 CD3z bearing HMC3 cells was observed (FIG. 15).

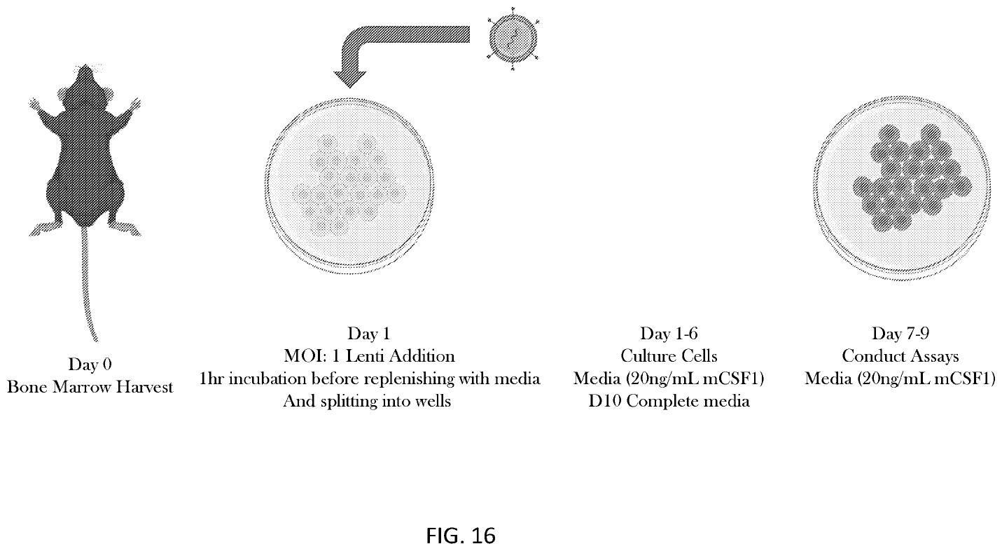

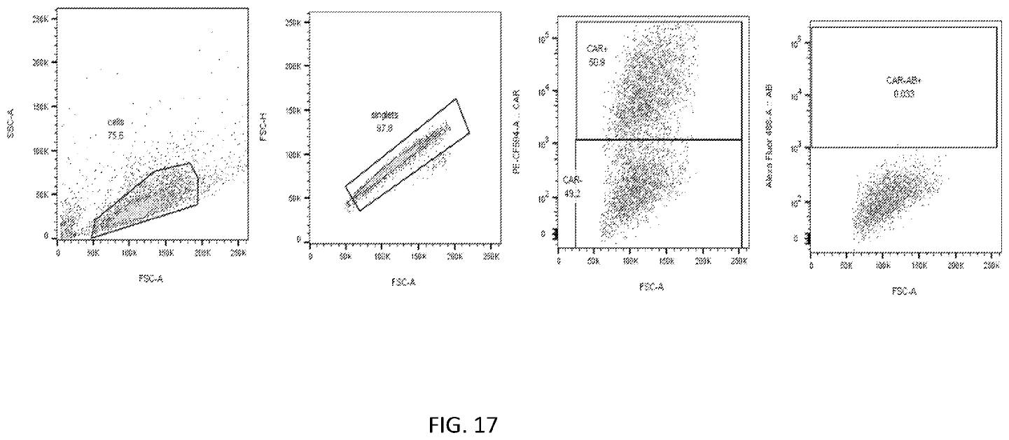

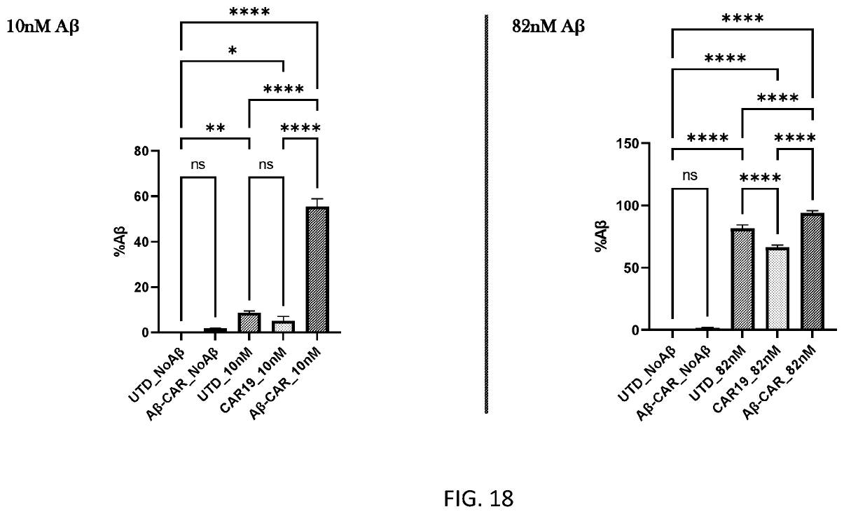

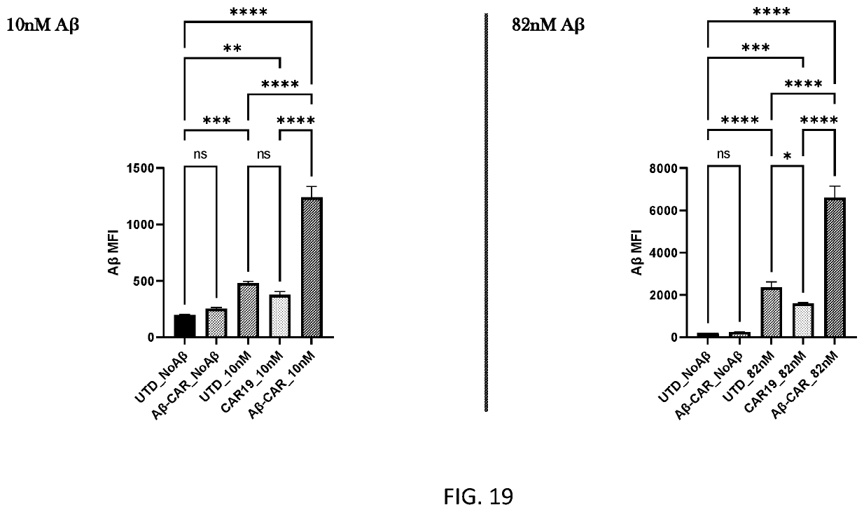

Schematic of protocol to express CAR constructs in murine bone marrow derived macrophages(BMDM) (FIG. 16). Anti-Aβ CAR murine BMDM Aβ flow assays were set up as follows: 100,000 cells per well (Murine CAR murine BMDM) were incubated in media containing fluorescently-labeled Aβ at a concentration of either 10 nM or 82 nM for 1 hr at 37 C. Amyloid beta media was washed off and Aβ uptake was measured via flow cytometry. Aβ uptake is measured in two ways: 1.) % CAR+ cells with Aβ (AF488+) and 2.) weighted average of Aβ (AF488+) as previously described by taking into account intensity by fraction of CAR+ cells that were Aβ+. A gating strategy (cells→singlets→CAR+/CAR-(mCherry)→Aβ+(AF488)) was used, demonstrating CAR gating and no Aβ uptake in no Aβ control, defining Aβ uptake threshold (FIG. 17). Results showed that Anti-Aβ CAR murine BMDMs had superior Aβ association compared to nonspecific CARs & untransduced cells as determined by % Aβ+ cells (FIG. 18). Anti-Aβ CAR murine BMDMs also had greater weighted Aβ MFI association compared to nonspecific CARs & untransduced cells (FIG. 19).

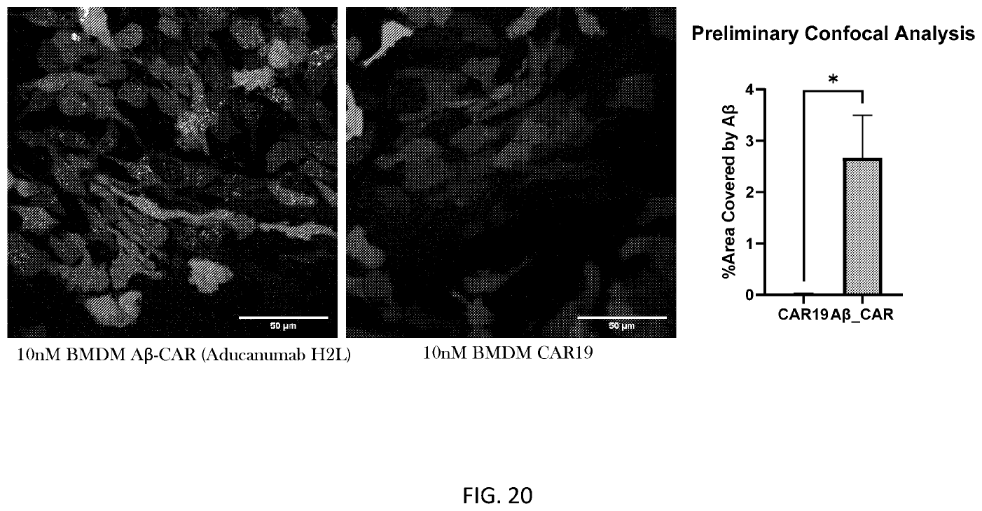

A BMDM confocal study was set up as follows: 200,000 cells were placed in each nunc dish and incubated with Aβ media (10 nM) for 1 hr @ 37° C. Media was washed off and cells visualized immediately. Anti-Aβ CAR murine BMDMs demonstrated superior Aβ internalization compared to nonspecific CAR murine BMDMs (FIG. 20). This validated that Anti-Aβ CARs drive specific uptake of Aβ compared to the CAR19 construct in primary murine macrophages (FIG. 20). CAR construct tested: Aducanumab H2L vs CAR19 CD3z. Statistical Analysis: Welch's t test.

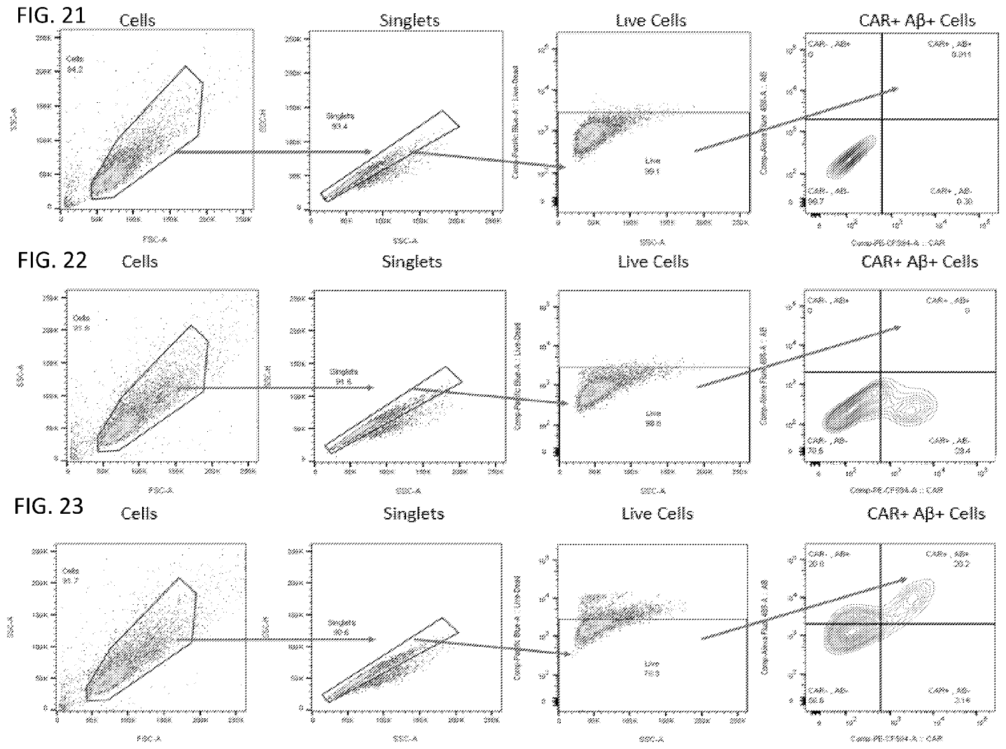

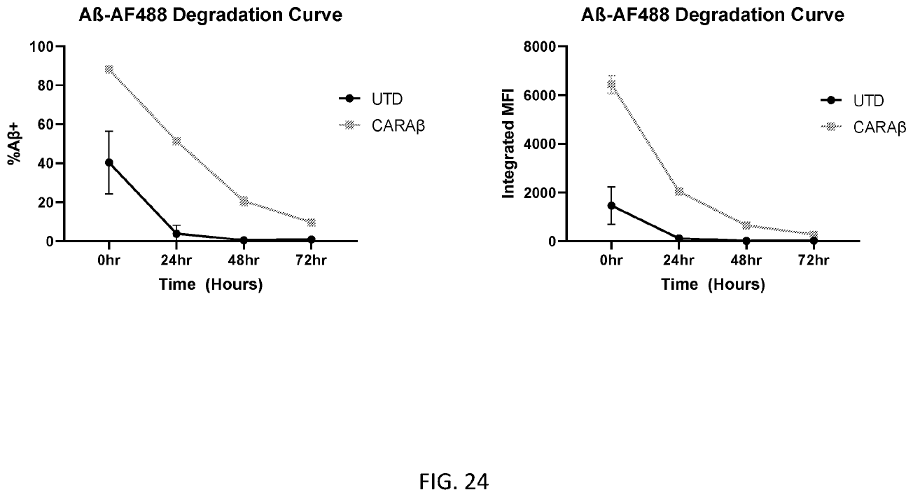

Flow Aβ degradation experiments were set up as follows: 200,000 CAR murine bone marrow derived macrophages were plated per well. Cells were exposed to media with fluorescently-labeled Aβ at a concentration of 40 nM. Media was washed off and Aβ signal was detected via flow cytometry. Constructs Tested: CAR Aducanumab (H2L) and untransduced. A gating strategy was used demonstrating that CAR− cells that are not exposed to Aβ are also Aβ− (cells→singlets→live cells→CAR+Aβ+) (FIG. 21). A gating strategy was used showing CAR+ cells without Aβ (FIG. 22) (cells→singlets→live cells→CAR+Aβ+). A gating strategy was used demonstrating that anti-Aβ (Aducanumab H2L) CAR bearing BMDMs uptake fluorescently-labeled Aβ at a higher proportion than untransduced BMDMs (FIG. 23) (cells→singlets→live cells→CAR+Aβ+). Evaluation of Aβ degradation via flow cytometry showed that Anti-Aβ (Aducanumab H2L) bearing CAR murine BMDMs uptake higher proportions of Aβ than untransduced BMDMs and are capable of degrading Aβ over time (FIG. 24). 200,000 bone marrow derived macrophages were plated per well in a 48 well plate. Cells were exposed to media containing 40 nM of fluorescently-labeled Aβ (AF488) for 1 hr at 37° C. Unbound amyloid beta was washed off and cells were analyzed via flow cytometry for amyloid beta signal (AF488) and CAR (mCherry) signal over time (0 hr, 24 hr, 48 hr, 72 hr).

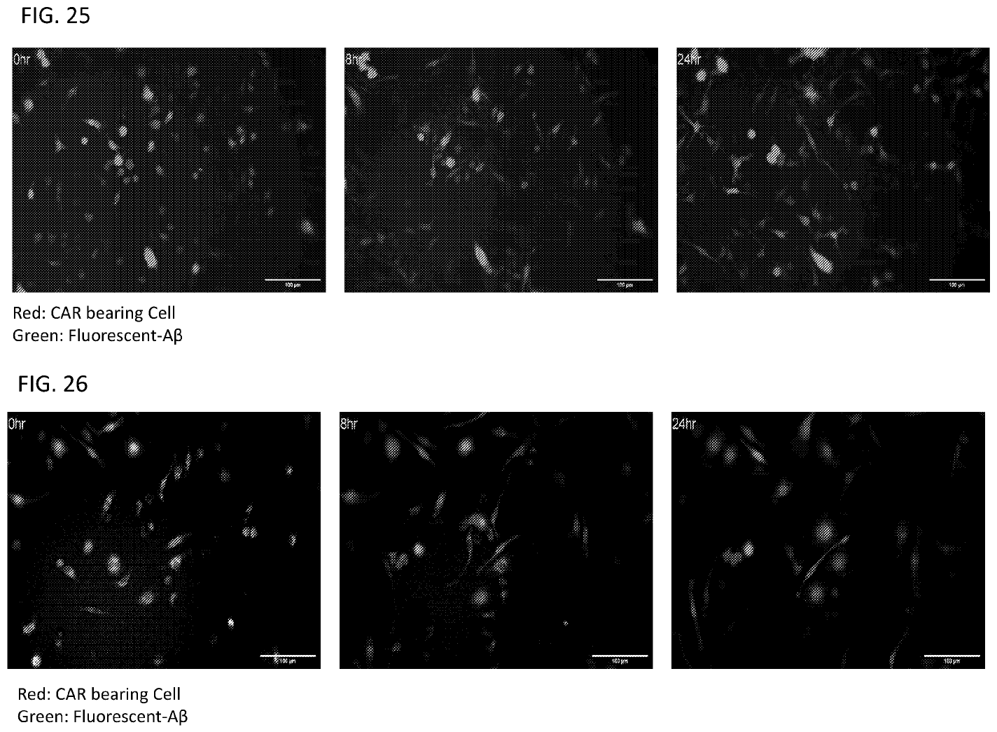

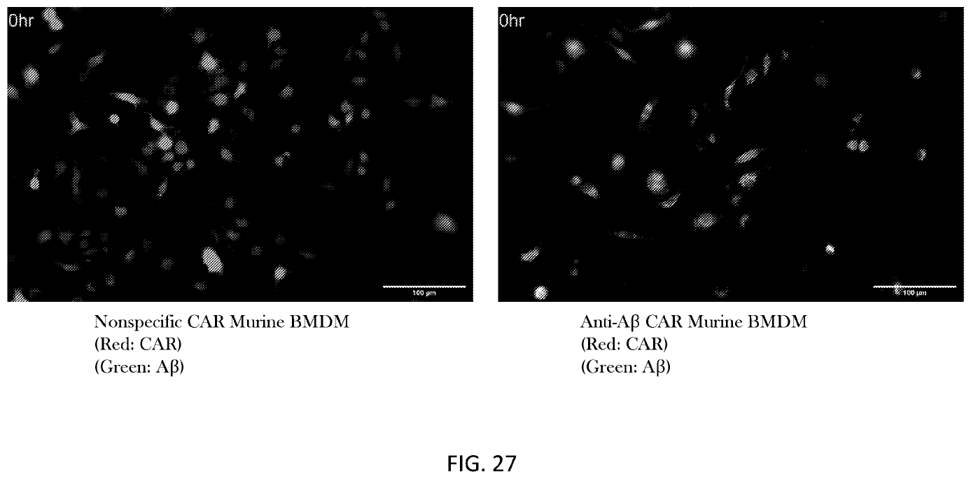

Live cell imaging (murine BMDM) was set up as follows: 100,000 cells per well were incubated with Aβ Media (10 nM) for 1 hr @ 37° C., and analyzed by widefield microscopy with images captured every 30 mins (red & green channel). The red channel is used to detect CAR expression via the mCherry reporter gene, and the green channel was used to detect amyloid beta via the AF488 conjugated fluorophore. The Aβ fluorescent signal was eliminated in 24 hours by anti-Aβ CAR murine BMDMs (FIG. 26). Fluorescent Aβ was not detectable in nonspecific CAR murine BMDMs (FIG. 25). Live cell imaging shows that murine BMDM CAR19 bearing cells do not uptake Aβ (FIG. 27).

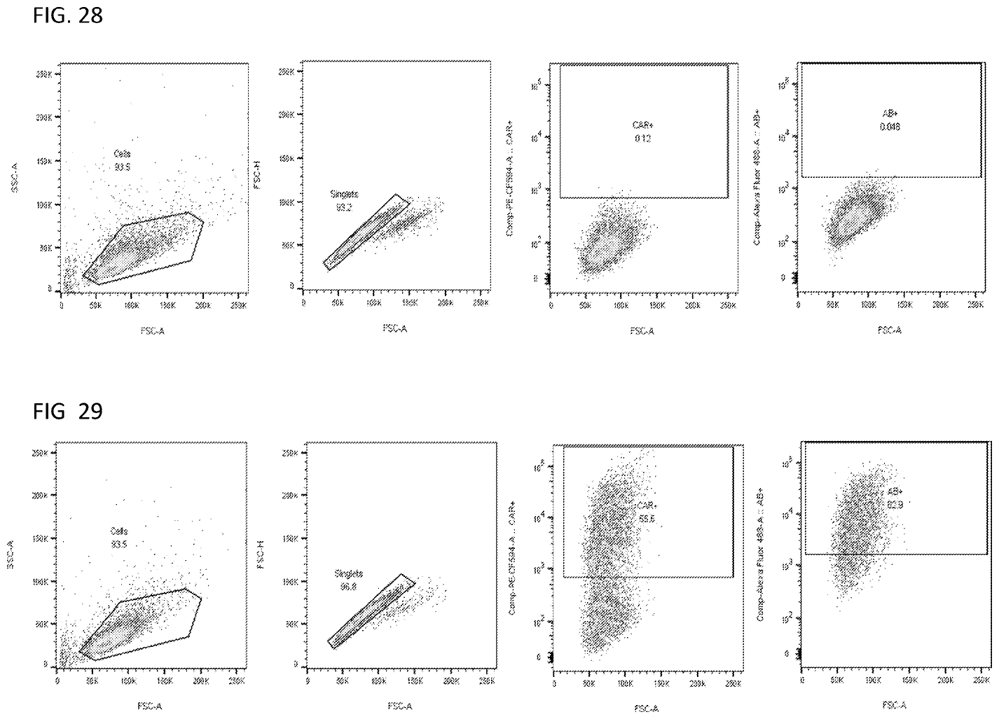

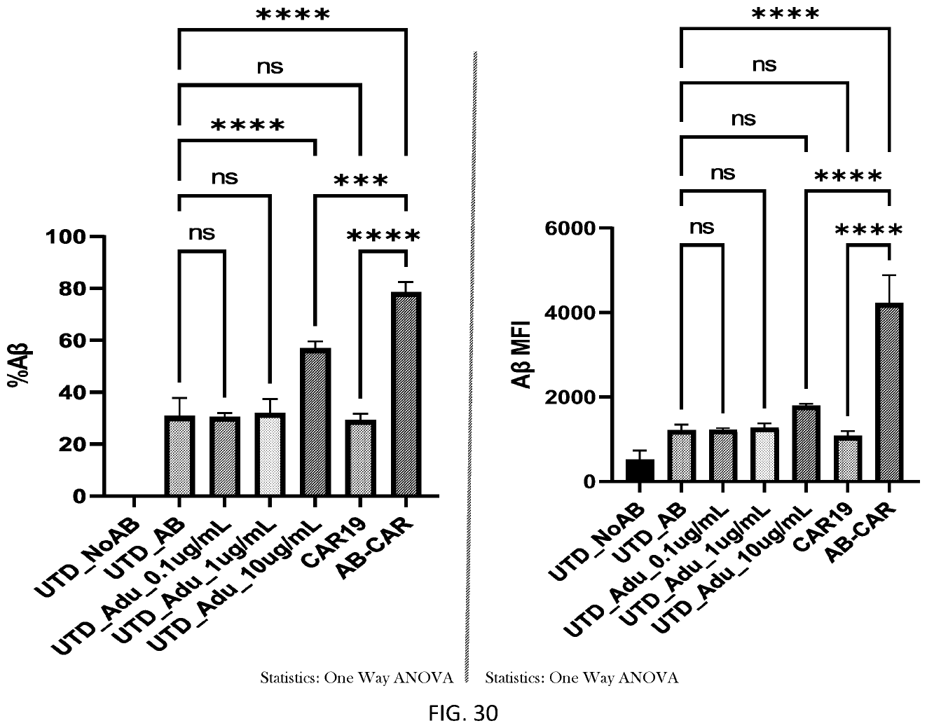

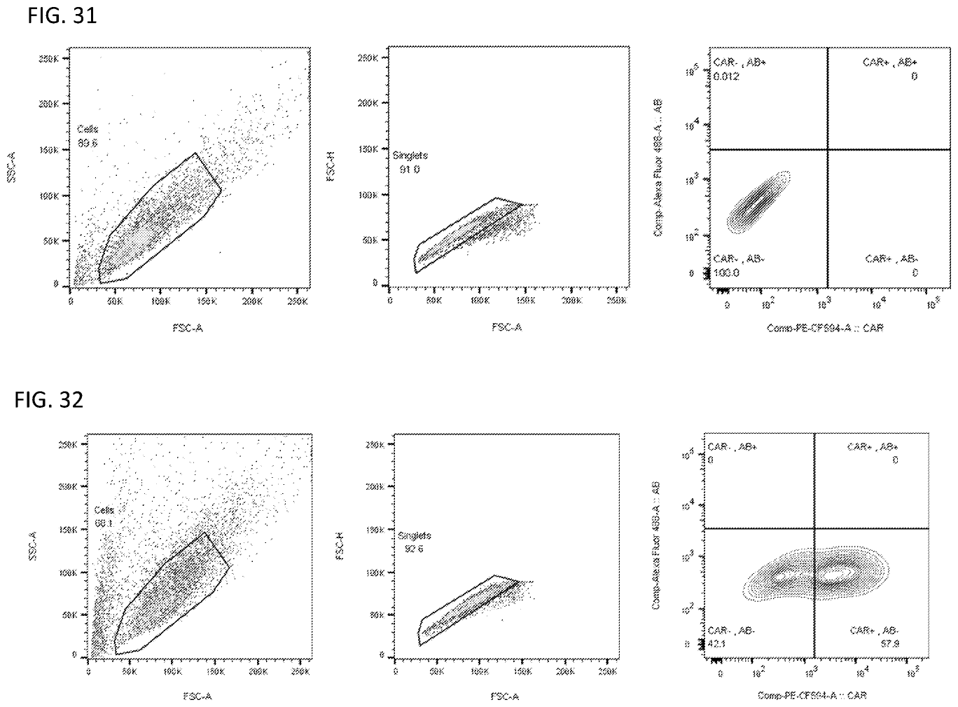

Aβ uptake was compared between CAR Aducanumab (H2L) BMDM and untransduced BMDMs+Aducanumab. 100,000 cells per well were used. Cells were exposed to media containing Aβ concentration: 40 nM. Untransduced cells were incubated with Aducanumab+Aβ for 1 hour @ 37° C. whereas CAR-BMDMs only received Aβ. Uptake was measured via Flow Cytometry as described herein (CAR reporter mCherry, Aβ is fluorescently-labeled). A gating strategy was used to determine CAR− and Aβ− gates in untransduced control without Aβ (FIG. 28). Gating demonstrated that a majority of CAR+ cells uptake Aβ (FIG. 31). Anti-Aβ CAR led to higher Aβ uptake than non-specific CARs and Aducanumab in primary murine BMDMs (FIG. 30). In this experiment, 100,000 murine bone marrow derived cells were plated per 48 well plate. Untransduced cells and CAR bearing cells were exposed to media containing 40 nM of fluorescently-labeled (AF488) amyloid beta for 1 hr at 37° C. Untransduced-Aducanumab treated conditions were performed as follows: aducanumab was complexed with the fluorescently-labeled amyloid beta at the specified concentrations of aducanumab (0.1 μg/mL, 1 μg/mL, 10 μg/mL) for 30 minutes prior to the 1 hour incubation with cells at 37° C. Association of amyloid beta was determined via flow cytometry via AF488 fluorescence. CAR expression was determined via the mCherry reporter gene.

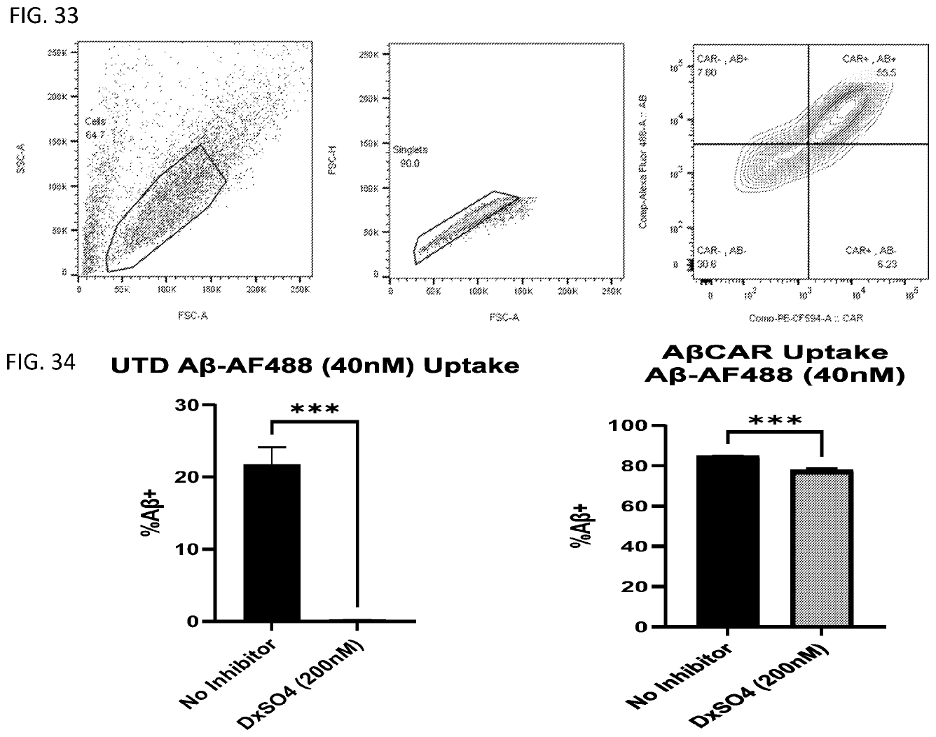

An Aβ uptake inhibitor study was performed. Dextran sulfate competitively inhibits MARCO and TREM2, which are known receptors for Aβ. This inhibitor was tested against the Aducanumab (H2L) CAR (mCherry) and untransduced cells. Flow gating schemes are shown in FIGS. 31-33 (cells→singlets→CAR (mCherry)/Aβ (AF488)). CAR-BMDMs and untransduced murine BMDMs were incubated with dextran sulfate, a competitive inhibitor for MARCO class A scavenger receptor and TREM2 (known binders of amyloid beta), at a concentration of 200 nM for 1 hour at 37° C. Then cells were exposed to media containing both dextran sulfate at a concentration of 200 nM and fluorescently-labeled amyloid beta at a concentration of 40 nM for 1 hour at 37° C. Aβ uptake was measured immediately post-incubation. The presence of the inhibitor significantly reduced the ability of untransduced cells to uptake amyloid beta, but not on CAR-BMDMs, suggesting the CAR construct presents a novel way to uptake amyloid beta (FIG. 34).

### Example 2: Engineering Macrophages to Recognize and Uptake Tau Protein Aggregates

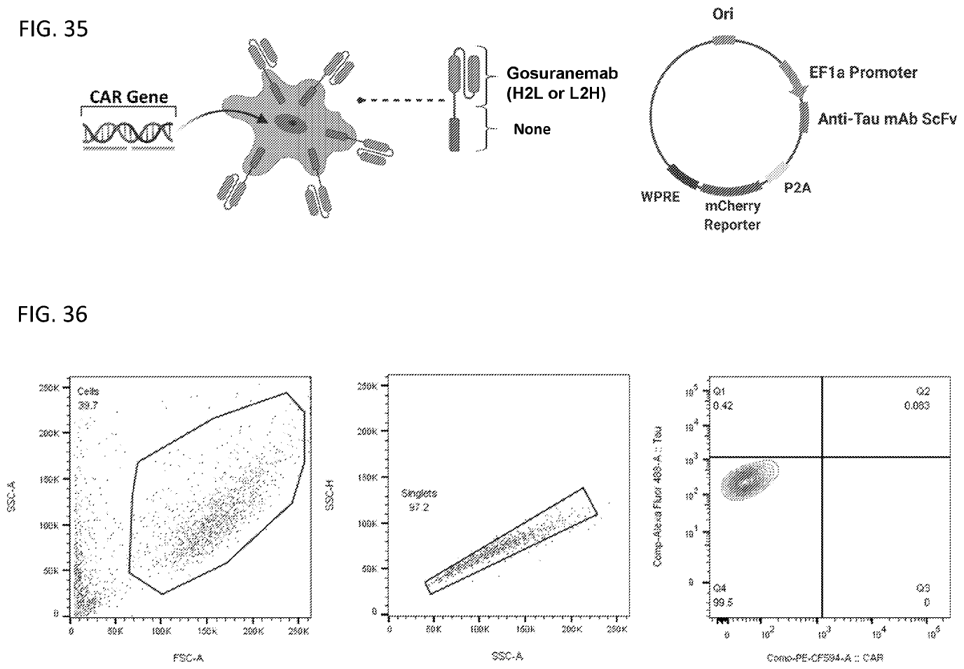

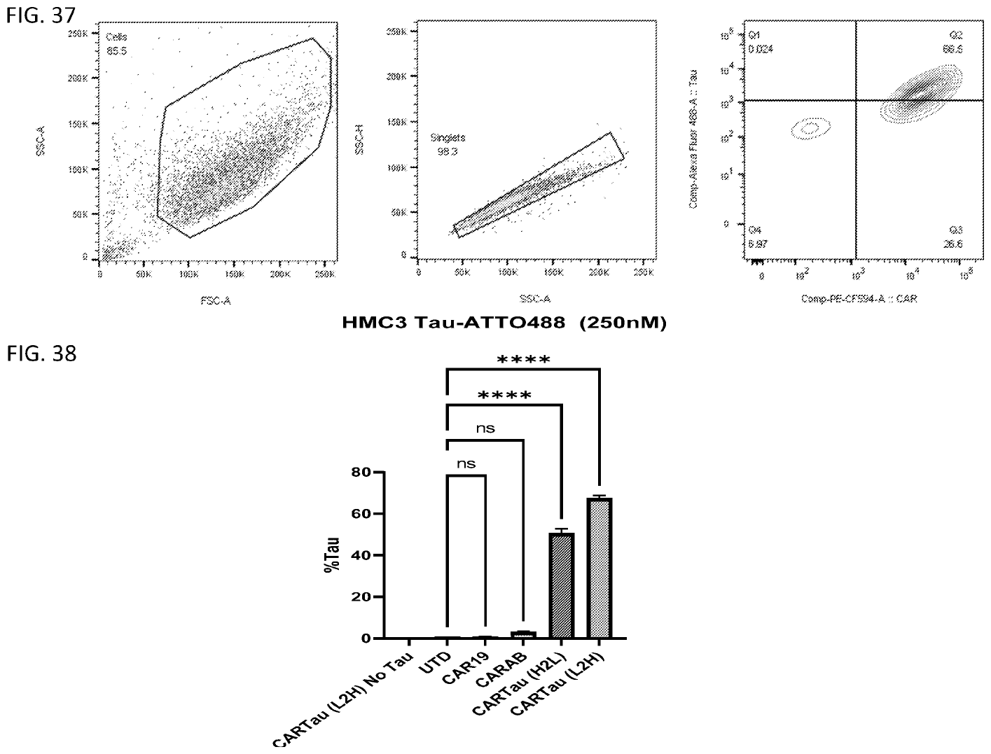

Tau accumulation is correlated with further advanced Alzheimer's disease, so a therapy targeting Tau would be useful for treating later-stage AD patients. Thus, macrophages were engineered recognize and uptake Tau protein aggregates. Anti-Tau CAR constructs were designed in a similar way as the anti-Aβ CARs (FIG. 35). The ScFv from an antibody which targets Tau (Gosuranemab) was used for the antigen binding domain. The CAR-Tau construct was first tested in the HMC3 cell line. Flow gating strategies are shown in FIGS. 36-37 (cells→singlets→CAR (mCherry)/Tau (ATTO-488).

Tau CARs outperformed CARAβ (Aducanumab H2L CAR, no signaling domain), CAR19 and UTDs in HMC3s (FIG. 38). HMC3 cell lines were developed with the Gosuneramab (H2L) and Gosuneramab (L2H) CAR constructs. Uptake of fluorescently-labeled Tau was compared to HMC3 cells expressing the Aducanumab (H2L) CAR (CARAβ), CAR19 and Untransduced HMC3 cells. 100,000 Cells were incubated with media containing fluorescently-labeled Tau at a concentration of 250 nM for 1 hour at 37° C. CAR Tau showed superior uptake of fluorescently-labeled Tau compared to other constructs demonstrating the specificity of the CAR.

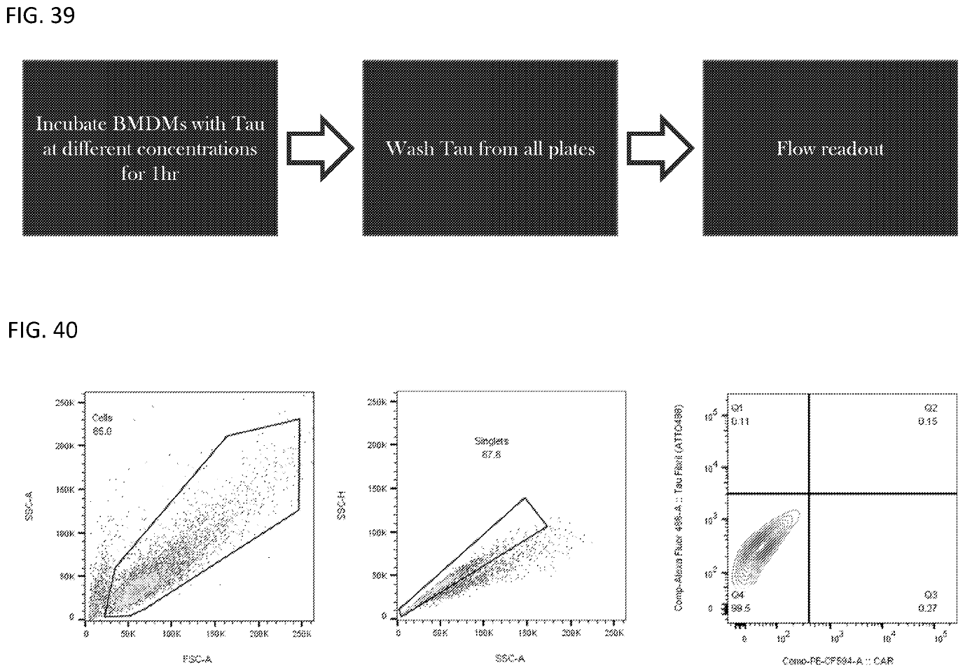

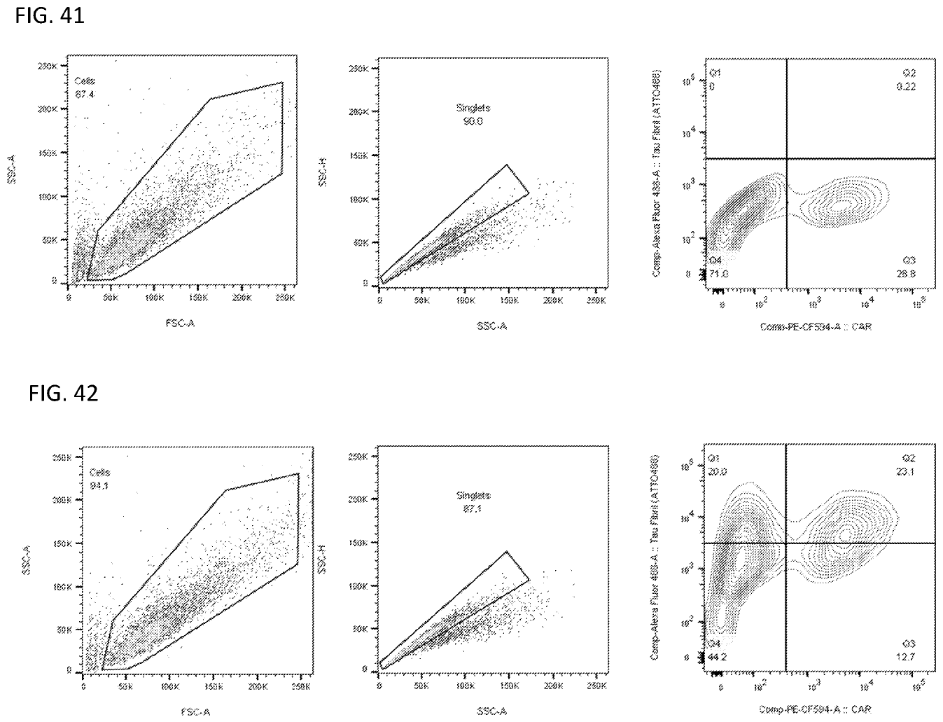

Tau assay with BMDMs set-up is illustrated in FIG. 39. Assay conditions were as follows: Plating parameters: 200,000 cells/well, Tau ATTO-488 media. Constructs tested: (H2L) Gosuranemab CAR, (L2H) Gosuranemab CAR, CAR19 CD3z, and Untransduced. Tau ATTO-488 concentrations tested: 250 nM, 50 nM, 10 nM, 2 nM, and 0 nM. Flow gating schemes and flow plots are shown in FIGS. 40-42.

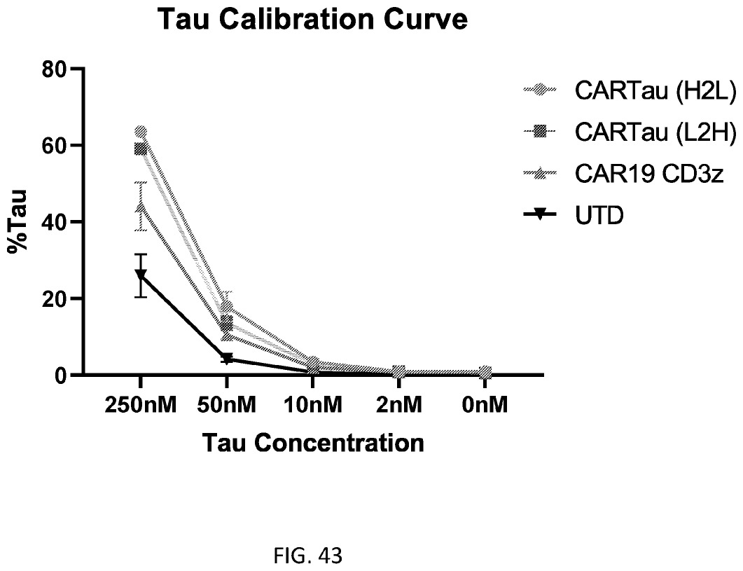

CARTau (H2L) and CARTau (L2H) outperform CAR19 and UTD at 250 nM (FIG. 43). Murine bone marrow macrophages were lentivirally transduced with CAR19 and CAR Tau constructs (Gosuranemab H2L & Gosuranemab L2H). Cells were incubated with media containing fluorescently-labeled Tau at various concentrations for 1 hour at 37° C. CAR Tau BMDMs exhibited greater uptake of Tau at 250 nM (FIG. 43).

### Example 3: Validation of CAR Functionality in Human Macrophages

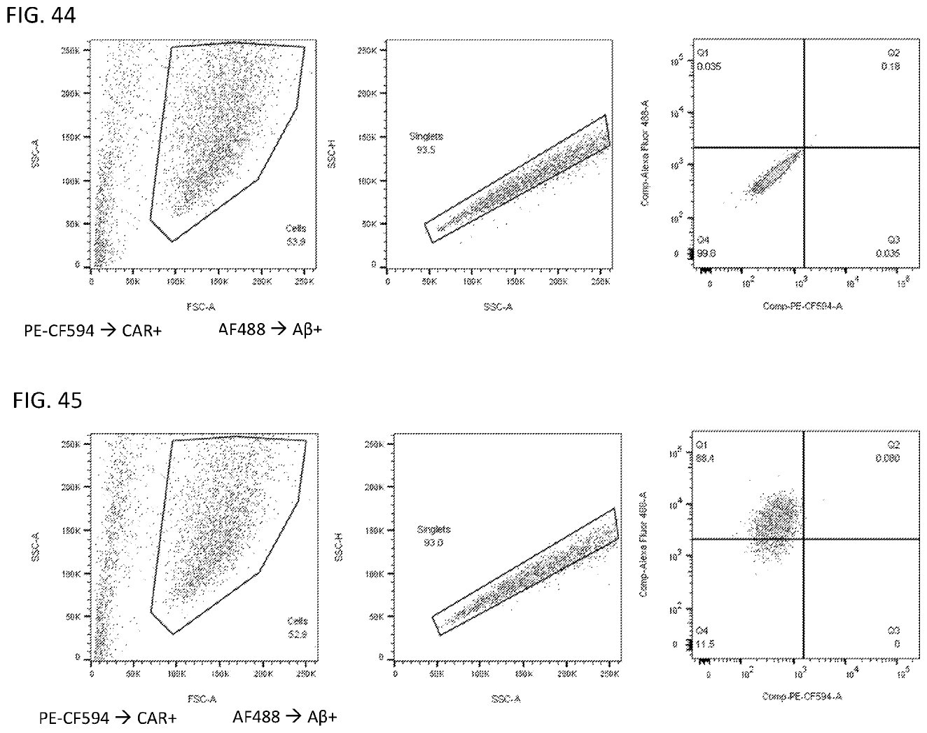

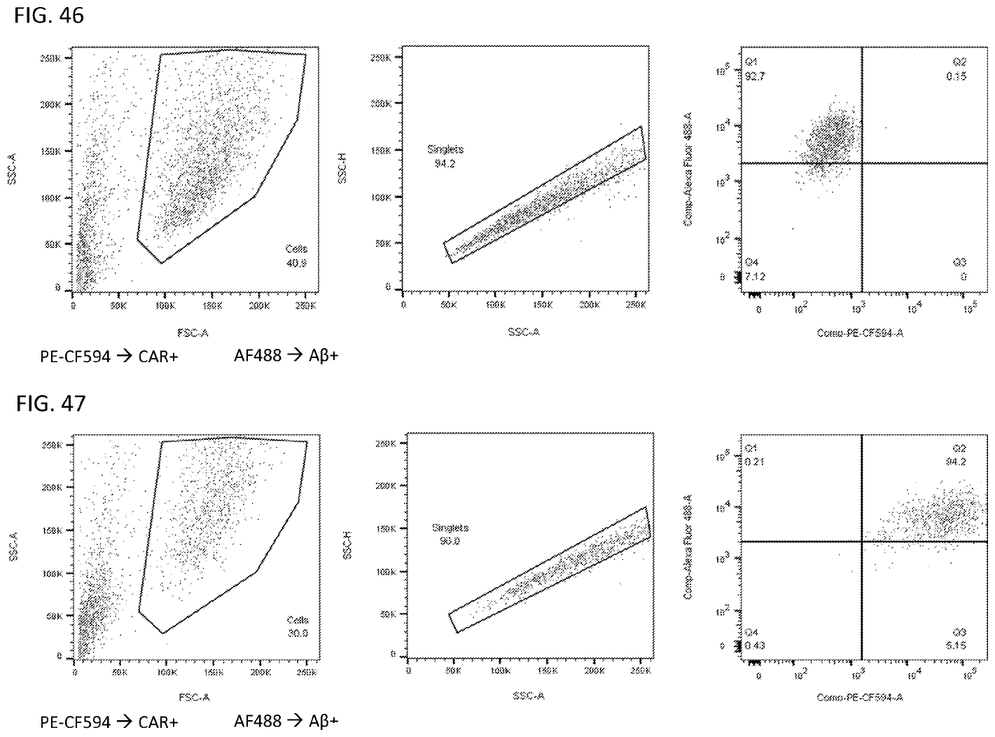

CAR functionality was validated in human macrophages. Assay conditions included: 50,000 cells per well, Aβ-AF488 (40 nM) exposure for 1 hour at 37° C., 500 μL of AB media, mixture of aducanumab+Aβ (1 hour pre-treated), and measured via fluorescence cytometry. Flow plots are shown in FIGS. 44-47.

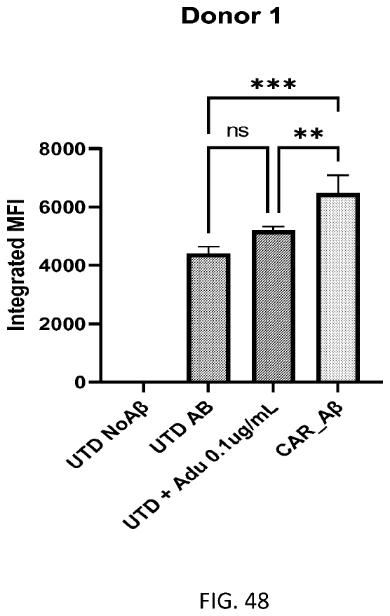

Aβ uptake was evaluated in human macrophages. Studies showed that anti-Aβ human macrophages uptake more amyloid than UTDs or Aducanumab macrophages (FIG. 48). Weighted MFI shows that Anti-Aβ CAR lentivirally transduced macrophages uptake more Aβ than untransduced macrophages or macrophages incubated with Aducanumab at a concentration of 0.1 ug/mL (FIG. 48).

### Example 4: Degradation of Amyloid Beta by CAR-Bearing Macrophages

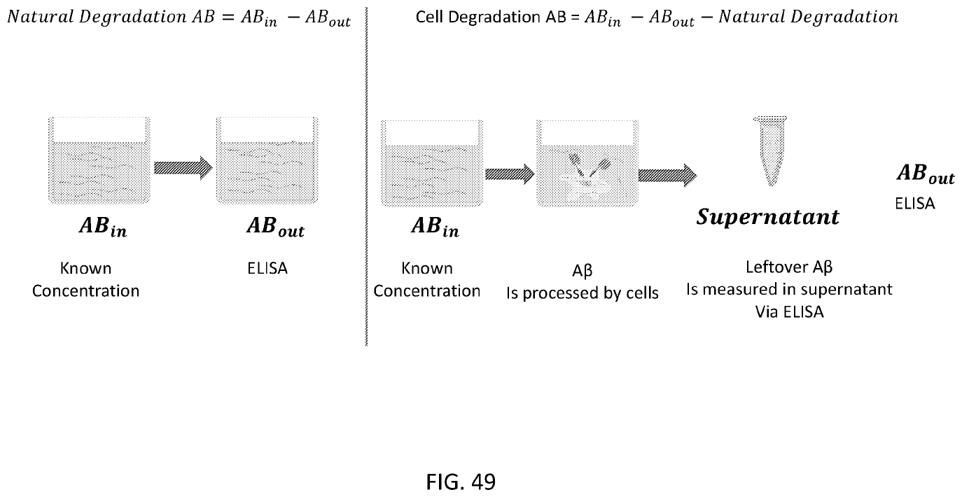

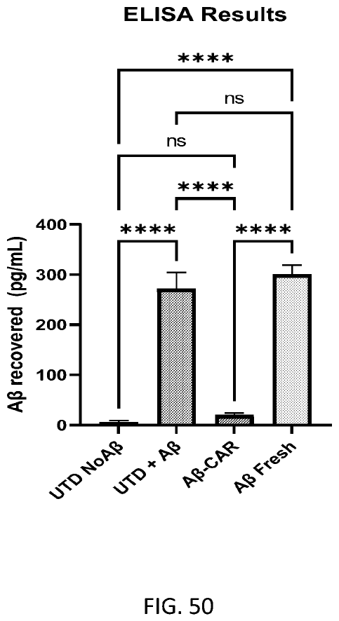

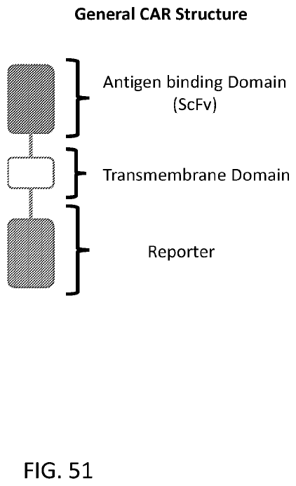

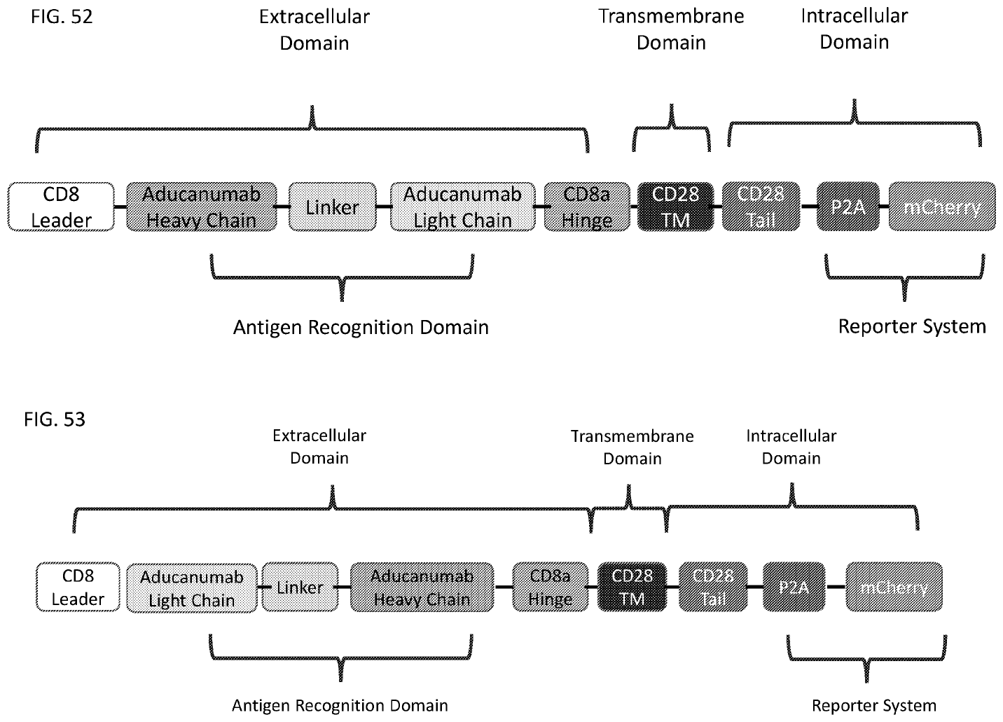

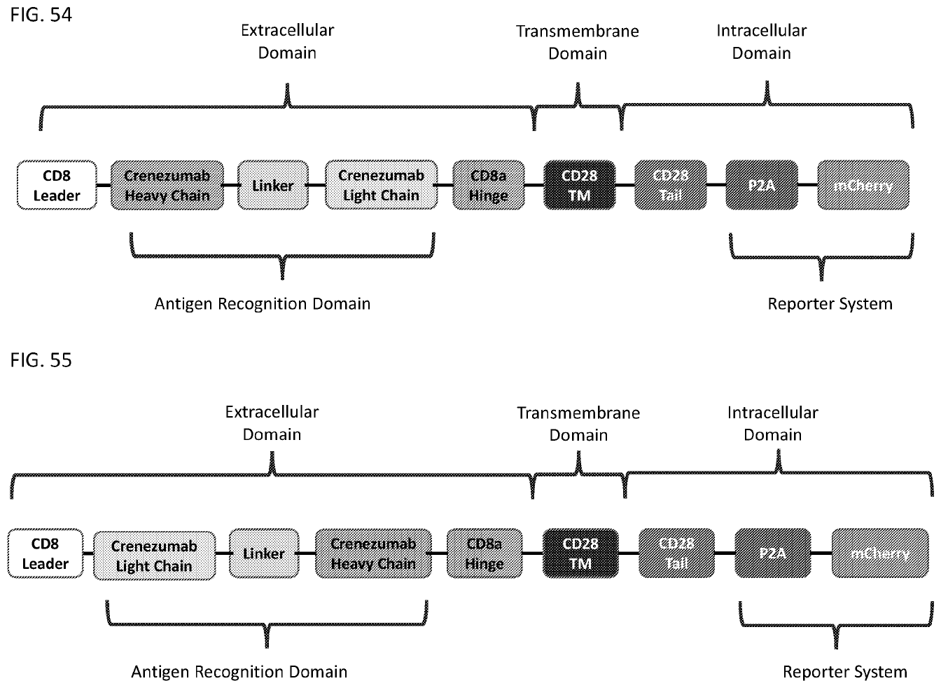

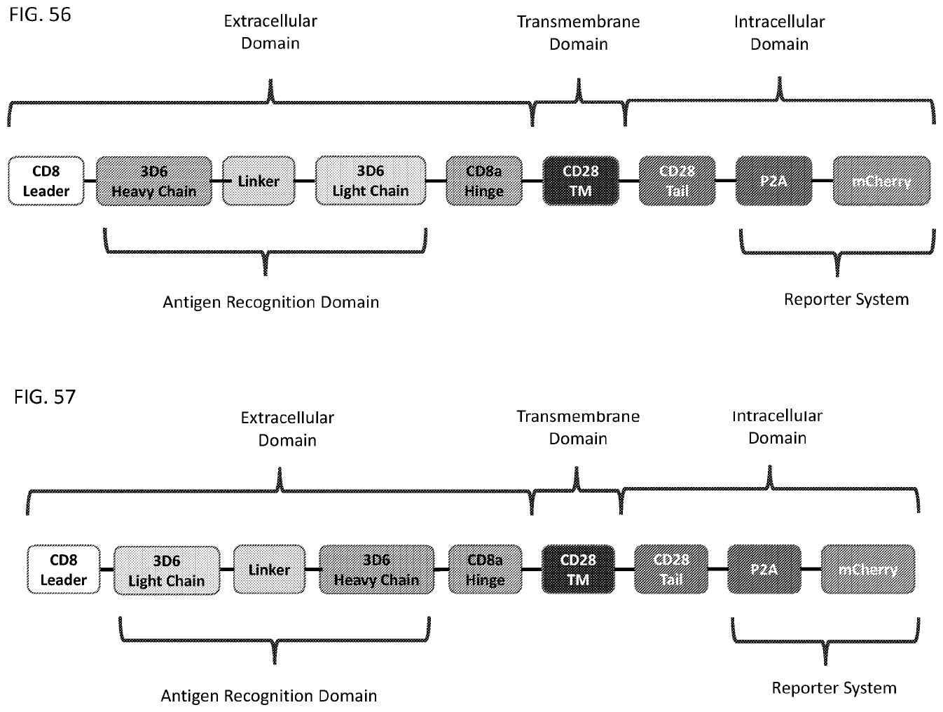

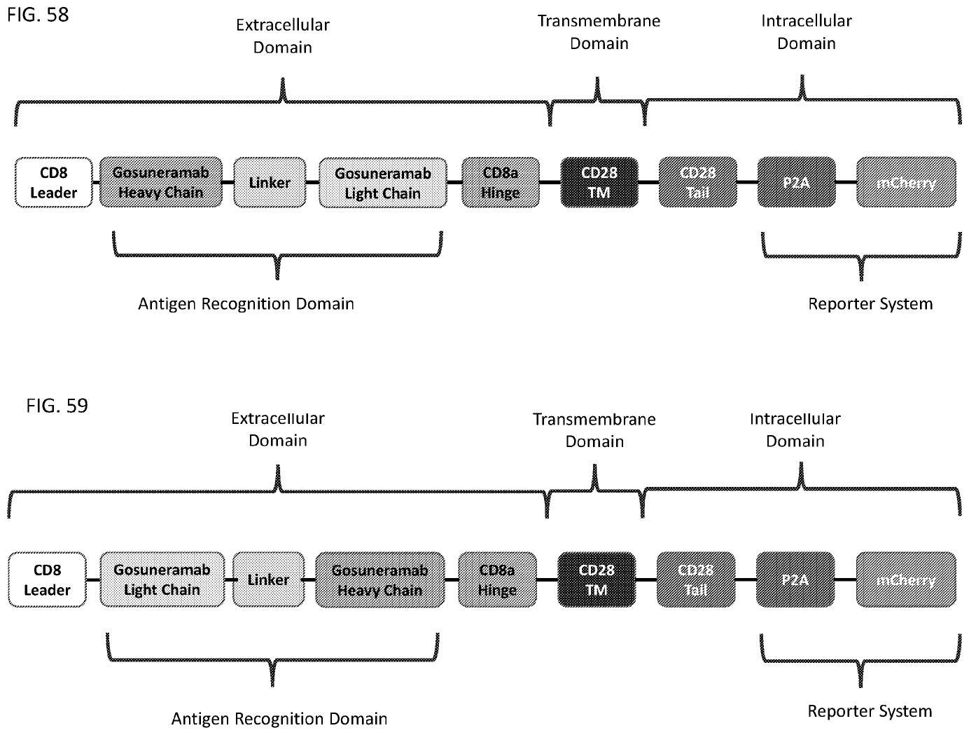

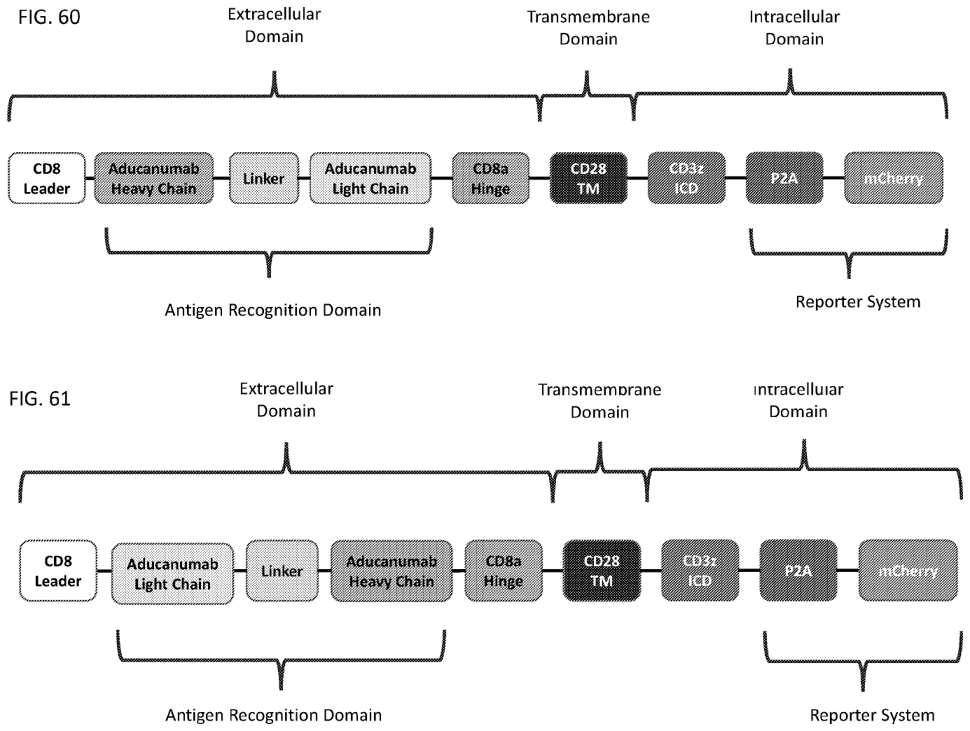

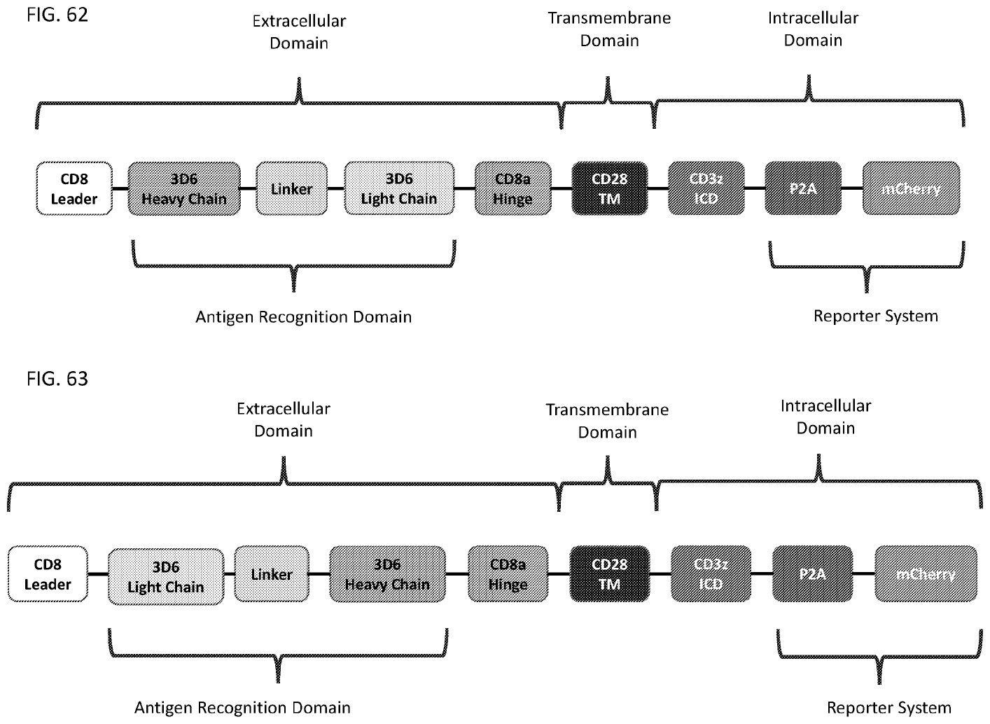

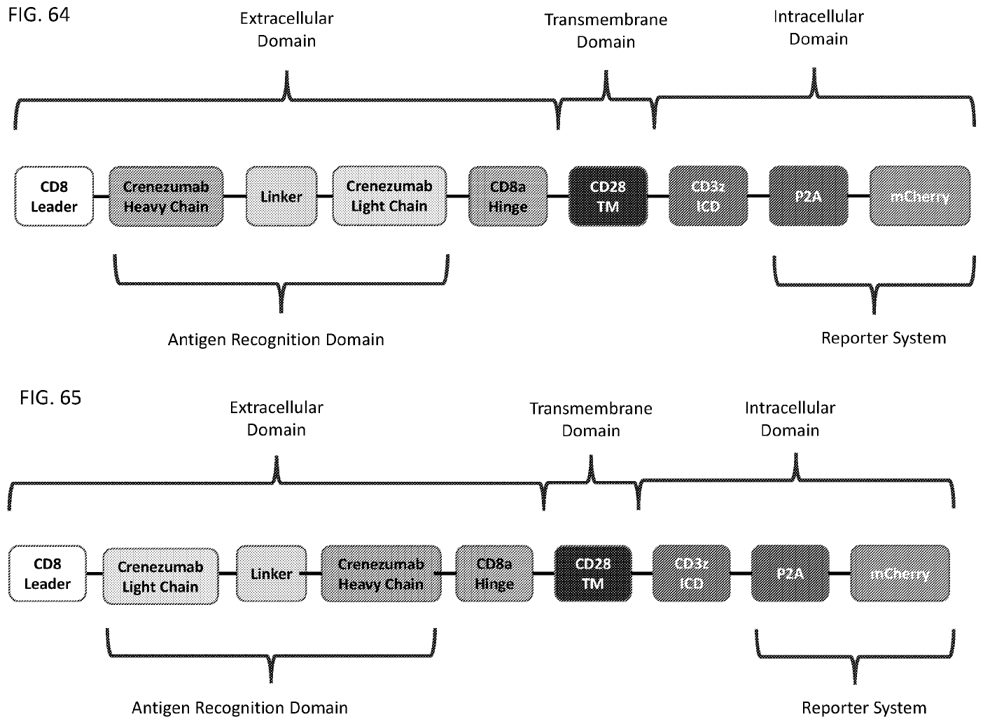

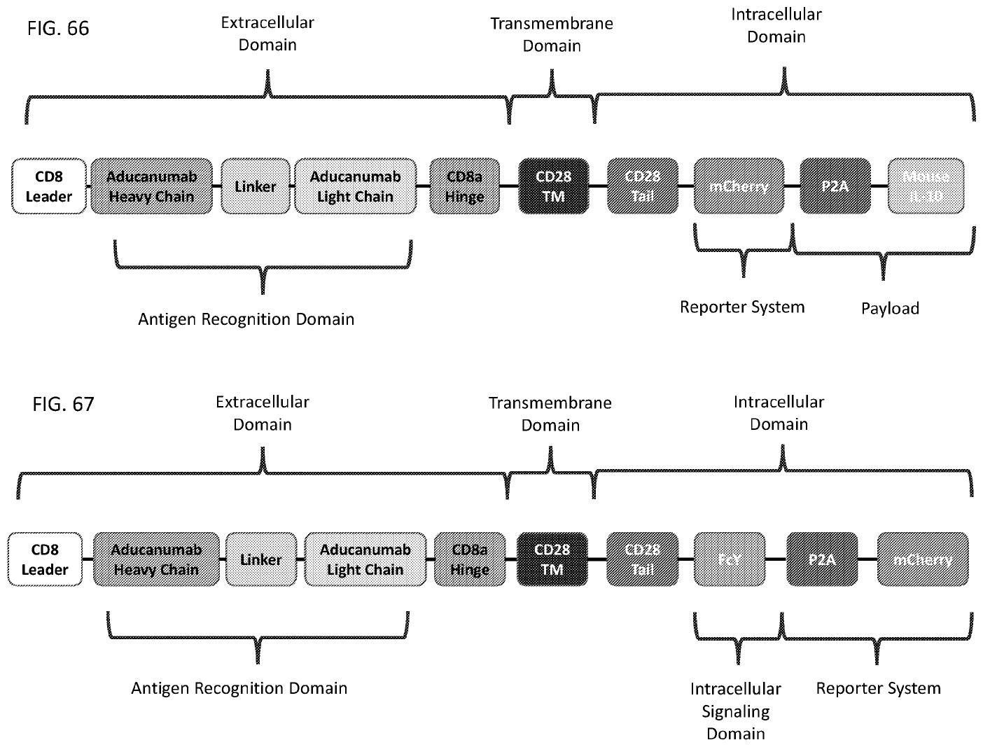

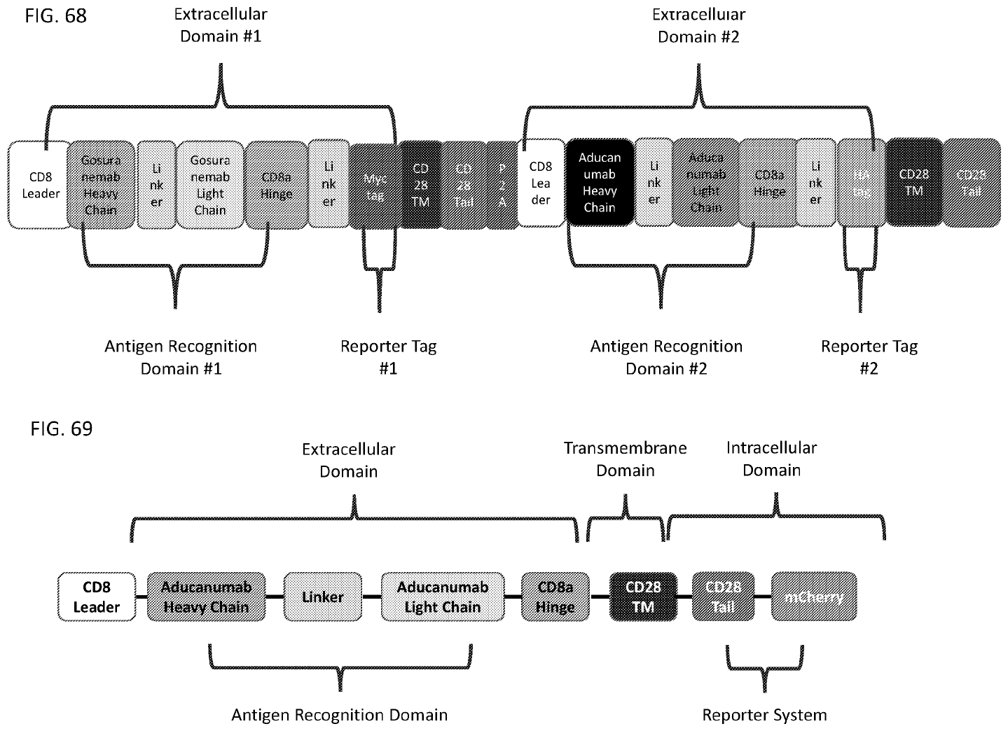

Schema for quantification of Aβ degradation by CAR-BMDMs using ELISA (FIG. 49). CAR-Aβ BMDMs cleared amyloid beta from supernatant more efficiently than UTDs (FIG. 50). 50,000 murine BMDM cells in 96 well plates (sorted to achieve 100% CAR expression) were exposed to 5 μM Aβ media for ˜36 hours. Aβ supernatant was measured via ELISA. Conditions: UTD No Aβ (n=3), UTD+Aβ (n=3), CAR-Aβ+Aβ (n=3), and fresh Aβ no cells (measure of initial Aβ). Results showed Aβ was significantly removed from the supernatant by anti-Aβ CAR bearing BMDMs (Aducanumab H2L) compared to untransduced cells (FIG. 50). Statistical Analysis: One Way ANOVA.

### Example 5: Engineering CAR Microglia to Deliver a Therapeutic Payload

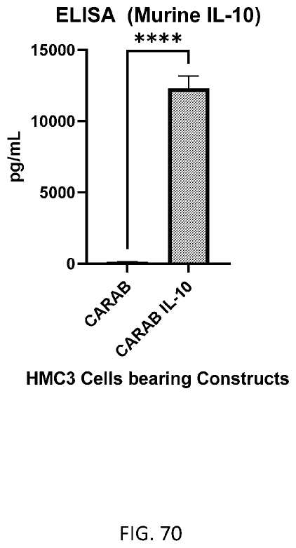

Delivery of a therapeutic payload via microglia like cells would be useful for treating AD patients at various stages depending on disease pathology. An exemplary therapeutic payload is an anti-inflammatory cytokine, which could reduce localized inflammation. As shown in FIG. 70, HMC3 cells were transduced via lentivirus at an MOI of 1 with constructs encoding an anti-Aβ CAR or an anti-Aβ CAR with a secretable murine IL10 payload and then sorted. 100,000 cells were cultured for 48 hours and IL10 was measured in the supernatant via ELISA after 48 hours. The cells bearing the IL10 payload secreted a detectable amount of murine IL10, whereas those not bearing the IL10 did not secrete a detectable amount of murine IL10.

### Example 6: Engineering Macrophages to Simultaneously Recognize Amyloid Beta and Tau Protein Aggregates

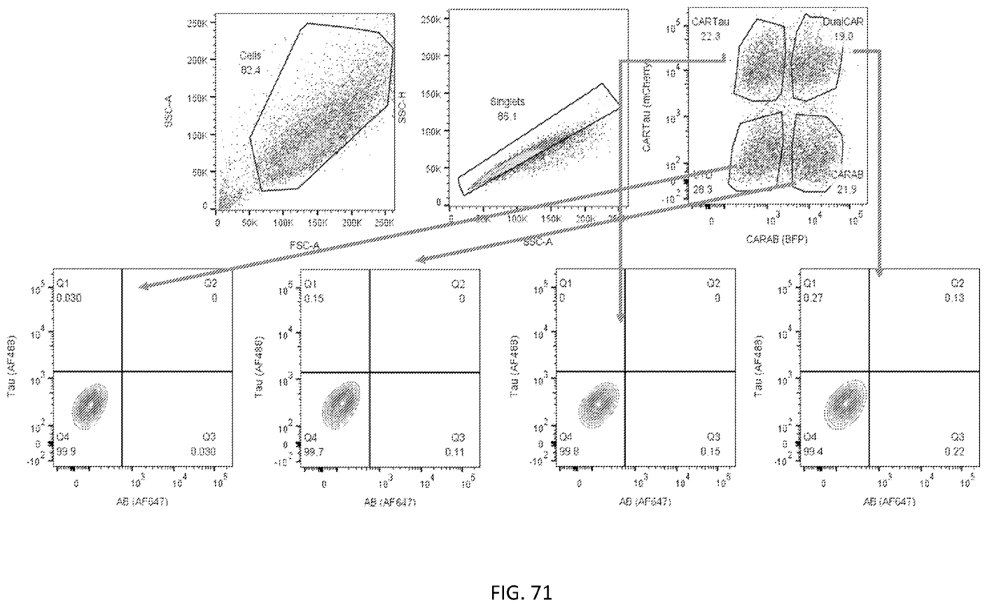

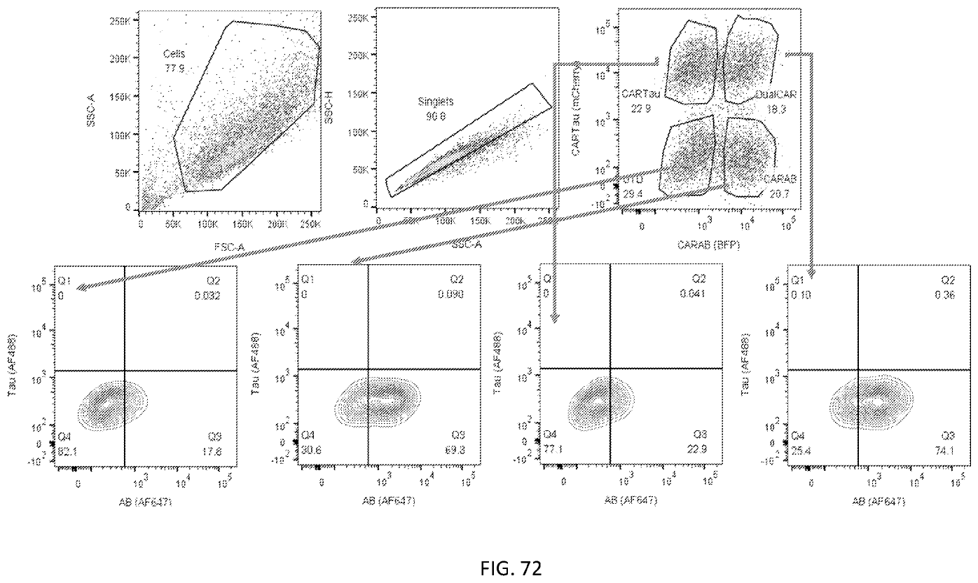

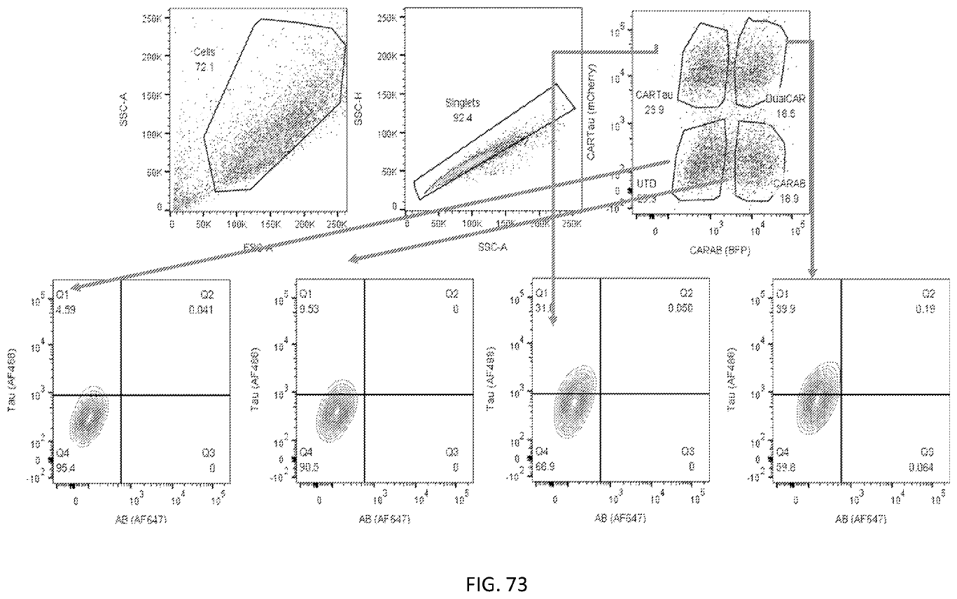

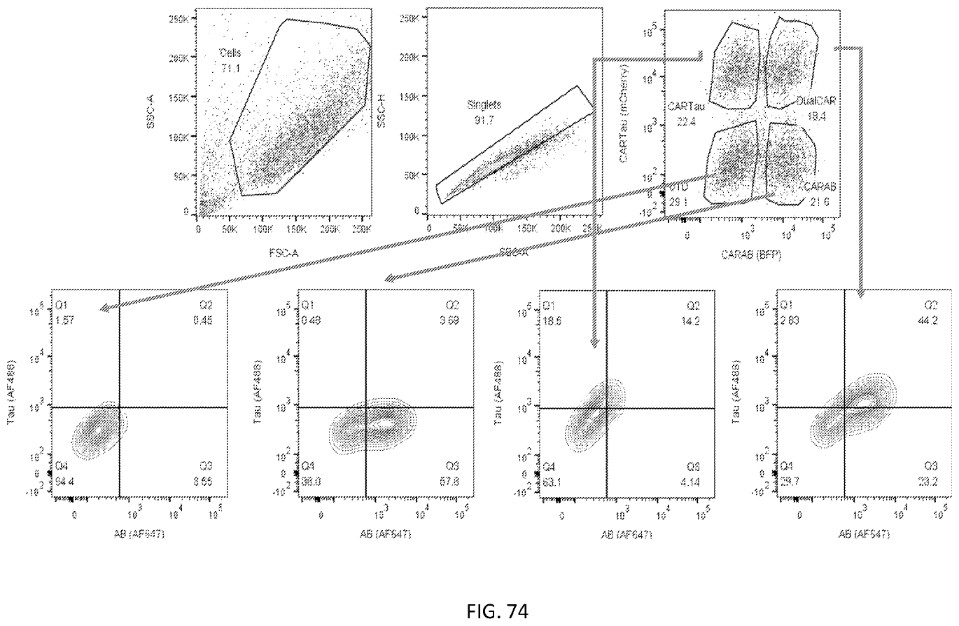

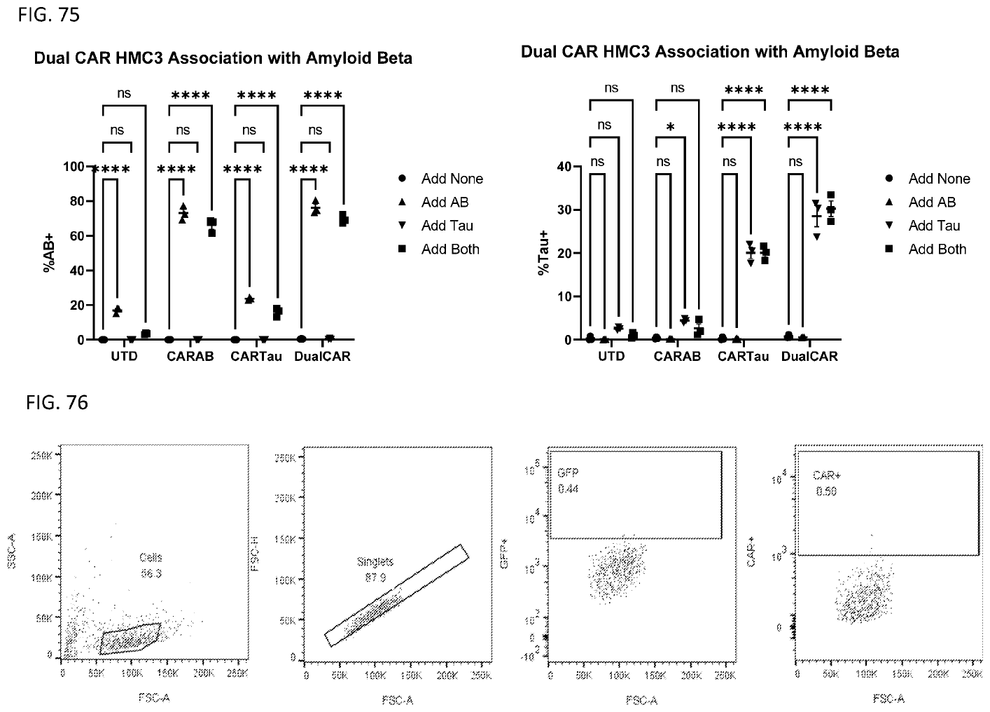

Simultaneous presence of Amyloid beta and Tau pathology is correlated with advanced Alzheimer's disease, so a therapy targeting both pathologies would be useful for treating later-stage AD patients. Thus, macrophages were engineered to recognize and phagocytose Amyloid Beta and Tau aggregates simultaneously by transducing cells with two CAR constructs, one comprising an ScFv from an antibody that targets amyloid beta (Aducanumab) as the antigen binding domain and the other comprising an scFv from an antibody that targets Tau (Gosuranemab) as the antigen binding domain. 100,000 cells comprising a mixture of HMC3 cells expressing either the anti-amyloid beta CAR, the anti-Tau CAR, both CARs, or neither CAR were incubated with media containing either 250 nM of fluorescently labeled amyloid beta (AF647), 50 nM fluorescently labeled Tau (AF488), both, or neither for 1 hour at 37° C. Amyloid beta-mediated phagocytosis was observed for cells comprising either the anti-amyloid beta CAR alone or cells comprising both the amyloid beta CAR and the Tau CAR. Similarly, Tau-mediated phagocytosis was observed for cells comprising either the anti-Tau CAR alone or cells comprising both the amyloid beta CAR and the Tau CAR. When exposed to both types of protein aggregates (Amyloid beta and Tau), only those cells expressing both the anti-amyloid beta CAR and the anti-Tau CAR phagocytosed both proteins simultaneously (FIG. 71-75).

### Example 7: Using Lipid Nanoparticles to Create Anti-Amyloid Beta CAR Macrophages

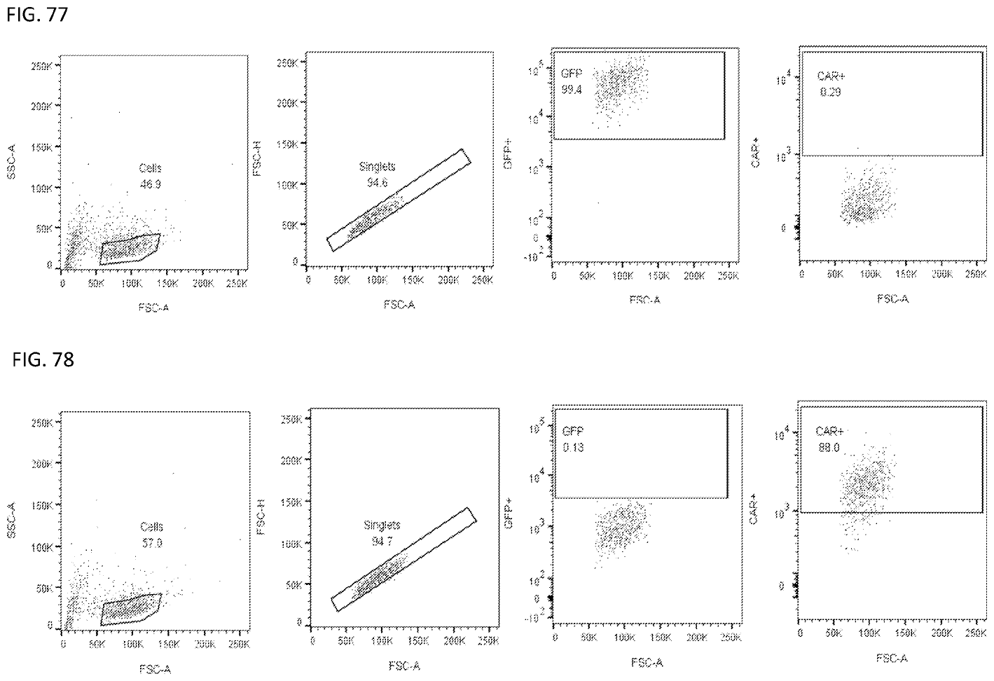

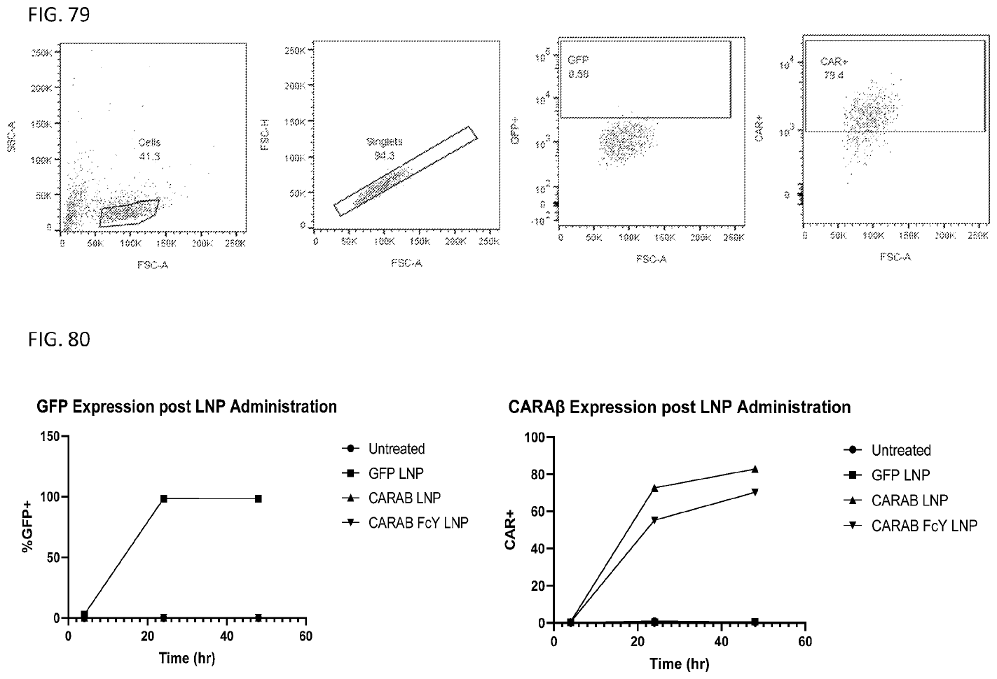

Generating CAR macrophages by delivering an mRNA payload encoding a CAR via lipid nanoparticles (LNPs) could be a useful way of generating CAR macrophages in vivo. One million murine macrophages were exposed to either 5 μg of LNPs loaded with mRNA encoding GFP, mRNA encoding an anti-amyloid beta CAR, or mRNA encoding an anti-amyloid CAR with a murine Fc signaling domain. GFP and CAR expression was measured at 4 hours, 24 hours, and 48 hours post LNP administration. Expression of GFP and CAR (mCherry reporter gene) was measured via flow cytometry. FIG. 76 shows GFP and CAR expression in untreated macrophages. As shown in FIG. 77, GFP-LNP treated macrophages exhibited GFP expression. As shown in FIG. 78, macrophages treated with the anti-amyloid beta CAR-LNP exhibited expression of the CAR. Additionally, as shown in FIG. 79, macrophages treated with the anti-amyloid beta CAR-Fcγ-LNP exhibited expression of the CAR. FIG. 80 shows graphs illustrating that macrophages exhibited stable expression of GFP and CAR after treatment with mRNA loaded LNPs.

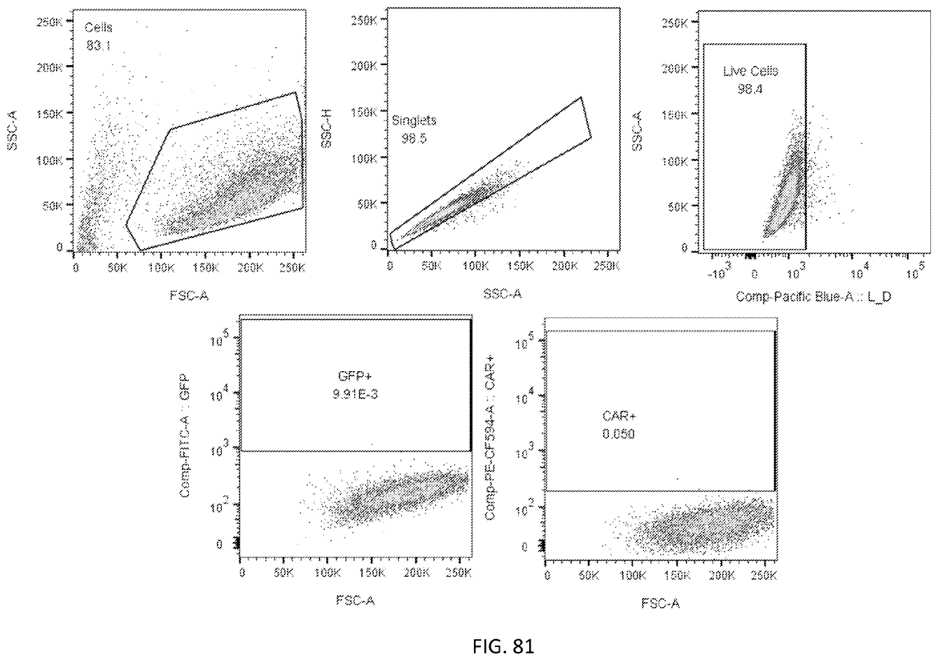

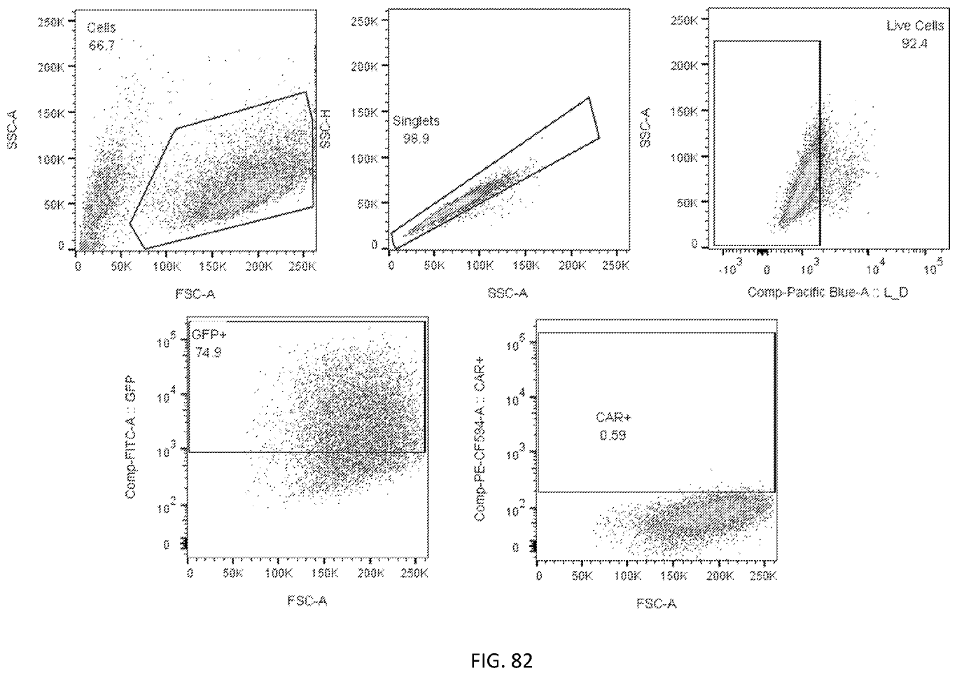

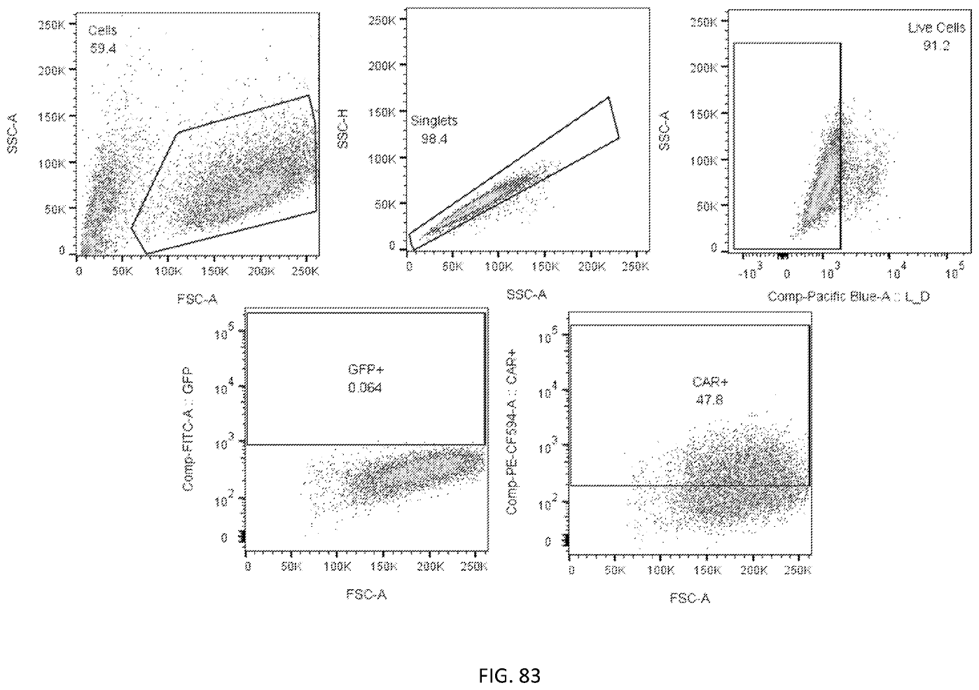

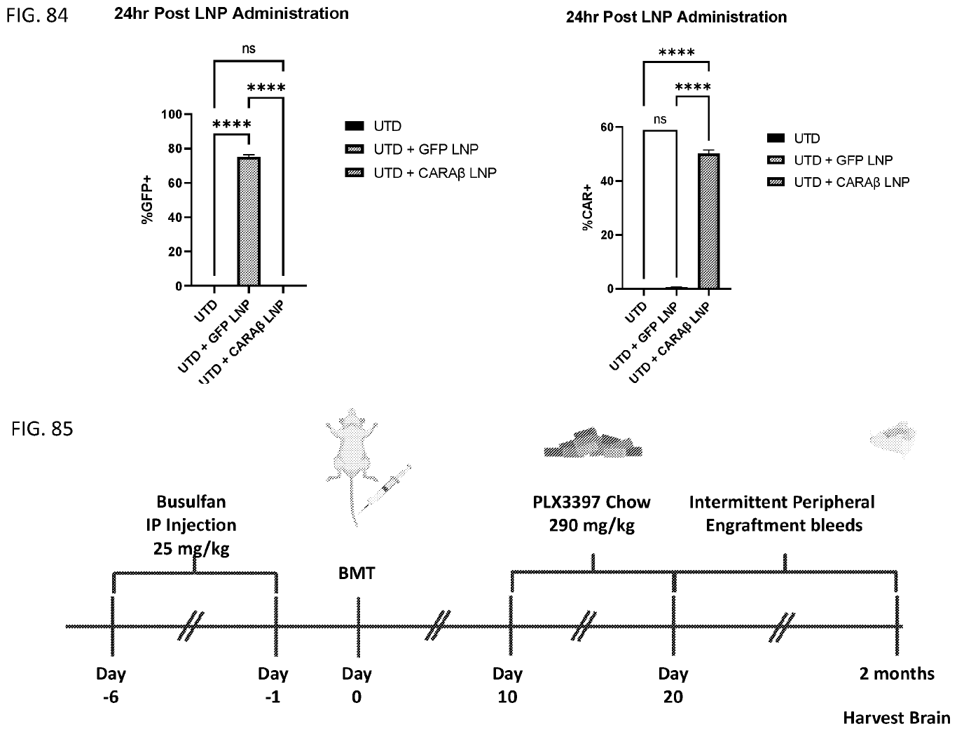

To determine if similar results could be achieved in human macrophages, 200,000 human macrophages were treated with 1 μg of LNPs containing either mRNA encoding GFP or mRNA encoding an anti-amyloid CAR. GFP and CAR (comprising an mCherry reporter gene) expression were measured via flow cytometry 24 hours post LNP administration. As shown in FIGS. 81-84, exposure to LNPs containing mRNA encoding GFP or a CAR led to GFP or CAR expression in human macrophages (FIGS. 81-84).

### Example 8: Engraftment of Anti-Amyloid Beta CAR-Bearing Hematopoietic Stem Cell Derived Cells

FIG. 85 shows an exemplary study schedule described herein. CAR-microglia like cells were engrafted in in wild-type and disease animal models (5×FAD mouse models). Chemotherapy (busulfan) was administered at a dosage of 25 mg/kg for 5 days starting one week prior to bone marrow transplantation. Bone marrow transplants were performed by systemic administration of CAR+ hematopoietic stem cells into host mice. Oral administration via a specialized diet containing a CSF1R inhibitor (PLX3397 at 290 mg/kg) was provided for 10 days after administration of the CAR+ hematopoietic stem cells, allowing the hematopoietic stem cells to be reconstituted to deplete endogenous macrophages. Inhibition of the CSF1R resulted in widespread macrophage/microglia depletion which was then repopulated by donor cells upon removal of the diet. Peripheral bleeds were then performed to evaluate peripheral engraftment of donor cells and CAR-expressing donor cells.

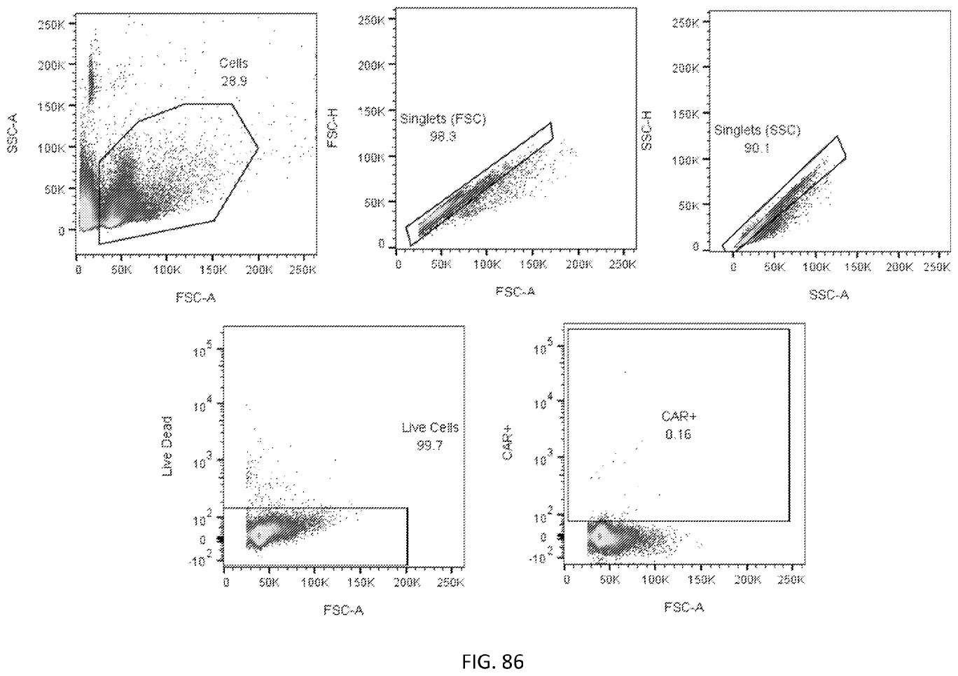

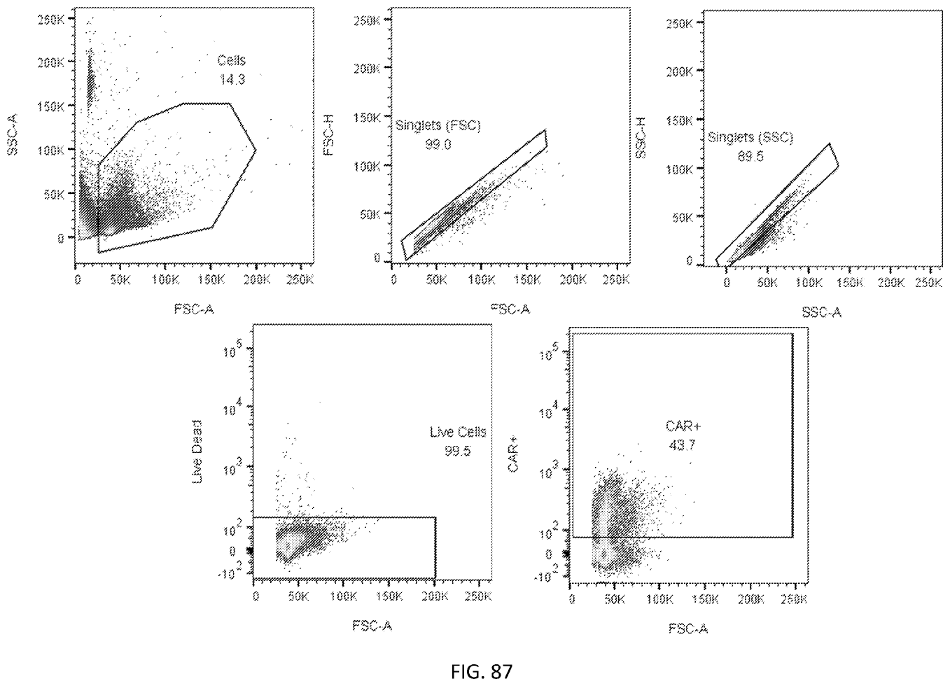

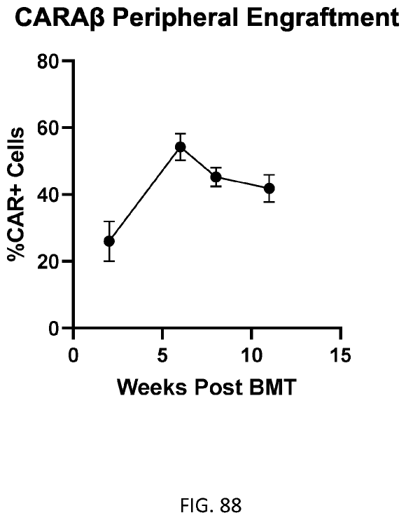

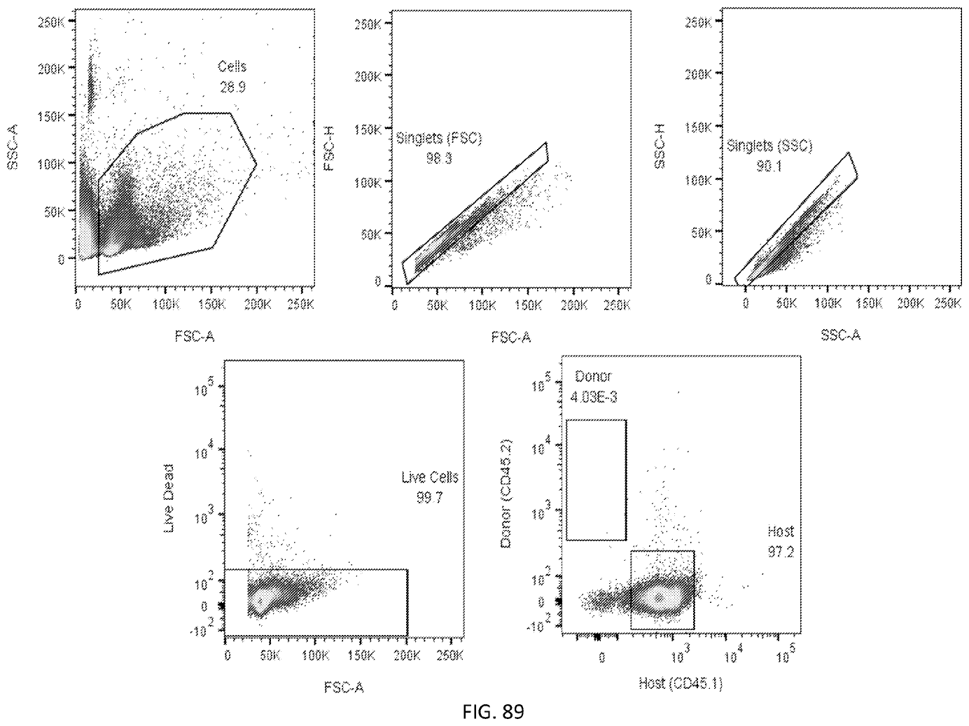

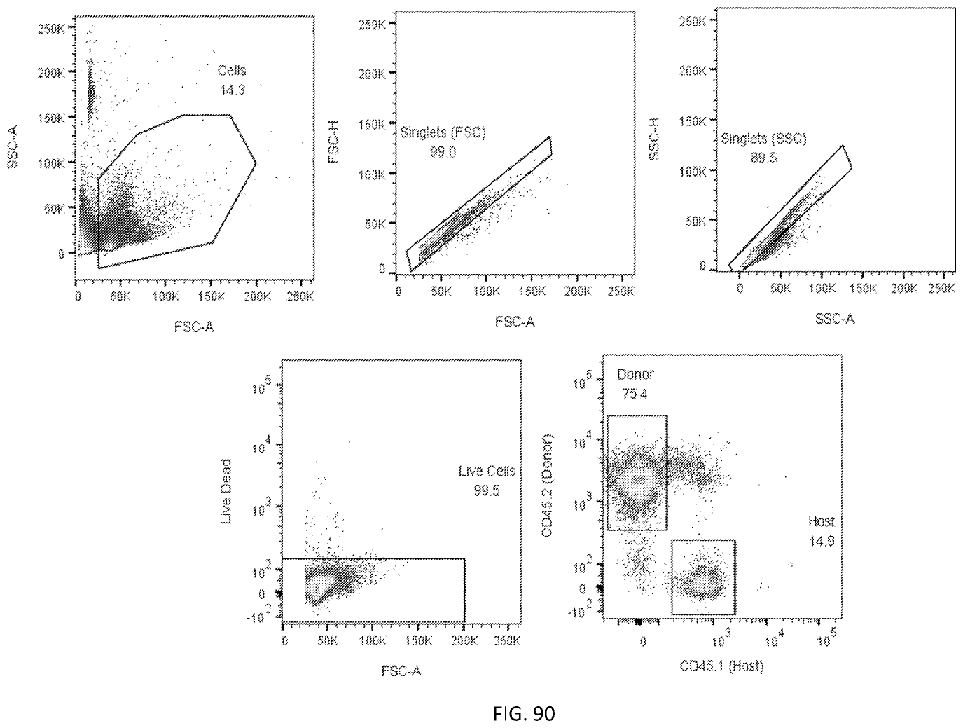

The following gating scheme was used to evaluate CAR expression in cells: cells→singlets→live cells→CAR+ cells. FIG. 86 demonstrates the use of the gating scheme to evaluate overall engraftment of CAR bearing cells in circulation in an untreated wild-type mouse. FIG. 87 demonstrates engraftment of donor derived CAR+ cells in circulation. FIG. 88 exhibits overall engraftment of CAR+ cells over a 3-month period post bone marrow transplant. FIG. 89 shows exemplary flow cytometry data for untreated mice wherein donor cells were CD45.2+ and host cells were CD45.1+. FIG. 90 shows exemplary flow cytometry data for mice treated with CAR Aβ HSCs, showing donor cell engraftment.

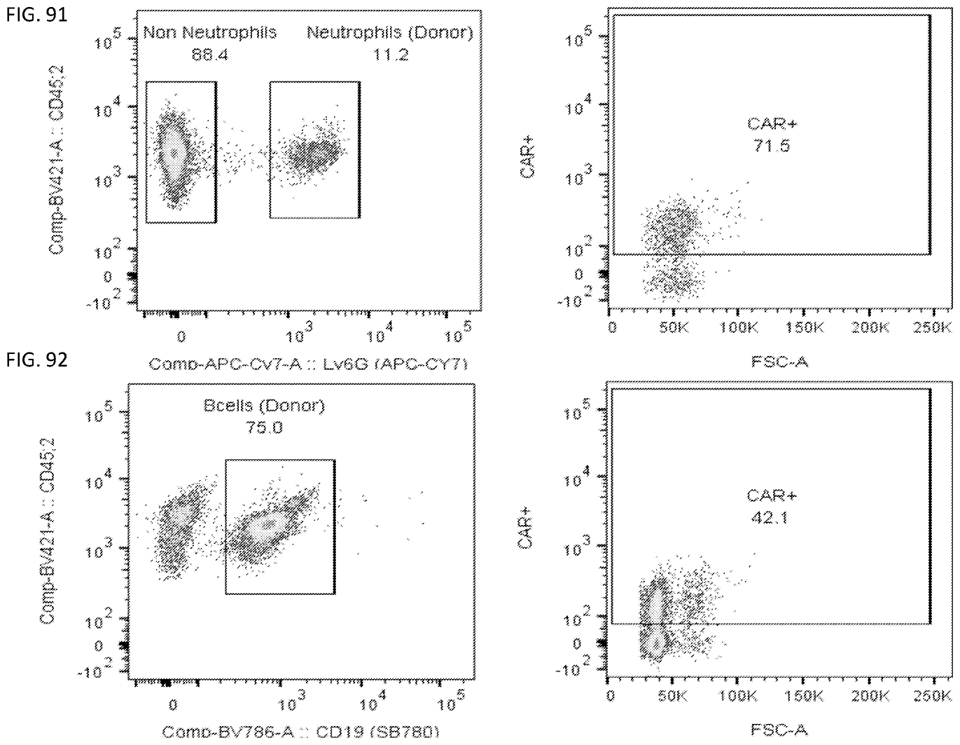

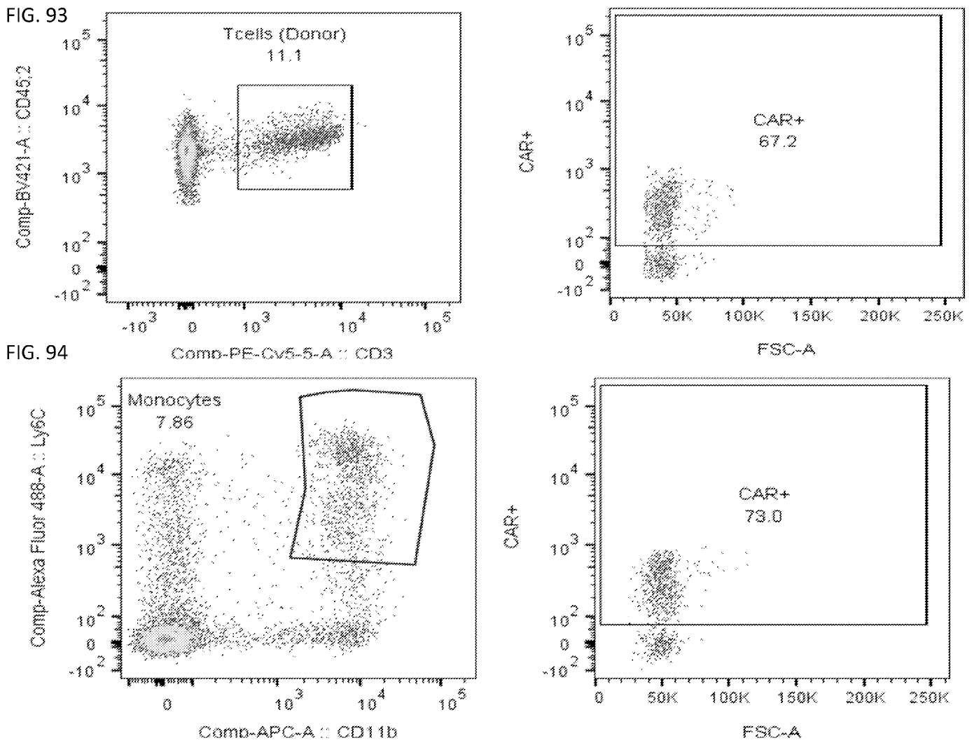

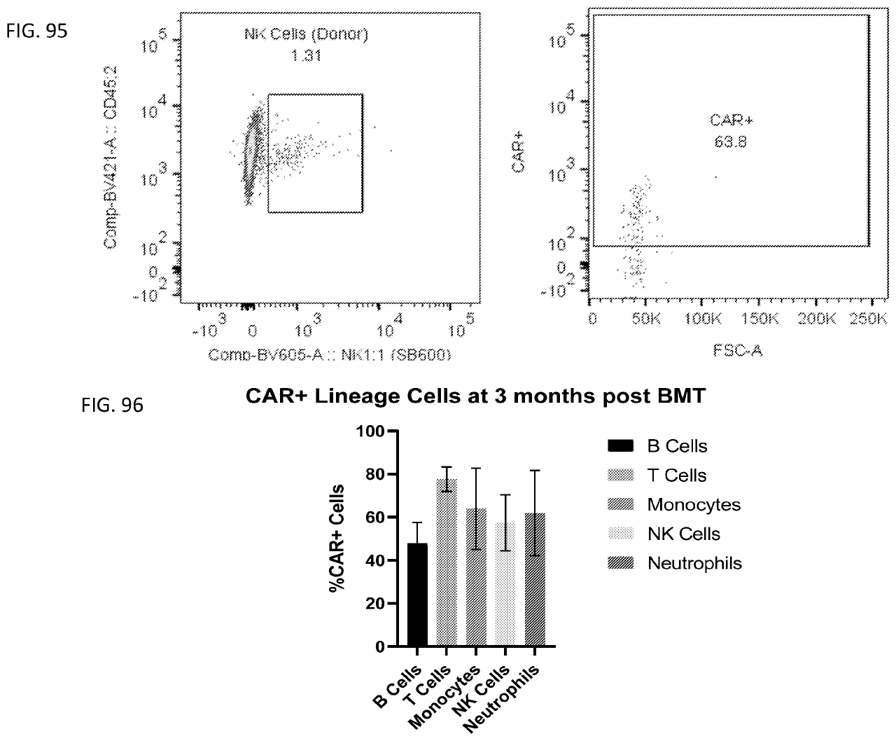

FIG. 91 shows exemplary data of neutrophil cells positive for the donor CAR. Neutrophils were gated by Ly6G+ after the CD45+ gate. FIG. 92 shows donor CAR+ B cells, wherein B cells were gated by CD19+ cells from the non-neutrophil fraction. FIG. 93 shows donor CAR+ T cells, wherein T cells were gated by CD3+ cells from the non-neutrophil fraction. FIG. 94 shows donor CAR+ monocytes wherein monocytes were gated by CD11+ and Ly6C+ cells from the non-neutrophil fraction. FIG. 95 shows donor CAR+NK cells wherein NK cells were NK1.1+ cells from the non-neutrophil fraction.

FIG. 96 shows a graph illustrating stable CAR+ expression by HSC-derived lineage cells in circulation after 3 months in a wild type mouse model.

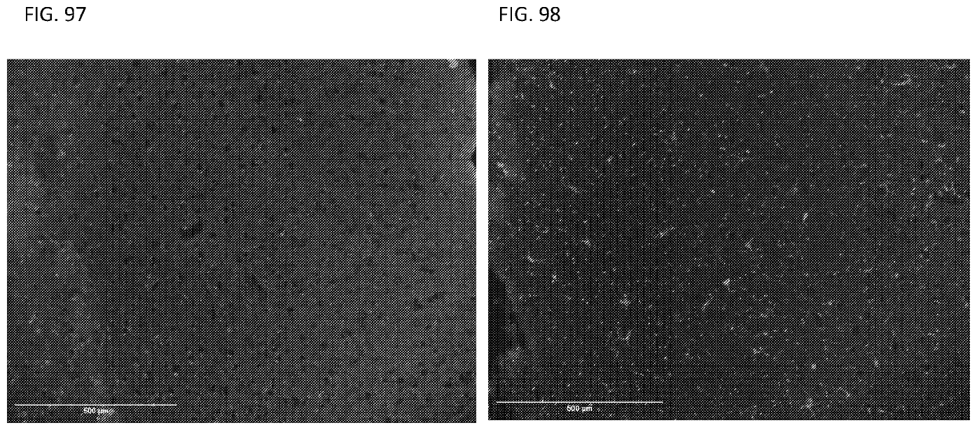

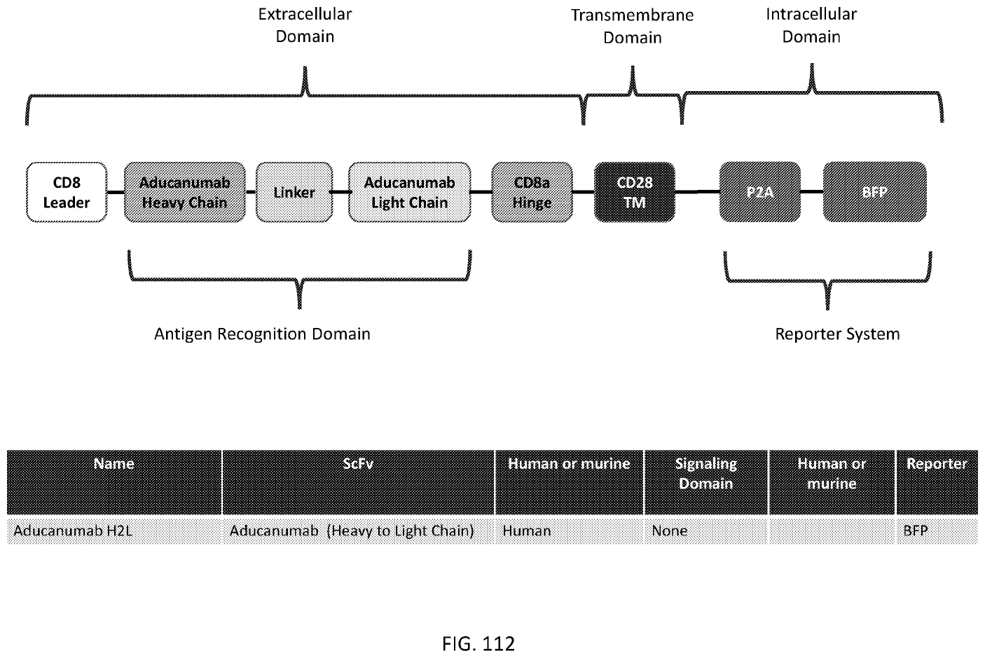

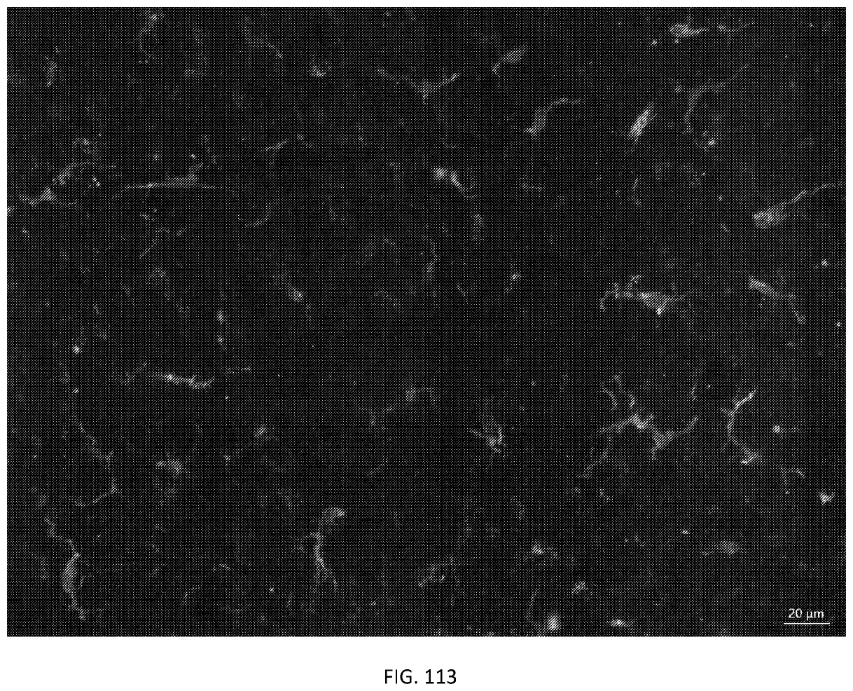

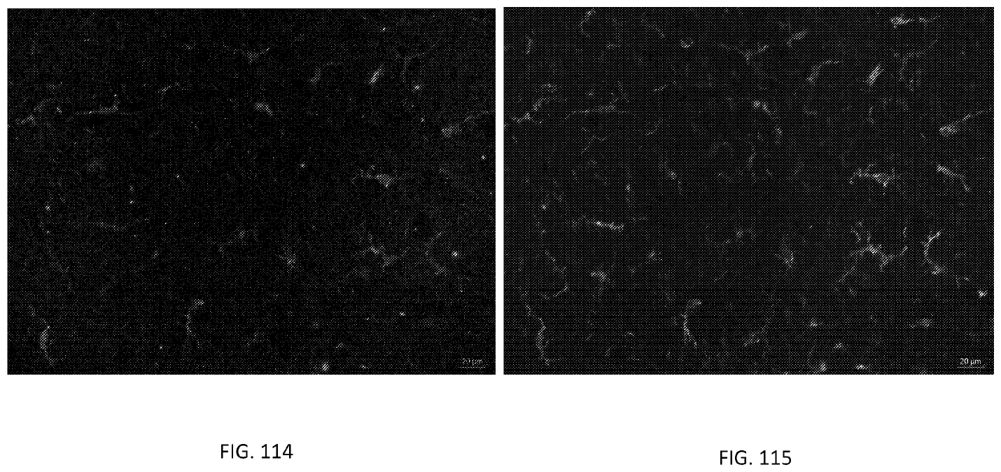

Mice were PBS perfused and organs were harvested and fixed in 4% PFA for a period of 24 hours. Organs were then preserved in 30% sucrose before embedding in OCT and subsequent sectioning in a cryostat (14 uM slices). Tissue sections were hydrated in PBS and exposed to blocking buffer (10% Donkey serum, 0.5% Triton-X) for 1 hour at room temperature. Sections were then stained with chicken anti-mCherry antibody at 1:1000 dilution and rabbit anti-Iba1 antibody at a 1:500 dilution overnight at 4° C. Tissue sections were then washed with PBS and exposed to donkey anti-chicken secondary antibodies fluorescently-labeled (594, 1:1000 dilution) and donkey anti-rabbit secondary fluorescently-labeled antibody (488, 1:1000 dilution) channel for 1 hour at room temperature. Sections were then PBS washed to remove unconjugated antibody and DAPI was added for nuclear staining prior to imaging. FIG. 97 and FIG. 98 are representative images of a sagittal section of a murine cortex at 20× magnification. The tissue was stained for anti-mCherry to identify CAR bearing cells. FIG. 97 shows no engraftment of CAR bearing cells in the brain. FIG. 98 shows engraftment of CAR-bearing macrophages in the brain subsequent to treatment with cells comprising the CAR. FIGS. 112-114 are representative images of a saggital section of murine midbrain at 40× magnification. The tissue was stained for anti-mCherry to identify CAR bearing cells and anti-Iba1 to identify macrophages/microglia. FIG. 112 shows that CARAβ expressing cells are also Iba1+, demonstrating that these cells are myeloid in origin (macrophages) and they also adopt microglia-like morphology. FIG. 113 is the same image as FIG. 112 with the red channel being isolated to highlight CARAβ expressing cells. FIG. 114 is the same image as FIG. 114 with the green channel being isolated to highlight Iba1+ cells. The overlap in cells expressing the CARAβ and Iba1 highlight the origin (hematopoietic myeloid cell) and that the CAR is expressed after tissue engraftment.

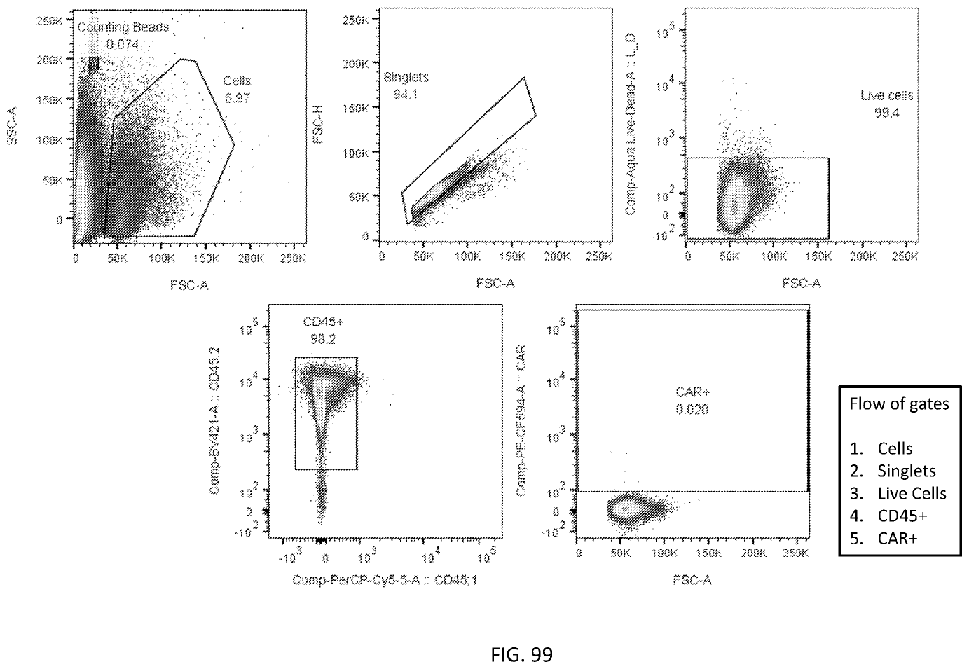

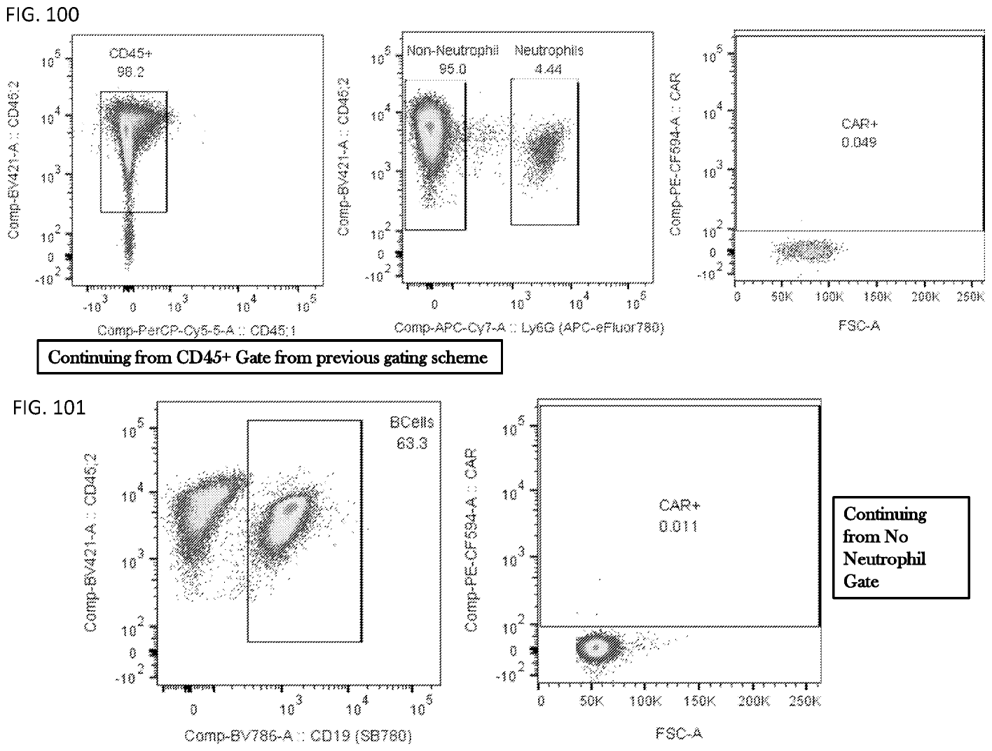

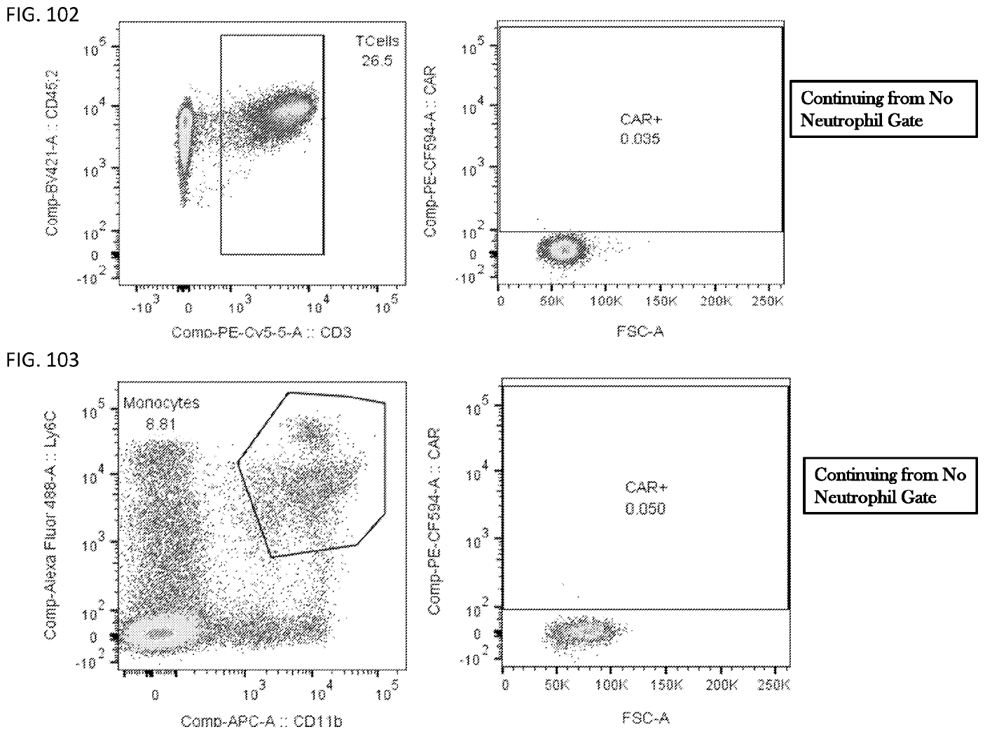

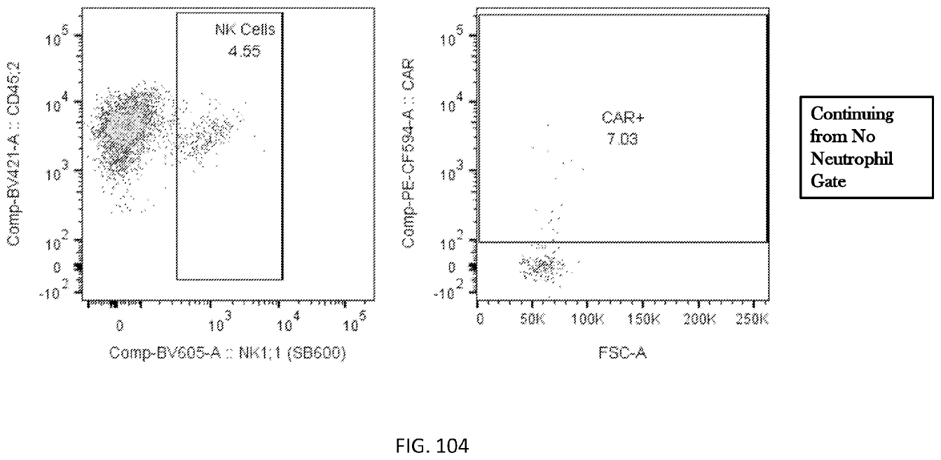

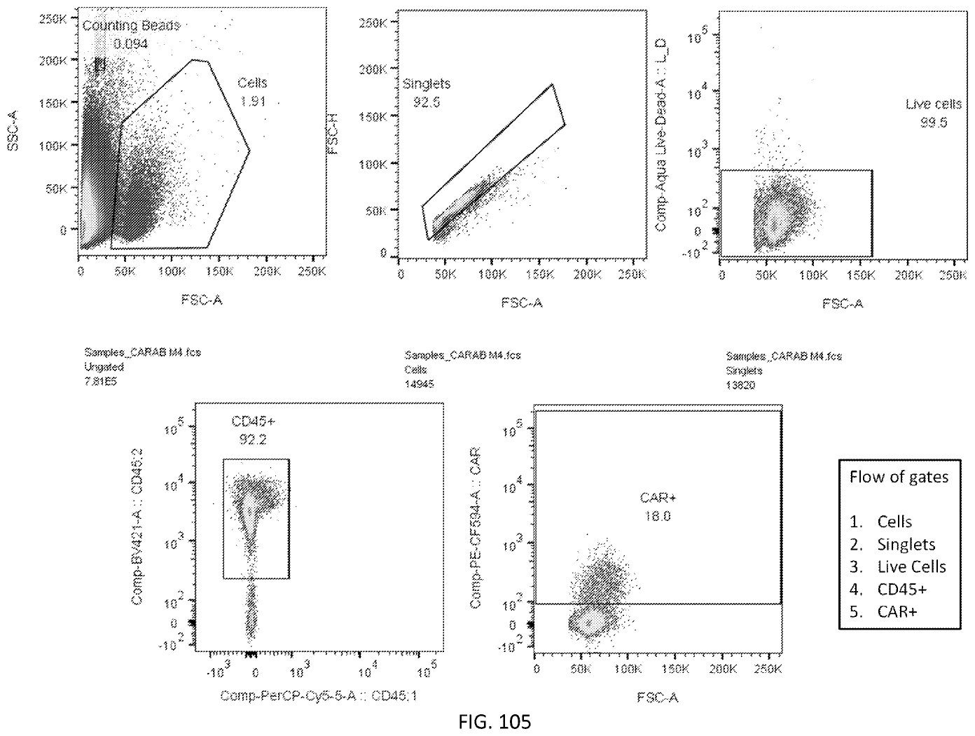

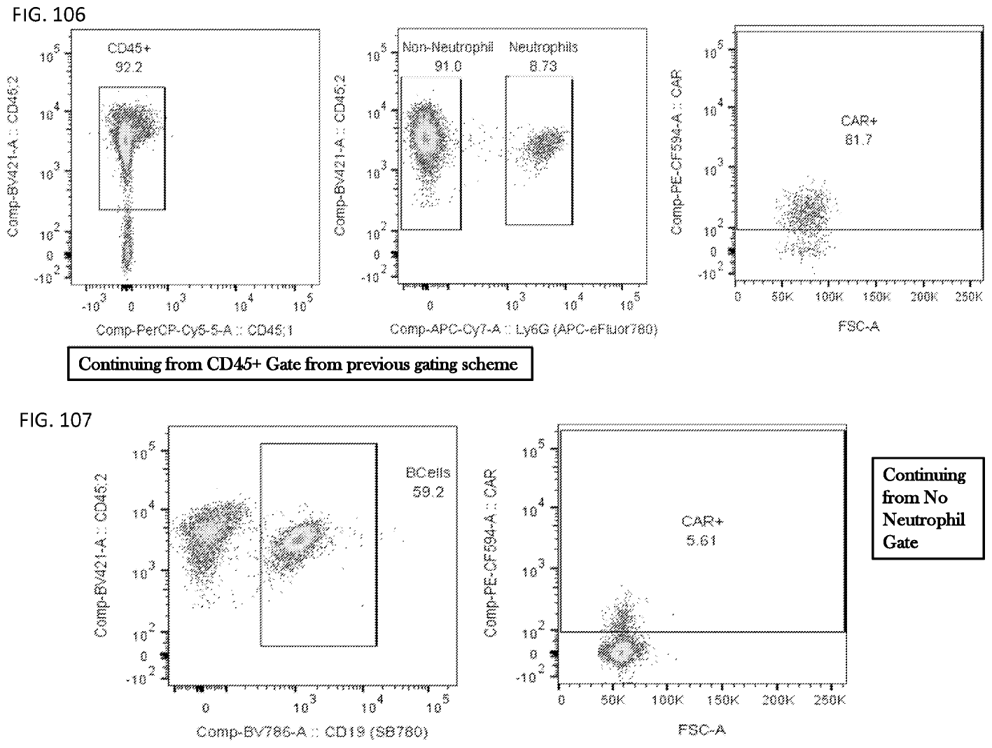

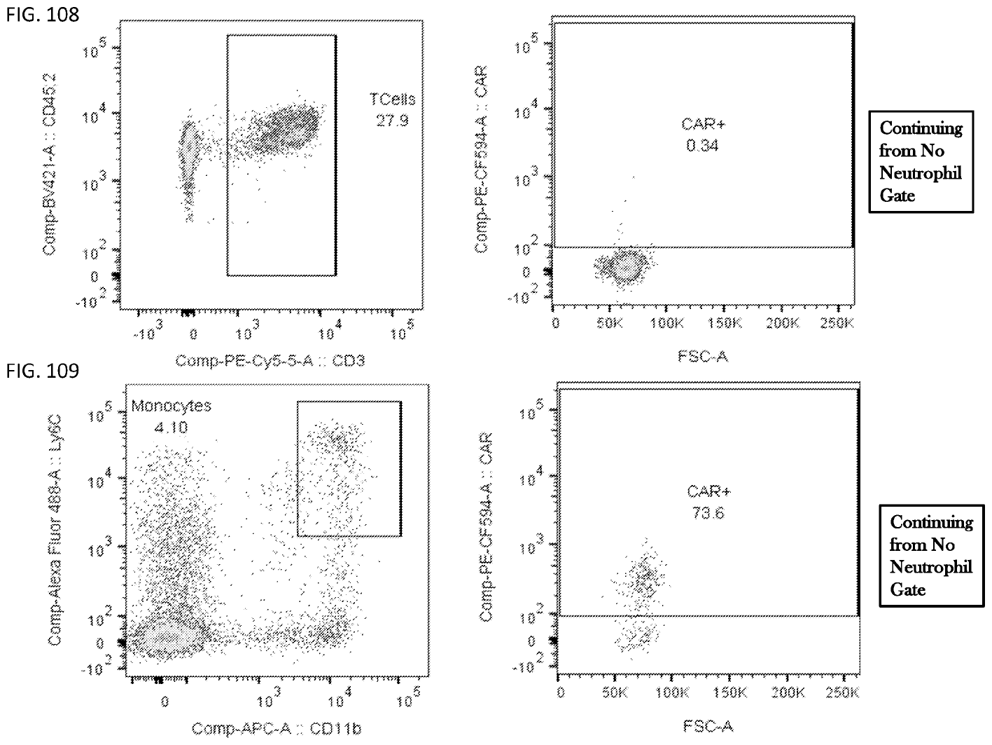

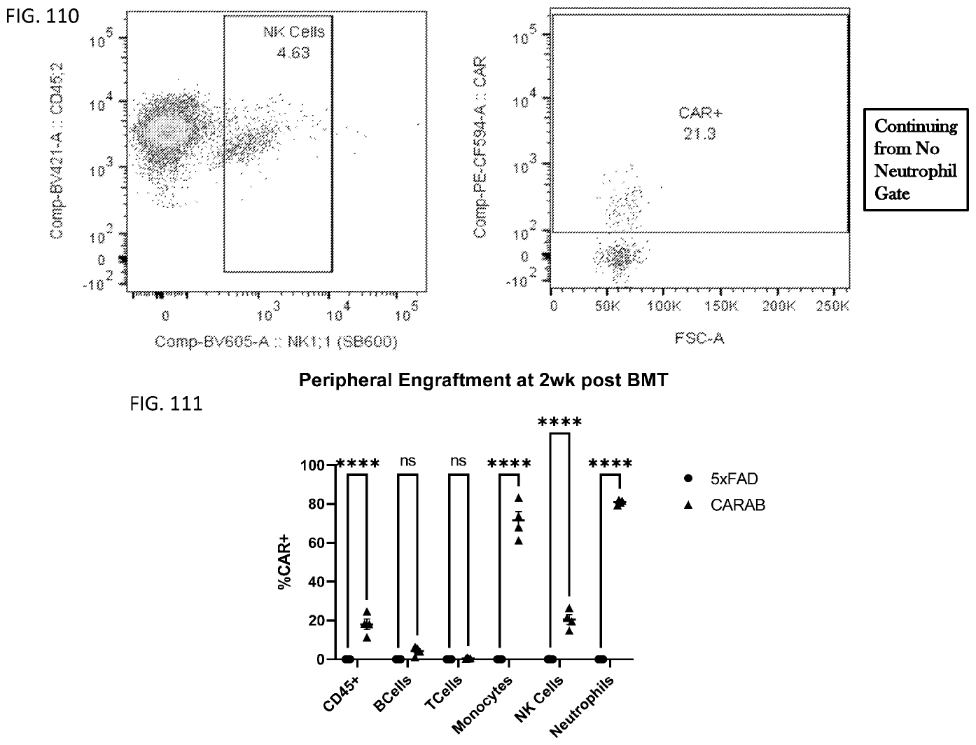

Engraftment studies were also conducted in a model of Alzheimer's disease. The following gating scheme was used to evaluate CAR expression in cells: cells→singlets→live cells→CAR+ cells. FIG. 99 illustrates a representative gating scheme for peripheral engraftment to evaluate overall CAR+ cell engraftment. FIG. 100-104 are representative gating schemes for the detection of CAR+ cells of hematopoietic lineage cells in an untreated 5×fad mouse with both donor and host cells being of the same CD45+ congenic strain. Donor and host cells were CD45.2+ cells. FIG. 105 shows a representative gating scheme of overall CAR+ engraftment of CD45+ in the periphery 2 weeks post bone marrow transplant. FIG. 106 is a representative gating scheme of engrafted CAR+ neutrophils. FIG. 107 is a representative gating scheme of engrafted CAR+ B cells post bone marrow transplant. FIG. 108 is a representative gating scheme of engrafted CAR+ T cells two weeks post bone marrow transplant. FIG. 109 is a representative gating scheme of CAR+ monocytes two weeks post bone marrow transplant. FIG. 110 is a representative gating scheme of engrafted CAR+NK cells post bone marrow transplant. FIG. 111 exhibits engrafted of CAR+ hematopoietic lineage cells in the periphery 2 weeks post bone marrow transplant.

### Enumerated Embodiments

The following enumerated embodiments are provided, the numbering of which is not to be construed as designating levels of importance.

Embodiment 1 provides a chimeric antigen receptor (CAR) comprising an antigen-binding domain and a transmembrane domain, wherein the antigen-binding domain binds amyloid beta (Aβ).

Embodiment 2 provides the CAR of embodiment 1, wherein the antigen-binding domain comprises a heavy chain variable region that comprises three heavy chain complementarity determining regions (HCDRs) and a light chain variable region that comprises three light chain complementarity determining regions (LCDRs), wherein at least one of the complementarity determining regions comprises any one of SEQ ID NOs: 1-6.

Embodiment 3 provides the CAR of any of embodiments 1-2, wherein HCDR1 comprises the amino acid sequence SYGMH (SEQ ID NO: 1), HCDR2 comprises the amino acid sequence VIWFDGTKKYYTDSVKG (SEQ ID NO: 2), HCDR3 comprises the amino acid sequence DRGIGARRGPYYMDV (SEQ ID NO: 3), LCDR1 comprises the amino acid sequence RASQSISSYLN (SEQ ID NO: 4), LCDR2 comprises the amino acid sequence ASSLQS (SEQ ID NO: 5), and LCDR3 comprises the amino acid sequence QQSYSTPLT (SEQ ID NO: 6).

Embodiment 4 provides the CAR of any of embodiments 1-3, wherein the heavy chain variable region (VH) of the antigen-binding domain comprises an amino acid sequence at least 80%, 85%, 90%, 95%, 96%, 97%, 98%, 99%, or 100% identical to SEQ ID NO: 7 and/or the light chain variable region (VL) comprises an amino acid sequence at least 80%, 85%, 90%, 95%, 96%, 97%, 98%, 99%, or 100% identical to SEQ ID NO: 8.

Embodiment 5 provides the CAR of any of embodiments 1-4, wherein VH of the antigen-binding domain is encoded by a nucleotide sequence at least 80%, 85%, 90%, 95%, 96%, 97%, 98%, 99%, or 100% identical to SEQ ID NO: 9 and/or the light chain variable region (VL) is encoded by a nucleotide sequence at least 80%, 85%, 90%, 95%, 96%, 97%, 98%, 99%, or 100% identical to SEQ ID NO: 10.

Embodiment 6 provides the CAR of any of embodiments 1-5, wherein the antigen-binding domain is a single-chain variable fragment (scFv) comprising an amino acid sequence at least 80%, 85%, 90%, 95%, 96%, 97%, 98%, 99%, or 100% identical to SEQ ID NO: 11 or SEQ ID NO: 12.

Embodiment 7 provides the CAR of any of embodiments 1-6, wherein the antigen-binding domain is a scFv encoded by a nucleotide sequence at least 80%, 85%, 90%, 95%, 96%, 97%, 98%, 99%, or 100% identical to SEQ ID NO: 13 or SEQ ID NO: 14.

Embodiment 8 provides the CAR of any of embodiments 1-7, wherein the CAR comprises an amino acid sequence at least 80%, 85%, 90%, 95%, 96%, 97%, 98%, 99%, or 100% identical to SEQ ID NO: 15 or SEQ ID NO: 16.

Embodiment 9 provides the CAR of any of embodiments 1-8, wherein the CAR is encoded by a nucleotide sequence at least 80%, 85%, 90%, 95%, 96%, 97%, 98%, 99%, or 100% identical to SEQ ID NO: 17 or SEQ ID NO: 18.

Embodiment 10 provides the CAR of embodiment 1, wherein the antigen-binding domain comprises a heavy chain variable region that comprises three HCDRs and a light chain variable region that comprises three LCDRs, wherein at least one of the complementarity determining regions comprises any one of SEQ ID NOs: 19-24.

Embodiment 11 provides the CAR of claim 10, wherein HCDR1 comprises the amino acid sequence GFTFSSYGMS (SEQ ID NO: 19), HCDR2 comprises the amino acid sequence SINSNGGSTYYPDSVK (SEQ ID NO: 20), HCDR3 comprises the amino acid sequence GDY (SEQ ID NO: 21), LCDR1 comprises the amino acid sequence RSSQSLVYSNGDTYLH (SEQ ID NO: 22), LCDR2 comprises the amino acid sequence KVSNRFS (SEQ ID NO: 23), and LCDR3 comprises the amino acid sequence SQSTHVPWT (SEQ ID NO: 24).

Embodiment 12 provides the CAR of any of embodiments 1, or 10-11, wherein the VH of the antigen-binding domain comprises an amino acid sequence at least 80%, 85%, 90%, 95%, 96%, 97%, 98%, 99%, or 100% identical to SEQ ID NO: 25 and/or the VL comprises an amino acid sequence at least 80%, 85%, 90%, 95%, 96%, 97%, 98%, 99%, or 100% identical to SEQ ID NO: 26.

Embodiment 13 provides the CAR of any of embodiments 1, or 10-12, wherein the VH of the antigen-binding domain is encoded by a nucleotide sequence at least 80%, 85%, 90%, 95%, 96%, 97%, 98%, 99%, or 100% identical to SEQ ID NO: 27 and/or the VL is encoded by a nucleotide sequence at least 80%, 85%, 90%, 95%, 96%, 97%, 98%, 99%, or 100% identical to SEQ ID NO: 28.

Embodiment 14 provides the CAR of any of embodiments 1, or 10-13, wherein the antigen-binding domain is a scFv comprising an amino acid sequence at least 80%, 85%, 90%, 95%, 96%, 97%, 98%, 99%, or 100% identical to SEQ ID NO: 29 or SEQ ID NO: 30.

Embodiment 15 provides the CAR of any of embodiments 1, or 10-14, wherein the antigen-binding domain is a scFv encoded by a nucleotide sequence at least 80%, 85%, 90%, 95%, 96%, 97%, 98%, 99%, or 100% identical to SEQ ID NO: 31 or SEQ ID NO: 32.

Embodiment 16 provides the CAR of any of embodiments 1, or 10-15, wherein the CAR comprises an amino acid sequence at least 80%, 85%, 90%, 95%, 96%, 97%, 98%, 99%, or 100% identical to SEQ ID NO: 33 or SEQ ID NO: 34.

Embodiment 17 provides the CAR of any of embodiments 1, or 10-16, wherein the CAR is encoded by a nucleotide sequence at least 80%, 85%, 90%, 95%, 96%, 97%, 98%, 99%, or 100% identical to SEQ ID NO: 35 or SEQ ID NO: 36.

Embodiment 18 provides the CAR of embodiment 1, wherein the antigen-binding domain comprises a heavy chain variable region that comprises three heavy chain complementarity determining regions (HCDRs) and a light chain variable region that comprises three light chain complementarity determining regions (LCDRs), wherein at least one of the complementarity determining regions comprises any one of SEQ ID NOs: 37-42.

Embodiment 19 provides the CAR of embodiments 1 or 18, wherein HCDR1 comprises the amino acid sequence NYGMS (SEQ ID NO: 37), HCDR2 comprises the amino acid sequence IRSGGGRTYYSDNVKGR (SEQ ID NO: 38), HCDR3 comprises the amino acid sequence YDHYSGSSDY (SEQ ID NO: 39), LCDR1 comprises the amino acid sequence KSSQSLLDSDGKTYLN (SEQ ID NO: 40), LCDR2 comprises the amino acid sequence LVSKLD (SEQ ID NO: 41), and LCDR3 comprises the amino acid sequence WQGTHFPRT (SEQ ID NO: 42).

Embodiment 20 provides the CAR of any of embodiments 1, or 18-19, wherein the VH of the antigen-binding domain comprises an amino acid sequence at least 80%, 85%, 90%, 95%, 96%, 97%, 98%, 99%, or 100% identical to SEQ ID NO: 43 and/or the VL comprises an amino acid sequence at least 80%, 85%, 90%, 95%, 96%, 97%, 98%, 99%, or 100% identical to SEQ ID NO: 44.

Embodiment 21 provides the CAR of any of embodiments 1, or 18-20, wherein the VH of the antigen-binding domain is encoded by a nucleotide sequence at least 80%, 85%, 90%, 95%, 96%, 97%, 98%, 99%, or 100% identical to SEQ ID NO: 45 and/or the VL is encoded by a nucleotide sequence at least 80%, 85%, 90%, 95%, 96%, 97%, 98%, 99%, or 100% identical to SEQ ID NO: 46.

Embodiment 22 provides the CAR of any of embodiments 1, or 18-21, wherein the antigen-binding domain is scFv comprising an amino acid sequence at least 80%, 85%, 90%, 95%, 96%, 97%, 98%, 99%, or 100% identical to SEQ ID NO: 47 or SEQ ID NO: 48.

Embodiment 23 provides the CAR of any of embodiments 1, or 18-22, wherein the antigen-binding domain is a scFv encoded by a nucleotide sequence at least 80%, 85%, 90%, 95%, 96%, 97%, 98%, 99%, or 100% identical to SEQ ID NO: 49 or SEQ ID NO: 50.

Embodiment 24 provides the CAR of any of embodiments 1, or 18-23, wherein the CAR comprises an amino acid sequence at least 80%, 85%, 90%, 95%, 96%, 97%, 98%, 99%, or 100% identical to SEQ ID NO: 51 or SEQ ID NO: 52.

Embodiment 25 provides the CAR of any of embodiments 1, or 18-24, wherein the CAR is encoded by a nucleotide sequence at least 80%, 85%, 90%, 95%, 96%, 97%, 98%, 99%, or 100% identical to SEQ ID NO: 53 or SEQ ID NO: 54.

Embodiment 26 provides a chimeric antigen receptor (CAR) comprising an antigen-binding domain and a transmembrane domain, wherein the antigen-binding domain binds Tau.

Embodiment 27 provides the CAR of embodiment 26, wherein the antigen-binding domain comprises a heavy chain variable region that comprises three heavy chain complementarity determining regions (HCDRs) and a light chain variable region that comprises three light chain complementarity determining regions (LCDRs), wherein at least one of the complementarity determining regions comprises any one of SEQ ID NOs: 55-60.

Embodiment 28 provides the CAR of embodiment 26 or 27, wherein HCDR1 comprises the amino acid sequence KYGMS (SEQ ID NO: 55), HCDR2 comprises the amino acid sequence ISSSGSRTYYPDSVKG (SEQ ID NO: 56), HCDR3 comprises the amino acid sequence WDGAMDY (SEQ ID NO: 57), LCDR1 comprises the amino acid sequence KSSQSIVHSNGNTYLE (SEQ ID NO: 58), LCDR2 comprises the amino acid sequence KVSNRF (SEQ ID NO: 59), and LCDR3 comprises the amino acid sequence FQGSLVPWA (SEQ ID NO: 60).

Embodiment 29 provides the CAR of any of embodiments 26-28, wherein the VH of the antigen-binding domain comprises an amino acid sequence at least 80%, 85%, 90%, 95%, 96%, 97%, 98%, 99%, or 100% identical to SEQ ID NO: 61 and/or VL comprises an amino acid sequence at least 80%, 85%, 90%, 95%, 96%, 97%, 98%, 99%, or 100% identical to SEQ ID NO: 62.

Embodiment 30 provides the CAR of any of embodiments 26-29, wherein the VH of the antigen-binding domain is encoded by a nucleotide sequence at least 80%, 85%, 90%, 95%, 96%, 97%, 98%, 99%, or 100% identical to SEQ ID NO: 63 and/or the VL is encoded by a nucleotide sequence at least 80%, 85%, 90%, 95%, 96%, 97%, 98%, 99%, or 100% identical to SEQ ID NO: 64.

Embodiment 31 provides the CAR of any of embodiments 26-30, wherein the antigen-binding domain is a scFv comprising an amino acid sequence at least 80%, 85%, 90%, 95%, 96%, 97%, 98%, 99%, or 100% identical to SEQ ID NO: 65 or SEQ ID NO: 66.

Embodiment 32 provides the CAR of any of embodiments 26-31, wherein the antigen-binding domain is a scFv encoded by a nucleotide sequence at least 80%, 85%, 90%, 95%, 96%, 97%, 98%, 99%, or 100% identical to SEQ ID NO: 67 or SEQ ID NO: 68.

Embodiment 33 provides the CAR of any of embodiments 26-32, wherein the CAR comprises an amino acid sequence at least 80%, 85%, 90%, 95%, 96%, 97%, 98%, 99%, or 100% identical to SEQ ID NO: 69 or SEQ ID NO: 70.

Embodiment 34 provides the CAR of any of embodiments 26-33, wherein the CAR is encoded by a nucleotide sequence at least 80%, 85%, 90%, 95%, 96%, 97%, 98%, 99%, or 100% identical to SEQ ID NO: 71 or SEQ ID NO: 72.

Embodiment 35 provides the CAR of any of any of the preceding embodiments, wherein the CAR does not contain an intracellular domain.

Embodiment 36 provides the CAR of any of embodiments 1-34, wherein the CAR further comprises an intracellular domain.

Embodiment 37 provides the CAR of embodiment 36, wherein the CAR comprises an amino acid sequence at least 80%, 85%, 90%, 95%, 96%, 97%, 98%, 99%, or 100% identical to any one of SEQ ID NOs: 73, 74, 77, 78, 81, or 82.

Embodiment 38 provides the CAR of embodiment 36 or 37, wherein the CAR is encoded by a nucleotide sequence at least 80%, 85%, 90%, 95%, 96%, 97%, 98%, 99%, or 100% identical to any one of SEQ ID NOs: 75, 76, 79, 80, 83, or 84.

Embodiment 39 provides a modified immune cell comprising the CAR of any of the preceding embodiments.

Embodiment 40 provides the modified immune cell of embodiment 39, further comprising a second CAR of any of the preceding embodiments.

Embodiment 41 provides the modified immune cell of embodiment 39 or 40, wherein the cell is a monocyte, macrophage, B cell, T cell, NK cell, neutrophil, or stem cell.

Embodiment 42 provides the pharmaceutical composition comprising the modified immune cell of any of embodiments 39-41, and a pharmaceutically acceptable carrier.

Embodiment 43 provides a method of treating a neurodegenerative disease in a subject in need thereof, the method comprising administering to the subject a therapeutically effective amount of the pharmaceutical composition of embodiment 42.

Embodiment 44 provides the method of embodiment 43, wherein the neurodegenerative disease is Alzheimer's Disease (AD) or a tauopathy.

Embodiment 45 provides the method of embodiment 43 or 44, further comprising depleting the endogenous microglia in the subject prior to administering the pharmaceutical composition.

Embodiment 46 provides a method of treating a neurodegenerative disease in a subject in need thereof, the method comprising: administering to the subject a therapeutically effective amount of a composition comprising a cell comprising a chimeric antigen receptor (CAR), wherein the CAR comprises an antigen binding domain that binds amyloid beta, and wherein the CAR does not comprise an intracellular domain, and wherein the cell is a monocyte, macrophage, dendritic cell, or stem cell.

Embodiment 47 provides the method of treating a neurodegenerative disease in a subject in need thereof, the method comprising: depleting the endogenous microglia in the subject, and administering to the subject a therapeutically effective amount of a composition comprising a cell comprising a chimeric antigen receptor (CAR), wherein the CAR comprises an antigen binding domain that binds amyloid beta, and wherein the CAR does not comprise an intracellular domain, and wherein the cell is a monocyte, macrophage, dendritic cell, or stem cell.

Embodiment 48 provides the method of embodiment 46 or 47, wherein the neurodegenerative disease is Alzheimer's Disease (AD) or a tauopathy.

Embodiment 49 provides the method of any of embodiments 43-48, further comprising wherein the CAR delivers a payload.

Embodiment 50 provides the method of any of embodiments 43-49, wherein the CAR is delivered via a lipid nanoparticle (LNP).

### OTHER EMBODIMENTS

The recitation of a listing of elements in any definition of a variable herein includes definitions of that variable as any single element or combinations (or subcombinations) of listed elements. The recitation of an embodiment herein includes that embodiment as any single embodiment or in combination with any other embodiments or portions thereof.

The disclosures of each and every patent, patent application, and publication cited herein are hereby incorporated herein by reference in their entirety. While this invention has been disclosed with reference to specific embodiments, it is apparent that other embodiments and variations of this invention may be devised by others skilled in the art without departing from the true spirit and scope of the invention. The appended claims are intended to be construed to include all such embodiments and equivalent variations.

1. A chimeric antigen receptor (CAR) comprising an antigen-binding domain and a transmembrane domain, wherein the antigen binding domain comprises a heavy chain variable region (VH) comprising three heavy chain complementarity determining regions (HCDR1, HCDR2, and HCDR3), and a light chain variable region (VL) comprising three light chain complementarity determining regions (LCDR1, LCDR2, and LCDR3), further wherein:
(i) the antigen-binding domain binds amyloid beta (Aβ) and the CAR further comprises an intracellular domain;
(ii) the antigen-binding domain binds Aβ and the CAR does not comprise an intracellular domain;
(iii) the antigen-binding domain binds Tau and the CAR further comprises an intracellular domain; or
(iv) the antigen-binding domain binds Tau and the CAR does not comprise an intracellular domain.

2. The CAR of claim 1, wherein the antigen-binding domain binds Aβ, further wherein at least one of the complementarity determining regions comprises any one of SEQ ID NOs: 1-6.

3. The CAR of claim 2, wherein HCDR1 comprises the amino acid sequence SYGMH (SEQ ID NO: 1), HCDR2 comprises the amino acid sequence VIWFDGTKKYYTDSVKG (SEQ ID NO: 2), HCDR3 comprises the amino acid sequence DRGIGARRGPYYMDV (SEQ ID NO: 3), LCDR1 comprises the amino acid sequence RASQSISSYLN (SEQ ID NO: 4), LCDR2 comprises the amino acid sequence ASSLQS (SEQ ID NO: 5), and LCDR3 comprises the amino acid sequence QQSYSTPLT (SEQ ID NO: 6).

4. The CAR of claim 1, wherein the antigen-binding domain binds Aβ, further wherein the VH comprises an amino acid sequence at least 80%, 85%, 90%, 95%, 96%, 97%, 98%, 99%, or 100% identical to SEQ ID NO: 7 and/or the VL comprises an amino acid sequence at least 80%, 85%, 90%, 95%, 96%, 97%, 98%, 99%, or 100% identical to SEQ ID NO: 8.

5. The CAR of claim 1, wherein the antigen-binding domain binds Aβ, further wherein the VH is encoded by a nucleotide sequence at least 80%, 85%, 90%, 95%, 96%, 97%, 98%, 99%, or 100% identical to SEQ ID NO: 9 and/or the VL is encoded by a nucleotide sequence at least 80%, 85%, 90%, 95%, 96%, 97%, 98%, 99%, or 100% identical to SEQ ID NO: 10.

6. The CAR of claim 1, wherein the antigen-binding domain binds Aβ, further wherein the antigen-binding domain is a single-chain variable fragment (scFv) comprising an amino acid sequence at least 80%, 85%, 90%, 95%, 96%, 97%, 98%, 99%, or 100% identical to SEQ ID NO: 11 or SEQ ID NO: 12.

7. The CAR of claim 1, wherein the antigen-binding domain binds Aβ, further wherein the antigen-binding domain is a scFv encoded by a nucleotide sequence at least 80%, 85%, 90%, 95%, 96%, 97%, 98%, 99%, or 100% identical to SEQ ID NO: 13 or SEQ ID NO: 14.

8. The CAR of claim 1, wherein the antigen-binding domain binds Aβ, further wherein the CAR comprises an amino acid sequence at least 80%, 85%, 90%, 95%, 96%, 97%, 98%, 99%, or 100% identical to SEQ ID NO: 15 or SEQ ID NO: 16.

9. The CAR of claim 1, wherein the antigen-binding domain binds Aβ, further wherein the CAR is encoded by a nucleotide sequence at least 80%, 85%, 90%, 95%, 96%, 97%, 98%, 99%, or 100% identical to SEQ ID NO: 17 or SEQ ID NO: 18.

10. The CAR of claim 1, wherein the antigen-binding domain binds Aβ, further wherein at least one of the complementarity determining regions comprises any one of SEQ ID NOs: 19-24.

11. The CAR of claim 10, wherein HCDR1 comprises the amino acid sequence GFTFSSYGMS (SEQ ID NO: 19), HCDR2 comprises the amino acid sequence SINSNGGSTYYPDSVK (SEQ ID NO: 20), HCDR3 comprises the amino acid sequence GDY (SEQ ID NO: 21), LCDR1 comprises the amino acid sequence RSSQSLVYSNGDTYLH (SEQ ID NO: 22), LCDR2 comprises the amino acid sequence KVSNRFS (SEQ ID NO: 23), and LCDR3 comprises the amino acid sequence SQSTHVPWT (SEQ ID NO: 24).

12. The CAR of claim 1, wherein the antigen-binding domain binds Aβ, further wherein the VH comprises an amino acid sequence at least 80%, 85%, 90%, 95%, 96%, 97%, 98%, 99%, or 100% identical to SEQ ID NO: 25 and/or the VL comprises an amino acid sequence at least 80%, 85%, 90%, 95%, 96%, 97%, 98%, 99%, or 100% identical to SEQ ID NO: 26.

13. The CAR of claim 1, wherein the antigen-binding domain binds Aβ, further wherein the VH is encoded by a nucleotide sequence at least 80%, 85%, 90%, 95%, 96%, 97%, 98%, 99%, or 100% identical to SEQ ID NO: 27 and/or the VL is encoded by a nucleotide sequence at least 80%, 85%, 90%, 95%, 96%, 97%, 98%, 99%, or 100% identical to SEQ ID NO: 28.

14. The CAR of claim 1, wherein the antigen-binding domain binds Aβ, further wherein the antigen-binding domain is a scFv comprising an amino acid sequence at least 80%, 85%, 90%, 95%, 96%, 97%, 98%, 99%, or 100% identical to SEQ ID NO: 29 or SEQ ID NO: 30.

15. The CAR of claim 1, wherein the antigen-binding domain binds Aβ, further wherein the antigen-binding domain is a scFv encoded by a nucleotide sequence at least 80%, 85%, 90%, 95%, 96%, 97%, 98%, 99%, or 100% identical to SEQ ID NO: 31 or SEQ ID NO: 32.

16. The CAR of claim 1, wherein the antigen-binding domain binds Aβ, further wherein the CAR comprises an amino acid sequence at least 80%, 85%, 90%, 95%, 96%, 97%, 98%, 99%, or 100% identical to SEQ ID NO: 33 or SEQ ID NO: 34.

17. The CAR of claim 1, wherein the antigen-binding domain binds Aβ, further wherein the CAR is encoded by a nucleotide sequence at least 80%, 85%, 90%, 95%, 96%, 97%, 98%, 99%, or 100% identical to SEQ ID NO: 35 or SEQ ID NO: 36.

18. The CAR of claim 1, wherein the antigen-binding domain binds A further wherein at least one of the complementarity determining regions comprises any one of SEQ ID NOs: 37-42.

19. The CAR of claim 18, wherein HCDR1 comprises the amino acid sequence NYGMS (SEQ ID NO: 37), HCDR2 comprises the amino acid sequence IRSGGGRTYYSDNVKGR (SEQ ID NO: 38), HCDR3 comprises the amino acid sequence YDHYSGSSDY (SEQ ID NO: 39), LCDR1 comprises the amino acid sequence KSSQSLLDSDGKTYLN (SEQ ID NO: 40), LCDR2 comprises the amino acid sequence LVSKLD (SEQ ID NO: 41), and LCDR3 comprises the amino acid sequence WQGTHFPRT (SEQ ID NO: 42).

20. The CAR of claim 1, wherein the antigen-binding domain binds Aβ, further wherein the VH comprises an amino acid sequence at least 80%, 85%, 90%, 95%, 96%, 97%, 98%, 99%, or 100% identical to SEQ ID NO: 43 and/or the VL comprises an amino acid sequence at least 80%, 85%, 90%, 95%, 96%, 97%, 98%, 99%, or 100% identical to SEQ ID NO: 44.

21. The CAR of claim 1, wherein the antigen-binding domain binds Aβ, further wherein the VH is encoded by a nucleotide sequence at least 80%, 85%, 90%, 95%, 96%, 97%, 98%, 99%, or 100% identical to SEQ ID NO: 45 and/or the VL is encoded by a nucleotide sequence at least 80%, 85%, 90%, 95%, 96%, 97%, 98%, 99%, or 100% identical to SEQ ID NO: 46.

22. The CAR of claim 1, wherein the antigen-binding domain binds Aβ, further wherein the antigen-binding domain is scFv comprising an amino acid sequence at least 80%, 85%, 90%, 95%, 96%, 97%, 98%, 99%, or 100% identical to SEQ ID NO: 47 or SEQ ID NO: 48.

23. The CAR of claim 1, wherein the antigen-binding domain binds Aβ, further wherein the antigen-binding domain is a scFv encoded by a nucleotide sequence at least 80%, 85%, 90%, 95%, 96%, 97%, 98%, 99%, or 100% identical to SEQ ID NO: 49 or SEQ ID NO: 50.

24. The CAR of claim 1, wherein the antigen-binding domain binds Aβ, further wherein the CAR comprises an amino acid sequence at least 80%, 85%, 90%, 95%, 96%, 97%, 98%, 99%, or 100% identical to SEQ ID NO: 51 or SEQ ID NO: 52.

25. The CAR of claim 1, wherein the antigen-binding domain binds Aβ, further wherein the CAR is encoded by a nucleotide sequence at least 80%, 85%, 90%, 95%, 96%, 97%, 98%, 99%, or 100% identical to SEQ ID NO: 53 or SEQ ID NO: 54.

26. (canceled)

27. The CAR of claim 1, wherein the antigen-binding domain binds Tau, further wherein at least one of the complementarity determining regions comprises any one of SEQ ID NOs: 55-60.

28. The CAR of claim 27, wherein HCDR1 comprises the amino acid sequence KYGMS (SEQ ID NO: 55), HCDR2 comprises the amino acid sequence ISSSGSRTYYPDSVKG (SEQ ID NO: 56), HCDR3 comprises the amino acid sequence WDGAMDY (SEQ ID NO: 57), LCDR1 comprises the amino acid sequence KSSQSIVHSNGNTYLE (SEQ ID NO: 58), LCDR2 comprises the amino acid sequence KVSNRF (SEQ ID NO: 59), and LCDR3 comprises the amino acid sequence FQGSLVPWA (SEQ ID NO: 60).

29. The CAR of claim 1, wherein the antigen-binding domain binds Tau, further wherein the VH comprises an amino acid sequence at least 80%, 85%, 90%, 95%, 96%, 97%, 98%, 99%, or 100% identical to SEQ ID NO: 61 and/or the VL comprises an amino acid sequence at least 80%, 85%, 90%, 95%, 96%, 97%, 98%, 99%, or 100% identical to SEQ ID NO: 62.

30. The CAR of claim 1, wherein the antigen-binding domain binds Tau, further wherein the VH is encoded by a nucleotide sequence at least 80%, 85%, 90%, 95%, 96%, 97%, 98%, 99%, or 100% identical to SEQ ID NO: 63 and/or the VL is encoded by a nucleotide sequence at least 80%, 85%, 90%, 95%, 96%, 97%, 98%, 99%, or 100% identical to SEQ ID NO: 64.

31. The CAR of claim 1, wherein the antigen-binding domain binds Tau, further wherein the antigen-binding domain is a scFv comprising an amino acid sequence at least 80%, 85%, 90%, 95%, 96%, 97%, 98%, 99%, or 100% identical to SEQ ID NO: 65 or SEQ ID NO: 66.

32. The CAR of claim 1, wherein the antigen-binding domain binds Tau, further wherein the antigen-binding domain is a scFv encoded by a nucleotide sequence at least 80%, 85%, 90%, 95%, 96%, 97%, 98%, 99%, or 100% identical to SEQ ID NO: 67 or SEQ ID NO: 68.

33. The CAR of claim 1, wherein the antigen-binding domain binds Tau, further wherein the CAR comprises an amino acid sequence at least 80%, 85%, 90%, 95%, 96%, 97%, 98%, 99%, or 100% identical to SEQ ID NO: 69 or SEQ ID NO: 70.

34. The CAR of claim 1, wherein the antigen-binding domain binds Tau, further wherein the CAR is encoded by a nucleotide sequence at least 80%, 85%, 90%, 95%, 96%, 97%, 98%, 99%, or 100% identical to SEQ ID NO: 71 or SEQ ID NO: 72.

35-36. (canceled)

37. The CAR of claim 1, wherein the CAR comprises an amino acid sequence at least 80%, 85%, 90%, 95%, 96%, 97%, 98%, 99%, or 100% identical to any one of SEQ ID NOs: 73, 74, 77, 78, 81, or 82.

38. The CAR of claim 1, wherein the CAR is encoded by a nucleotide sequence at least 80%, 85%, 90%, 95%, 96%, 97%, 98%, 99%, or 100% identical to any one of SEQ ID NOs: 75, 76, 79, 80, 83, or 84.

39. A modified immune cell comprising a first CAR, wherein the first CAR is the CAR of claim 1,
optionally wherein the modified immune cell further comprises a second CAR, wherein the second CAR is the CAR of claim 1, further wherein the first CAR and the second CAR are not the same CAR.

40. (canceled)

41. The modified immune cell of claim 39, wherein the cell is a monocyte, macrophage, B cell, T cell, NK cell, neutrophil, or stem cell.

42. A pharmaceutical composition comprising the modified immune cell of claim 39, and a pharmaceutically acceptable carrier.

43. A method of treating a neurodegenerative disease in a subject in need thereof, the method comprising administering to the subject a therapeutically effective amount of the pharmaceutical composition of claim 42.

44. The method of claim 43, wherein the neurodegenerative disease is Alzheimer's Disease (AD) or a tauopathy.

45. The method of claim 43, further comprising depleting the endogenous microglia in the subject prior to administering the pharmaceutical composition.

46. A method of treating a neurodegenerative disease in a subject in need thereof, the method comprising:
administering to the subject a therapeutically effective amount of a composition comprising a cell comprising a chimeric antigen receptor (CAR), wherein the CAR comprises an antigen binding domain that binds amyloid beta, and wherein the CAR does not comprise an intracellular domain, and
wherein the cell is a monocyte, macrophage, dendritic cell, or stem cell;
optionally wherein the method further comprises depleting the endogenous microglia in the subject prior to the administering.

47. (canceled)

48. The method of claim 46, wherein the neurodegenerative disease is Alzheimer's Disease (AD) or a tauopathy.

49. The method ofany of claim 43, wherein the CAR delivers a payload.

50. The method of claim 43, wherein the CAR is delivered via a lipid nanoparticle (LNP).

51. The method of claim 46, wherein the CAR delivers a payload.

52. The method of claim 46, wherein the CAR is delivered via a lipid nanoparticle (LNP).
